(19) United States
(12) Patent Application Publication
(10) Pub. No.: US 2005/0272036 A1
(43) Pub. Date: Dec. 8, 2005

(54) Ketones

(76) Inventors: Peter John Barton, Macclesfield (GB); David Clarke, Macclesfield (GB); Christopher Davies, Macclesfield (GB); Rodney Hargreaves, Macclesfield (GB); Janet Pease, Macclesfield (GB); Maureen Rankine, Macclesfield (GB)

Correspondence Address: FISH & NEAVE IP GROUP;ROPES & GRAY LLP, ONE INTERNATIONAL PLACE, BOSTON, MA, 02110-2624, US

(21) Appl. No.: 10/522,225

(22) PCT Filed: Jul. 23, 2003
(86) PCT No.: PCT/GB03/03171

§ 371 Date: Jan. 24, 2005

(30) Foreign Application Priority Data
Jul. 27, 2002 (GB) 0217433.2
Dec. 24, 2002 (GB) 0230318.8

(51) Int. Cl. C12Q 1/68

(52) U.S. Cl. 435/6

(57) ABSTRACT

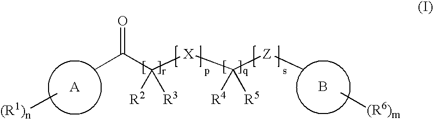

Compounds of formula (I): wherein variable groups are as defined within; for use in the inhibition of 11βHSD1 are described.

This invention relates to chemical compounds, or pharmaceutically acceptable salts hereof. These compounds possess human 11-β-hydroxysteroid dehydrogenase type 1 enzyme (11βHSD1) inhibitory activity and accordingly have value in the treatment of disease states including metabolic syndrome and are useful in methods of treatment of a warm-blooded animal, such as man. The invention also relates to processes for the manufacture of said compounds, to pharmaceutical compositions containing them and to their use in the manufacture of medicaments to inhibit 11βHSD1 in a warm-blooded animal, such as man.

Glucocorticoids (cortisol in man, corticosterone in rodents) are counter regulatory hormones i.e. they oppose the actions of insulin (Dallman M F, Strack A M, Akana S F et al. 1993; Front Neuroendocrinol 14, 303-347). They regulate the expression of hepatic enzymes involved in gluconeogenesis and increase substrate supply by releasing glycerol from adipose tissue (increased lipolysis) and amino acids from muscle (decreased protein synthesis and increased protein degradation). Glucocorticoids are also important in the differentiation of pre-adipocytes into mature adipocytes which are able to store triglycerides (Bujalska I J et al. 1999; Endocrinology 140, 3188-3196). This may be critical in disease states where glucocorticoids induced by “stress” are associated with central obesity which itself is a strong risk factor for type 2 diabetes, hypertension and cardiovascular disease (Bjorntorp P & Rosmond R 2000; Int. J. Obesity 24, S80-S85)

It is now well established that glucocorticoid activity is controlled not simply by secretion of cortisol but also at the tissue level by intracellular interconversion of active cortisol and inactive cortisone by the 11-beta hydroxysteroid dehydrogenases, 11βHSD1 (which activates cortisone) and 11βHSD2 (which inactivates cortisol) (Sandeep T C & Walker B R 2001 Trends in Endocrinol & Metab. 12,446-453). That this mechanism may be important in man was initially shown using carbenoxolone (an anti-ulcer drug which inhibits both 11βHSD1 and 2) treatment which (Walker B R et al. 1995; J. Clin. Endocrinol. Metab. 80, 3155-3159) leads to increased insulin sensitivity indicating that 11βHSD1 may well be regulating the effects of insulin by decreasing tissue levels of active glucocorticoids (Walker B R et al. 1995; J. Clin. Endocrinol. Metab. 80, 3155-3159).

Clinically, Cushing's syndrome is associated with cortisol excess which in turn is associated with glucose intolerance, central obesity (caused by stimulation of pre-adipocyte differentiation in this depot), dyslipidaemia and hypertension. Cushing's syndrome shows a number of clear parallels with metabolic syndrome. Even though the metabolic syndrome is not generally associated with excess circulating cortisol levels (Jessop D S et al. 2001; J. Clin. Endocrinol. Metab. 86, 4109-4114) abnormally high 11βHSD1 activity within tissues would be expected to have the same effect. In obese men it was shown that despite having similar or lower plasma cortisol levels than lean controls, 11βHSD1 activity in subcutaneous fat was greatly enhanced (Rask E et al. 2001; J. Clin. Endocrinol. Metab. 1418-1421). Furthermore, the central fat, associated with the metabolic syndrome expresses much higher levels of 11βHSD1 activity than subcutaneous fat (Bujalska I J et al. 1997; Lancet 349, 1210-1213). Thus there appears to be a link between glucocorticoids, 11βHSD1 and the metabolic syndrome.

11βHSD1 knock-out mice show attenuated glucocorticoid-induced activation of gluconeogenic enzymes in response to fasting and lower plasma glucose levels in response to stress or obesity (Kotelevtsev Y et al. 1997; Proc. Natl. Acad. Sci USA 94, 14924-14929) indicating the utility of inhibition of 11βHSD1 in lowering of plasma glucose and hepatic glucose output in type 2 diabetes. Furthermore, these mice express an anti-atherogenic lipoprotein profile, having low triglycerides, increased HDL cholesterol and increased apo-lipoprotein AI levels. (Morton N M et al. 2001; J. Biol. Chem. 276, 41293-41300). This phenotype is due to an increased hepatic expression of enzymes of fat catabolism and PPARα. Again this indicates the utility of 11βHSD1 inhibition in treatment of the dyslipidaemia of the metabolic syndrome.

The most convincing demonstration of a link between the metabolic syndrome and 11βHSD1 comes from recent studies of transgenic mice over-expressing 11βHSD1 (Masuzaki H et al. 2001; Science 294, 2166-2170). When expressed under the control of an adipose specific promoter, 11βHSD1 transgenic mice have high adipose levels of corticosterone, central obesity, insulin resistant diabetes, hyperlipidaemia and hyperphagia. Most importantly, the increased levels of 11βHSD1 activity in the fat of these mice are similar to those seen in obese subjects. Hepatic 11βHSD1 activity and plasma corticosterone levels were normal, however, hepatic portal vein levels of corticosterone were increased 3 fold and it is thought that this is the cause of the metabolic effects in liver.

Overall it is now clear that the complete metabolic syndrome can be mimicked in mice simply by overexpressing 11βHSD1 in fat alone at levels similar to those in obese man.

11βHSD1 tissue distribution is widespread and overlapping with that of the glucocorticoid receptor. Thus, 11βHSD1 inhibition could potentially oppose the effects of glucocorticoids in a number of physiological/pathological roles. 11βHSD1 is present in human skeletal muscle and glucocorticoid opposition to the anabolic effects of insulin on protein turnover and glucose metabolism are well documented (Whorwood C B et al. 2001; J. Clin. Endocrinol. Metab. 86, 2296-2308). Skeletal muscle must therefore be an important target for 11βHSD1 based therapy.

Glucocorticoids also decrease insulin secretion and this could exacerbate the effects of glucocorticoid induced insulin resistance. Pancreatic islets express 11βHSD1 and carbenoxolone can inhibit the effects of 11-dehydocorticosterone on insulin release (Davani B et al. 2000; J. Biol. Chem. 275, 34841-34844). Thus in treatment of diabetes 11βHSD1 inhibitors may not only act at the tissue level on insulin resistance but also increase insulin secretion itself.

Skeletal development and bone function is also regulated by glucocorticoid action. 11βHSD1 is present in human bone osteoclasts and osteoblasts and treatment of healthy volunteers with carbenoxolone showed a decrease in bone resorption markers with no change in bone formation markers (Cooper M S et al 2000; Bone 27, 375-381). Inhibition of 11βHSD1 activity in bone could be used as a protective mechanism in treatment of osteoporosis.

Glucocorticoids may also be involved in diseases of the eye such as glaucoma. 11βHSD1 has been shown to affect intraocular pressure in man and inhibition of 11βHSD1 may be expected to alleviate the increased intraocular pressure associated with glaucoma (Rauz S et al. 2001; Investigative Opthalmology & Visual Science 42, 2037-2042).

There appears to be a convincing link between 11βHSD1 and the metabolic syndrome both in rodents and in humans. Evidence suggests that a drug which specifically inhibits 11βHSD1 in type 2 obese diabetic patients will lower blood glucose by reducing hepatic gluconeogenesis, reduce central obesity, improve the atherogenic lipoprotein phenotype, lower blood pressure and reduce insulin resistance. Insulin effects in muscle will be enhanced and insulin secretion from the beta cells of the islet may also be increased.

Currently there are two main recognised definitions of metabolic syndrome.

1) The Adult Treatment Panel (ATP III 2001 JMA) definition of metabolic syndrome indicates that it is present if the patient has three or more of the following symptoms:
Waist measuring at least 40 inches (102 cm) for men, 35 inches (88 cm) for women;
Serum triglyceride levels of at least 150 mg/dl (1.69 mmol/l);
HDL cholesterol levels of less than 40 mg/dl (1.04 mmol/l) in men, less than 50 mg/dl (1.29 mmol/l) in women;
Blood pressure of at least 135/80 mm Hg; and/or
Blood sugar (serum glucose) of at least 110 mg/dl (6.1 mmol/l).

2) The WHO consultation has recommended the following definition which does not imply causal relationships and is suggested as a working definition to be improved upon in due course:
The patient has at least one of the following conditions: glucose intolerance, impaired glucose tolerance (IGT) or diabetes mellitus and/or insulin resistance; together with two or more of the following:
   Raised Arterial Pressure;
   Raised plasma triglycerides
   Central Obesity
   Microalbuminuria

We have found that the compounds defined in the present invention, or a pharmaceutically acceptable salt thereof, are effective 11βHSD1 inhibitors, and accordingly have value in the treatment of disease states associated with metabolic syndrome.

Accordingly there is provided the use of a compound of formula (I): 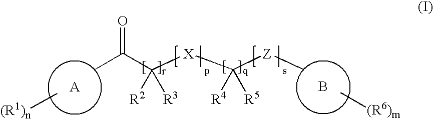
wherein:

Ring A is selected from aryl or heteroaryl;

R¹ is selected from halo, nitro, cyano, hydroxy, amino, carboxy, carbamoyl, mercapto, sulphamoyl, C₁₋₆alkyl, C₂₋₆alkenyl, C₂₋₆alkynyl, C₁₋₆alkoxy, C₁₋₆alkanoyl, C₁₋₆alkanoyloxy, N-(C₁₋₆alkyl)amino, N,N-(C₁₋₆allyl)₂amino, C₁₋₆alkanoylamino, N-(C₁₋₆yl)carbamoyl, N,N-(C₁₋₆alkyl)₂carbamoyl, C₁₋₆alkylS(O)ₐ wherein a is 0 to 2, C₁₋₆alkoxycarbonyl, N-(C₁₋₆alkyl)sulphamoyl, N,N-(C₁₋₆allyl)₂sulphamoyl, C₁₋₆alkylsulphonylamino, carbocyclyl, heterocyclyl, carbocyclylC₀₋₆alkylene-Y— and heterocyclylC₀₋₆alkylene-Y—; or two R¹ on adjacent carbons may form an oxyC₁₋₄alkoxy group or a C₃₋₅alkylene group; wherein R¹ may be optionally substituted on carbon by one or more groups selected from R⁷; and wherein if said heterocyclyl contains an —NH— moiety that nitrogen may be optionally substituted by a group selected from R⁸;

n is 0-3; wherein the values of R¹ may be the same or different;

R², R³, R⁴ and R⁵ are independently selected from hydrogen, hydroxy, amino, cyano, C₁₋₄alkyl, C₁₋₄alkoxy, N-(C₁₋₄alkyl)amino, N,N—(C₁₋₄alkyl)₂amino, C₁₋₄alkylS(O)ₐ wherein a is 0 to 2, C₁₋₄alkoxycarbonyl, C₁₋₄alkoxycarbonylamino, C₁₋₄alkanoyloxy, carbocyclyl, heterocyclyl, carbocyclylC₁₋₄alkyl and heterocyclylC₁₋₄alkyl; or R² and R³ together form oxo or a spiro attached heterocyclyl; wherein R², R³, R⁴ and R⁵ may be independently optionally substituted on carbon by one or more groups selected from R⁹; and wherein if said heterocyclyl contains an —NH— moiety that nitrogen may be optionally substituted by a group selected from R¹⁰;

X and Z are independently selected from —CR¹¹R¹²—, —S(O)ₐ—, —O—, —NR¹³—, —C(O)—, —C(O)NR¹⁴—, —NR¹⁵C(O)—, —OC(O)—, —C(O)O—, —SO₂NR¹⁶— or —NR¹⁶SO₂—; wherein a is 0 to 2;

r is 1 or 2;

q is 0 or 1;

p is 0 or 1;

s is 0 or 1;

Ring B is carbocyclyl or heterocyclyl; wherein if said heterocyclyl contains an —NH— moiety that nitrogen may be optionally substituted by a group selected from R¹⁷;

R⁶ is a substituent on carbon and is selected from halo, nitro, cyano, hydroxy, amino, carboxy, carbamoyl, mercapto, sulphamoyl, trifluoromethyl, trifluoromethoxy, C₁₋₄alkyl, C₂₋₄alkenyl, C₂₋₄alkynyl, C₁₋₄alkoxy, C₁₋₄alkanoyl, C₁₋₄alkanoyloxy, N—(C₁₋₄alkyl)amino, N,N-(C₁₋₄alkyl)₂amino, C₁₋₄alkanoylamino, N—(C₁₋₄alkyl)carbamoyl, N,N—(C₁₋₄alkyl)₂carbamoyl, C₁₋₄alkylS(O)ₐ wherein a is 0 to 2, C₁₋₄alkoxycarbonyl, N—(C₁₋₄alkyl)sulphamoyl, N,N—(C₁₋₄allyl)₂sulphamoyl, C₁₋₄alkylsulphonylamino, carbocyclyl, heterocyclyl, carbocyclylC₀₋₄alkylene-Y— and heterocyclylC₀₋₄alkylene-Y—; wherein R⁶ may be optionally substituted on carbon by one or more groups selected from R¹⁸; and wherein if said heterocyclyl contains an —NH— moiety that nitrogen may be optionally substituted by a group selected from R¹⁹;

m is 0-3; wherein the values of R⁶ may be the same or different;

Y is —S(O)ₐ—, —O—, —NR²⁰, —C(O)—, —C(O)NR²¹—, —NR²²C(O)— or —SO₂NR²³—; wherein a is 0 to 2;

R⁷, R⁹ and R¹⁸ are independently selected from halo, nitro, cyano, hydroxy, amino, carboxy, carbamoyl, mercapto, sulphamoyl, trifluoromethyl, trifluoromethoxy, C₁₋₄alkyl, C₂₋₄alkenyl, C₂₋₄alkynyl, C₁₋₄alkoxy, C₂₋₄alkanoyl, C₁₋₄alkanoyloxy, N—(C₁₋₄alkyl)amino, N,N—(C₁₋₄alkyl)₂amino, C₁₋₄alkanoylamino, N—(C₁₋₄alkyl)carbamoyl, N,N—(C₁₋₄alkyl)₂carbamoyl, C₁₋₄alkylS(O)ₐ wherein a is 0 to 2, C₁₋₄alkoxycarbonyl, N—(C₁₋₄alkyl)sulphamoyl, N,N—(C₁₋₄alkyl)₂sulphamoyl, C₁₋₄alkylsulphonylamino, carbocyclyl and heterocyclyl; wherein R⁷, R⁹ and R¹⁸ may be independently optionally substituted on carbon by one or more R²⁶;

R¹¹ and R¹² are independently selected from hydrogen, hydroxy, amino, cyano, C₁₋₄alkyl, C₁₋₄alkoxy, N—(C₁₋₄alkyl)amino, N,N—(C₁₋₄alkyl)₂amino, carbocyclyl, heterocyclyl carbocyclylC₁₋₄alkyl, heterocyclylC₁₋₄alkyl; wherein R¹¹ and R¹² may be independently optionally substituted on carbon by one or more groups selected from R²⁴; and wherein if said heterocyclyl contains an —NH— moiety that nitrogen may be optionally substituted by a group selected from R²⁵;

R²⁴ is selected from halo, nitro, cyano, hydroxy, amino, carboxy, carbamoyl, mercapto, sulphamoyl, trifluoromethyl, trifluoromethoxy, C₁₋₄alkyl, C₂₋₄alkenyl, C₂₋₄alkynyl, C₁₋₄alkoxy, C₁₋₄alkanoyl, C₁₋₄alkanoyloxy, N—(C₁₋₄alkyl)amino, N,N—(C₁₋₄alkyl)₂amino, C₁₋₄alkanoylamino, N—(C₁₋₄alkyl)carbamoyl, N,N—(C₁₋₄alkyl)₂carbamoyl, C₁₋₄alkylS(O)ₐ wherein a is 0 to 2, C₁₋₄alkoxycarbonyl, N—(C₁₋₄alkyl)sulphamoyl, N,N—(C₁₋₄alkyl)₂sulphamoyl and C₁₋₄alkylsulphonylamino;

R⁸, R¹⁰, R¹⁷, R¹⁹ and R²⁵ are independently selected from C₁₋₄alkyl, C₁₋₄alkanoyl, C₁₋₄alkylsulphonyl, C₁₋₄alkoxycarbonyl, carbamoyl, N—(C₁₋₄alkyl)carbamoyl, N,N—(C₁₋₄alkyl)carbamoyl, benzyl, benzyloxycarbonyl, benzoyl, carbocyclyl, heterocyclyl and phenylsulphonyl; wherein R⁸, R¹⁰, R¹⁷, R¹⁹ and R²⁵ may be independently optionally substituted on carbon by one or more R²⁷;

R¹³, R¹⁴, R¹⁵, R¹⁶, R²⁰, R²¹, R²² and R²³ are independently selected from hydrogen, phenyl, C₁₋₄alkylsulphonyl and C₁₋₄alkyl;

R²⁶ and R²⁷ are independently selected from selected from halo, nitro, cyano, hydroxy, trifluoromethoxy, trifluoromethyl, amino, carboxy, carbamoyl, mercapto, sulphamoyl, methyl, ethyl, methoxy, ethoxy, acetyl, acetoxy, methylamino, ethylamino, dimethylamino, diethylamino, N-methyl-N-ethylamino, acetylamino, N-methylcarbamoyl, N-ethylcarbamoyl, N,N-dimethylcarbamoyl, N,N-diethylcarbamoyl, N-methyl-N-ethylcarbamoyl, methylthio, ethylthio, methylsulphinyl, ethylsulphinyl, mesyl, ethylsulphonyl, methoxycarbonyl, ethoxycarbonyl, N-methylsulphamoyl, N-ethylsulphamoyl, N,N-dimethylsulphamoyl, N,N-diethylsulphamoyl or N-methyl-N-ethylsulphamoyl;

or a pharmaceutically acceptable salt thereof;

in the manufacture of a medicament for use in the inhibition of 11βHSD1;

with the proviso that said compound is not (1-methyl-1-pyrid-3-ylethyl)-(pyrid-3-yl)-ketone.

A to a further feature of the invention there is provided the use of a compound of formula (I′): 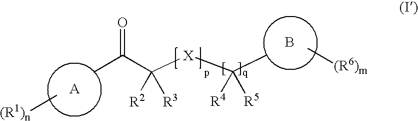
wherein:

Ring A is selected from aryl or heteroaryl;

R¹ is selected from halo, nitro, cyano, hydroxy, amino, carboxy, carbamoyl, mercapto, sulphamoyl, C₁₋₄alkyl, C₂₋₄alkenyl, C₂₋₄alkynyl, C₁₋₄alkoxy, C₁₋₄alkanoyl, C₁₋₄alkanoyloxy, N—(C₁₋₄alkyl)amino, N,N—(C₁₋₄alkyl)₂amino, C₁₋₄alkanoylamino, N—(C₁₋₄alkyl)carbamoyl, N,N—(C₁₋₄alkyl)₂carbamoyl, C₁₋₄alkylS(O)ₐ wherein a is 0 to 2, C₁₋₄alkoxycarbonyl, N—(C₁₋₄alkyl)sulphamoyl, N,N—(C₁₋₄alkyl)₂sulphamoyl, C₁₋₄alkylsulphonylamino, carbocyclyl, heterocyclyl, carbocyclylC₀₋₄alkylene-Y— and heterocyclylC₀₋₄alkylene-Y—; or two R¹ on adjacent carbons may form an oxyC₁₋₄alkoxy group; wherein R¹ may be optionally substituted on carbon by one or more groups selected from R⁷; and wherein if said heterocyclyl contains an —NH— moiety that nitrogen may be optionally substituted by a group selected from R⁸;

n is 0-3; wherein the values of R¹ may be the same or different;

R², R³, R⁴ and R⁵ are independently selected from hydrogen, hydroxy, amino, cyano, C₁₋₄alkyl, C₁₋₄alkoxy, N—(C₁₋₄alkyl)amino, N,N—(C₁₋₄alkyl)₂amino, carbocyclyl, heterocyclyl, carbocyclylC₁₋₄alkyl, heterocyclylC₁₋₄alkyl; wherein R², R³, R⁴ and R⁵ may be independently optionally substituted on carbon by one or more groups selected from R⁹; and wherein if said heterocyclyl contains an —NH— moiety that nitrogen may be optionally substituted by a group selected from R¹⁰;

X is —CR¹¹R¹²—, —S(O)ₐ—, —O—, —NR¹³—, —C(O)—, —C(O)NR¹⁴—, —NR¹⁵C(O)—, —SO₂NR¹⁶— or —NR¹⁶SO₂—; wherein a is 0 to 2;

q is 0 or 1;

p is 0 or 1;

Ring B is carbocyclyl or heterocyclyl; wherein if said heterocyclyl contains an —NH— moiety that nitrogen may be optionally substituted by a group selected from R¹⁷;

R⁶ is a substituent on carbon and is selected from halo, nitro, cyano, hydroxy, amino, carboxy, carbamoyl, mercapto, sulphamoyl, trifluoromethyl, trifluoromethoxy, C₁₋₄alkyl, C₂₋₄alkenyl, C₂₋₄alkynyl, C₁₋₄alkoxy, C₁₋₄alkanoyl, C₁₋₄alkanoyloxy, N—(C₁₋₄alkyl)amino, N,N—(C₁₋₄alkyl)₂amino, C₁₋₄alkanoylamino, N—(C₁₋₄alkyl)carbamoyl, N,N—(C₁₋₄alkyl)₂carbamoyl, C₁₋₄alkylS(O)ₐ wherein a is 0 to 2, C₁₋₄alkoxycarbonyl, N—(C₁₋₄alkyl)sulphamoyl, N,N—(C₁₋₄alkyl)₂sulphamoyl, C₁₋₄alkylsulphonylamino, carbocyclyl, heterocyclyl, carbocyclylC₀₋₄alkylene-Y— and heterocyclylC₀₋₄alkylene-Y—; wherein R⁶ may be optionally substituted on carbon by one or more groups selected from R¹⁸; and wherein if said heterocyclyl contains an —NH— moiety that nitrogen may be optionally substituted by a group selected from R¹⁹;

m is 0-3; wherein the values of R⁶ may be the same or different;

Y is —S(O)ₐ—, —O—, —NR²⁰—, —C(O), —C(O)NR²¹—, —NR²²C(O)— or —SO₂NR²³—; wherein a is 0 to 2;

R⁷, R⁹ and R¹⁸ are independently selected from halo, nitro, cyano, hydroxy, amino, carboxy, carbamoyl, mercapto, sulphamoyl, trifluoromethyl, trifluoromethoxy, C₁₋₄alkyl, C₂₋₄alkenyl, C₂₋₄alkynyl, C₁₋₄alkoxy, C₁₋₄alkanoyl, C₁₋₄alkanoyloxy, N—(C₁₋₄alkyl)amino, N,N—(C₁₋₄alkyl)₂amino, C₁₋₄alkanoylamino, N—(C₁₋₄alkyl)carbamoyl, N,N—(C₁₋₄alkyl)₂carbamoyl, C₁₋₄alkylS(O)ₐ wherein a is 0 to 2, C₁₋₄alkoxycarbonyl, N—(C₁₋₄alkyl)sulphamoyl, N,N—(C₁₋₄alkyl)₂sulphamoyl, C₁₋₄alkylsulphonylamino, carbocyclyl and heterocyclyl; wherein R⁷, R⁹ and R¹⁸ may be independently optionally substituted on carbon by one or more R²⁶;

R¹¹ and R¹² are independently selected from hydrogen, hydroxy, amino, cyano, C₁₋₄alkyl, C₁₋₄alkoxy, N—(C₁₋₄alkyl)amino, N,N—(C₁₋₄alkyl)₂amino, carbocyclyl, heterocyclyl carbocyclylC₁₋₄alkyl, heterocyclylC₁₋₄alkyl; wherein R⁹ and R¹⁰ may be independently optionally substituted on carbon by one or more groups selected from R²⁴; and wherein if said heterocyclyl contains an —NH— moiety that nitrogen may be optionally substituted by a group selected from R²⁵;

R²⁴ is selected from halo, nitro, cyano, hydroxy, amino, carboxy, carbamoyl, mercapto, sulphamoyl, trifluoromethyl, trifluoromethoxy, C₂₋₄alkyl, C₂₋₄alkenyl, C₂₋₄alkynyl, C₁₋₄alkoxy, C₁₋₄alkanoyl, C₁₋₄alkanoyloxy, N—(C₁₋₄alkyl)amino, N,N—(C₁₋₄alkyl)₂amino, C₁₋₄alkanoylamino, N—(C₁₋₄alkyl)carbamoyl, N,N—(C₁₋₄alkyl)₂carbamoyl, C₁₋₄alkylS(O)ₐ wherein a is 0 to 2, C₁₋₄alkoxycarbonyl, N—(C₁₋₄alkyl)sulphamoyl, N,N—(C₁₋₄alkyl)₂sulphamoyl and C₁₋₄alkylsulphonylamino;

R⁸, R¹⁰, R¹⁷, R¹⁹ and R²⁵ are independently selected from C₁₋₄alkyl, C₁₋₄alkanoyl, C₁₋₄alkylsulphonyl, C₁₋₄alkoxycarbonyl, carbamoyl, N—(C₁₋₄alkyl)carbamoyl, N,N—(C₁₋₄alkyl)carbamoyl, benzyl, benzyloxycarbonyl, benzoyl and phenylsulphonyl;

R¹³, R¹⁴, R¹⁵, R¹⁶, R²⁰, R²¹, R²² and R²³ are independently selected from hydrogen, phenyl and C₁₋₄alkyl;

R²⁶ is selected from halo, nitro, cyano, hydroxy, trifluoromethoxy, trifluoromethyl, amino, carboxy, carbamoyl, mercapto, sulphamoyl, methyl, ethyl, methoxy, ethoxy, acetyl, acetoxy, methylamino, ethylamino, dimethylamino, diethylamino, N-methyl-N-ethylamino, acetylamino, N-methylcarbamoyl, N-ethylcarbamoyl, N,N-dimethylcarbamoyl, N,N-diethylcarbamoyl, N-methyl-N-ethylcarbamoyl, methylthio, ethylthio, methylsulphinyl, ethylsulphinyl, mesyl, ethylsulphonyl, methoxycarbonyl, ethoxycarbonyl, N-methylsulphamoyl, N-ethylsulphamoyl, N,N-dimethylsulphamoyl, N,N-diethylsulphamoyl or N-methyl-N-ethylsulphamoyl;

or a pharmaceutically acceptable salt thereof,

in the manufacture of a medicament for use in the inhibition of 11βHSD1.

According to a further feature of the invention there is provided the use of a compound of formula (I″): 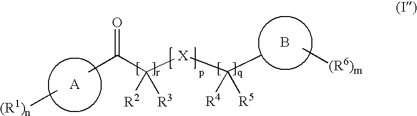
wherein:

Ring A is selected from aryl or heteroaryl;

R¹ is selected from halo, nitro, cyano, hydroxy, amino, carboxy, carbamoyl, mercapto, sulphamoyl, C₁₋₆alkyl, C₂₋₆alkenyl, C₂₋₆alkynyl, C₁₋₆alkoxy, C₁₋₆alkanoyl, C₁₋₆alkanoyloxy, N—(C₁₋₆alkyl)amino, N,N—(C₁₋₆alkyl)₂amino, C₁₋₆alkanoylamino, N—(C₁₋₆alkyl)carbamoyl, N,N—(C₁₋₆alkyl)₂carbamoyl, C₁₋₆alkylS(O)ₐ wherein a is 0 to 2, C₁₋₆alkoxycarbonyl, N—(C₁₋₆alkyl)sulphamoyl, N,N—(C₁₋₆alkyl)₂sulphamoyl, C₁₋₆alkylsulphonylamino, carbocyclyl, heterocyclyl, carbocyclylC₀₋₆alkylene-Y— and heterocyclylC₀₋₆alkylene-Y—; or two R¹ on adjacent carbons may form an oxyC₁₋₄alkoxy group; wherein R¹ may be optionally substituted on carbon by one or more groups selected from R⁷; and wherein if said heterocyclyl contains an —NH— moiety that nitrogen may be optionally substituted by a group selected from R⁸;

n is 0-3; wherein the values of R¹ may be the same or different;

R², R³, R⁴ and R⁵ are independently selected from hydrogen, hydroxy, amino, cyano, C₁₋₄alkyl, C₁₋₄alkoxy, N—(C₁₋₄alkyl)amino, N,N—(C₁₋₄alkyl)₂amino, C₁₋₄alkylS(O)ₐ wherein a is 0 to 2, C₁₋₄alkoxycarbonyl, carbocyclyl, heterocyclyl, carbocyclylC₁₋₄alkyl and heterocyclylC₁₋₄alkyl; or R² and R³ together form oxo; wherein R², R³, R⁴ and R⁵ may be independently optionally substituted on carbon by one or more groups selected from R⁹; and wherein if said heterocyclyl contains an —NH— moiety that nitrogen may be optionally substituted by a group selected from R¹⁰;

X is —CR¹¹R¹²—, —S(O)ₐ—, —)—, NR¹³—, —C(O), —C(O)NR¹⁴—, —NR¹⁵C(O)—, —SO₂NR¹⁶— or —NR¹⁶SO₂—; wherein a is 0 to 2;

r is 1 or 2;

q is 0 or 1;

p is 0 or 1;

Ring B is carbocyclyl or heterocyclyl; wherein if said heterocyclyl contains an —NH— moiety that nitrogen may be optionally substituted by a group selected from R¹⁷;

R⁶ is a substituent on carbon and is selected from halo, nitro, cyano, hydroxy, amino, carboxy, carbamoyl, mercapto, sulphamoyl, trifluoromethyl, trifluoromethoxy, C₁₋₄alkyl, C₂₋₄alkenyl, C₂₋₄alkynyl, C₁₋₄alkoxy, C₁₋₄alkanoyl, C₁₋₄alkanoyloxy, N—(C₁₋₄alkyl)amino, N,N—(C₁₋₄alkyl)₂amino, C₁₋₄alkanoylamino, N—(C₁₋₄alkyl)carbamoyl, N,N—(C₁₋₄alkyl)₂carbamoyl, C₁₋₄alkylS(O)ₐ wherein a is 0 to 2, C₁₋₄alkoxycarbonyl, N—(C₁₋₄alkyl)sulphamoyl, N,N—(C₁₋₄alkyl)₂sulphamoyl, C₁₋₄alkylsulphonylamino, carbocyclyl, heterocyclyl, carbocyclylC₀₋₄alkylene-Y— and heterocyclylC₀₋₄alkylene-Y—; wherein R⁶ may be optionally substituted on carbon by one or more groups selected from R¹⁸; and wherein if said heterocyclyl contains an —NH— moiety that nitrogen may be optionally substituted by a group selected from R¹⁹;

m is 0-3; wherein the values of R⁶ may be the same or different;

Y is —S(O)ₐ—, —O—, —NR²⁰ —, —C(O), —C(O)NR²¹—, —NR²²C(O)— or —SO₂NR²³—; wherein a is 0 to 2;

R⁷, R⁹ and R¹⁸ are independently selected from halo, nitro, cyano, hydroxy, amino, carboxy, carbamoyl, mercapto, sulphamoyl, trifluoromethyl, trifluoromethoxy, C₁₋₄alkyl, C₂₋₄alkenyl, C₂₋₄alkynyl, C₁₋₄alkoxy, C₁₋₄alkanoyl, C₁₋₄alkanoyloxy, N—(C₁₋₄alkyl)amino, N,N—(C₁₋₄alkyl)₂amino, C₁₋₄alkanoylamino, N—(C₁₋₄alkyl)carbamoyl, N,N—(C₁₋₄alkyl)₂carbamoyl, C₁₋₄alkylS(O)ₐ wherein a is 0 to 2, C₁₋₄alkoxycarbonyl, N—(C₁₋₄alkyl)sulphamoyl, N,N—(C₁₋₄alkyl)₂sulphamoyl, C₁₋₄alkylsulphonylamino, carbocyclyl and heterocyclyl; wherein R⁷, R⁹ and R¹⁸ may be independently optionally substituted on carbon by one or more R²⁶;

R¹¹ and R¹² are independently selected from hydrogen, hydroxy, amino, cyano, C₁₋₄alkyl, C₁₋₄alkoxy, N—(C₁₋₄alkyl)amino, N,N—(C₁₋₄alkyl)₂amino, carbocyclyl, heterocyclyl carbocyclylC₁₋₄alkyl, heterocyclylC₁₋₄alkyl; wherein R¹¹ and R¹² may be independently optionally substituted on carbon by one or more groups selected from R²⁴; and wherein if said heterocyclyl contains an —NH— moiety that nitrogen may be optionally substituted by a group selected from R²⁵;

R²⁴ is selected from halo, nitro, cyano, hydroxy, amino, carboxy, carbamoyl, mercapto, sulphamoyl, trifluoromethyl, trifluoromethoxy, C₁₋₄alkyl, C₂₋₄alkenyl, C₂₋₄alkynyl, C₁₋₄alkoxy, C₁₋₄alkanoyl, C₁₋₄alkanoyloxy, N—(C₁₋₄alkyl)amino, N,N—(C₁₋₄alkyl)₂amino, C₁₋₄alkanoylamino, N—(C₁₋₄alkyl)carbamoyl, N,N—(C₁₋₄alkyl)₂carbamoyl, C₁₋₄alkylS(O)ₐ wherein a is 0 to 2, C₁₋₄alkoxycarbonyl, N—(C₁₋₄alkyl)sulphamoyl, N,N—(C₁₋₄alkyl)₂sulphamoyl and C₁₋₄alkylsulphonylamino;

R⁸, R¹⁰, R¹⁷, R¹⁹ and R²⁵ are independently selected from C₁₋₄alkyl, C₁₋₄alkanoyl, C₁₋₄alkylsulphonyl, C₁₋₄alkoxycarbonyl, carbamoyl, N—(C₁₋₄alkyl)carbamoyl, N,N—(C₁₋₄alkyl)carbamoyl, benzyl, benzyloxycarbonyl, benzoyl, heterocyclyl and phenylsulphonyl;

R¹³, R¹⁴, R¹⁵, R¹⁶, R²⁰, R²¹, R²² and R²³ are independently selected from hydrogen, phenyl, C₁₋₄alkylsulphonyl and C₁₋₄alkyl;

R²⁶ is selected from halo, nitro, cyano, hydroxy, trifluoromethoxy, trifluoromethyl, amino, carboxy, carbamoyl, mercapto, sulphamoyl, methyl, ethyl, methoxy, ethoxy, acetyl, acetoxy, methylamino, ethylamino, dimethylamino, diethylamino, N-methyl-N-ethylamino, acetylamino, N-methylcarbamoyl, N-ethylcarbamoyl, N,N-dimethylcarbamoyl, N,N-diethylcarbamoyl, N-methyl-N-ethylcarbamoyl, methylthio, ethylthio, methylsulphinyl, ethylsulphinyl, mesyl, ethylsulphonyl, methoxycarbonyl, ethoxycarbonyl, N-methylsulphamoyl, N-ethylsulphamoyl, N,N-dimethylsulphamoyl, N,N-diethylsulphamoyl or N-methyl-N-ethylsulphamoyl;

or a pharmaceutically acceptable salt thereof;

in the manufacture of a medicament for use in the inhibition of 11βHSD1;

with the proviso that said compound is not (1-methyl-1-pyrid-3-ylethyl)-(pyrid-3-yl)-ketone.

According to a further feature of the invention there is provided a compound of formula (Ia): 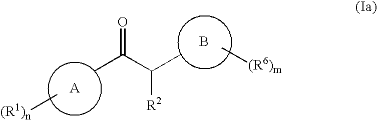
wherein:

Ring A is selected from furanyl, thienyl or pyridyl;

R¹ is selected from halo, nitro, cyano, hydroxy, amino, carboxy, carbamoyl, mercapto, sulphamoyl, C₁₋₄alkyl, C₂₋₄alkenyl, C₂₋₄alkynyl, C₁₋₄alkoxy, C₁₋₄alkanoyl, C₁₋₄alkanoyloxy, N—(C₁₋₄alkyl)amino, N,N—(C₁₋₄alkyl)₂amino, C₁₋₄alkanoylamino, N—(C₁₋₄alkyl)carbamoyl, N,N—(C₁₋₄alkyl)₂carbamoyl, C₁₋₄alkylS(O)ₐ wherein a is 0 to 2, C₁₋₄alkoxycarbonyl, N—(C₁₋₄alkyl)sulphamoyl, N,N—(C₁₋₄alkyl)₂sulphamoyl, C₁₋₄alkylsulphonylamino, carbocyclyl, heterocyclyl, carbocyclylC₀₋₄alkylene-Y— and heterocyclylC₀₋₄alkylene-Y—; or two R¹ on adjacent carbons may form an oxyC₁₋₄alkoxy group; wherein R¹ may be optionally substituted on carbon by one or more groups selected from R⁷; and wherein if said heterocyclyl contains an —NH— moiety that nitrogen may be optionally substituted by a group selected from R⁸;

n is 0-3; wherein the values of R¹ may be the same or different;

R² is selected from amino, C₁₋₃alkoxy and N—(C₁₋₃alkyl)amino; wherein R² may be optionally substituted on carbon by one or more groups selected from R⁹;

Ring B is 3-6 membered aryl or a 3-6 membered heteroaryl; wherein if said heteroaryl contains an —NH— moiety that nitrogen may be optionally substituted by a group selected from R¹⁷;

R⁶ is a substituent on carbon and is selected from halo, nitro, cyano, hydroxy, amino, carboxy, carbamoyl, mercapto, sulphamoyl, trifluoromethyl, trifluoromethoxy, C₁₋₄alkyl, C₂₋₄alkenyl, C₂₋₄alkynyl, C₁₋₄alkoxy, C₁₋₄alkanoyl, C₁₋₄alkanoyloxy, N—(C₁₋₄alkyl)amino, N,N—(C₁₋₄alkyl)₂amino, C₁₋₄alkanoylamino, N—(C₁₋₄alkyl)carbamoyl, N,N—(C₁₋₄alkyl)2carbamoyl, C₁₋₄alkylS(O)ₐ wherein a is 0 to 2, C₁₋₄alkoxycarbonyl, N—(C₁₋₄alkyl)sulphamoyl, N,N—(C₁₋₄alkyl)₂sulphamoyl, C₁₋₄alkylsulphonylamino, carbocyclyl and heterocyclyl; wherein R⁶ may be optionally substituted on carbon by one or more groups selected from R¹⁸; and wherein if said heterocyclyl contains an —NH— moiety that nitrogen may be optionally substituted by a group selected from R¹⁹;

m is 0-3; wherein the values of R⁶ may be the same or different;

Y is —S(O)ₐ—, —O—, —NR²⁰—, —C(O), —C(O)NR²¹—, —NR²²C(O)— or —SO₂NR²³—; wherein a is 0to2;

R⁷, R⁹ and R¹⁸ are independently selected from halo, nitro, cyano, hydroxy, amino, carboxy, carbamoyl, mercapto, sulphamoyl, trifluoromethyl, trifluoromethoxy, C₁₋₄alkyl, C₂₋₄alkenyl, C₂₋₄alkynyl, C₁₋₄alkoxy, C₁₋₄alkanoyl, C₁₋₄alkanoyloxy, N—(C₁₋₄alkyl)amino, N,N—(C₁₋₄alkyl)₂amino, C₁₋₄alkanoylamino, N—(C₁₋₄alkyl)carbamoyl, N,N—(C₁₋₄alkyl)₂carbamoyl, C₁₋₄alkylS(O)a wherein a is 0 to 2, C₁₋₄alkoxycarbonyl, N—(C₁₋₄alkyl)sulphamoyl, N,N—(C₁₋₄alkyl)₂sulphamoyl, C₁₋₄alkylsulphonylamino, carbocyclyl and heterocyclyl;

R⁸, R¹⁷ and R¹⁹ are independently selected from C₁₋₄alkyl, C₁₋₄alkanoyl, C₁₋₄alkylsulphonyl, C₁₋₄alkoxycarbonyl, carbamoyl, N—(C₁₋₄alkyl)carbamoyl, N,N—(C₁₋₄alkyl)carbamoyl, benzyl, benzyloxycarbonyl, benzoyl and phenylsulphonyl;

R²⁰, R²¹, R²² and R²³ are independently selected from hydrogen and C₁₋₄alkyl; or a pharmaceutically acceptable salt thereof;

with the proviso that said compound is not (α-methoxybenzyl)-(pyrid-4-yl)-ketone, (α-aminobenzyl)-(pyrid-3-yl)-ketone, [1-(fur-2-yl)-1-(ethoxy)methyl]-(fur-2-yl)-ketone or [1-(fur-2-yl)-1-(methoxy)methyl]-(fur-2-yl)-ketone.

According to a further feature of the invention there is provided a compound of formula (Ib): 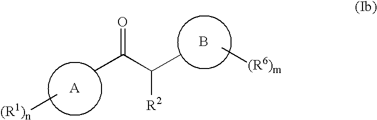
wherein:

Ring A is thiazolyl;

R¹ is selected from halo, nitro, cyano, hydroxy, amino, carboxy, carbamoyl, mercapto, sulphamoyl, C₁₋₄alkyl, C₂₋₄alkenyl, C₂₋₄alkynyl, C₁₋₄alkoxy, C₁₋₄alkanoyl, C₁₋₄alkanoyloxy, N—(C₁₋₄alkyl)amino, N,N—(C₁₋₄alkyl)₂amino, C₁₋₄alkanoylamino, N—(C₁₋₄alkyl)carbamoyl, N,N—(C₁₋₄alkyl)₂carbamoyl, C₁₋₄alkylS(O)ₐ wherein a is 0 to 2, C₁₋₄alkoxycarbonyl, N—(C₁₋₄alkyl)sulphamoyl, N,N—(C₁₋₄alkyl)₂sulphamoyl, C₁₋₄alkylsulphonylamino, carbocyclyl, heterocyclyl, carbocyclylC₀₋₄alkylene-Y— and heterocyclylC₀₋₄alkylene-Y—; or two R¹ on adjacent carbons may form an oxyC₁₋₄alkoxy group; wherein R¹ may be optionally substituted on carbon by one or more groups selected from R⁷; and wherein if said heterocyclyl contains an —NH— moiety that nitrogen may be optionally substituted by a group selected from R⁸;

n is 0-3; wherein the values of R¹ may be the same or different;

R² is selected from hydroxy, amino, C₁₋₃alkoxy and N—(C₁₋₃alkyl)amino; wherein R² may be optionally substituted on carbon by one or more groups selected from R⁹;

Ring B is 3-6 membered aryl or a 3-6 membered heteroaryl; wherein if said heteroaryl contains an —NH— moiety that nitrogen may be optionally substituted by a group selected from R¹⁷;

R⁶ is a substituent on carbon and is selected from halo, nitro, cyano, hydroxy, amino, carboxy, carbamoyl, mercapto, sulphamoyl, trifluoromethyl, trifluoromethoxy, C₁₋₄alkyl, C₂₋₄alkenyl, C₂₋₄alkynyl, C₁₋₄alkoxy, C₁₋₄alkanoyl, C₁₋₄alkanoyloxy, N—(C₁₋₄alkyl)amino, N,N—(C₁₋₄alkyl)₂amino, C₁₋₄alkanoylamino, N—(C₁₋₄alkyl)carbamoyl, N,N—(C₁₋₄alkyl)₂carbamoyl, C₁₋₄alkylS(O)ₐ wherein a is 0 to 2, C₁₋₄alkoxycarbonyl, N—(C₁₋₄alkyl)sulphamoyl, N,N—(C₁₋₄alkyl)₂sulphamoyl, C₁₋₄alkylsulphonylamino, carbocyclyl and heterocyclyl; wherein R⁶ may be optionally substituted on carbon by one or more groups selected from R¹⁸; and wherein if said heterocyclyl contains an —NH— moiety that nitrogen may be optionally substituted by a group selected from R¹⁹;

m is 0-3; wherein the values of R⁶ may be the same or different;

Y is —S(O)ₐ—, —O—, —NR₂₀—, —C(O), —C(O)NR²¹—, —NR²²C(O)— or —SO₂NR²³—; wherein a is 0 to 2;

R⁷, R⁹ and R¹⁸ are independently selected from halo, nitro, cyano, hydroxy, amino, carboxy, carbamoyl, mercapto, sulphamoyl, trifluoromethyl, trifluoromethoxy, C₁₋₄alkyl, C₂₋₄alkenyl, C₂₋₄alkynyl, C₁₋₄alkoxy, C₁₋₄alkanoyl, C₁₋₄alkanoyloxy, N—(C₁₋₄alkyl)amino, N,N—(C₁₋₄alkyl)₂amino, C₁₋₄alkanoylamino, N—(C₁₋₄alkyl)carbamoyl, N,N—(C₁₋₄alkyl)₂carbamoyl, C₁₋₄alkylS(O)ₐ wherein a is 0 to 2, C₁₋₄alkoxycarbonyl, N—(C₁₋₄alkyl)sulphamoyl, N,N—(C₁₋₄alkyl)₂sulphamoyl, C₁₋₄alkylsulphonylamino, carbocyclyl and heterocyclyl;

R⁸, R¹⁷ and R¹⁹ are independently selected from C₁₋₄alkyl, C₁₋₄alkanoyl, C₁₋₄alkylsulphonyl, C₁₋₄alkoxycarbonyl, carbamoyl, N—(C₁₋₄alkyl)carbamoyl, N,N—(C₁₋₄alkyl)carbamoyl, benzyl, benzyloxycarbonyl, benzoyl and phenylsulphonyl;

R²⁰, R²¹, R²² and R²³ are independently selected from hydrogen and C₁₋₄alkyl;

or a pharmaceutically acceptable salt thereof.

According to a further feature of the invention there is provided a compound of formula (Ic): 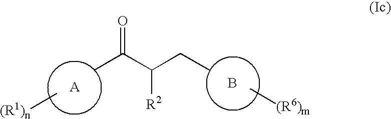
wherein:

Ring A is selected from furyl, thienyl, thiazolyl and pyridyl;

R¹ is selected from halo, nitro, cyano, hydroxy, amino, carboxy, carbamoyl, mercapto, sulphamoyl, C₁₋₄alkyl, C₂₋₄alkenyl, C₂₋₄alkynyl, C₁₋₄alkoxy, C₁₋₄alkanoyl, C₁₋₄alkanoyloxy, N—(C₁₋₄alkyl)amino, N,N—(C₁₋₄alkyl)₂amino, C₁₋₄alkanoylamino, N—(C₁₋₄alkyl)carbamoyl, N,N—(C₁₋₄alkyl)₂carbamoyl, C₁₋₄alkylS(O)ₐ wherein a is 0 to 2, C₁₋₄alkoxycarbonyl, N—(C₁₋₄alkyl)sulphamoyl, N,N—(C₁₋₄alkyl)₂sulphamoyl, C₁₋₄alkylsulphonylamino, carbocyclyl, heterocyclyl, carbocyclylC₀₋₄aLkylene-Y— and heterocyclylC₀₋₄alkylene-Y—; or two R¹ on adjacent carbons may form an oxyC₁₋₄alkoxy group; wherein R¹ may be optionally substituted on carbon by one or more groups selected from R⁷; and wherein if said heterocyclyl contains an —NH— moiety that nitrogen may be optionally substituted by a group selected from R⁸;

n is 0-3; wherein the values of R¹ may be the same or different;

R² is selected from 3-6 membered aryl or carbon linked 3-6 membered heteroaryl; wherein R² may be independently optionally substituted on carbon by one or more groups selected from R⁹; and wherein if said heteroaryl contains an —NH— moiety that nitrogen may be optionally substituted by a group selected from R¹⁰;

Ring B is 3-6 membered aryl or a carbon linked 3-6 membered heteroaryl; wherein if said heteroaryl contains an —NH— moiety that nitrogen may be optionally substituted by a group selected from R¹⁷;

R⁶ is a substituent on carbon and is selected from halo, nitro, cyano, hydroxy, amino, carboxy, carbamoyl, mercapto, sulphamoyl, trifluoromethyl, trifluoromethoxy, C₁₋₄alkyl, C₂₋₄alkenyl, C₂₋₄alkynyl, C₁₋₄alkoxy, C₁₋₄alkanoyl, C₁₋₄alkanoyloxy, N—(C₁₋₄alkyl)amino, N,N—(C₁₋₄alkyl)₂amino, C₁₋₄alkanoylamino, N—(C₁₋₄alkyl)carbamoyl, N,N—(C₁₋₄alkyl)₂carbamoyl, C₁₋₄alkylS(O)ₐ wherein a is 0 to 2, C₁₋₄alkoxycarbonyl, N—(C₁₋₄alkyl)sulphamoyl, N,N—(C₁₋₄alkyl)₂sulphamoyl, C₁₋₄alkylsulphonylamino, carbocyclyl and heterocyclyl; wherein R⁶ may be optionally substituted on carbon by one or more groups selected from R¹⁸; and wherein if said heterocyclyl contains an —NH— moiety that nitrogen may be optionally substituted by a group selected from R¹⁹;

m is 0-3; wherein the values of R⁶ may be the same or different;

Y is —S(O)ₐ—, —O—, —NR₂₀—, —C(O), —C(O)NR²¹—, —NR²²C(O)— or —SO₂NR²³—; wherein a is 0 to 2;

R⁷, R⁹ and R¹⁸ are independently selected from halo, nitro, cyano, hydroxy, amino, carboxy, carbamoyl, mercapto, sulphamoyl, trifluoromethyl, trifluoromethoxy, C₁₋₄alkyl, C₂₋₄alkenyl, C₂₋₄alkynyl, C₁₋₄alkoxy, C₁₋₄alkanoyl, C₁₋₄alkanoyloxy, N—(C₁₋₄alkyl)amino, N,N—(C₁₋₄alkyl)₂amino, C₁₋₄alkanoylamino, N—(C₁₋₄alkyl)carbamoyl, N,N—(C₁₋₄alkyl)₂carbamoyl, C₁₋₄alkylS(O)ₐ wherein a is 0 to 2, C₁₋₄alkoxycarbonyl, N—(C₁₋₄alkyl)sulphamoyl, N,N—(C₁₋₄alkyl)₂sulphamoyl, C₁₋₄alkylsulphonylamino, carbocyclyl and heterocyclyl;

R⁸, R¹⁰, R¹⁷ and R¹⁹ are independently selected from C₁₋₄alkyl, C₁₋₄alkanoyl, C₁₋₄alkylsulphonyl, C₁₋₄alkoxycarbonyl, carbamoyl, N—(C₁₋₄alkyl)carbamoyl, N,N—(C₁₋₄alkyl)carbamoyl, benzyl, benzyloxycarbonyl, benzoyl and phenylsulphonyl;

R²⁰, R²¹, R²² and R²³ are independently selected from hydrogen and C₁₋₄alkyl; or a pharmaceutically acceptable salt thereof;

with the proviso that said compound is not
[1-(pyrazin-2-yl)-2-(2-fluorophenyl)ethyl]-(fur-2-yl)-ketone,
[1-(pyrazin-2-yl)-2-(4-chlorophenyl)ethyl]-(fur-2-yl)-ketone,
[2-(pyridin-3-yl)-1-(2,4-dichlorophenyl)ethyl]-(pyrid-3-yl)-ketone,
[2-(fur-2-yl)-1-(2,4-dichlorophenyl)ethyl]-(pyrid-3-yl)-ketone,
[2-(4-nitrophenyl)-1-(2,4-dichlorophenyl)ethyl]-(pyrid-3-yl)-ketone,
[2-(thien-2-yl)-1-(2,4-dichlorophenyl)ethyl]-(pyrid-3-yl)-ketone,
[2-(phenyl)-1-(2,4-dichlorophenyl)ethyl]-(pyrid-3-yl)-ketone or
[2-(4-chlorophenyl)-1-(pyrazin-2-yl)ethyl]-(pyrid-3-yl)-ketone.

According to a further feature of the invention there is provided a compound of formula (Id): 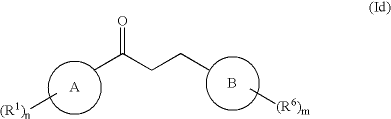
wherein:

Ring A is thiazolyl;

R¹ is selected from halo, nitro, cyano, hydroxy, amino, carboxy, carbamoyl, mercapto, sulphamoyl, C₁₋₄alkyl, C₂₋₄alkenyl, C₂₋₄alkynyl, C₁₋₄alkoxy, C₁₋₄alkanoyl, C₁₋₄alkanoyloxy, N—(C₁₋₄alkyl)amino, N,N—(C₁₋₄alkyl)₂amino, C₁₋₄alkanoylamino, N—(C₁₋₄alkyl)carbamoyl, N,N—(C₁₋₄alkyl)₂carbamoyl, C₁₋₄alkylS(O)ₐ wherein a is 0 to 2, C₁₋₄alkoxycarbonyl, N—(C₁₋₄alkyl)sulphamoyl, N,N—(C₁₋₄alkyl)₂sulphamoyl, C₁₋₄alkylsulphonylamino, carbocyclyl, heterocyclyl, carbocyclylC₀₋₄alkylene-Y— and heterocyclylC₀₋₄alkylene-Y—; or two R¹ on adjacent carbons may form an oxyC₁₋₄alkoxy group; wherein R¹ may be optionally substituted on carbon by one or more groups selected from R⁷; and wherein if said heterocyclyl contains an —NH— moiety that nitrogen may be optionally substituted by a group selected from R⁸;

n is 0-3; wherein the values of R¹ may be the same or different;

Ring B is 3-6 membered aryl or a 3-6 membered heteroaryl; wherein if said heteroaryl contains an —NH— moiety that nitrogen may be optionally substituted by a group selected from R¹⁷;

R⁶ is a substituent on carbon and is selected from halo, nitro, cyano, hydroxy, amino, carboxy, carbamoyl, mercapto, sulphamoyl, trifluoromethyl, trifluoromethoxy, C₁₋₄alkyl, C₂₋₄alkenyl, C₂₋₄alkynyl, C₁₋₄alkoxy, C₁₋₄alkanoyl, C₁₋₄alkanoyloxy, N—(C₁₋₄alkyl)amino, N,N—(C₁₋₄alkyl)₂amino, C₁₋₄alkanoylamino, N—(C₁₋₄alkyl)carbamoyl, N,N—(C₁₋₄alkyl)₂carbamoyl, C₁₋₄alkylS(O)ₐ wherein a is 0 to 2, C₁₋₄alkoxycarbonyl, N—(C₁₋₄alkyl)sulphamoyl, N,N—(C₁₋₄alkyl)₂sulphamoyl, C₁₋₄alkylsulphonylamino, carbocyclyl and heterocyclyl; wherein R⁶ may be optionally substituted on carbon by one or more groups selected from R¹⁸; and wherein if said heterocyclyl contains an —NH— moiety that nitrogen may be optionally substituted by a group selected from R¹⁹;

m is 0-3; wherein the values of R⁶ may be the same or different;

Y is —S(O)ₐ—, —O—, —NR²⁰—, —C(O), —C(O)NR²¹—, —NR²²C(O)— or —SO₂NR²³—; wherein a is 0 to 2;

R⁷ and R¹⁸ are independently selected from halo, nitro, cyano, hydroxy, amino, carboxy, carbamoyl, mercapto, sulphamoyl, trifluoromethyl, trifluoromethoxy, C₁₋₄alkyl, C₂₋₄alkenyl, C₂₋₄alkynyl, C₁₋₄alkoxy, C₁₋₄alkanoyl, C₁₋₄alkanoyloxy, N—(C₁₋₄alkyl)amino, N,N—(C₁₋₄alkyl)₂amino, C₁₋₄alkanoylamino, N—(C₁₋₄alkyl)carbamoyl, N,N—(C₁₋₄alkyl)₂carbamoyl, C₁₋₄alkylS(O)ₐ wherein a is 0 to 2, C₁₋₄alkoxycarbonyl, N—(C₁₋₄alkyl)sulphamoyl, N,N—(C₁₋₄alkyl)₂sulphamoyl, C₁₋₄alkylsulphonylamino, carbocyclyl and heterocyclyl;

R⁸, R¹⁷ and R¹⁹ are independently selected from C₁₋₄alkyl, C₁₋₄alkanoyl, C₁₋₄alkylsulphonyl, C₁₋₄alkoxycarbonyl, carbamoyl, N—(C₁₋₄alkyl)carbamoyl, N,N—(C₁₋₄alkyl)carbamoyl, benzyl, benzyloxycarbonyl, benzoyl and phenylsulphonyl;

R²⁰, R²¹, R²² and R²³ are independently selected from hydrogen and C₁₋₄alkyl;

or a pharmaceutically acceptable salt thereof;

with the proviso that said compound is not (phenethyl)-(5-aminothiazol-4-yl)-ketone.

According to a further feature of the invention there is provided a compound of formula (Ie): 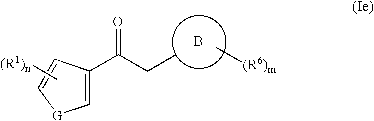
wherein:

G is O or S;

R¹ is selected from fluoro, chloro, bromo, sulphamoyl, methyl, methoxy, ethoxy, acetyl or thiomethyl;

n is 0-3; wherein the values of R¹ may be the same or different;

Ring B is 3-6 membered aryl or a 3-6 membered carbon linked heteroaryl; wherein if said heteroaryl contains an —NH— moiety that nitrogen may be optionally substituted by a group selected from R¹⁷;

R⁶ is a substituent on carbon and is selected from halo, nitro, cyano, hydroxy, amino, carboxy, carbamoyl, mercapto, sulphamoyl, trifluoromethyl, trifluoromethoxy, C₁₋₄alkyl, C₂₋₄alkenyl, C₂₋₄alkynyl, C₁₋₄alkoxy, C₁₋₄alkanoyI, C₁₋₄alkanoyloxy, N—(C₁₋₄alkyl)amino, N,N—(C₁₋₄alkyl)₂amino, C₁₋₄alkanoylamino, N—(C₁₋₄alkyl)carbamoyl, N,N—(C₁₋₄alkyl)₂carbamoyl, C₁₋₄alkylS(O)ₐ wherein a is 0 to 2, C₁₋₄alkoxycarbonyl, N—(C₁₋₄alkyl)sulphamoyl, N,N—(C₁₋₄alkyl)₂sulphamoyl, C₁₋₄alkylsulphonylamino, carbocyclyl and heterocyclyl; wherein R⁶ may be optionally substituted on carbon by one or more groups selected from R¹⁸; and wherein if said heterocyclyl contains an —NH— moiety that nitrogen may be optionally substituted by a group selected from R¹⁹;

m is 0-3; wherein the values of R⁶ may be the same or different;

R¹⁸ is selected from halo, nitro, cyano, hydroxy, amino, carboxy, carbamoyl, mercapto, sulphamoyl, trifluoromethyl, trifluoromethoxy, C₁₋₄alkyl, C₂₋₄alkenyl, C₂₋₄alkynyl, C₁₋₄alkoxy, C₁₋₄alkanoyl, C₁₋₄alkanoyloxy, N—(C₁₋₄alkyl)amino, N,N—(C₁₋₄alkyl)₂amino, C₁₋₄alkanoylamino, N—(C₁₋₄alkyl)carbamoyl, N,N—(C₁₋₄alkyl)₂carbamoyl, C₁₋₄alkylS(O)ₐ wherein a is 0 to 2, C₁₋₄alkoxycarbonyl, N—(C₁₋₄alkyl)sulphamoyl, N,N—(C₁₋₄alkyl)₂sulphamoyl, C₁₋₄alkylsulphonylamino, carbocyclyl and heterocyclyl;

R¹⁷ and R¹⁹ are independently selected from C₁₋₄alkyl, C₁₋₄alkanoyl, C₁₋₄alkylsulphonyl, C₁₋₄alkoxycarbonyl, carbamoyl, N—(C₁₋₄alkyl)carbamoyl, N,N—(C₁₋₄alkyl)carbamoyl, benzyl, benzyloxycarbonyl, benzoyl and phenylsulphonyl;

or a pharmaceutically acceptable salt thereof;

with the proviso that said compound is not
(2,5-dimethylthien-3-yl)-(2,5-dimethylthien-3-ylmethyl)-ketone;
(2,5-dichlorothien-3-yl)-(benzyl)-ketone; (2,4,5-trichlorothien-3-yl)-(benzyl)-ketone;
(4-bromothien-3-yl)-(2-nitrobenzyl)-ketone; (2-methylfur-3-yl)-(benzyl)-ketone; or
(2,5-dimethylthien-3-yl)-(5-chlorothien-2-ylmethyl)-ketone.

According to a further feature of the invention there is provided a compound of formula (If): 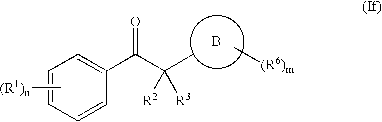
wherein:

R¹ is selected from fluoro, chloro, bromo, sulphamoyl, methyl, methoxy, ethoxy, acetyl or thiomethyl;

n is 0-3; wherein the values of R¹ may be the same or different;

R² is N—(C₁₋₄alkyl)amino; wherein R² may be optionally substituted on carbon by one or more groups selected from R⁹;

R³ is selected from hydrogen or C₁₋₄alkyl; wherein R³ may be optionally substituted on carbon by one or more groups selected from R⁹;

Ring B is carbocyclyl or heterocyclyl; wherein if said heterocyclyl contains an —NH— moiety that nitrogen may be optionally substituted by a group selected from R¹⁷;

R⁶ is a substituent on carbon and is selected from halo, nitro, cyano, hydroxy, amino, carboxy, carbamoyl, mercapto, sulphamoyl, trifluoromethyl, trifluoromethoxy, C₁₋₄alkyl, C₂₋₄alkenyl, C₂₋₄alkynyl, C₁₋₄alkoxy, C₁₋₄alkanoyl, C₁₋₄alkanoyloxy, N—(C₁₋₄alkyl)amino, N,N—(C₁₋₄alkyl)₂amino, C₁₋₄alkanoylamino, N—(C₁₋₄alkyl)carbamoyl, N,N—(C₁₋₄alkyl)₂carbamoyl, C₁₋₄alkylS(O)ₐ wherein a is 0 to 2, C₁₋₄alkoxycarbonyl, N—(C₁₋₄alkyl)sulphamoyl, N,N—(C₁₋₄alkyl)₂sulphamoyl, C₁₋₄alkylsulphonylamino, carbocyclyl and heterocyclyl; wherein R⁶ may be optionally substituted on carbon by one or more groups selected from R¹⁸; and wherein if said heterocyclyl contains an —NH— moiety that nitrogen may be optionally substituted by a group selected from R¹⁹;

m is 0-3; wherein the values of R⁶ may be the same or different;

R⁹ and R¹⁸ are independently selected from halo, nitro, cyano, hydroxy, amino, carboxy, carbamoyl, mercapto, sulphamoyl, trifluoromethyl, trifluoromethoxy, C₁₋₄alkyl, C₂₋₄alkenyl, C₂₋₄alkynyl, C₁₋₄alkoxy, C₁₋₄alkanoyl, C₁₋₄alkanoyloxy, N—(C₁₋₄alkyl)amino, N,N—(C₁₋₄alkyl)₂amino, C₁₋₄alkanoylamino, N—(C₁₋₄alkyl)carbamoyl, N,N—(C₁₋₄alkyl)₂carbamoyl, C₁₋₄alkylS(O)ₐ wherein a is 0 to 2, C₁₋₄alkoxycarbonyl, N—(C₁₋₄alkyl)sulphamoyl, N,N—(C₁₋₄alkyl)₂sulphamoyl and C₁₋₄alkylsulphonylamino;

R¹⁷ and R¹⁹ are independently selected from C₁₋₄alkyl, C₁₋₄alkanoyl, C₁₋₄alkylsulphonyl, C₁₋₄alkoxycarbonyl, carbamoyl, N—(C₁₋₄alkyl)carbamoyl, N,N—(C₁₋₄alkyl)carbamoyl, benzyl, benzyloxycarbonyl, benzoyl and phenylsulphonyl;

or a pharmaceutically acceptable salt thereof;

with the proviso that said compound is not
(4-methoxyphenyl)-[α-(1-hydroxyprop-2-ylamino)-4-methoxybenzyl]-ketone;
(4-methoxyphenyl)-[α-(butylamino)-4-methoxybenzyl]-ketone;
(4-methoxyphenyl)-[α-(ethylamino)benzyl]-ketone;
(4-methoxyphenyl)-[α-(1-hydroxybut-2-ylamino)benzyl]-ketone;
(4-methoxyphenyl)-[α-(1-hydroxybut-2-ylamino)-4-methoxybenzyl]-ketone;
[3,4-dimethoxy-6-(methoxycarbonylmethyl)phenyl]-[α-(methylamino)benzyl]-ketone;
(4-methoxyphenyl)-[α-(butylamino)benzyl]-ketone;
(4-methoxyphenyl)-[α-(1-hydroxyethylamino)-4-methoxybenzyl]-ketone; or
(4-methoxyphenyl)-[α-(1-hydroxyethylamino)benzyl]-ketone.

According to a further feature of the invention there is provided a compound of formula (Ig): 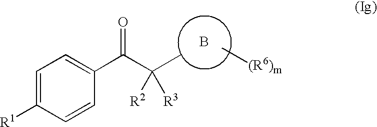
wherein:

R¹ is selected from fluoro, chloro or methyl;

R² is C₁₋₄alkoxy; wherein R² may be optionally substituted on carbon by one or more groups selected from R⁹;

R³ is selected from hydrogen or C₁₋₄alkyl; wherein R³ may be optionally substituted on carbon by one or more groups selected from R⁹;

Ring B is carbocyclyl or a carbon linked heterocyclyl; wherein if said heterocyclyl contains an —NH— moiety that nitrogen may be optionally substituted by a group selected from R¹⁷;

R⁶ is a substituent on carbon and is selected from halo, nitro, cyano, hydroxy, amino, carboxy, carbamoyl, mercapto, sulphamoyl, trifluoromethyl, trifluoromethoxy, C₁₋₄alkyl, C₂₋₄alkenyl, C₂₋₄alkynyl, C₁₋₄alkoxy, C₁₋₄alkanoyl, C₁₋₄alkanoyloxy, N—(C₁₋₄alkyl)amino, N,N—(C₁₋₄alkyl)₂amino, C₁₋₄alkanoylamino, N—(C₁₋₄alkyl)carbamoyl, N,N—(C₁₋₄alkyl)₂carbamoyl, C₁₋₄alkylS(O)ₐ wherein a is 0 to 2, C₁₋₄alkoxycarbonyl, N—(C₁₋₄alkyl)sulphamoyl, N,N—(C₁₋₄alkyl)₂sulphamoyl, C₁₋₄alkylsulphonylamino, carbocyclyl and heterocyclyl; wherein R⁶ may be optionally substituted on carbon by one or more groups selected from R¹⁸; and wherein if said heterocyclyl contains an —NH— moiety that nitrogen may be optionally substituted by a group selected from R¹⁹;

m is 0-3; wherein the values of R⁶ may be the same or different;

R⁹ and R¹⁸ are independently selected from halo, nitro, cyano, hydroxy, amino, carboxy, carbamoyl, mercapto, sulphamoyl, trifluoromethyl, trifluoromethoxy, C₁₋₄alkyl, C₂₋₄alkenyl, C₂₋₄alkynyl, C₁₋₄alkoxy, C₁₋₄alkanoyl, C₁₋₄alkanoyloxy, N—(C₁₋₄alkyl)amino, N,N—(C₁₋₄alkyl)₂amino, C₁₋₄alkanoylamino, N—(C₁₋₄alkyl)carbamoyl, N,N—(C₁₋₄alkyl)₂carbamoyl, C₁₋₄alkylS(O)ₐ wherein a is 0 to 2, C₁₋₄alkoxycarbonyl, N—(C₁₋₄alkyl)sulphamoyl, N,N—(C₁₋₄alkyl)₂sulphamoyl, C₁₋₄alkylsulphonylamino, carbocyclyl and heterocyclyl;

R¹⁷ and R¹⁹ are independently selected from C₁₋₄alkyl, C₁₋₄alkanoyl, C₁₋₄alkylsulphonyl, C₁₋₄alkoxycarbonyl, carbamoyl, N—(C₁₋₄alkyl)carbamoyl, N,N—(C₁₋₄alkyl)carbamoyl, benzyl, benzyloxycarbonyl, benzoyl and phenylsulphonyl;

or a pharmaceutically acceptable salt thereof;

with the proviso that said compound is not (4-methylphenyl)-(α-methoxybenzyl)-ketone;
(4-chlorophenyl)-(α-ethoxy-2-chlorobenzyl)-ketone;
(4-chlorophenyl)-[1-(3-nitroimidazo[1,2-a]pyridin-8-yl)-1-(methoxy)methyl]-ketone;
(4-methylphenyl)-(α-methoxy-α-methylbenzyl)-ketone;
(2,4,6-trimethylphenyl)-(α-methoxy-α-methyl-2,4,6-trimethylbenzyl)-ketone;
(2,4-dichlorophenyl)-(α-methoxybenzyl)-ketone;
(4-fluorophenyl)-(α-methoxybenzyl)-ketone;
(4-methylphenyl)-(α-methoxy-4-methylbenzyl)-ketone;
(4-methylphenyl)-(α-t-butoxy-4-methylbenzyl)-ketone;
(3-nitro-4-chlorophenyl)-(α-methoxy-3-nitro-4-chlorobenzyl)-ketone;
(4-methylphenyl)-(α-but-2-yloxybenzyl)-ketone;
(4-chlorophenyl)-(α-isopropoxy-4-chlorobenzyl)-ketone;
(4-chlorophenyl)-(α-isopropoxybenzyl)-ketone;
(4-methylphenyl)-(α-isopropoxybenzyl)-ketone;
(4-methylphenyl)-(α-isopropoxy-4-methylbenzyl)-ketone;
(4-chlorophenyl)-(α-methoxybenzyl)-ketone;
(4-chlorophenyl)-(α-methoxy-4-chlorobenzyl)-ketone; or
(4-chlorophenyl)-(α-methoxy-α-methyl-4-chlorobenzyl)-ketone.

Accordingly to a further feature of the invention there is provided a compound of formula (Ih): 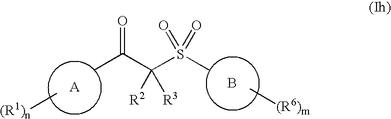
wherein:

Ring A is selected from furyl, thienyl, thiazolyl and pyridyl;

R¹ is selected from halo, nitro, cyano, hydroxy, amino, carboxy, carbamoyl, mercapto, sulphamoyl, C₁₋₄alkyl, C₂₋₄alkenyl, C₂₋₄alkynyl, C₁₋₄alkoxy, C₁₋₄alkanoyl, C₁₋₄alkanoyloxy, N—(C₁₋₄alkyl)amino, N,N—(C₁₋₄alkyl)₂amino, C₁₋₄alkanoylamino, N—(C₁₋₄alkyl)carbamoyl, N,N—(C₁₋₄alkyl)₂carbamoyl, C₁₋₄alkylS(O)ₐ wherein a is 0 to 2, C₁₋₄alkoxycarbonyl, N—(C₁₋₄alkyl)sulphamoyl, N,N—(C₁₋₄alkyl)₂sulphamoyl, C₁₋₄alkylsulphonylamino, carbocyclyl, heterocyclyl, carbocyclylC₀₋₄alkylene-Y— and heterocyclylC₀₋₄alkylene-Y—; or two R¹ on adjacent carbons may form an oxyC₁₋₄alkoxy group; wherein R¹ may be optionally substituted on carbon by one or more groups selected from R⁷; and wherein if said heterocyclyl contains an —NH— moiety that nitrogen may be optionally substituted by a group selected from R⁸;

n is 0-3; wherein the values of R¹ may be the same or different;

R² and R³ are independently selected from hydrogen, hydroxy, amino, cyano, C₁₋₄alkyl, C₁₋₄alkoxy, N—(C₁₋₄alkyl)amino, N,N—(C₁₋₄alkyl)₂amino, carbocyclyl, heterocyclyl, carbocyclylC₁₋₄alkyl and heterocyclylC₁₋₄alkyl; wherein R² and R³ may be independently optionally substituted on carbon by one or more groups selected from R⁹; and wherein if said heterocyclyl contains an —NH— moiety that nitrogen may be optionally substituted by a group selected from R¹⁰;

q is 0 or 1;

p is 0 or 1;

Ring B is a heterocyclyl linked to the sulphonyl of formula (Ih) via a nitrogen atom; wherein if said heterocyclyl contains an —NH— moiety that nitrogen may be optionally substituted by a group selected from R¹⁷;

R⁶ is a substituent on carbon and is selected from halo, nitro, cyano, hydroxy, amino, carboxy, carbamoyl, mercapto, sulphamoyl, trifluoromethyl, trifluoromethoxy, C₁₋₄alkyl, C₂₋₄alkenyl, C₂₋₄alkynyl, C₁₋₄alkoxy, C₁₋₄alkanoyl, C₁₋₄alkanoyloxy, N—(C₁₋₄alkyl)amino, N,N—(C₁₋₄alkyl)₂amino, C₁₋₄alkanoylamino, N—(C₁₋₄alkyl)carbamoyl, N,N—(C₁₋₄alkyl)₂carbamoyl, C₁₋₄alkylS(O)ₐ wherein a is 0 to 2, C₁₋₄alkoxycarbonyl, N—(C₁₋₄alkyl)sulphamoyl, N,N—(C₁₋₄alkyl)₂sulphamoyl, C₁₋₄alkylsulphonylamino, carbocyclyl, heterocyclyl, carbocyclylC₀₋₄alkylene-Y— and heterocyclylC₀₋₄alkylene-Y—; wherein R⁶ may be optionally substituted on carbon by one or more groups selected from R¹⁸; and wherein if said heterocyclyl contains an —NH— moiety that nitrogen may be optionally substituted by a group selected from R¹⁹;

m is 0-3; wherein the values of R⁶ may be the same or different;

Y is —S(O)ₐ—, —O—, —NR²⁰—, —C(O), —C(O)NR²¹—, —NR²²C(O)— or —SO₂NR²³—; wherein a is 0 to 2;

R⁷, R⁹ and R¹⁸ are independently selected from halo, nitro, cyano, hydroxy, amino, carboxy, carbamoyl, mercapto, sulphamoyl, trifluoromethyl, trifluoromethoxy, C₁₋₄alkyl, C₂₋₄alkenyl, C₂₋₄alkynyl, C₁₋₄alkoxy, C₁₋₄alkanoyl, C₁₋₄alkanoyloxy, N—(C₁₋₄alkyl)amino, N,N—(C₁₋₄alkyl)₂amino, C₁₋₄alkanoylamino, N—(C₁₋₄alkyl)carbamoyl, N,N—(C₁₋₄alkyl)₂carbamoyl, C₁₋₄alkylS(O)ₐ wherein a is 0 to 2, C₁₋₄alkoxycarbonyl, N—(C₁₋₄alkyl)sulphamoyl, N,N—(C₁₋₄alkyl)₂sulphamoyl, C₁₋₄alkylsulphonylamino, carbocyclyl and heterocyclyl; wherein R⁷, R⁹ and R¹⁸ may be independently optionally substituted on carbon by one or more R²⁶;

R²⁴ is selected from halo, nitro, cyano, hydroxy, amino, carboxy, carbamoyl, mercapto, sulphamoyl, trifluoromethyl, trifluoromethoxy, C₁₋₄alkyl, C₂₋₄alkenyl, C₂₋₄alkynyl, C₁₋₄alkoxy, C₁₋₄alkanoyl, C₁₋₄alkanoyloxy, N—(C₁₋₄alkyl)amino, N,N—(C₁₋₄alkyl)₂amino, C₁₋₄alkanoylamino, N—(C₁₋₄alkyl)carbamoyl, N,N—(C₁₋₄alkyl)₂carbamoyl, C₁₋₄alkylS(O)ₐ wherein a is 0 to 2, C₁₋₄alkoxycarbonyl, N—(C₁₋₄alkyl)sulphamoyl, N,N—(C₁₋₄alkyl)₂sulphamoyl and C₁₋₄alkylsulphonylamino;

R⁸, R¹⁰, R¹⁷, R¹⁹ and R²⁵ are independently selected from C₁₄alkyl, C₁₋₄alkanoyl, C₁₋₄alkylsulphonyl, C₁₋₄alkoxycarbonyl, carbamoyl, N—(C₁₋₄alkyl)carbamoyl, N,N—(C₁₋₄alkyl)carbamoyl, benzyl, benzyloxycarbonyl, benzoyl and phenylsulphonyl;

R²⁰, R²¹, R²² and R²³ are independently selected from hydrogen, phenyl and C₁₋₄alkyl;

R²⁶ is selected from halo, nitro, cyano, hydroxy, trifluoromethoxy, trifluoromethyl, amino, carboxy, carbamoyl, mercapto, sulphamoyl, methyl, ethyl, methoxy, ethoxy, acetyl, acetoxy, methylamino, ethylamino, dimethylamino, diethylamino, N-methyl-N-ethylamino, acetylamino, N-methylcarbamoyl, N-ethylcarbamoyl, N,N-dimethylcarbamoyl, N,N-diethylcarbamoyl, N-methyl-N-ethylcarbamoyl, methylthio, ethylthio, methylsulphinyl, ethylsulphinyl, mesyl, ethylsulphonyl, methoxycarbonyl, ethoxycarbonyl, N-methylsulphamoyl, N-ethylsulphamoyl, N,N-dimethylsulphamoyl, N,N-diethylsulphamoyl or N-methyl-N-ethylsulphamoyl;

or a pharmaceutically acceptable salt thereof,

with the proviso that said compound is not
(2-nitrofur-5-yl)-(morpholinosulphonylmethyl)-ketone.

According to a further feature of the invention there is provided a compound of formula (Ii): 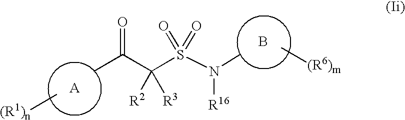
wherein:

Ring A is selected from furyl, thienyl, thiazolyl and pyridyl;

R¹ is selected from halo, nitro, cyano, hydroxy, amino, carboxy, carbamoyl, mercapto, sulphamoyl, C₁₋₄alkyl, C₂₋₄alkenyl, C₂₋₄alkynyl, C₁₋₄alkoxy, C₁₋₄alkanoyl, C₁₋₄alkanoyloxy, N—(C₁₋₄alkyl)amino, N,N-(C₁₋₄alkyl)₂amino, C₁₋₄alkanoylamino, N—(C₁₋₄alkyl)carbamoyl, N,N—(C₁₋₄alkyl)₂carbamoyl, C₁₋₄alkylS(O)ₐ wherein a is 0 to 2, C₁₋₄alkoxycarbonyl, N—(C₁₋₄alkyl)sulphamoyl, N,N—(C₁₋₄alkyl)₂sulphamoyl, C₁₋₄alkylsulphonylamino, carbocyclyl, heterocyclyl, carbocyclylC₀₋₄alkylene-Y— and heterocyclylC₀₋₄alkylene-Y—; or two R¹ on adjacent carbons may form an oxyC₁₋₄alkoxy group; wherein R¹ may be optionally substituted on carbon by one or more groups selected from R⁷; and wherein if said heterocyclyl contains an —NH— moiety that nitrogen may be optionally substituted by a group selected from R⁸;

n is 0-3; wherein the values of R¹ may be the same or different;

R² and R³ are independently selected from hydrogen, hydroxy, amino, cyano, C₁₋₄alkyl, C₁₋₄alkoxy, N—(C₁₋₄alkyl)amino, N,N—(C₁₋₄alkyl)₂amino, carbocyclyl, heterocyclyl, carbocyclylC₁₋₄alkyl and heterocyclylC₁₋₄alkyl; wherein R² and R³ may be independently optionally substituted on carbon by one or more groups selected from R⁹; and wherein if said heterocyclyl contains an —NH— moiety that nitrogen may be optionally substituted by a group selected from R¹⁰;

q is 0 or 1;

p is 0 or 1;

Ring B is carbocyclyl or heterocyclyl; wherein if said heterocyclyl contains an —NH— moiety that nitrogen may be optionally substituted by a group selected from R¹⁷;

R⁶ is a substituent on carbon and is selected from halo, nitro, cyano, hydroxy, amino, carboxy, carbamoyl, mercapto, sulphamoyl, trifluoromethyl, trifluoromethoxy, C₁₋₄alkyl, C₂₋₄alkenyl, C₂₋₄alkynyl, C₁₋₄alkoxy, C₁₋₄alkanoyl, C₁₋₄alkanoyloxy, N—(C₁₋₄alkyl)amino, N,N—(C₁₋₄alkyl)₂amino, C₁₋₄alkanoylamino, N—(C₁₋₄alkyl)carbamoyl, N,N—(C₁₋₄alkyl)₂carbamoyl, C₁₋₄alkylS(O)ₐ wherein a is 0 to 2, C₁₋₄alkoxycarbonyl, N—(C₁₋₄alkyl)sulphamoyl, N,N—(C₁₋₄alkyl)₂sulphamoyl, C₁₋₄alkylsulphonylamino, carbocyclyl, heterocyclyl, carbocyclylC₀₋₄alkylene-Y— and heterocyclylC₀₋₄alkylene-Y—; wherein R⁶ may be optionally substituted on carbon by one or more groups selected from R¹⁸; and wherein if said heterocyclyl contains an —NH— moiety that nitrogen may be optionally substituted by a group selected from R¹⁹;

m is 0-3; wherein the values of R⁶ may be the same or different;

Y is —S(O)ₐ—, —O—, —NR²⁰—, —C(O), —C(O)NR²¹—, —NR²²C(O)— or —SO₂NR²³—; wherein a is 0 to 2;

R⁷, R⁹ and R¹⁸ are independently selected from halo, nitro, cyano, hydroxy, amino, carboxy, carbamoyl, mercapto, sulphamoyl, trifluoromethyl, trifluoromethoxy, C₁₋₄alkyl, C₂₋₄alkenyl, C₂₋₄alkynyl, C₁₋₄alkoxy, C₁₋₄alkanoyl, C₁₋₄alkanoyloxy, N—(C₁₋₄alkyl)amino, N,N—(C₁₋₄alkyl)₂amino, C₁₋₄alkanoylamino, N—(C₁₋₄alkyl)carbamoyl, N,N—(C₁₋₄alkyl)₂carbamoyl, C₁₋₄alkylS(O)ₐ wherein a is 0 to 2, C₁₋₄alkoxycarbonyl, N—(C₁₋₄alkyl)sulphamoyl, N,N—(C₁₋₄alkyl)₂sulphamoyl, C₁₋₄alkylsulphonylamino, carbocyclyl and heterocyclyl; wherein R⁷, R⁹ and R¹⁸ may be independently optionally substituted on carbon by one or more R²⁶;

R²⁴ is selected from halo, nitro, cyano, hydroxy, amino, carboxy, carbamoyl, mercapto, sulphamoyl, trifluoromethyl, trifluoromethoxy, C₁₋₄alkyl, C₂₋₄alkenyl, C₂₋₄alkynyl, C₁₋₄alkoxy, C₁₋₄alkanoyl, C₁₋₄alkanoyloxy, N—(C₁₋₄alkyl)amino, N,N—(C₁₋₄alkyl)₂amino, C₁₋₄alkanoylamino, N—(C₁₋₄alkyl)carbamoyl, N,N—(C₁₋₄alkyl)₂carbamoyl, C₁₋₄alkylS(O)ₐ wherein a is 0 to 2, C₁₋₄alkoxycarbonyl, N—(C₁₋₄alkyl)sulphamoyl, N,N—(C₁₋₄alkyl)₂sulphamoyl and C₁₋₄alkylsulphonylamino;

R⁸, R¹⁰, R¹⁷, R¹⁹ and R²⁵ are independently selected from C₁₋₄alkyl, C₁₋₄alkanoyl, C₁₋₄alkylsulphonyl, C₁₋₄alkoxycarbonyl, carbamoyl, N—(C₁₋₄alkyl)carbamoyl, N,N—(C₁₋₄alkyl)carbamoyl, benzyl, benzyloxycarbonyl, benzoyl and phenylsulphonyl;

R¹⁶, R²⁰, R²¹, R²² and R²³ are independently selected from hydrogen, phenyl and C₁₋₄alkyl;

R²⁶ is selected from halo, nitro, cyano, hydroxy, trifluoromethoxy, trifluoromethyl, amino, carboxy, carbamoyl, mercapto, sulphamoyl, methyl, ethyl, methoxy, ethoxy, acetyl, acetoxy, methylamino, ethylamino, dimethylamino, diethylamino, N-methyl-N-ethylamino, acetylamino, N-methylcarbamoyl, N-ethylcarbamoyl, N,N-dimethylcarbamoyl, N,N-diethylcarbamoyl, N-methyl-N-ethylcarbamoyl, methylthio, ethylthio, methylsulphinyl, ethylsulphinyl, mesyl, ethylsulphonyl, methoxycarbonyl, ethoxycarbonyl, N-methylsulphamoyl, N-ethylsulphamoyl, N,N-dimethylsulphamoyl, N,N-diethylsulphamoyl or N-methyl-N-ethylsulphamoyl;

or a pharmaceutically acceptable salt thereof.

According to a further feature of the invention, there is provided a compound of formula (Ij): 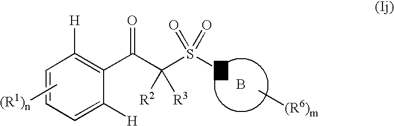
wherein:

R¹ is selected from halo, nitro, cyano, hydroxy, amino, carboxy, carbamoyl, mercapto, sulphamoyl, C₁₋₆alkyl, C₂₋₆alkenyl, C₂₋₆alkynyl, C₁₋₆alkoxy, C₁₋₆alkanoyl, C₁₋₆alkanoyloxy, N—(C₁₋₆alkyl)amino, N,N—(C₁₋₆alkyl)₂amino, C₁₋₆alkanoylamino, N—(C₁₋₆alkyl)carbamoyl, N,N—(C₁₋₆alkyl)₂carbamoyl, C₁₋₆alkylS(O)ₐ wherein a is 0 to 2, C₁₋₆alkoxycarbonyl, N—(C₁₋₆alkyl)sulphamoyl, N,N—(C₁₋₆alkyl)₂sulphamoyl, C₁₋₆alkylsulphonylamino, carbocyclyl, heterocyclyl, carbocyclylC₀₋₆alkylene-Y— and heterocyclylC₀₋₆alkylene-Y—; or two R¹ on adjacent carbons may form an oxyC₁₋₄alkoxy group or a C₃₋₅alkylene group; wherein R¹ may be optionally substituted on carbon by one or more groups selected from R⁷; and wherein if said heterocyclyl contains an —NH— moiety that nitrogen may be optionally substituted by a group selected from R⁸;

n is 0-3; wherein the values of R¹ may be the same or different;

R² and R³ are independently selected from hydrogen, hydroxy, amino, cyano, C₁₋₄alkyl, C₁₋₄alkoxy, N—(C₁₋₄alkyl)amino, N,N—(C₁₋₄alkyl)₂amino, C₁₋₄alkylS(O)ₐ wherein a is 0 to 2, C₁₋₄alkoxycarbonyl, C₁₋₄alkoxycarbonylamino, C₁₋₄alkanoyloxy, carbocyclyl, heterocyclyl, carbocyclylC₁₋₄alkyl and heterocyclylC₁₋₄alkyl; or R² and R³ together form oxo or a spiro attached heterocyclyl; wherein R² and R³ may be independently optionally substituted on carbon by one or more groups selected from R⁹; and wherein if said heterocyclyl contains an —NH— moiety that nitrogen may be optionally substituted by a group selected from R¹⁰;

Ring B is a heterocyclyl linked to the sulphonyl of formula (Ij) via a nitrogen atom; wherein if said heterocyclyl contains an —NH— moiety that nitrogen may be optionally substituted by a group selected from R¹⁷;

R⁶ is a substituent on carbon and is selected from halo, nitro, cyano, hydroxy, amino, carboxy, carbamoyl, mercapto, sulphamoyl, trifluoromethyl, trifluoromethoxy, C₁₋₄alkyl, C₂₋₄alkenyl, C₂₋₄alkynyl, C₁₋₄alkoxy, C₁₋₄alkanoyl, C₁₋₄alkanoyloxy, N—(C₁₋₄alkyl)amino, N,N—(C₁₋₄alkyl)₂amino, C₁₋₄alkanoylamino, N—(C₁₋₄alkyl)carbamoyl, N,N—(C₁₋₄alkyl)₂carbamoyl, C₁₋₄alkylS(O)ₐ wherein a is 0 to 2, C₁₋₄alkoxycarbonyl, N—(C₁₋₄alkyl)sulphamoyl, N,N—(C₁₋₄alkyl)₂sulphamoyl, C₁₋₄alkylsulphonylamino, carbocyclyl, heterocyclyl, carbocyclylC₀₋₄alkylene-Y— and heterocyclylC₀₋₄alkylene-Y—; wherein R⁶ may be optionally substituted on carbon by one or more groups selected from R¹⁸; and wherein if said heterocyclyl contains an —NH— moiety that nitrogen may be optionally substituted by a group selected from R¹⁹,

m is 0-3; wherein the values of R⁶ may be the same or different;

Y is —S(O)ₐ—, —O—, —NR²⁰—, —C(O)—, —C(O)NR²¹—, —NR²²C(O)— or —SO₂NR²³—; wherein a is 0 to 2;

R⁷, R⁹ and R¹⁸ are independently selected from halo, nitro, cyano, hydroxy, amino, carboxy, carbamoyl, mercapto, sulphamoyl, trifluoromethyl, trifluoromethoxy, C₁₋₄alkyl, C₂₋₄alkenyl, C₂₋₄alkynyl, C₁₋₄alkoxy, C₁₋₄alkanoyl, C₁₋₄alkanoyloxy, N—(C₁₋₄alkyl)amino, N,N—(C₁₋₄alkyl)₂amino, C₁₋₄alkanoylamino, N—(C₁₋₄alkyl)carbamoyl, N,N—(C₁₋₄alkyl)₂carbamoyl, C₁₋₄alkylS(O)ₐ wherein a is 0 to 2, C₁₋₄alkoxycarbonyl, N—(C₁₋₄alkyl)sulphamoyl, N,N—(C₁₋₄alkyl)₂sulphamoyl, C₁₋₄alkylsulphonylamino, carbocyclyl and heterocyclyl; wherein R⁷, R⁹ and R¹⁸ may be independently optionally substituted on carbon by one or more R²⁶;

R⁸, R¹⁰, R¹⁷ and R¹⁹ are independently selected from C₁₋₄alkyl, C₁₋₄alkanoyl, C₁₋₄alkylsulphonyl, C₁₋₄alkoxycarbonyl, carbamoyl, N—(C₁₋₄alkyl)carbamoyl, N,N—(C₁₋₄alkyl)carbamoyl, benzyl, benzyloxycarbonyl, benzoyl, carbocyclyl, heterocyclyl and phenylsulphonyl; wherein R⁸, R¹⁰, R¹⁷ and R¹⁹ may be independently optionally substituted on carbon by one or more R²⁷;

R²⁰, R²¹, R²² and R²³ are independently selected from hydrogen, phenyl, C₁₋₄alkylsulphonyl and C₁₋₄alkyl;

R²⁶ and R²⁷ are independently selected from selected from halo, nitro, cyano, hydroxy, trifluoromethoxy, trifluoromethyl, amino, carboxy, carbamoyl, mercapto, sulphamoyl, methyl, ethyl, methoxy, ethoxy, acetyl, acetoxy, methylamino, ethylamino, dimethylamino, diethylamino, N-methyl-N-ethylamino, acetylamino, N-methylcarbamoyl, N-ethylcarbamoyl, N,N-dimethylcarbamoyl, N,N-diethylcarbamoyl, N-methyl-N-ethylcarbamoyl, methylthio, ethylthio, methylsulphinyl, ethylsulphinyl, mesyl, ethylsulphonyl, methoxycarbonyl, ethoxycarbonyl, N-methylsulphamoyl, N-ethylsulphamoyl, N,N-dimethylsulphamoyl, N,N-diethylsulphamoyl or N-methyl-N-ethylsulphamoyl;

or a pharmaceutically acceptable salt thereof;

with the proviso that said compound is not
(phenyl)-[α-(pyrrolidin-1-ylsulphonyl)benzyl]-ketone;
(phenyl)-[α-(morpholinosulphonyl)benzyl]-ketone;
(4-carbamoylphenyl)-[4-(5-chloropyridin-2-yloxy)piperidin-1-ylsulphonylmethyl]-ketone;
(4-carbamoylphenyl)-[4-(4-fluorophenyl)piperidin-1-ylsulphonylmethyl]-ketone;
(4-fluorophenyl)-[4-(5-chloropyridin-2-yloxy)piperidin-1-ylsulphonylmethyl]-ketone;
(phenyl)-[4-(5-chloropyridin-2-yloxy)piperidin-1-ylsulphonylmethyl]-ketone;
(4-chlorophenyl)-(piperazin-1-ylsulphonylmethyl)-ketone;
(4-chlorophenyl)-[4-(t-butoxycarbonyl)piperazin-1-ylsulphonylmethyl]-ketone;
(4-hydroxyphenyl)-(morpholinosulphonylmethyl)-ketone; or
(phenyl)-(1,2,3,4-tetrahydroisoquinolin-2-ylsulphonylmethyl)-ketone; and with the proviso that when R² and R³ are hydrogen, m is 0 and Ring B is 4-methylpiperazin-1-yl, then (R¹)ₙ is not hydrogen, 4-fluoro, 4-nitro, 3,4-dimethoxy, 4-methoxy, 4-t-butyl, 4-trifluoromethyl or 4-chloro; and with the proviso that when R² and R³ are hydrogen, m is 0 and Ring B is morpholino then (R¹)ₙ is not hydrogen, 4-dimethylamino, 4-nitro, 4-methoxy, 4-t-butyl, 4-trifluoromethyl, 4-fluoro or 4-chloro.

According to a further feature of the invention, there is provided a compound of formula (Ik): 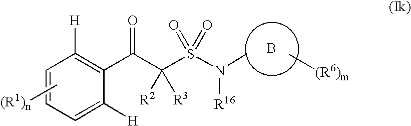
wherein:

R¹ is selected from halo, nitro, cyano, hydroxy, amino, carboxy, carbamoyl, mercapto, sulphamoyl, C₁₋₆alkyl, C₂₋₆alkenyl, C₂₋₆alkynyl, C₁₋₆alkoxy, C₁₋₆alkanoyl, C1-6alkanoyloxy, N—(C₁₋₆alkyl)amino, N,N—(C₁₋₆alkyl)₂amino, C₁₋₆alkanoylamino, N—(C₁₋₆alkyl)carbamoyl, N,N—(C₁₋₆alkyl)₂carbamoyl, C₁₋₆alkylS(O)ₐ wherein a is 0 to 2, C₁₋₆alkoxycarbonyl, N—(C₁₋₆alkyl)sulphamoyl, N,N—(C₁₋₆alkyl)₂sulphamoyl, C₁₋₆alkylsulphonylamino, carbocyclyl, heterocyclyl, carbocyclylC₀₋₆alkylene-Y— and heterocyclylC₀₋₆alkylene-Y—; or two R¹ on adjacent carbons may form an oxyC₁₋₄alkoxy group or a C₃₋₅alkylene group; wherein R¹ may be optionally substituted on carbon by one or more groups selected from R⁷; and wherein if said heterocyclyl contains an —NH— moiety that nitrogen may be optionally substituted by a group selected from R⁸;

n is 0-3; wherein the values of R¹ may be the same or different;

R² and R³ are independently selected from hydrogen, hydroxy, amino, cyano, C₁₋₄alkyl, C₁₋₄alkoxy, N—(C₁₋₄alkyl)amino, N,N—(C₁₋₄alkyl)₂amino, C₁₋₄alkylS(O)ₐ wherein a is 0 to 2, C₁₋₄alkoxycarbonyl, C₁₋₄alkoxycarbonylamino, C₁₋₄alkanoyloxy, carbocyclyl, heterocyclyl, carbocyclylC₁₋₄alkyl and heterocyclylC₁₋₄alkyl; or R² and R³ together form oxo or a spiro attached heterocyclyl; wherein R² and R³ may be independently optionally substituted on carbon by one or more groups selected from R⁹; and wherein if said heterocyclyl contains an —NH— moiety that nitrogen may be optionally substituted by a group selected from R¹⁰;

Ring B is carbocyclyl or heterocyclyl; wherein if said heterocyclyl contains an —NH— moiety that nitrogen may be optionally substituted by a group selected from R¹⁷;

R⁶ is a substituent on carbon and is selected from halo, nitro, cyano, hydroxy, amino, carboxy, carbamoyl, mercapto, sulphamoyl, trifluoromethyl, trifluoromethoxy, C₁₋₄alkyl, C₂₋₄alkenyl, C₂₋₄alkynyl, C₁₋₄alkoxy, C₁₋₄alkanoyl, C₁₋₄alkanoyloxy, N—(C₁₋₄alkyl)amino, N,N—(C₁₋₄alkyl)₂amino, C₁₋₄alkanoylamino, N—(C₁₋₄alkyl)carbamoyl, N,N—(C₁₋₄alkyl)₂carbamoyl, C₁₋₄alkylS(O)ₐ wherein a is 0 to 2, C₁₋₄alkoxycarbonyl, N—(C₁₋₄alkyl)sulphamoyl, N,N—(C₁₋₄alkyl)₂sulphamoyl, C₁₋₄alkylsulphonylamino, carbocyclyl, heterocyclyl, carbocyclylC₀₋₄alkylene-Y— and heterocyclylC₀₋₄alkylene-Y—; wherein R⁶ may be optionally substituted on carbon by one or more groups selected from R¹⁸; and wherein if said heterocyclyl contains an —NH— moiety that nitrogen may be optionally substituted by a group selected from R¹⁹;

m is 0-3; wherein the values of R⁶ may be the same or different;

Y is —S(O)ₐ—, —O—, —NR²⁰—, —C(O)—, —C(O)NR²¹—, —NR²²C(O)— or —SO₂NR²³—; wherein a is 0 to 2;

R⁷, R⁹ and R¹⁸ are independently selected from halo, nitro, cyano, hydroxy, amino, carboxy, carbamoyl, mercapto, sulphamoyl, trifluoromethyl, trifluoromethoxy, C₁₋₄alkyl, C₂₋₄alkenyl, C₂₋₄alkynyl, C₁₋₄alkoxy, C₁₋₄alkanoyl, C₁₋₄alkanoyloxy, N—(C₁₋₄alkyl)amino, N,N—(C₁₋₄alkyl)₂amino, C₁₋₄alkanoylamino, N—(C₁₋₄alkyl)carbamoyl, N,N-(C₁₋₄alkyl)₂carbamoyl, C₁₋₄alkylS(O)ₐ wherein a is 0 to 2, C₁₋₄alkoxycarbonyl, N—(C₁₋₄alkyl)sulphamoyl, N,N—(C₁₋₄alkyl)₂sulphamoyl, C₁₋₄alkylsulphonylamino, carbocyclyl and heterocyclyl; wherein R⁷, R⁹ and R¹⁸ may be independently optionally substituted on carbon by one or more R²⁶;

R⁸, R¹⁰, R¹⁷ and R¹⁹ are independently selected from C₁₋₄alkyl, C₁₋₄alkanoyl, C₁₋₄alkylsulphonyl, C₁₋₄alkoxycarbonyl, carbamoyl, N—(C₁₋₄alkyl)carbamoyl, N,N—(C₁₋₄alkyl)carbamoyl, benzyl, benzyloxycarbonyl, benzoyl, carbocyclyl, heterocyclyl and phenylsulphonyl; wherein R⁸, R¹⁰, R¹⁷ and R¹⁹ may be independently optionally substituted on carbon by one or more R²⁷;

R¹⁶, R²⁰, R²¹, R²² and R²³ are independently selected from hydrogen, phenyl, C₁₋₄alkylsulphonyl and C₁₋₄alkyl;

R²⁶ and R²⁷ are independently selected from selected from halo, nitro, cyano, hydroxy, trifluoromethoxy, trifluoromethyl, amino, carboxy, carbamoyl, mercapto, sulphamoyl, methyl, ethyl, methoxy, ethoxy, acetyl, acetoxy, methylamino, ethylamino, dimethylamino, diethylamino, N-methyl-N-ethylamino, acetylamino, N-methylcarbamoyl, N-ethylcarbamoyl, N,N-dimethylcarbamoyl, N,N-diethylcarbamoyl, N-methyl-N-ethylcarbamoyl, methylthio, ethylthio, methylsulphinyl, ethylsulphinyl, mesyl, ethylsulphonyl, methoxycarbonyl, ethoxycarbonyl, N-methylsulphamoyl, N-ethylsulphamoyl, N,N-dimethylsulphamoyl, N,N-diethylsulphamoyl or N-methyl-N-ethylsulphamoyl;

or a pharmaceutically acceptable salt thereof;

with the proviso that said compound is not
(phenyl)-(5-methylpyrazol-3-ylaminosulphonylmethyl)-ketone;
(phenyl)-[(2-methyl-6-methoxy-2,3-dihydrobenzofuran-4-yl)aminosulphonylmethyl]-ketone;
(phenyl)-(1-phenyl-3-methylpyrazol-5-ylaminosulphonylmethyl)-ketone;
(phenyl)-[1-(cyclohexyl-N-methylaminosulphonyl)ethyl]-ketone;
(phenyl)-[1-(phenyl-N-methylaminosulphonyl)ethyl]-ketone;
(phenyl)-(cyclohexylaminosulphonylmethyl)-ketone;
(phenyl)-[(2-phenyl-4-acetyl-5-methylimidazol-3-yl]-N-methylaminosulphonylmethyl]-ketone; (phenyl)-[(2-phenyl-4-acetyl-5-methylimidazol-3-yl]aminosulphonylmethyl]-ketone;
(phenyl)-(2,4,5,6,7,8-hexahydrocycloheptapyrazol-3-ylaminosulphonylmethyl]-ketone;
(phenyl)-(4,5,6,7-tetrahydro-2H-indazol-3-ylaminosulphonylmethyl]-ketone;
(phenyl)-[(4-phenyl-5-methylpyrazol-3-yl)aminosulphonylmethyl]-ketone;
(phenyl)-[3-(1-carboxymethyl-3-methyl-4-oxo-1,2,3,4-tetrahydrophthalazin-2-yl)anilinosulphonylmethyl]-ketone;
(phenyl)-{3-[1-(methoxycarbonylmethyl)-3-methyl-4-oxo-1,2,3,4-tetrahydrophthalazin-2-yl]anilinosulphonylmethyl}-ketone; (phenyl)-(4-methylanilinosulphonylmethyl)-ketone;
(phenyl)-(2-benzoyl-4-chloroanilinosulphonylmethyl)-ketone;
(phenyl)-(2,3-dimethylanilinosulphonylmethyl)-ketone;
(phenyl)-(3,4-dimethylanilinosulphonylmethyl)-ketone;
(phenyl)-(3-methylanilinosulphonylmethyl)-ketone;
(phenyl)-(3-methoxyanilinosulphonylmethyl)-ketone;
(phenyl)-(anilinosulphonylmethyl)-ketone; (phenyl)-(2-acetylanilinosulphonylmethyl)-ketone;
or (phenyl)-[α-(N-ethylanilinosulphonyl)benzyl]-ketone.

According to a further feature of the invention, there is provided a compound of formula (Il): 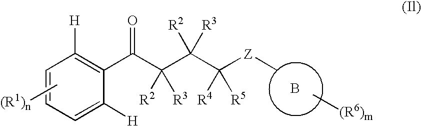
wherein:

R¹ is selected from halo, nitro, cyano, hydroxy, amino, carboxy, carbamoyl, mercapto, sulphamoyl, C₁₋₆alkyl, C₂₋₆alkenyl, C₂₋₆alkynyl, C₁₋₆alkoxy, C₁₋₆alkanoyl, C₁₋₆alkanoyloxy, N—(C₁₋₆alkyl)amino, N,N—(C₁₋₆alkyl)₂amino, C₁₋₆alkanoylamino, N—(C₁₋₄alkyl)carbamoyl, N,N—(C₁₋₆alkyl)₂carbamoyl, C₁₋₆alkylS(O)ₐ wherein a is 0 to 2, C₁₋₆alkoxycarbonyl, N—(C₁₋₆alkyl)sulphamoyl, N,N—(C₁₋₆alkyl)₂sulphamoyl, C₁₋₆alkylsulphonylamino, carbocyclyl, heterocyclyl, carbocyclylC₀₋₆alkylene-Y— and heterocyclylC₀₋₆alkylene-Y—; or two R¹ on adjacent carbons may form an oxyC₁₋₄alkoxy group or a C₃₋₅alkylene group; wherein R¹ may be optionally substituted on carbon by one or more groups selected from R⁷; and wherein if said heterocyclyl contains an —NH— moiety that nitrogen may be optionally substituted by a group selected from R⁸;

n is 0-3; wherein the values of R¹ may be the same or different;

R², R³, R⁴ and R⁵ are independently selected from hydrogen, hydroxy, amino, cyano, C₁₋₄alkyl, C₁₋₄alkoxy, N—(C₁₋₄alkyl)amino, N,N—(C₁₋₄alkyl)₂amino, C₁₋₄alkylS(O)ₐ wherein a is 0 to 2, C₁₋₄alkoxycarbonyl, C₁₋₄alkoxycarbonylamino, C₁₋₄alkanoyloxy, carbocyclyl, heterocyclyl, carbocyclylC₁₋₄alkyl and heterocyclylC₁₋₄alkyl; or R² and R³ together form oxo or a spiro attached heterocyclyl; wherein R², R³, R⁴ and R⁵ may be independently optionally substituted on carbon by one or more groups selected from R⁹; and wherein if said heterocyclyl contains an —NH— moiety that nitrogen may be optionally substituted by a group selected from R¹⁰;

Z is selected from —NR¹⁵C(O)— or —NR¹⁶SO₂—;

Ring B is carbocyclyl or heterocyclyl; wherein if said heterocyclyl contains an —NH— moiety that nitrogen may be optionally substituted by a group selected from R¹⁷;

R⁶ is a substituent on carbon and is selected from halo, nitro, cyano, hydroxy, amino, carboxy, carbamoyl, mercapto, sulphamoyl, trifluoromethyl, trifluoromethoxy, C₁₋₄alkyl, C₂₋₄alkenyl, C₂₋₄alkyl, C₁₋₄alkoxy, C₁₋₄alkanoyl, C₁₋₄alkanoyloxy, N—(C₁₋₄alkyl)amino, N,N—(C₁₋₄alkyl)₂amino, C₁₋₄alkanoylamino, N—(C₁₋₄alkyl)carbamoyl, N,N—(C₁₋₄alkyl)₂carbamoyl, C₁₋₄alkylS(O)ₐ wherein a is 0 to 2, C₁₋₄alkoxycarbonyl, N—(C₁₋₄alkyl)sulphamoyl, N,N—(C₁₋₄alkyl)₂sulphamoyl, C₁₋₄alkylsulphonylamino, carbocyclyl, heterocyclyl, carbocyclylC₀₋₄alkylene-Y— and heterocyclylC₀₋₄alkylene-Y—; wherein R⁶ may be optionally substituted on carbon by one or more groups selected from R¹⁸; and wherein if said heterocyclyl contains an —NH— moiety that nitrogen may be optionally substituted by a group selected from R¹⁹;

m is 0-3; wherein the values of R⁶ may be the same or different;

Y is —S(O)ₐ—, —O—, —NR²⁰—, —C(O)—, —C(O)NR²¹—, —NR²²C(O)— or —SO₂NR²³—; wherein a is 0 to 2;

R⁷, R⁹ and R¹⁸ are independently selected from halo, nitro, cyano, hydroxy, amino, carboxy, carbamoyl, mercapto, sulphamoyl, trifluoromethyl, trifluoromethoxy, C₁₋₄alkyl, C₂₋₄alkenyl, C₂₋₄alkynyl, C₁₋₄alkoxy, C₁₋₄alkanoyl, C₁₋₄alkanoyloxy, N—(C₁₋₄alkyl)amino, N,N—(C₁₋₄alkyl)₂amino, C₁₋₄alkanoylamino, N—(C₁₋₄alkyl)carbamoyl, N,N—(C₁₋₄alkyl)₂carbamoyl, C₁₋₄alkylS(O)ₐ wherein a is 0 to 2, C₁₋₄alkoxycarbonyl, N—(C₁₋₄alkyl)sulphamoyl, N,N—(C₁₋₄alkyl)₂sulphamoyl, C₁₋₄alkylsulphonylamino, carbocyclyl and heterocyclyl; wherein R⁷, R⁹ and R¹⁸ may be independently optionally substituted on carbon by one or more R²⁶;

R⁸, R¹⁰, R¹⁷ and R¹⁹ are independently selected from C₁₋₄alkyl, C₁₋₄alkanoyl, C₁₋₄alkylsulphonyl, C₁₋₄alkoxycarbonyl, carbamoyl, N—(C₁₋₄alkyl)carbamoyl, N,N—(C₁₋₄alkyl)carbamoyl, benzyl, benzyloxycarbonyl, benzoyl, carbocyclyl, heterocyclyl and phenylsulphonyl; wherein R⁸, R¹⁰, R¹⁷ and R¹⁹ may be independently optionally substituted on carbon by one or more R²⁷;

R¹⁵, R¹⁶, R²⁰, R²¹, R²² and R²³ are independently selected from hydrogen, phenyl, C₁₋₄alkylsulphonyl and C₁₋₄alkyl;

R²⁶ and R²⁷ are independently selected from selected from halo, nitro, cyano, hydroxy, trifluoromethoxy, trifluoromethyl, amino, carboxy, carbamoyl, mercapto, sulphamoyl, methyl, ethyl, methoxy, ethoxy, acetyl, acetoxy, methylamino, ethylamino, dimethylamino, diethylamino, N-methyl-N-ethylamino, acetylamino, N-methylcarbamoyl, N-ethylcarbamoyl, N,N-dimethylcarbamoyl, N,N-diethylcarbamoyl, N-methyl-N-ethylcarbamoyl, methylthio, ethylthio, methylsulphinyl, ethylsulphinyl, mesyl, ethylsulphonyl, methoxycarbonyl, ethoxycarbonyl, N-methylsulphamoyl, N-ethylsulphamoyl, N,N-dimethylsulphamoyl, N,N-diethylsulphamoyl or N-methyl-N-ethylsulphamoyl;

or a pharmaceutically acceptable salt thereof;

with the proviso that said compound is not
(4-chlorophenyl)-[3-methyl-2-(benzoylamino)butyrylmethyl]-ketone;
(phenyl)-[(S)-4-(4-methylphenylsuphonylamino)but-2-yl]-ketone;
(phenyl)-{3-[2-(methoxycarbonyl)phenylsulphonylamino]propyl}-ketone;
(4-bromophenyl)-(benzoylaminopropyl)-ketone;
(4-fluorophenyl)-[4-(benzoylamino)but-2-yl]-ketone;
(phenyl)-[4-(benzoylamino)but-2-yl]-ketone;
(phenyl)-[α-(benzoylaminoethyl)benzyl]-ketone;
(phenyl)-[4-(benzoylamino)-2-methylbut-2-yl]-ketone;
(phenyl)-[α-(benzoylaminoethyl)-α-phenylbenzyl]-ketone;
(4-methoxyphenyl)-{3-[N-(4-methylphenylsulphonyl)-N-phenylamino]propyl}-ketone;
(phenyl)-[3-(benzoylamino)-2-phenylpropyl]-ketone;
(phenyl)-{3-[N-(4-methylphenylsulphonyl)-N-phenylamino]propyl}-ketone;
(4-hydroxyphenyl)-[3-(benzoylamino)propyl]-ketone;
(4-methoxyphenyl)-[3-(benzoylamino)propyl]-ketone;
(3,4-dimethoxyphenyl)-[3-(benzoylamino)propyl]-ketone;
(phenyl)[3-(benzoylamino)propyl]-ketone; or
(phenyl)-[3-(4-nitrobenzoylamino)propyl]-ketone.

According to a further feature of the invention, there is provided a compound of formula (Im): 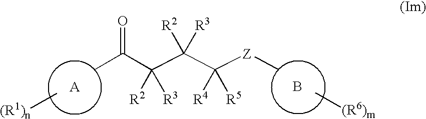
wherein:

Ring A is pyridyl, thiazolyl, thienyl or furyl;

R¹ is selected from halo, nitro, cyano, hydroxy, amino, carboxy, carbamoyl, mercapto, sulphamoyl, C₁₋₆alkyl, C₂₋₆alkenyl, C₂₋₆alkynyl, C₁₋₆alkoxy, C₁₋₆alkanoyl, C₁₋₆alkanoyloxy, N—(C₁₋₆alkyl)amino, N,N—(C₁₋₆alkyl)₂amino, C₁₋₆alkanoylamino, N—(C₁₋₆alkyl)carbamoyl, N,N—(C₁₋₆alkyl)₂carbamoyl, C₁₋₆alkylS(O)ₐ wherein a is 0 to 2, C₁₋₆alkoxycarbonyl, N—(C₁₋₆alkyl)sulphamoyl, N,N—(C₁₋₆alkyl)₂sulphamoyl, C₁₋₆alkylsulphonylamino, carbocyclyl, heterocyclyl, carbocyclylC₀₋₆alkylene-Y— and heterocyclylC₀₋₆alkylene-Y—; or two R¹ on adjacent carbons may form an oxyC₁₋₄alkoxy group or a C₃₋₅alkylene group; wherein R¹ may be optionally substituted on carbon by one or more groups selected from R⁷; and wherein if said heterocyclyl contains an —NH— moiety that nitrogen may be optionally substituted by a group selected from R⁸;

n is 0-3; wherein the values of R¹ may be the same or different;

R², R³, R⁴ and R⁵ are independently selected from hydrogen, hydroxy, amino, cyano, C₁₋₄alkyl, C₁₋₄alkoxy, N—(C₁₋₄alkyl)amino, N,N—(C₁₋₄alkyl)₂amino, C₁₋₄alkylS(O)ₐ wherein a is 0 to 2, C₁₋₄alkoxycarbonyl, C₁₋₄alkoxycarbonylamino, C₁₋₄alkanoyloxy, carbocyclyl, heterocyclyl, carbocyclylC₁₋₄alkyl and heterocyclylC₁₋₄alkyl; or R² and R³ together form oxo or a spiro attached heterocyclyl; wherein R², R³, R⁴ and R⁵ may be independently optionally substituted on carbon by one or more groups selected from R⁹; and wherein if said heterocyclyl contains an —NH— moiety that nitrogen may be optionally substituted by a group selected from R¹⁰;

Z is selected from —NR¹⁵C(O)— or —NR¹⁶SO₂—;

Ring B is carbocyclyl or heterocyclyl; wherein if said heterocyclyl contains an —NH— moiety that nitrogen may be optionally substituted by a group selected from R¹⁷;

R⁶ is a substituent on carbon and is selected from halo, nitro, cyano, hydroxy, amino, carboxy, carbamoyl, mercapto, sulphamoyl, trifluoromethyl, trifluoromethoxy, C₁₋₄alkyl, C₂₋₄alkenyl, C₂₋₄alkynyl, C₁₋₄alkoxy, C₁₋₄alkanoyl, C₁₋₄alkanoyloxy, N—(C₁₋₄alkyl)amino, N,N—(C₁₋₄alkyl)₂amino, C₁₋₄alkanoylamino, N—(C₁₋₄alkyl)carbamoyl, N,N—(C₁₋₄alkyl)₂carbamoyl, C₁₋₄alkylS(O)ₐ wherein a is 0 to 2, C₁₋₄alkoxycarbonyl, N—(C₁₋₄alkyl)sulphamoyl, N,N—(C₁₋₄alkyl)₂sulphamoyl, C₁₋₄alkylsulphonylamino, carbocyclyl, heterocyclyl, carbocyclylC₀₋₄alkylene-Y— and heterocyclylC₀₋₄alkylene-Y—; wherein R⁶ may be optionally substituted on carbon by one or more groups selected from R¹⁸; and wherein if said heterocyclyl contains an —NH— moiety that nitrogen may be optionally substituted by a group selected from R¹⁹;

m is 0-3; wherein the values of R⁶ may be the same or different;

Y is —S(O)ₐ—, —O—, —NR²⁰—, —C(O)—, —C(O)NR²¹—, —NR²²C(O)— or —SO₂NR²³—; wherein a is 0 to 2;

R⁷, R⁹ and R¹⁸ are independently selected from halo, nitro, cyano, hydroxy, amino, carboxy, carbamoyl, mercapto, sulphamoyl, trifluoromethyl, trifluoromethoxy, C₁₋₄alkyl, C₂₋₄alkenyl, C₂₋₄alkynyl, C₁₋₄alkoxy, C₁₋₄alkanoyl, C₁₋₄alkanoyloxy, N—(C₁₋₄alkyl)amino, N,N—(C₁₋₄alkyl)₂amino, C₁₋₄alkanoylamino, N—(C₁₋₄alkyl)carbamoyl, N,N—(C₁₋₄alkyl)₂carbamoyl, C₁₋₄alkylS(O)ₐ wherein a is 0 to 2, C₁₋₄alkoxycarbonyl, N—(C₁₋₄alkyl)sulphamoyl, N,N—(C₁₋₄alkyl)₂sulphamoyl, C₁₋₄alkylsulphonylamino, carbocyclyl and heterocyclyl; wherein R⁷, R⁹ and R¹⁸ may be independently optionally substituted on carbon by one or more R²⁶;

R⁸, R¹⁰, R¹⁷ and R¹⁹ are independently selected from C₁₋₄alkyl, C₁₋₄alkanoyl, C₁₋₄alkylsulphonyl, C₁₋₄alkoxycarbonyl, carbamoyl, N—(C₁₋₄alkyl)carbamoyl, N,N—(C₁₋₄alkyl)carbamoyl, benzyl, benzyloxycarbonyl, benzoyl, carbocyclyl, heterocyclyl and phenylsulphonyl; wherein R⁸, R¹⁰, R¹⁷ and R¹⁹ may be independently optionally substituted on carbon by one or more R²⁷;

R¹⁵, R¹⁶, R²⁰, R²¹, R²² and R²³ are independently selected from hydrogen, phenyl, C₁₋₄alkylsulphonyl and C₁₋₄alkyl;

R²⁶ and R²⁷ are independently selected from selected from halo, nitro, cyano, hydroxy, trifluoromethoxy, trifluoromethyl, amino, carboxy, carbamoyl, mercapto, sulphamoyl, methyl, ethyl, methoxy, ethoxy, acetyl, acetoxy, methylamino, ethylamino, dimethylamino, diethylamino, N-methyl-N-ethylamino, acetylamino, N-methylcarbamoyl, N-ethylcarbamoyl, N,N-dimethylcarbamoyl, N,N-diethylcarbamoyl, N-methyl-N-ethylcarbamoyl, methylthio, ethylthio, methylsulphinyl, ethylsulphinyl, mesyl, ethylsulphonyl, methoxycarbonyl, ethoxycarbonyl, N-methylsulphamoyl, N-ethylsulphamoyl, N,N-dimethylsulphamoyl, N,N-diethylsulphamoyl or N-methyl-N-ethylsulphamoyl;

or a pharmaceutically acceptable salt thereof;

with the proviso that said compound is not (pyrid-3-yl)-[3-(benzoylamino)propyl]-ketone; or (pyrid-2-yl)-{3-[N-(benzoyl)-N-(ethyl)amino]propyl}-ketone.

In this specification, where particular groups for, or the use of etc., compounds of formula (I) are referred to, it is to be understood that this also refers to compounds of formula (I′) and (I″).

In this specification the term “alkyl” includes both straight and branched chain alkyl groups but references to individual alkyl groups such as “propyl” are specific for the straight chain version only. For example, “C₁₋₄alkyl” includes propyl, isopropyl and t-butyl. However, references to individual alkyl groups such as ‘propyl’ are specific for the straight chained version only and references to individual branched chain alkyl groups such as ‘isopropyl’ are specific for the branched chain version only. A similar convention applies to other radicals therefore “carbocyclylC₁₋₄alkyl” includes 1-carbocyclylpropyl, 2-carbocyclylethyl and 3-carbocyclylbutyl. The term “halo” refers to fluoro, chloro, bromo and iodo.

Where optional substituents are chosen from “one or more” groups it is to be understood that this definition includes all substituents being chosen from one of the specified groups or the substituents being chosen from two or more of the specified groups.

“Heteroaryl” is a totally unsaturated, mono or bicyclic ring containing 3-12 atoms of which at least one atom is chosen from nitrogen, sulphur or oxygen, which may, unless otherwise specified, be carbon or nitrogen linked. Suitably “heteroaryl” refers to a totally unsaturated, monocyclic ring containing 5 or 6 atoms or a bicyclic ring containing 8-10 atoms of which at least one atom is chosen from nitrogen, sulphur or oxygen, which may, unless otherwise specified, be carbon or nitrogen linked. Examples and suitable values of the term “heteroaryl” are thienyl, furyl, thiazolyl, pyrazolyl, isoxazolyl, imidazolyl, pyrrolyl, thiadiazolyl, isothiazolyl, triazolyl, pyranyl, indolyl, pyrimidyl, pyrazinyl, pyridazinyl, benzothienyl, pyridyl and quinolyl. Particularly “heteroaryl” refers to thienyl, furyl, thiazolyl, pyridyl, benzothienyl, imidazolyl or pyrazolyl.

“3-6 Membered heteroaryl” is a totally unsaturated, mono or bicyclic ring containing 3-6 atoms of which at least one atom is chosen from nitrogen, sulphur or oxygen, which may, unless otherwise specified, be carbon or nitrogen linked. Suitably “3-6 membered heteroaryl” refers to a totally unsaturated, monocyclic ring containing 5 or 6 atoms of which at least one atom is chosen from nitrogen, sulphur or oxygen, which may, unless otherwise specified, be carbon or nitrogen linked. Examples and suitable values of the term “3-6 membered heteroaryl” are thienyl, furyl, thiazolyl, pyrazolyl, isoxazolyl, imidazolyl, pyrrolyl, thiadiazolyl, isothiazolyl, triazolyl, pyranyl, pyrimidyl, pyrazinyl, pyridazinyl and pyridyl. Particularly “heteroaryl” refers to thienyl, furyl, thiazolyl, pyridyl, benzothienyl, imidazolyl or pyrazolyl.

“Aryl” is a totally unsaturated, mono or bicyclic carbon ring that contains 3-12 atoms. Suitably “aryl” is a monocyclic ring containing 5 or 6 atoms or a bicyclic ring containing 9 or 10 atoms. Suitable values for “aryl” include phenyl or naphthyl. Particularly “aryl” is phenyl.

“3-6 Membered aryl” is a totally unsaturated, mono or bicyclic carbon ring that contains 3-6 atoms. Suitably “3-6 membered aryl” is a monocyclic ring containing 5 or 6 atoms. Suitable values for “3-6 membered aryl” include phenyl.

A “heterocyclyl” is a saturated, partially saturated or unsaturated, mono or bicyclic ring containing 3-12 atoms of which at least one atom is chosen from nitrogen, sulphur or oxygen, which may, unless otherwise specified, be carbon or nitrogen linked, wherein a —CH₂— group can optionally be replaced by a —C(O)— or a ring sulphur atom may be optionally oxidised to form the S-oxides. Preferably a “heterocyclyl” is a saturated, partially saturated or unsaturated, mono or bicyclic ring containing 5 or 6 atoms of which at least one atom is chosen from nitrogen, sulphur or oxygen, which may, unless otherwise specified, be carbon or nitrogen linked, wherein a —CH₂— group can optionally be replaced by a —C(O)— or a ring sulphur atom may be optionally oxidised to form S-oxide(s). Examples and suitable values of the term “heterocyclyl” are thienyl, piperidinyl, morpholinyl, furyl, thiazolyl, pyridyl, imidazolyl, 1,2,4-triazolyl, thiomorpholinyl, coumarinyl, pyrimidinyl, phthalidyl, pyrazolyl, pyrazinyl, pyridazinyl, benzothienyl, benzimidazolyl, tetrahydrofuryl, [1,2,4]triazolo[4,3-a]pyrimidinyl, piperidinyl, indolyl, 1,3-benzodioxolyl and pyrrolidinyl.

A “spiro attached heterocyclyl” is formed when R² and R³ together form a heterocyclyl when the carbon atom to which both R² and R³ are attached (marked with a star in —C(O)—C*R²R³—X) is also included in the heterocycle. I.e. this atom is common to both the heterocycle and the chain depicted in formula (I). An example of this is: 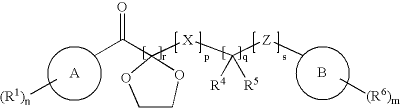
where, in this instance, the “spiro attached heterocyclyl” is 1,3-dioxolan-2-yl.

A “carbocyclyl” is a saturated, partially saturated or unsaturated, mono or bicyclic carbon ring that contains 3-12 atoms; wherein a —CH₂— group can optionally be replaced by a —C(O)—. Preferably “carbocyclyl” is a monocyclic ring containing 5 or 6 atoms or a bicyclic ring containing 9 or 10 atoms. Suitable values for “carbocyclyl” include cyclopropyl, cyclobutyl, 1-oxocyclopentyl, cyclopentyl, cyclopentenyl, cyclohexyl, cyclohexenyl, phenyl, naphthyl, tetralinyl, indanyl or 1-oxoindanyl. Particularly “carbocyclyl” is cyclohexyl, phenyl, naphthyl or 2-6-dioxocyclohexyl.

An example of “C₁₋₄alkanoyloxy” is acetoxy. Examples of “C₁₋₄alkoxycarbonyl” include methoxycarbonyl, ethoxycarbonyl, n- and t-butoxycarbonyl. Examples of “C₁₋₄alkoxy” include methoxy, ethoxy and propoxy. Examples of “oxyC₁₋₄alkoxy” include oxymethoxy, oxyethoxy and oxyropoxy. Examples of “C₁₋₄alkanoylamino” include formamido, acetamido and propionylamino. Examples of and “C₁₋₄alkylS(O)ₐ wherein a is 0 to 2” include methylthio, ethylthio, methylsulphinyl, ethylsulphinyl, mesyl and ethylsulphonyl. Examples of and “C₁₋₄alkylsulphonyl” include mesyl and ethylsulphonyl. Examples of “C₁₋₄alkanoyl” include C₁₋₃alkanoyl, propionyl and acetyl. Examples of “N—(C₁₋₄alkyl)amino” include methylamino and ethylamino. Examples of “N,N—(C₁₋₄alkyl)₂amino” include di-N-methylamino, di-(N-ethyl)amino and N-ethyl-N-methylamino. Examples of “C₂₋₄alkenyl” are vinyl, alkyl and 1-propenyl. Examples of “C₂₋₄alkynyl” are ethynyl, 1-propynyl and 2-propynyl. Examples of “N—(C₁₋₄alkyl)sulphamoyl” are N—(C₁₋₃alkyl)sulphamoyl, N-(methyl)sulphamoyl and N-(ethyl)sulphamoyl. Examples of “N—(C₁₋₄alkyl)₂sulphamoyl” are N,N-(dimethyl)sulphamoyl and N-(methyl)-N-(ethyl)sulphamoyl. Examples of “N—(C₁₋₄alkyl)carbamoyl” are methylaminocarbonyl and ethylaminocarbonyl. Examples of “N,N-(C₁₋₄alkyl)₂carbamoyl” are dimethylaminocarbonyl and methylethylaminocarbonyl. Examples of “C₁₋₄alkylsulphonylamino” are mesylamino and ethylsulphonylamino. Examples of “C₀₋₄alkylene” are a direct bond, methylene and ethylene. Examples of “C₃₋₅alkylene” are propylene and butylene. Examples of “C₁₋₄alkoxycarbonylamino” are methoxycarbonylamino and propoxycarbonylamino.

A suitable pharmaceutically acceptable salt of a compound of the invention is, for example, an acid-addition salt of a compound of the invention which is sufficiently basic, for example, an acid-addition salt with, for example, an inorganic or organic acid, for example hydrochloric, hydrobromic, sulphuric, phosphoric, trifluoroacetic, citric or maleic acid. In addition a suitable pharmaceutically acceptable salt of a compound of the invention which is sufficiently acidic is an alkali metal salt, for example a sodium or potassium salt, an alkaline earth metal salt, for example a calcium or magnesium salt, an ammonium salt or a salt with an organic base which affords a physiologically-acceptable cation, for example a salt with methylamine, dimethylamine, trimethylamine, piperidine, morpholine or tris-(2-hydroxyethyl)amine.

Some compounds of the formula (I) may have chiral centres and/or geometric isomeric centres (E- and Z-isomers), and it is to be understood that the invention encompasses all such optical, diastereoisomers and geometric isomers that possess 11βHSD1 inhibitory activity.

The invention relates to any and all tautomeric forms of the compounds of the formula (I) that possess 11βHSD1 inhibitory activity.

It is also to be understood that certain compounds of the formula (I) can exist in solvated as well as unsolvated forms such as, for example, hydrated forms. It is to be understood that the invention encompasses all such solvated forms which possess 11βHSD1 inhibitory activity.

Particular values of variable groups are as follows. Such values may be used where appropriate with any of the definitions, claims or embodiments defined hereinbefore or hereinafter.

Ring A is selected from aryl.

Ring A is heteroaryl.

Ring A is selected from phenyl, naphthyl, thienyl, furyl, thiazolyl, pyridyl, benzothienyl, imidazolyl or pyrazolyl.

Ring A is selected from phenyl, naphthyl, thienyl, furyl, thiazolyl, pyridyl or imidazolyl.

Ring A is selected from phenyl, naphthyl, thienyl, furyl, thiazolyl, pyridyl, imidazolyl, benzothiazolyl or benzothienyl.

Ring A is selected from phenyl, naphth-2-yl, thien-2-yl, thien-3-yl, fur-2-yl, thiazol-2-yl, pyrid-2-yl, pyrid-3-yl, pyrid-4-yl, benzothien-3-yl, imidazol-2-yl or pyrazol-1-yl.

Ring A is selected from phenyl, naphth-2-yl, thien-2-yl, fur-2-yl, thiazol-2-yl, pyrid-2-yl, pyrid-3-yl, pyrid-4-yl or imidazol-2-yl.

Ring A is selected from phenyl, naphth-2-yl, thien-2-yl, fur-2-yl, thiazol-2-yl, pyrid-2-yl, pyrid-3-yl, pyrid-4, imidazol-2-yl, benzothien-2-yl or benzothiazol-2-yl.

Ring A is selected from phenyl, thien-2-yl, thien-3-yl, fur-2-yl, thiazol-2-yl, pyrid-2-yl, benzothien-3-yl, imidazol-2-yl or pyrazol-1-yl.

Ring A is phenyl substituted at the position para to the ketone.

Ring A is not substituted in the positions ortho to the ketone.

Ring A is phenyl with hydrogens in the two positions ortho to the ketone.

Ring A is phenyl with hydrogens in the two positions ortho to the ketone and a substituent para to the ketone.

R¹ is selected from halo, cyano, hydroxy, C₁₋₄alkyl, C₁₋₄alkoxy, N,N—(C₁₋₄alkyl)₂amino, C₁₋₄alkylS(O)ₐ wherein a is 0, carbocyclyl, carbocyclylC₀₋₄alkylene-Y— and heterocyclylC₀₋₄alkylene-Y—; or two R¹ on adjacent carbons may form an oxyC₁₋₄alkoxy group; wherein R¹ may be optionally substituted on carbon by one or more groups selected from R⁷;

Y is —S(O)ₐ— or —O—; wherein a is 0 to 2; and

R⁷ is halo.

R¹ is selected from halo, cyano, hydroxy, C₁₋₆alkyl, C₁₋₆alkoxy, N,N—(C₁₋₆alkyl)₂amino, C₁₋₆alkylsulphonylamino, carbocyclyl and heterocyclylC₀₋₆alkylene-Y—; or two R¹ on adjacent carbons may form an oxyC₁₋₄alkoxy group; wherein R¹ may be optionally substituted on carbon by one or more groups selected from R⁷;

Y is —S(O)ₐ—, or —O—; wherein a is 0 to 2; and

R⁷ is halo.

R¹ is selected from fluoro, chloro, bromo, cyano, hydroxy, methyl, t-butyl, trifluoromethyl, methoxy, ethoxy, butoxy, dimethylamino, methylthio, 4-chlorophenyl, benzyloxy, morpholinosulphonyl and tetrahydrofur-2-yloxy; or two R¹ on adjacent carbons may form oxymethyleneoxy.

R¹ is selected from fluoro, chloro, bromo, iodo, cyano, hydroxy, methyl, pentyl, trifluoromethyl, methoxy, dimethylamino, methylsulphonylamino, phenyl, morpholinosulphonyl and tetrahydropyran-2-yloxy; or two R¹ on adjacent carbons may form oxymethyleneoxy.

R¹ is selected from fluoro, chloro, bromo, iodo, cyano, hydroxy, methyl, pentyl, trifluoromethyl, methoxy, isopropoxy, dimethylamino, methylsulphonylamino, phenyl, morpholinosulphonyl and tetrahydropyran-2-yloxy; or two R¹ on adjacent carbons may form oxymethyleneoxy.

R¹ is selected from fluoro, chloro, bromo, cyano, methyl, trifluoromethyl, methoxy and ethoxy.

n is 0-2; wherein the values of R¹ may be the same or different.

n is 0-1.

n is 0.

n is 1.

n is 2.

r is 1.

r is 2.

R², R³, R⁴ and R⁵ are independently selected from hydrogen, hydroxy, amino, cyano, C₁₋₄alkyl, C₁₋₄alkoxy, N—(C₁₋₄alkyl)amino, N,N—(C₁₋₄alkyl)₂amino, carbocyclyl, heterocyclyl, carbocyclylC₁₋₄alkyl, heterocyclylC₁₋₄alkyl; wherein R², R³, R⁴ and R⁵ may be independently optionally substituted on carbon by one or more groups selected from R⁹;

R⁹ is selected from halo, nitro, cyano, trifluoromethyl, C₁₋₄alkyl, C₁₋₄alkoxy, N—(C₁₋₄alkyl)amino, N,N—(C₁₋₄alkyl)₂amino, C₁₋₄alkoxycarbonyl and carbocyclyl; wherein R⁹ may be optionally substituted on carbon by one or more R²⁶; wherein

R²⁶ is hydroxy.

R², R³, R⁴ and R⁵ are independently selected from hydrogen, hydroxy, C₁₋₄alkyl, C₁₋₄alkoxy, N—(C₁₋₄alkyl)amino, carbocyclyl, carbocyclylC₁₋₄alkyl and heterocyclylC₁₋₄alkyl; wherein R², R³, R⁴ and R⁵ may be independently optionally substituted on carbon by one or more groups selected from R⁹; wherein

R⁹ is selected from halo, cyano, C₁₋₄alkyl and N,N—(C₁₋₄alkyl)₂amino.

R², R³, R⁴ and R⁵ are independently selected from hydrogen, hydroxy, amino, cyano, methyl, ethyl, propyl, isopropyl, ethoxy, isobutoxy, cyanomethyl, ethylaminomethyl, propylaminomethyl, isopropylaminomethyl, N,N-dimethylaminomethyl, N,N-diethylaminomethyl, N,N-dipropylaminomethyl, N,N-diisopropylaminomethyl, 2-hydroxyethylaminomethyl, methylamino, ethylamino, propylamino, isopropylamino, 2-hydroxyethylamino, 2-(N,N-diethylamino)ethylamino, 3-(N,N-dimethylamino)propylamino, N,N-dipropylamino, phenyl, 2-fluorophenyl, 2-chlorophenyl, 3-chlorophenyl, 4-chlorophenyl, 4-methylphenyl, 4-methoxyphenyl, 3,4-dimethoxyphenyl, piperidin-1-yl, indol-1-yl, 1,3-benzodioxol-5-yl, benzyl, α-cyanobenzyl, 2-fluorobenzyl, 2-nitrobenzyl, 2-ethoxycarbonylbenzyl, 3-nitrobenzyl, 3-trifluoromethylbenzyl, 3-methoxycarbonylbenzyl, 4-fluorobenzyl, 4-chlorobenzyl, 4-nitrobenzyl, 4-methoxycarbonylbenzyl, 2,4-dichlorobenzyl, 3-nitro-6-methoxybenzyl, benzylamino, phenethylaminopyrrolidin-1-ylmethyl, piperidin-1-ylmethyl, morpholinomethyl, 5-nitrofur-2-ylmethyl, 2-methylthiazol-4-ylmethyl, 2-chlorothiazol-5-ylmethyl, pyrid-2-ylmethyl, pyrid-3-ylmethyl, pyrid-4-ylmethyl.

R², R³, R⁴ and R⁵ are independently selected from hydrogen, hydroxy, methyl, ethyl, cyanomethyl, diisopropylaminomethyl, methoxy, ethoxy, isopropoxy, ethylamino, isopropylamino, methylamino, phenyl, 4-fluorophenyl, 4-chlorophenyl, 2,4-dichlorophenyl, ⁴-methylphenyl, benzyl, 4-chlorobenzyl, 2-fluorobenzyl, 4-fluorobenzyl and 2-chlorothiazol-5-ylmethyl.

R², R³, R⁴ and R⁵ are independently selected from hydrogen, hydroxy, methyl, ethyl, cyanomethyl, diisopropylaminomethyl, methoxy, ethoxy, isopropoxy, ethylamino, isopropylamino, methylamino, phenyl, 4-fluorophenyl, 4-chlorophenyl, 2,4-dichlorophenyl, 4-methylphenyl, benzyl, 4-chlorobenzyl, 2-fluorobenzyl, 4-fluorobenzyl, phenethyl and 2-chlorothiazol-5-ylmethyl.

R², R³, R⁴ and R⁵ are independently selected from hydrogen, hydroxy, amino, cyano, methyl, ethyl, propyl, isopropyl, ethoxy, cyanomethyl, methylamino, ethylamino, propylamino, isopropylamino, piperidin-1-yl, benzyl, 4-fluorobenzyl, 4-chlorobenzyl, 4-methoxycarbonylbenzyl, 2,4-dichlorobenzyl, benzylamino, piperidin-1-ylmethyl, morpholinomethyl, 2-methylthiazol-4-ylmethyl, 2-chlorothiazol-5-ylmethyl, pyrid-2-ylmethyl, pyrid-3-ylmethyl and pyrid-4-ylmethyl.

R² and R³ are not both methyl.

One of R² and R³ is hydrogen.

One of R² and R³ is selected from hydrogen, hydroxy, amino, cyano, C₁₋₄alkyl, C₁₋₄alkoxy, N—(C₁₋₄alkyl)amino, N,N—(C₁₋₄alkyl)₂amino, carbocyclyl, heterocyclyl, carbocyclylC₁₋₄alkyl and heterocyclylC₁₋₄alkyl; and the other is selected from hydrogen, hydroxy, amino, cyano, C₁₋₄alkoxy, N—(C₁₋₄alkyl)amino, N,N—(C₁₋₄alkyl)₂amino, carbocyclyl, heterocyclyl, carbocyclylC₁₋₄alkyl and heterocyclylC₁₋₄alkyl; wherein R² and R³ may be independently optionally substituted on carbon by one or more groups selected from R⁹; and wherein if said heterocyclyl contains an —NH— moiety that nitrogen may be optionally substituted by a group selected from R¹⁰.

X is —S(O)ₐ—, —O—, —NR¹³—, —NR¹⁵C(O)—, —SO₂NR¹⁶— or —NR¹⁶SO₂—; wherein a is 0 or 2;

R¹³, R¹⁵ and R¹⁶ are independently selected from hydrogen, phenyl and C₁₋₄alkyl.

X is —S(O)ₐ—, —O—, —NR¹³—, —NR¹⁵C(O)—, —SO₂NR¹⁶— or —NR¹⁶SO₂—; wherein a is 0 or 2; and

R¹³, R¹⁵ and R¹⁶ are independently selected from hydrogen, phenyl, C₁₋₄alkylsulphonyl and C₁₋₄alkyl.

X is —S—, —S(O)₂—, —O—, —NH—, —NMe-, —NHC(O)—, —SO₂NMe- or —NPhSO₂-.

X is —S—, —S(O)₂—, —O—, —NMe-, —NEt, —N(iPr)—, —N(SO₂Me)-, —NHC(O)—, —NPhC(O)—, —SO₂NH—, —SO₂NMe-, —SO₂NEt-, —SO₂N(iPr)—, —NMeSO₂—, or —NEtSO₂—.

X is —S(O)₂—, —O—, —NH—, —NMe-, —NHC(O)—, —SO₂NMe- or —NPhSO₂—.

X is —SO₂NR¹⁶—.

X is —S(O)ₐ—; wherein a is 2 and Ring B is a nitrogen linked heterocyclyl.

q is 0.

q is 1.

p is 0.

p is 1.

Ring B is carbocyclyl.

Ring B is heterocyclyl.

Ring B is phenyl, thien-2-yl, thien-3-yl, piperidin-1-yl, morpholino, morpholin-2-yl, 4-benzylmorpholin-2-yl, naphth-1-yl, naphth-2-yl, 2,6-dioxocyclohex-1-yl, cyclohexyl, pyridin-2-yl, pyridin-3-yl, pyridin-4-yl, imidazol-1-yl, 1-methylimidazol-2-yl, 1,2,4-triazol-1-yl, thiomorpholino, coumarin-7-yl, pyrimidin-2-yl, phthalid-3-yl, pyrazin-2-yl, pyridazin-3-yl, benzimidazol-1-yl or [1,2,4]triazolo[4,3-a]pyrimidin-5-yl.

R¹⁷ is selected from C₁₋₄alkyl or benzyl.

Ring B is phenyl, thienyl, furyl, thiazolyl, piperidinyl, piperazinyl, morpholinyl, naphthyl, cyclohexyl, pyridyl, imidazolyl, 1,2,4-triazolyl, 1,3-benzodioxolyl, thiomorpholinyl, pyrimidinyl, pyrazinyl, pyridazinyl, benzimidazolyl or pyrimidinyl; wherein if Ring B contains an —NH— moiety, that nitrogen may be optionally substituted by a group selected from R¹⁷;

R¹⁷ is C₁₋₄alkyl or benzyl.

Ring B is phenyl, thienyl, furyl, thiazolyl, piperidinyl, piperazinyl, pyrrolidinyl, 1,3-dihydroisoindolyl, morpholinyl, naphthyl, cyclohexyl, pyridyl, imidazolyl, 1,2,4-triazolyl, 1,3-benzodioxolyl, thiomorpholinyl, pyrimidinyl, pyrazinyl, pyridazinyl, benzimidazolyl or pyrimidinyl; wherein if Ring B contains an —NH— moiety, that nitrogen may be optionally substituted by a group selected from R¹⁷;

R¹⁷ is C₁₋₄alkyl or benzyl; wherein R¹⁷ may be optionally substituted on carbon by one or more R²⁷; wherein

R²⁷ is methoxy.

Ring B is phenyl, thienyl, piperidinyl, morpholinyl, naphthyl, 2,6-dioxocyclohexyl, cyclohexyl, pyridyl, imidazolyl, 1,2,4-triazolyl, thiomorpholinyl, coumarinyl, pyrimidinyl, phthalidyl, pyrazinyl, pyridazinyl, benzimidazolyl or [1,2,4]triazolo[4,3-a]pyrimidinyl; wherein if said imidazolyl or morpholinyl is linked via a carbon it may be optionally substituted on the —NH— by a group selected from R¹⁷.

Ring B is phenyl, thien-2-yl, fur-2-yl, thiazol-4-yl, thiazol-5-yl, thien-3-yl, piperidin-1-yl, 4-methylpiperazin-1-yl, morpholino, N-benzylmorpholin-1-yl, naphth-2-yl, cyclohexyl, pyridin-2-yl, pyridin-3-yl, pyridin-4-yl, imidazol-1-yl, 1-methylimidazol-2-yl, 1,2,4-triazol-1-yl, 1,3-benzodioxol-5-yl, thiomorpholino, pyrimidin-2-yl, pyrazin-2-yl, pyridazin-3-yl, benzimidazol-1-yl, benzimidazol-2-yl, 1-methylbenzimidazol-2-yl or pyrimidin-2-yl.

Ring B is phenyl, thien-2-yl, fur-2-yl, thiazol-4-yl, thiazol-5-yl, thien-3-yl, piperidin-1-yl, 4-methylpiperazin-1-yl, pyrrolidin-1-yl, 1,3-dihydroisoindol-2-yl, morpholino, N-benzylmorpholin-1-yl, naphth-2-yl, cyclohexyl, pyridin-2-yl, pyridin-3-yl, pyridin-4-yl, imidazol-1-yl, 1-methylimidazol-2-yl, 1,2,4-triazol-1-yl, 1,3-benzodioxol-5-yl, thiomorpholino, pyrimidin-2-yl, pyrazin-2-yl, pyridazin-3-yl, benzimidazol-1-yl, benzimidazol-2-yl, 1-methylbenzimidazol-2-yl or pyrimidin-2-yl; wherein if Ring B contains an —NH— moiety, that nitrogen may be optionally substituted by a group selected from R¹⁷;

R¹⁷ is 2-methoxyethyl, isopropyl or benzyl.

Ring B is phenyl, thien-2-yl, thien-3-yl, piperidin-1-yl, morpholino, morpholin-2-yl, 4-benzylmorpholin-2-yl, pyridin-2-yl, pyridin-3-yl, pyridin-4-yl, thiomorpholino, pyrimidin-2-yl, phthalid-3-yl, pyrazin-2-yl, pyridazin-3-yl, benzimidazol-1-yl or [1,2,4]triazolo[4,3-a]pyrimidin-5-yl.

Ring B is phenyl substituted at the position para to —(CR⁴R⁵)_{q}—.

R⁶ is a substituent on carbon and is selected from halo, cyano, hydroxy, amino, carbamoyl, trifluoromethyl, C₁₋₄alkyl, C₁₋₄alkoxy, C₁₋₄alkanoyl, N—(C₁₋₄alkyl)amino, C₁₋₄alkylS(O)ₐ wherein a is 0 or 2, carbocyclyl, heterocyclyl and heterocyclylC₀₋₄alkylene-Y—; wherein R⁶ may be optionally substituted on carbon by one or more groups selected from R¹⁸;

Y is —S(O)₂—;

R¹⁸ is selected from halo, cyano, hydroxy, carbocyclyl and heterocyclyl.

R⁶ is a substituent on carbon and is selected from halo, nitro, cyano, carbamoyl, C₁₋₄alkyl, C₁₋₄alkoxy, C₁₋₄alkanoyl, N,N—(C₁₋₄alkyl)₂amino, C₁₋₄alkanoylamino, N—(C₁₋₄alkyl)carbamoyl, N,N—(C₁₋₄alkyl)₂carbamoyl, C₁₋₄alkylS(O)ₐ wherein a is 0 or 2, C₁₋₄alkoxycarbonyl, N,N—(C₁₋₄alkyl)₂sulphamoyl, carbocyclyl, heterocyclyl and carbocyclylC₀₋₄alkylene-Y—; wherein R⁶ may be optionally substituted on carbon by one or more groups selected from R¹⁸; and wherein if said heterocyclyl contains an —NH— moiety that nitrogen may be optionally substituted by a group selected from R¹⁹;

Y is —C(O) or —C(O)NR²¹—;

R¹⁸ is selected from halo, cyano, hydroxy, C₁₋₄alkoxy and heterocyclyl;

R¹⁹ is heterocyclyl; and

R²¹ is hydrogen.

R⁶ is a substituent on carbon and is selected from halo, hydroxy, nitro, cyano, carbamoyl, C₁₋₄alkyl, C₁₋₄alkoxy, C₁₋₄alkanoyl, N,N—(C₁₋₄alkyl)₂amino, C₁₋₄alkanoylamino, N—(C₁₋₄alkyl)carbamoyl, N,N—(C₁₋₄alkyl)₂carbamoyl, C₁₋₄alkylS(O)ₐ wherein a is 0 or 2, C₁₋₄alkoxycarbonyl, N,N—(C₁₋₄alkyl)₂sulphamoyl, carbocyclyl, heterocyclyl and carbocyclylC₀₋₄alkylene-Y—; wherein R⁶ may be optionally substituted on carbon by one or more groups selected from R¹⁸; and wherein if said heterocyclyl contains an —NH— moiety that nitrogen may be optionally substituted by a group selected from R¹⁹;

Y is —C(O) or —C(O)NR²¹—;

R¹⁸ is selected from halo, cyano, hydroxy, C₁₋₄alkoxy and heterocyclyl;

R¹⁹ is heterocyclyl; and

R²¹ is hydrogen.

R⁶ is a substituent on carbon and is selected from fluoro, chloro, bromo, cyano, hydroxy, amino, carbamoyl, trifluoromethyl, methyl, t-butyl, cyanomethyl, methoxy, ethoxy, acetyl, 2-hydroxyethylamino, methylthio, mesyl, phenyl, 4-fluorophenyl, 2-thiazolin-2-yl, morpholinomethyl, and piperidin-1-ylsulphonyl.

R⁶ is a substituent on carbon and is selected from fluoro, chloro, bromo, iodo, nitro, cyano, carbamoyl, methyl, propyl, isopropyl, butyl, t-butyl, hydroxymethyl, cyanomethyl, morpholinomethyl, methoxy, ethoxy, 2-methoxyethoxy, acetyl, diethylamino, acetylamino, N-(isopropyl)carbamoyl, N-(isobutyl)carbamoyl, N,N-dimethylcarbamoyl, methoxymethylthio, methylthio, mesyl, methoxycarbonyl, ethoxycarbonyl, N,N-dimethylsulphamoyl, phenyl, cyclopentyl, 4-fluorophenyl, anilinocarbonyl, 4-(pyrid-4-yl)piperazin-1-yl, 2-thiazolin-2-yl, morpholino and 4-chlorobenzoyl.

R⁶ is a substituent on carbon and is selected from fluoro, chloro, bromo, iodo, hydroxy, nitro, cyano, carbamoyl, methyl, propyl, isopropyl, butyl, t-butyl, hydroxymethyl, cyanomethyl, morpholinomethyl, 2-hydroxyethyl, methoxy, ethoxy, 2-methoxyethoxy, acetyl, diethylamino, acetylamino, N-(isopropyl)carbamoyl, N-(isobutyl)carbamoyl, N,N-dimethylcarbamoyl, methoxymethylthio, methylthio, mesyl, methoxycarbonyl, ethoxycarbonyl, N,N-dimethylsulphamoyl, phenyl, cyclopentyl, 4-fluorophenyl, anilinocarbonyl, 4-(pyrid-4-yl)piperazin-1-yl, 2-thiazolin-2-yl, morpholino and 4-chlorobenzoyl.

R⁶ is a substituent on carbon and is selected from fluoro, chloro, cyano, carbamoyl, trifluoromethyl, methyl, cyanomethyl, methoxy, ethoxy, acetyl, 2-hydroxyethylamino, mesyl, 4-fluorophenyl, 2-thiazolin-2-yl, morpholinomethyl and piperidin-1-ylsulphonyl.

m is 0-2; wherein the values of R⁶ may be the same or different.

m is 0 or 1.

m is 0.

m is 1.

Therefore in a further aspect of the invention there is provided the use of a compound of formula (I) (as depicted above) wherein:

Ring A is selected from phenyl, naphthyl, thienyl, furyl, thiazolyl, pyridyl, benzothienyl, imidazolyl or pyrazolyl;

R¹ is selected from halo, cyano, hydroxy, C₁₋₄alkyl, C₁₋₄alkoxy, N,N—(C₁₋₄alkyl)₂amino, C₁₋₄alkylS(O)ₐ wherein a is 0, carbocyclyl, carbocyclylC₀₋₄alkylene-Y— and heterocyclylC₀₋₄alkylene-Y—; or two R¹ on adjacent carbons may form an oxyC₁₋₄alkoxy group; wherein R¹ may be optionally substituted on carbon by one or more groups selected from R⁷;

Y is —S(O)ₐ— or —O—; wherein a is 0 to 2; and

R⁷ is halo;

n is 0-3; wherein the values of R¹ may be the same or different;

r is 1;

s is 0;

R², R³, R⁴ and R⁵ are independently selected from hydrogen, hydroxy, amino, cyano, C₁₋₄alkyl, C₁₋₄alkoxy, N—(C₁₋₄alkyl)amino, N,N—(C₁₋₄alkyl)₂amino, carbocyclyl, heterocyclyl, carbocyclylC₁₋₄alkyl, heterocyclylC₁₋₄alkyl; wherein R², R³, R⁴ and R⁵ may be independently optionally substituted on carbon by one or more groups selected from R⁹;

R⁹ is selected from halo, nitro, cyano, trifluoromethyl, C₁₋₄alkyl, C₁₋₄alkoxy, N—(C₁₋₄alkyl)amino, N,N—(C₁₋₄alkyl)₂amino, C₁₋₄alkoxycarbonyl and carbocyclyl; wherein R⁹ may be optionally substituted on carbon by one or more R²⁶; wherein

R²⁶ is hydroxy;

X is —S(O)ₐ—, —O—, —NR¹³—, —NR¹⁵C(O)—, —SO₂NR¹⁶— or —NR¹⁶SO₂—; wherein a is 0 or 2;

R¹³, R¹⁵ and R¹⁶ are independently selected from hydrogen, phenyl and C₁₋₄alkyl;

q is 0 or 1;

p is 0 or 1;

Ring B is phenyl, thienyl, piperidinyl, morpholinyl, naphthyl, 2,6-dioxocyclohexyl, cyclohexyl, pyridyl, imidazolyl, 1,2,4-triazolyl, thiomorpholinyl, coumarinyl, pyrimidinyl, phthalidyl, pyrazinyl, pyridazinyl, benzimidazolyl or [1,2,4]triazolo[4,3-a]pyrimidinyl; wherein if said imidazolyl or morpholinyl is linked via a carbon it may be optionally substituted on the —NH— by a group selected from R¹⁷;

R¹⁷ is selected from C₁₋₄alkyl or benzyl;

R⁶ is a substituent on carbon and is selected from halo, cyano, hydroxy, amino, carbamoyl, trifluoromethyl, C₁₋₄alkyl, C₁₋₄alkoxy, C₁₋₄alkanoyl, N—(C₁₋₄alkyl)amino, C₁₋₄alkylS(O)ₐ wherein a is 0 or 2, carbocyclyl, heterocyclyl and heterocyclylC₀₋₄alkylene-Y—, wherein R⁶ may be optionally substituted on carbon by one or more groups selected from R¹⁸;

Y is —S(O)₂—;

R¹⁸ is selected from halo, cyano, hydroxy, carbocyclyl and heterocyclyl; and

m is 0-3; wherein the values of R⁶ may be the same or different.

or a pharmaceutically acceptable salt thereof;

in the manufacture of a medicament for use in the inhibition of 11βHSD1.

Therefore in a further aspect of the invention there is provided the use of a compound of formula (I) (as depicted above) wherein:

Ring A is selected from phenyl, naphth-2-yl, thien-2-yl, thien-3-yl, fur-2-yl, thiazol-2-yl, pyrid-2-yl, pyrid-3-yl, pyrid-4-yl, benzothien-3-yl, imidazol-2-yl or pyrazol-1-yl;

R¹ is selected from fluoro, chloro, bromo, cyano, hydroxy, methyl, t-butyl, trifluoromethyl, methoxy, ethoxy, butoxy, dimethylamino, methylthio, 4-chlorophenyl, benzyloxy, morpholinosulphonyl and tetrahydrofur-2-yloxy; or two R¹ on adjacent carbons may form oxymethyleneoxy;

n is 0-3; wherein the values of R¹ may be the same or different;

r is 1;

s is 0;

R², R³, R⁴ and R⁵ are independently selected from hydrogen, hydroxy, amino, cyano, methyl, ethyl, propyl, isopropyl, ethoxy, isobutoxy, cyanomethyl, ethylaminomethyl, propylaminomethyl, isopropylaminomethyl, N,N-dimethylaminomethyl, N,N-diethylaminomethyl, N,N-dipropylaminomethyl, N,N-diisopropylaminomethyl, 2-hydroxyethylaminomethyl, methylamino, ethylamino, propylamino, isopropylamino, 2-hydroxyethylamino, 2-(N,N-diethylamino)ethylamino, 3-(N,N-dimethylamino)propylamino, N,N-dipropylamino, phenyl, 2-fluorophenyl, 2-chlorophenyl, 3-chlorophenyl, 4-chlorophenyl, 4-methylphenyl, 4-methoxyphenyl, 3,4-dimethoxyphenyl, piperidin-1-yl, indol-1-yl, 1,3-benzodioxol-5-yl, benzyl, α-cyanobenzyl, 2-fluorobenzyl, 2-nitrobenzyl, 2-ethoxycarbonylbenzyl, 3-nitrobenzyl, 3-trifluoromethylbenzyl, 3-methoxycarbonylbenzyl, 4-fluorobenzyl, 4-chlorobenzyl, 4-nitrobenzyl, 4-methoxycarbonylbenzyl, 2,4-dichlorobenzyl, 3-nitro-6-methoxybenzyl, benzylamino, phenethylaminopyrrolidin-1-ylmethyl, piperidin-1-ylmethyl, morpholinomethyl, 5-nitrofur-2-ylmethyl, 2-methylthiazol-4-ylmethyl, 2-chlorothiazol-5-ylmethyl, pyrid-2-ylmethyl, pyrid-3-ylmethyl, pyrid-4-ylmethyl;

X is —S—, —S(O)₂—, —O—, —NH—, —NMe-, —NHC(O)—, —SO₂NMe- or —NPhSO₂—;

q is 0 or 1;

p is 0 or 1;

Ring B is phenyl, thien-2-yl, thien-3-yl, piperidin-1-yl, morpholino, morpholin-2-yl, 4-benzylmorpholin-2-yl, naphth-1-yl, naphth-2-yl, 2,6-dioxocyclohex-1-yl, cyclohexyl, pyridin-2-yl, pyridin-3-yl, pyridin-4-yl, imidazol-1-yl, 1-methylimidazol-2-yl, 1,2,4-triazol-1-yl, thiomorpholino, coumarin-7-yl, pyrimidin-2-yl, phthalid-3-yl, pyrazin-2-yl, pyridazin-3-yl, benzimidazol-1-yl or [1,2,4]triazolo[4,3-a]pyrimidin-5-yl;

R⁶ is a substituent on carbon and is selected from fluoro, chloro, bromo, cyano, hydroxy, amino, carbamoyl, trifluoromethyl, methyl, t-butyl, cyanomethyl, methoxy, ethoxy, acetyl, 2-hydroxyethylamino, methylthio, mesyl, phenyl, 4-fluorophenyl, 2-thiazolin-2-yl, morpholinomethyl, and piperidin-1-ylsulphonyl;

m is 0-3; wherein the values of R⁶ may be the same or different;

or a pharmaceutically acceptable salt thereof;

in the manufacture of a medicament for use in the inhibition of 11βHSD1.

Therefore in a further aspect of the invention there is provided the use of a compound of formula (I) (as depicted above) wherein:

Ring A is selected from phenyl, naphthyl, thienyl, furyl, thiazolyl, pyridyl or imidazolyl;

R¹ is selected from halo, cyano, hydroxy, C₁₋₆alkyl, C₁₋₆alkoxy, N,N—(C₁₋₆alkyl)₂amino, C₁₋₆alkylsulphonylamino, carbocyclyl and heterocyclylC₀₋₆alkylene-Y—; or two R¹ on adjacent carbons may form an oxyC₁₋₄alkoxy group; wherein R¹ may be optionally substituted on carbon by one or more groups selected from R⁷;

Y is —S(O)ₐ—, or —O—; wherein a is 0 to 2; and

R⁷ is halo;

n is 0-3; wherein the values of R¹ may be the same or different;

r is 1 or 2;

R², R³, R⁴ and R⁵ are independently selected from hydrogen, hydroxy, C₁₋₄alkyl, C₁₋₄alkoxy, N—(C₁₋₄alkyl)amino, carbocyclyl, carbocyclylC₁₋₄alkyl and heterocyclylC₁₋₄alkyl; wherein R², R³, R⁴ and R⁵ may be independently optionally substituted on carbon by one or more groups selected from R⁹; wherein

R⁹ is selected from halo, cyano, C₁₋₄alkyl and N,N-(C₁₋₄alkyl)₂amino;

X is —S(O)ₐ—, —O—, —NR¹³—, —NR¹⁵C(O)—, —SO₂NR¹⁶— or —NR¹⁶SO₂—; wherein a is 0 or 2; and

R¹³, R¹⁵ and R¹⁶ are independently selected from hydrogen, phenyl, C₁₋₄alkylsulphonyl and C₁₋₄alkyl;

q is 0 or 1;

p is 0 or 1;

Ring B is phenyl, thienyl, furyl, thiazolyl, piperidinyl, piperazinyl, morpholinyl, naphthyl, cyclohexyl, pyridyl, imidazolyl, 1,2,4-triazolyl, 1,3-benzodioxolyl, thiomorpholinyl, pyrimidinyl, pyrazinyl, pyridazinyl, benzimidazolyl or pyrimidinyl; wherein if Ring B contains an —NH— moiety, that nitrogen may be optionally substituted by a group selected from R¹⁷;

R¹⁷ is C₁₋₄alkyl or benzyl;

R⁶ is a substituent on carbon and is selected from halo, nitro, cyano, carbamoyl, C₁₋₄alkyl, C₁₋₄alkoxy, C₁₋₄alkanoyl, N,N—(C₁₋₄alkyl)₂amino, C₁₋₄alkanoylamino, N—(C₁₋₄alkyl)carbamoyl, N,N—(C₁₋₄alkyl)₂carbamoyl, C₁₋₄alkylS(O)ₐ wherein a is 0 or 2, C₁₋₄alkoxycarbonyl, N,N—(C₁₋₄alkyl)₂sulphamoyl, carbocyclyl, heterocyclyl and carbocyclylC₀₋₄alkylene-Y—; wherein R⁶ may be optionally substituted on carbon by one or more groups selected from R¹⁸; and wherein if said heterocyclyl contains an —NH— moiety that nitrogen may be optionally substituted by a group selected from R¹⁹;

Y is —C(O) or —C(O)NR²¹—;

R¹⁸ is selected from halo, cyano, hydroxy, C₁₋₄alkoxy and heterocyclyl;

R¹⁹ is heterocyclyl; and

R²¹ is hydrogen;

m is 0-3; wherein the values of R⁶ may be the same or different;

or a pharmaceutically acceptable salt thereof;

in the manufacture of a medicament for use in the inhibition of 11βHSD1.

Therefore in a further aspect of the invention there is provided the use of a compound of formula (I) (as depicted above) wherein:

Ring A is selected from phenyl, naphthyl, thienyl, furyl, thiazolyl, pyridyl, imidazolyl, benzothiazolyl or benzothienyl;

R¹ is selected from halo, cyano, hydroxy, C₁₋₆alkyl, C₁₋₆alkoxy, N,N—(C₁₋₆alkyl)₂amino, C₁₋₆alkylsulphonylamino, carbocyclyl and heterocyclylC₀₋₆alkylene-Y—; or two R¹ on adjacent carbons may form an oxyC₁₋₄alkoxy group; wherein R¹ may be optionally substituted on carbon by one or more groups selected from R⁷;

Y is —S(O)ₐ—, or —O—; wherein a is 0 to 2; and

R⁷ is halo.

n is 0-3; wherein the values of R¹ may be the same or different;

r is 1 or 2;

s is 0;

R², R³, R⁴ and R⁵ are independently selected from hydrogen, hydroxy, C₁₋₄alkyl, C₁₋₄alkoxy, N—(C₁₋₄alkyl)amino, carbocyclyl, carbocyclylC₁₋₄alkyl and heterocyclylC₁₋₄alkyl; wherein R², R³, R⁴ and R⁵ may be independently optionally substituted on carbon by one or more groups selected from R⁹; wherein

R⁹ is selected from halo, cyano, C₁₋₄alkyl and N,N-(C₁₋₄alkyl)₂amino.

X is —S(O)ₐ—, —O—, —NR¹³—, —NR¹⁵C(O)—, —SO₂NR¹⁶— or —NR⁶SO₂—; wherein a is 0 or 2; and

R¹³, R¹⁵ and R¹⁶ are independently selected from hydrogen, phenyl, C₁₋₄alkylsulphonyl and C₁₋₄alkyl;

q is 0 or 1;

p is 0 or 1;

Ring B is phenyl, thienyl, furyl, thiazolyl, piperidinyl, piperazinyl, pyrrolidinyl, 1,3-dihydroisoindolyl, morpholinyl, naphthyl, cyclohexyl, pyridyl, imidazolyl, 1,2,4-triazolyl, 1,3-benzodioxolyl, thiomorpholinyl, pyrimidinyl, pyrazinyl, pyridazinyl, benzimidazolyl or pyrimidinyl; wherein if Ring B contains an —NH— moiety, that nitrogen may be optionally substituted by a group selected from R¹⁷;

R¹⁷ is C₁₋₄alkyl or benzyl; wherein R¹⁷ may be optionally substituted on carbon by one or more R²⁷; wherein

R²⁷ is methoxy;

R⁶ is a substituent on carbon and is selected from halo, hydroxy, nitro, cyano, carbamoyl, C₁₋₄alkyl, C₁₋₄alkoxy, C₁₋₄alkanoyl, N,N—(C₁₋₄alkyl)₂amino, C₁₋₄alkanoylamino, N—(C₁₋₄alkyl)carbamoyl, N,N—(C₁₋₄alkyl)₂carbamoyl, C₁₋₄alkylS(O)ₐ wherein a is 0 or 2, C₁₋₄alkoxycarbonyl, N,N—(C₁₋₄alkyl)₂sulphamoyl, carbocyclyl, heterocyclyl and carbocyclylC₀₋₄alkylene-Y—; wherein R⁶ may be optionally substituted on carbon by one or more groups selected from R¹⁸; and wherein if said heterocyclyl contains an —NH— moiety that nitrogen may be optionally substituted by a group selected from R¹⁹,

Y is —C(O) or —C(O)NR²¹—;

R¹⁸ is selected from halo, cyano, hydroxy, C₁₋₄alkoxy and heterocyclyl;

R¹⁹ is heterocyclyl; and

R²¹ is hydrogen;

m is 0-3; wherein the values of R⁶ may be the same or different;

or a pharmaceutically acceptable salt thereof;

in the manufacture of a medicament for use in the inhibition of 11βHSD1;

with the proviso that said compound is not (1-methyl-1-pyrid-3-ylethyl)-(pyrid-3-yl)-ketone.

In another aspect of the present invention, a suitable compound of the invention, or a pharmaceutically acceptable salt thereof, is selected from Group A:
(benzyl)-[4-(morpholinosulphonyl)phenyl]-ketone;
(2-methylpyrid-5-yloxymethyl)-(phenyl)-ketone;
[2-(3-chlorophenyl)-2-(1,2,4-triazol-1-yl)ethyl]-(phenyl)-ketone;
(4-chlorobenzyl)-(2-bromophenyl)-ketone;
(4-chlorobenzyl)-(3-bromophenyl)-ketone;
(3,4-dichlorobenzyl)-(3,4-dichlorophenyl)-ketone;
[α-(4-fluorobenzyl)benzyl]-(pyrid-3-yl)-ketone;
{α-[3-(N,N-dimethylamino)propylamino]benzyl}-(pyrid-3-yl)-ketone;
(2,4-dibromophenoxymethyl)-(phenyl)-ketone;
[α-(cyclohexylamino)-4-chlorobenzyl]-(4-chlorophenyl)-ketone;
[1-(cyclohexylamino)-1-(1,3-benzodioxol-5-yl)methyl]-(1,3-benzodioxol-5-yl)-ketone;
[α-(cyclohexylamino)-3,4-dimethoxybenzyl]-(2-chlorophenyl)-ketone;
[α-(cyclohexylamino)-4-methylbenzyl]-(4-methylphenyl)-ketone;
[α-(cyclohexylamino)-2-chlorobenzyl]-(2-chlorophenyl)-ketone;
[α-hydroxy-α-(N,N-dipropylaminomethyl)benzyl]-(phenyl)-ketone;
[α-hydroxy-α-(N,N-diisopropylaminomethyl)benzyl]-(phenyl)-ketone;
[α-hydroxy-α-(N,N-diethylaminomethyl)-4-methoxybenzyl]-(4-methoxyphenyl)-ketone;
[α-hydroxy-α-(N,N-diethylaminomethyl)-4-methylbenzyl]-(4-methylphenyl)-ketone;
{α-hydroxy-α-[2-(hydroxyethyl)aminomethyl]benzyl}-(phenyl)-ketone;
[α-hydroxy-α-(propylaminomethyl)benzyl]-(phenyl)-ketone;
[α-hydroxy-α-(isopropylaminomethyl)benzyl]-(phenyl)-ketone;
(phthalid-3-ylmethyl)-(4-chlorophenyl)-ketone;
[2-(3-trifluoromethylphenyl)-1-(1,2,4-triazol-1-yl)ethyl]-(4-fluorophenyl)-ketone;
[2-(4-nitrophenyl)-1-(1,2,4-triazol-1-yl)ethyl]-(4-chlorophenyl)-ketone;
[2-(2-fluorophenyl)-1-(1,2,4-triazol-1-yl)ethyl]-(4-chlorophenyl)-ketone;
[2-(2,4-dichlorophenyl)-1-(1,2,4-triazol-1-yl)ethyl]-(phenyl)-ketone;
(4-bromobenzyl)-(4-fluorophenyl)-ketone;
[2-(4-fluorophenyl)-1-(pyrazin-2-yl)ethyl]-(phenyl)-ketone;
(phthalid-3-ylmethyl)-(4-fluorophenyl)-ketone;
[2-(2-fluorophenyl)-1-(pyrazin-2-yl)ethyl]-(fur-2-yl)-ketone;
[2-(4-chlorophenyl)-1-(pyrid-3-yl)ethyl]-(4-chlorophenyl)-ketone;
[2-(2,4-dichlorophenyl)-1-(pyridazin-3-yl)ethyl]-(phenyl)-ketone;
[2-(4-chlorophenyl)-1-(pyridazin-3-yl)ethyl]-(phenyl)-ketone;
[2-(4-chlorophenyl)-1-(pyrazin-2-yl)ethyl]-(pyrid-3-yl)-ketone;
[2-(4-chlorophenyl)-1-(pyrazin-2-yl)ethyl]-(fur-2-yl)-ketone;
(3,4-dichlorobenzyl)-(4-chlorophenyl)-ketone;
(2-fluorobenzyl)-(4-chlorophenyl)-ketone;
[2-(4-fluorophenyl)-1-(pyrazin-2-yl)ethyl]-(4-chlorophenyl)-ketone;
[2-(1,2,4-triazol-1-yl)-3-methyl)butyl]-(phenyl)-ketone;
[2-(4-chlorophenyl)-1-(phenyl)ethyl]-(pyrid-3-yl)-ketone;
[2-(2-fluorophenyl)-1-(1,2,4-triazol-1-yl)ethyl]-(thien-2-yl)-ketone;
[2-(phenyl)-1-(imidazol-1-yl)ethyl]-(4-chlorophenyl)-ketone;
[1-methyl-1-(1,2,4-triazol-1-yl)ethyl]-(4-chlorophenyl)-ketone;
[2-(2-aminophenylthio)-2-(4-methoxyphenyl)ethyl)-(4-methoxyphenyl)-ketone;
[2-(2,4-dichlorophenyl)-1-(1,2,4-triazol-1-yl)ethyl]-(2-chlorothien-5-yl)-ketone;
[1-(hydroxy)-1-(thien-3-yl)methyl]-(thien-3-yl)-ketone;
(α-hydroxybenzyl)-(4-t-butylphenyl)-ketone;
[2-(4-chlorophenyl)-1-(4-methylphenyl)ethyl]-(pyrid-3-yl)-ketone;
[(7-methyl[1,2,4]triazolo[4,3-a]pyrimidin-5-yl)oxymethyl]-(4-chlorophenyl)-ketone;
(4-phenyl-2,6-dioxocyclohexylmethyl)-(4-bromophenyl)-ketone;
(α-ethoxy-α-ethylaminomethylbenzyl)-(phenyl)-ketone;
[α-(2-oxocyclopentyl)benzyl]-(phenyl)-ketone;
[α-(5-chloropyrimidin-2-yl)benzyl]-(phenyl)-ketone;
(phenoxymethyl)-(3,5-dimethyl-2,3-dihydro-pyrazol-2-yl)-ketone;
[2-(piperidin-1-yl)-1-(4-methylphenylsulphonyl)]-(phenyl)-ketone;
(benzimidazol-1-ylmethyl)-(4-bromophenyl)-ketone;
{[2-(2-hydroxyethylamino)-benzimidazol-1-yl]methyl}-(thien-2-yl)-ketone;
(2-carbamoylphenoxymethyl)-(4-bromophenyl)-ketone;
(morpholin-2-ylmethyl)-(phenyl)-ketone;
(pyrimidin-2-ylsulphanylmethyl)-(4-bromophenyl)-ketone;
(4-acetylbenzyl)-(4-chlorophenyl)-ketone;
[(1-methylimidazol-2-yl)sulphanylmethyl]-(4-chlorophenyl)-ketone;
(benzimidazol-1-ylmethyl)-(2,4-dichlorophenyl)-ketone;
(4-methylbenzyl)-[4-(tetrahydropyran-2-yloxy)phenyl]-ketone;
(phenylsulphonylmethyl)-(pyrid-2-yl)-ketone;
(4-chlorophenoxymethyl)-(3,5-difluorophenyl)-ketone;
[(1-(naphth-2-yl)-1-(hydroxy)methyl]-(4-dimethylaminophenyl)-ketone;
(α-hydroxy-4-methoxybenzyl)-(naphth-2-yl)-ketone;
(4-chlorophenethyl)-(2,4-difluorophenyl)-ketone;
(4-fluorophenoxymethyl)-(4-chlorophenyl)-ketone;
(phenoxymethyl)-(4-trifluoromethyl-2-fluorophenyl)-ketone;
[1-methyl-1-(1,2,4-triazol-1-yl)ethyl]-(4-trifluoromethyl-2-fluorophenyl)-ketone;
(4-fluorophenethyl)-(4-trifluoromethylphenyl)-ketone;
(4-fluorophenethyl)-(2,4-difluorophenyl)-ketone;
(4-fluorophenethyl)-(4-chlorophenyl)-ketone;
(benzyl)-(3,4-dichlorophenyl)-ketone;
[4-(piperdin-1-ylsulphonyl)phenoxymethyl]-(phenyl)-ketone;
[2-(morpholinomethyl)-3,5-dimethylphenoxymethyl]-(phenyl)-ketone;
(phenylsulphonylmethyl)-(3,4-dihydroxyphenyl)-ketone; and
(4-methylphenylsulphonylmethyl)-(4-chloro-3-methylphenyl)-ketone.

In a further aspect of the invention, there is provided the use of a compound or a pharmaceutically acceptable salt thereof, selected from Group B:
(2,2-diphenylethyl)-(phenyl)-ketone;
(1,2-diphenylethyl)-(4-chlorophenyl)-ketone;
(1-phenylpropyl)-(phenyl)-ketone;
[2-(piperidin-1-yl)-1-(phenyl)ethyl]-(phenyl)-ketone;
[2-(morpholino)-1-(phenyl)ethyl]-(phenyl)-ketone;
[2-(dimethylamino)-1-(phenyl)ethyl]-(phenyl)-ketone;
[2-(phenyl)-1-(imidazol-1-yl)ethyl]-(phenyl)-ketone;
(1,2-diphenylethyl)-(phenyl)-ketone;
(α-propylbenzyl)-(phenyl)-ketone;
[α-(cyanomethyl)benzyl]-(phenyl)-ketone;
[N-(4-methylphenylsulphonyl)anilinomethyl]-(phenyl)-ketone;
(phenylsulphonylmethyl)-(phenyl)-ketone;
[(1-methylimidazol-2-yl)sulphanylmethyl]-(4-bromophenyl)-ketone;
(4-methylphenylsulphonylmethyl)-(4-bromophenyl)-ketone;
(4-chlorophenylsulphanylmethyl)-(phenyl)-ketone;
(4-chlorophenylsulphonylmethyl)-(phenyl)-ketone;
(phenylsulphonylmethyl)-(4-methoxyphenyl)-ketone;
(phenylsulphonylmethyl)-(4-methylphenyl)-ketone;
(4-methylphenylsulphonylmethyl)-(4-chlorophenyl)-ketone;
(4-chlorophenylsulphonylmethyl)-(4-bromophenyl)-ketone;
(benzylsulphonylmethyl)-(phenyl)-ketone;
(2-carbamoylphenoxymethyl)-(phenyl)-ketone;
(naphth-2-yloxymethyl)-(phenyl)-ketone;
(phenoxymethyl)-(phenyl)-ketone;
(4-chlorophenoxymethyl)-(phenyl)-ketone;
(phenoxymethyl)-(4-chlorophenyl)-ketone;
(4-cyanophenoxymethyl)-(phenyl)-ketone;
(4-t-butylphenoxymethyl)-(4-chlorophenyl)-ketone;
(N-methylanilinomethyl)-(phenyl)-ketone;
(4-chlorobenzamidomethyl)-(4-bromophenyl)-ketone;
[1-(cyano)-1-(thien-2-yl)ethyl]-(phenyl)-ketone;
(phenethyl)-(4-bromophenyl)-ketone;
[2-(2-methoxyphenyl)ethyl]-(phenyl)-ketone;
(2-(cyano)-2-(phenyl)ethyl]-(phenyl)-ketone;
(phenethyl)-(phenyl)-ketone;
(Phenethyl)-(2-methoxyphenyl)-ketone;
(3,4-dimethoxyphenethyl)-(phenyl)-ketone;
(Phenethyl)-(4-chlorophenyl)-ketone;
(α-hydroxybenzyl)-(phenyl)-ketone;
(α-hydroxy-4-chlorobenzyl)-(4-chlorophenyl)-ketone;
[α-hydroxy-αx-(N,N-diethylaminomethyl)benzyl]-(phenyl)-ketone;
[α-hydroxy-α-(piperidin-1-ylmethyl)benzyl]-(phenyl)-ketone;
[α-hydroxy-α-(N,N-dimethylaminomethyl)benzyl]-(phenyl)-ketone;
[α-hydroxy-α-(morpholinomethyl)benzyl]-(phenyl)-ketone;
(α-hydroxy-4-chlorobenzyl)-(4-methoxyphenyl)-ketone;
(α-ethoxybenzyl)-(phenyl)-ketone;
(α-hydroxy-α-ethylbenzyl)-(phenyl)-ketone;
(α-hydroxybenzyl)-(4-methoxyphenyl)-ketone;
[1-(1,2,4-triazol-1-yl)-1-(ethoxy)methyl]-(4-chlorophenyl)-ketone;
[1-(thien-2-yl)-1-(hydroxy)methyl]-(thien-2-yl)-ketone;
(α-hydroxybenzyl)-(4-methoxyphenyl)-ketone;
(α-hydroxy-4-methoxybenzyl)-(phenyl)-ketone;
(α-isopropoxybenzyl)-(phenyl)-ketone;
(α-isobutoxybenzyl)-(phenyl)-ketone;
(α-aminobenzyl)-(4-chlorophenyl)-ketone;
(α-[2-(N,N-diethylamino)ethylamino]benzyl)-(phenyl)-ketone;
(α-isopropylaminobenzyl)-(phenyl)-ketone;
[α-(piperidin-1-yl)-4-chlorobenzyl]-(4-chlorophenyl)-ketone;
[α-(benzylamino)benzyl]-(Phenyl)-ketone;
[α-(4-chloroanilino)benzyl]-(phenyl)-ketone;
[α-(cyclohexylamino)benzyl]-(phenyl)-ketone;
[α-(N,N-dipropylamino)benzyl]-(phenyl)-ketone;
[α-(2-hydroxyethylamino)benzyl]-(phenyl)-ketone;
[α-(phenethylamino)benzyl]-(phenyl)-ketone;
[α-(ethylamino)benzyl]-(phenyl)-ketone;
[α-(propylamino)benzyl]-(phenyl)-ketone;
[α-(methylamino)benzyl]-(phenyl)-ketone;
[α-(anilino)benzyl]-(fur-2-yl)-ketone;
[1-(benzimidazol-1-yl)-1-(anilino)methyl-(phenyl)-ketone;
(4-chlorobenzyl)-(phenyl)-ketone;
(benzyl)-(4-ethoxyphenyl)-ketone;
(4-methoxybenzyl)-(4-methoxyphenyl)-ketone;
(benzyl)-(4-methylphenyl)-ketone;
[4-(benzyl)morpholin-2-ylmethyl]-(phenyl)-ketone;
(pyrid-2-ylmethyl)-(4-chlorophenyl)-ketone;
(2-chlorobenzyl)-(4-chlorophenyl)-ketone;
(4-chlorobenzyl)-(4-chlorophenyl)-ketone;
(pyrid-3-ylmethyl)-(4-chlorophenyl)-ketone;
(4-bromobenzyl)-(4-chlorophenyl)-ketone;
(2,4-dichlorobenzyl)-(4-chlorophenyl)-ketone;
(4-chlorobenzyl)-(4-methylphenyl)-ketone;
(4-chlorobenzyl)-(4-bromophenyl)-ketone;
(benzyl)-(2-chlorophenyl)-ketone;
(4-methoxybenzyl)-(phenyl)-ketone;
(α-methylbenzyl)-(phenyl)-ketone;
(benzyl)-[4-(4-chlorophenyl)phenyl]-ketone;
(4-fluorobenzyl)-(4-bromophenyl)-ketone;
(4-chlorobenzyl)-(4-methoxyphenyl)-ketone;
(4-methylbenzyl)-(4-methoxyphenyl)-ketone;
(pyrid-2-ylmethyl)-(phenyl)-ketone;
(α,α-dimethylbenzyl)-(phenyl)-ketone;
(4-methylbenzyl)-(pyrid-3-yl)-ketone;
(pyrid-4-ylmethyl)-(pyrid-4-yl)-ketone;
(4-methoxybenzyl)-(4-bromophenyl)-ketone;
(4-methylthiobenzyl)-(4-fluorophenyl)-ketone;
(benzyl)-(4-benzyloxyphenyl)-ketone;
(4-fluorobenzyl)-(4-fluorophenyl)-ketone;
(α-methylbenzyl)-(phenyl)-ketone;
(4-methoxybenzyl)-(4-fluorophenyl)-ketone;
(thiomorpholinomethyl)-(thianaphthen-3-yl)-(phenyl)-ketone;
(benzyl)-(4-butoxyphenyl)-ketone;
(2,2-diphenylethyl)-(2,4,6-trimethylphenyl)-ketone;
[2-(2-hydroxyphenyl)-2-phenylethyl]-(phenyl)-ketone;
(cyclohexylmethyl)-(phenyl)-ketone;
(benzyl)-(2-bromothien-5-yl)-ketone;
(1,2-diphenyl-2-cyanoethyl)-(phenyl)-ketone;
(4-methoxybenzyl)-(3-bromophenyl)-ketone;
(α-hydroxybenzyl)-(3-methoxyphenyl)-ketone;
[α-(pyrrolidin-1-ylmethyl)benzyl]-(phenyl)-ketone;
[α-(pyridin-2-ylamino)-4-methoxybenzyl]-(4-methoxyphenyl)-ketone;
(4-chlorobenzyl)-(4-fluorophenyl)-ketone;
(benzyl)-[4-(tetrahydropyran-2-yloxy)phenyl]-ketone;
(4-chlorophenylsulphonylmethyl)-(4-chlorophenyl)-ketone;
(4-methyl-α-hydroxybenzyl)-(4-chlorophenyl)-ketone;
(4-methylbenzyl)-(4-chlorophenyl)-ketone;
(4-fluoro-α-hydroxybenzyl)-(4-fluorophenyl)-ketone;
(4-methoxy-α-hydroxybenzyl)-(4-methoxyphenyl)-ketone;
(α-methyl-α-hydroxybenzyl)-(phenyl)-ketone;
(1-methyl-1-morpholinoethyl)-(4-methylsulphanylphenyl)-ketone;
[2-(phenylsulphonyl)-2-(phenyl)ethyl]-(phenyl)-ketone
(1,3-diphenylprop-2-yl)-(phenyl)-ketone;
(naphth-1-yloxymethyl)-(phenyl)-ketone;
(phenoxymethyl)-(4-methylphenyl)-ketone;
(4-methylcoumarin-7-yloxymethyl)-(4-methoxyphenyl)-ketone;
(imidazol-1-ylmethyl)-(2-chlorothien-5-yl)-ketone;
(thien-2-ylsulphonylmethyl)-(4-chlorophenyl)-ketone;
(1-methylimidazol-2-ylsulphanylmethyl)-(3,4-difluorophenyl)-ketone;
(1-methylimidazol-2-ylsulphonylmethyl)-(4-chlorophenyl)-ketone;
(3-trifluoromethylpyrid-6-ylsulphonylmethyl)-(4-chlorophenyl)-ketone;
(4-methyl-α-hydroxybenzyl)-(4-methylphenyl)-ketone;
(4-bromophenoxymethyl)-(phenyl)-ketone;
(4-ethoxyanilinomethyl)-(4-methylphenyl)-ketone;
(2,4,6-trichlorophenoxymethyl)-(phenyl)-ketone; and
in the manufacture of a medicament for use in the inhibition of 11βHSD1.

In another aspect of the present invention, a suitable compound of the invention, or a pharmaceutically acceptable salt thereof, is selected from Group C:
(phenyl)-[2-(piperidin-1-yl)-2-(2-chlorophenyl)ethyl]-ketone;
(phenyl)-(2,4,5-trichlorophenoxymethyl)-ketone;
(phenyl)-[([α-hydroxy-α-(butylaminomethyl)benzyl]-ketone;
(4-fluorophenyl)-[2-(3,4-dichlorophenyl)-1-(1,2,4-triazol-1-yl)ethyl]-ketone;
(thien-2-yl)-[2-(4-fluorophenyl)-1-(1,2,4-triazol-1-yl)ethyl]-ketone;
(phenyl)-[2-(3-nitrophenyl)-2-(2-aminophenylthio)ethyl]-ketone;
(pyrid-3-yl)-[α-(t-butanonylmethyl)-4-chlorobenzyl]-ketone;
(4-methoxyphenyl)-[4-(4-fluorobenzoyl)piperidin-1-ylmethyl]-ketone;
(phenyl)-(2-nitro-4-chorophenoxymethyl)-ketone;
(phenyl)-(2,6-dibromo-3-ethoxycarbonylphenoxymethyl)-ketone;
(4-bromophenyl)-(4-nitrophenoxymethyl)-ketone; and
(phenyl)-(4-nitrophenoxymethyl)-ketone.

In a further aspect of the invention, there is provided the use of a compound or a pharmaceutically acceptable salt thereof, selected from Group D:
(phenyl)-(benzoyl)-ketone;
(phenyl)-[2-(4-methoxyphenyl)-2-cyanoethyl]-ketone;
(phenyl)-[2-(phenyl)-2-(2-methoxyethylthio)ethyl]-ketone;
(4-methylphenyl)-(4-methylbenzoyl)-ketone;
(phenyl)-[2-(2-chlorophenyl)-2-cyanoethyl]-ketone;
(phenyl)-(4-methylphenylsulphonylmethyl)-ketone;
(4-chlorophenyl)-[2-(phenyl)-2-cyanoethyl]-ketone;
(phenyl)-[2-(pyrrolidin-1-yl)-2-(phenyl)ethyl]-ketone;
(4-bromophenyl)-[2-piperidin-1-yl)-2-(phenyl)ethyl]-ketone;
(phenyl)-[α-(alkylamino)benzyl]-ketone;
(phenyl)-[α-phenyl-α-hydroxybenzyl]-ketone;
(Phenyl)-[2-(3-methoxyanilino)-2-(phenyl)ethyl]-ketone;
(phenyl)-[α-phenyl-α-hydroxy-4-methoxybenzyl]-ketone;
(4-fluorophenyl)-[2-(4-chlorophenyl)-1-(1,2,4-triazol-1-yl)ethyl]-ketone;
(4-chlorophenyl)-[2-(4-cyanophenyl)-1-(1,2,4-triazol-1-yl)ethyl]-ketone;
(phenyl)-[2-(morpholino)-2-(phenyl)ethyl]-ketone;
(4-fluorophenyl)-[2-(2-fluorophenyl)-1-(1,2,4-triazol-1-yl)ethyl]-ketone;
(phenyl)-[2-(4-fluorophenyl)-1-(1,2,4-triazol-1-yl)ethyl]-ketone;
(phenyl)-[2-(phenyl)-1-(1,2,4-triazol-1-yl)ethyl]-ketone;
(4-chlorophenyl)-[2-(phenyl)-1-(1,2,4-triazol-1-yl)ethyl]-ketone;
(phenyl)-(benzylsulphinylmethyl)-ketone;
(5-chlorothien-2-yl)-[2-(3,4-dichlorophenyl)-1-(1,2,4-triazol-1-yl)ethyl]-ketone;
(phenyl)-[2-(cyano)-2-(4-chlorophenyl)ethyl]-ketone;
(thien-2-yl)-[2-(phenyl)-1-(1,2,4-triazol-1-yl)ethyl]-ketone;
(4-hydroxyphenyl)-(benzoyl)-ketone;
(phenyl)-[2-(morpholino)-1-(benzyl)ethyl]-ketone;
(phenyl)-[2-(2-methoxyphenyl)-2-(2-aminophenylthio)ethyl]-ketone;
(phenyl)-[2-(1,3-benzodioxol-5-yl)-2-(2-aminophenylthio)ethyl]-ketone;
(phenyl)-[2-(4-fluorobenzoyl)-2-(1,2,4-triazol-1-yl)ethyl]-ketone;
(3,4-dimethylphenyl)-[4-(4-fluorobenzoyl)piperidin-1-ylmethyl]-ketone;
(4-methoxyphenyl)-(α-methylbenzyl)-ketone;
(4-methoxyphenyl)-(3-methylbenzyl)-ketone;
(3-methyl-4-methoxyphenyl)-(benzyl)-ketone;
(4-fluorophenyl)-(benzimidazol-1-ylmethyl)-ketone;
(phenyl)-(1-methyl-1-imidazol-1-ylethyl)-ketone;
(phenyl)-(2-methylaminobenzimidazol-1-ylmethyl)-ketone;
(4-chlorophenyl)-(2,4-dichlorophenoxymethyl)-ketone;
(4-chlorophenyl)-(2,4,6-trichlorophenoxymethyl)-ketone;
(4-bromophenyl)-[2-(trifluoromethyl)benzoylaminomethyl]-ketone;
(4-bromophenyl)-(α-homopiperidin-1-ylbenzyl)-ketone;
(4-chlorophenyl)-(4-chloroanilinomethyl)-ketone;
(phenyl)-[N-(benzoyl)anilinomethyl]-ketone;
(phenyl)-(3-methylindol-1-ylmethyl)-ketone;
(phenyl)-(2,4-dichlorobenzoylaminomethyl)-ketone;
(phenyl)-[2-(phenyl)-1-(ethoxycarbonyl)ethyl]-ketone;
(4-chlorophenyl)-(4-chlorobenzoylaminomethyl)-ketone; and
(4-chlorophenyl)-(2-fluorobenzoylaminomethyl)-ketone;
in the manufacture of a medicament for use in the inhibition of 11βHSD 1.

In another aspect of the present invention, a suitable compound of the invention, or a pharmaceutically acceptable salt thereof, is selected from Group E:
(phenyl)-[1-(2-chloroanilinocarbonyl)-2-phenylethyl]-ketone;
(phenyl)-[2-(anilinocarbonyl)-1,2-diphenylethyl]-ketone;
(4-chlorophenyl)-[2-(3,5-dichlorophenyl)-1-(1,2,4-triazol-1-yl)propyl]-ketone;
(2,4-dichlorophenyl)-[2,2-diphenyl-1-(S)-(1,2,4-triazol-1-yl)ethyl]-ketone;
(phenyl)-[(N-methylanilinocarbonyl)methylthiomethyl]-ketone;
(1,2,3,4-tetrahydronaphth-6-yl)-[1-(morpholinomethyl)ethyl]-ketone;
(4-fluorophenyl)-[3-(4,6-dimethoxypyrimidin-2-ylamino)propyl]-ketone;
(phenyl)-{1-[1-(3-trifluoromethylphenyl)piperazin-4-yl]ethyl}-ketone; and
(4-fluorophenyl)-(3-anilinopropyl)-ketone.

In a further aspect of the invention, there is provided the use of a compound or a pharmaceutically acceptable salt thereof, selected from Group F:
(phenyl)-(1-phenyl-1-{4-[5-(3-bromophenyl)-1,3,4-oxadiazol-2-yl]benzoyloxy}methyl)-ketone;
(phenyl)-[2-(4-fluorophenyl)-1-(imidazol-1-yl)propyl]-ketone;
(thien-2-yl)-[(thien-2-ylcarbonyl)methylthiomethyl]-ketone;
(phenyl)-[4-amino-5-(benzoyl)thiazol-2-ylthiomethyl]-ketone;
(phenyl)-(3-benzoyl-2-pyridin-4-ylpropyl)-ketone;
(phenyl)-(2-benzoyl-1-phenylethyl)-ketone;
(phenyl)-(3-phenyl-5-methylisoxazol-4-ylcarbonyloxymethyl)-ketone;
(phenyl)-(2-phenyl-1,3-dioxolan-2-yl)-ketone;
(phenyl)-[α-(2,5-dioxopyrrolidin-1-yl)benzyl]-ketone;
(phenyl)-[α-(amino)benzyl]-ketone;
(4-bromophenyl)-[2-(4-chlorophenyl)-1-(1,2,4-triazol-1-yl)propyl]-ketone;
(4-bromophenyl)-(2-phenyl-2-cyanoethyl)-ketone;
(4-chlorophenyl)-(benzimidazol-2-ylthiomethyl)-ketone;
(phenyl)-(2-oxobenzoxazol-3-ylmethyl)-ketone;
(phenyl)-(4-methylbenzyl)-ketone;
(phenyl)-(4-phenyl-1,3-dioxolan-4-yl)-ketone;
(4-chlorophenyl)-(pyridin-2-ylthiomethyl)-ketone;
(phenyl)-[α-(3-carboxypropoxy)benzyl]-ketone;
(phenyl)-(6-methyl-1,2,3,4-tetrahydroquinolin-1-ylmethyl)-ketone;
(phenyl)-[α-(4-fluorobenzoyloxy)benzyl]-ketone;
(4-methylphenyl)-[α-(benzylcarbonyloxy)benzyl]-ketone;
(4-fluorophenyl)-[α-(morpholino)benzyl]-ketone;
(4-bromophenyl)-[α-(ethoxycarbonylamino)benzyl]-ketone;
(phenyl)-[α-(acetyloxy)benzyl]-ketone;
(1,3-benzodioxol-5-yl)-[α-(hydroxy)benzyl]-ketone;
(phenyl)-[α-(methoxy)benzyl]-ketone;
(phenyl)-{α-[2-(ethoxy)ethylamino]benzyl}-ketone;
(phenyl)-(2-phenyl-1,3-dioxan-2-yl)-ketone;
(4-methoxyphenyl)-(α-bromo-4-methoxybenzyl)-ketone;
(phenyl)-[2-phenyl-2-(2-aminophenylthio)ethyl]-ketone;
(4-bromophenyl)-(pyridin-2-ylthiomethyl)-ketone;
(4-methoxyphenyl)-(α-bromobenzyl)-ketone;
(4-methoxyphenyl)-[α-(2-bromobenzoyloxy)benzyl]-ketone;
(4-methoxyphenyl)-[α-(pyridine-4-ylcarbonyloxy)benzyl]-ketone; and
(phenyl)-[α-(benzoyloxy)benzyl]-ketone;
in the manufacture of a medicament for use in the inhibition of 11βHSD1.

In another aspect of the invention, preferred compounds of the invention are any one of the Examples or a pharmaceutically acceptable salt thereof.

In another aspect of the invention, preferred compounds of the invention are Examples 9, 15, 25, 60, 62, 70, 71, 73, 95, 122, 197 or 198, or a pharmaceutically acceptable salt thereof.

In another aspect of the invention, preferred compounds of the invention are any one of the Reference Examples or a pharmaceutically acceptable salt thereof.

In another aspect of the invention, preferred compounds of the invention are a Reference Examples 20, 27, 35 or 54, or a pharmaceutically acceptable salt thereof.

Another aspect of the present invention provides a process for preparing a compound of formula (I) or a pharmaceutically acceptable salt thereof which process (wherein variable groups are, unless otherwise specified, as defined in formula (I)) comprises of:
Process 1): reacting a compound of formula (II): 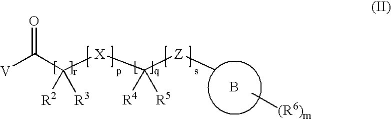
wherein V is a displaceable group; with an organometallic reagent of formula (III): 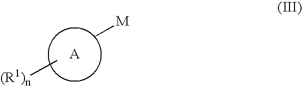
wherein M is a metal reagent;
Process 2): for compounds of formula (I) wherein r is 1 and one of R² and R³ is hydroxy; reacting a compound of formula (IV): 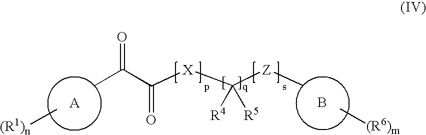
with a compound of formula (V) or (VI):
R²M (V)
R³M (VI)
wherein M is a metal reagent;
Process 3): for compounds of formula (I) wherein one of R⁴ and R⁵ is hydroxy; reacting a compound of formula (VII): 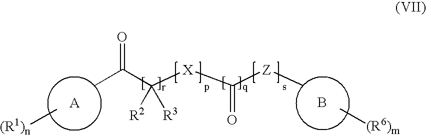
with a compound of formula (VIII) or (IX):
R⁴M (VIII)
R⁵M (IX)
wherein M is a metal reagent; Process 4): for compounds of formula (I) wherein p is 0, q is 1, r is 1, s is 0 and R³ and R⁵ are hydrogen; hydrogenating compound of formula (X): 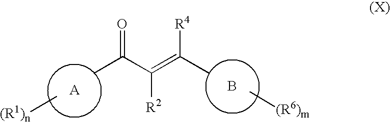
(or its corresponding Z isomer);
Process 5) for compounds of formula (I) wherein p is 1, X is —SO₂—, r is 1, s is 0 and q is 0; reacting a compound of formula (XI): 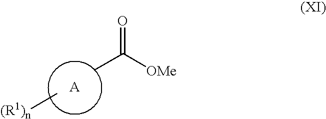
with a compound of formula (XII): 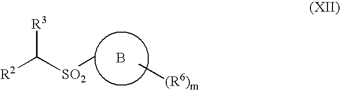
Process 6) for compounds of formula (I) wherein Ring A is a nitrogen linked heteroaryl; reacting a compound of formula (II), wherein V is hydroxy, or an activated derivative thereof forming an activated acid, with a compound of formula (XIII): 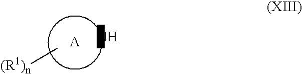
Process 7) for compounds of formula (I) wherein X is —C(O)NR¹⁴—; reacting an acid of formula (XIV): 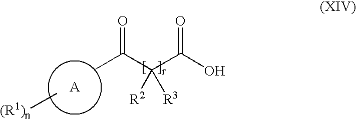
or an activated derivative thereof; with an amine of formula (XV): 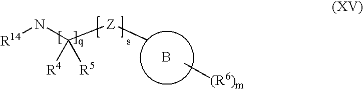
Process 8) for compounds of formula (I) wherein X is —NR¹⁵C(O)—; reacting an amine of formula (XVI): 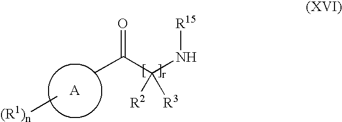
with an acid of formula (XVII): 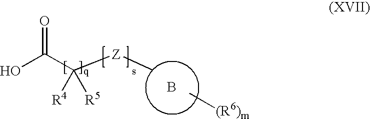
or an activated derivative thereof;
Process 9) for compounds of formula (I) wherein X is —SO₂NR¹⁶—; reacting a compound of formula (XVIII): 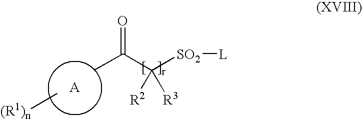
wherein L is a displaceable group; with an amine of formula (XIX): 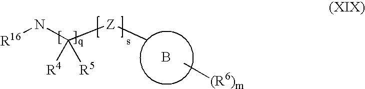
Process 10) for compounds of formula (I) wherein X is —NR¹⁶SO₂—; reacting an amine of formula (XX): 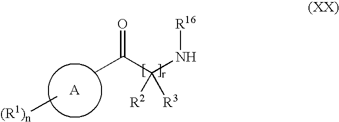
with a compound of formula (XXI): 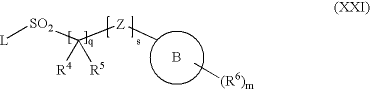
wherein L is a displaceable group;
Process 11) for compounds of formula (I) wherein X is —O—, —NR¹³— or —S—; reacting a compound of formula (XX): 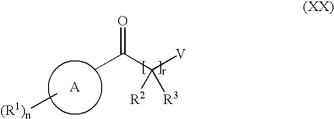
wherein V is —OH, —NR¹³H or —SH; with a compound of formula (XXI): 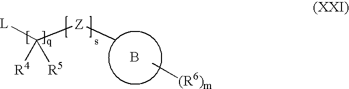
wherein L is a displaceable group;
Process 12) for compounds of formula (I) wherein X is —O—, —NR¹³— or —S—; reacting a compound of formula (XX): 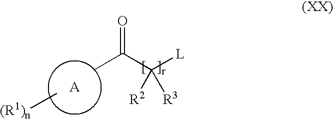
wherein L is a displaceable group; with a compound of formula (XXI): 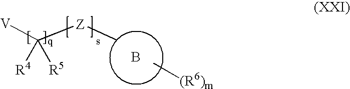
wherein V is —OH, —NR¹³H or —SH;
and thereafter if necessary or desirable:
i) converting a compound of the formula (I) into another compound of the formula (I);
ii) removing any protecting groups;
iii) forming a pharmaceutically acceptable salt thereof.

The skilled reader will appreciate that any of the processes described herein above for preparation of various “X” groups are equally applicable to the corresponding groups in “Z”.

L is a displaceable group, suitable values for L include halo, particularly chloro or bromo, or mesyloxy.

V is a displaceable group, suitable values for V include the Weinreb amide N-methyl-N-methoxyamine.

M is a metal reagent. Suitable values for M include Grignard reagents such as MgBr and lithium.

Suitable activated acid derivatives include acid halides, for example acid chlorides, and active esters, for example pentafluorophenyl esters.

The reactions described above may be performed under standard conditions. The intermediates described above are commercially available, are known in the art or may be prepared by known procedures.

It will be appreciated that certain of the various ring substituents in the compounds of the present invention may be introduced by standard aromatic substitution reactions or generated by conventional functional group modifications either prior to or immediately following the processes mentioned above, and as such are included in the process aspect of the invention. Such reactions and modifications include, for example, introduction of a substituent by means of an aromatic substitution reaction, reduction of substituents, alkylation of substituents and oxidation of substituents. The reagents and reaction conditions for such procedures are well known in the chemical art. Particular examples of aromatic substitution reactions include the introduction of a nitro group using concentrated nitric acid, the introduction of an acyl group using, for example, an acyl halide and Lewis acid (such as aluminium trichloride) under Friedel Crafts conditions; the introduction of an alkyl group using an alkyl halide and Lewis acid (such as aluminium trichloride) under Friedel Crafts conditions; and the introduction of a halogeno group. Particular examples of modifications include the reduction of a nitro group to an amino group by for example, catalytic hydrogenation with a nickel catalyst or treatment with iron in the presence of hydrochloric acid with heating; oxidation of alkylthio to alkylsulphinyl or alkylsulphonyl.

It will also be appreciated that in some of the reactions mentioned herein it may be necessary/desirable to protect any sensitive groups in the compounds. The instances where protection is necessary or desirable and suitable methods for protection are known to those skilled in the art. Conventional protecting groups may be used in accordance with standard practice (for illustration see T. W. Green, Protective Groups in Organic Synthesis, John Wiley and Sons, 1991). Thus, if reactants include groups such as amino, carboxy or hydroxy it may be desirable to protect the group in some of the reactions mentioned herein.

A suitable protecting group for an amino or alkylamino group is, for example, an acyl group, for example an alkanoyl group such as acetyl, an alkoxycarbonyl group, for example a methoxycarbonyl, ethoxycarbonyl or t-butoxycarbonyl group, an arylmethoxycarbonyl group, for example benzyloxycarbonyl, or an aroyl group, for example benzoyl. The deprotection conditions for the above protecting groups necessarily vary with the choice of protecting group. Thus, for example, an acyl group such as an alkanoyl or alkoxycarbonyl group or an aroyl group may be removed for example, by hydrolysis with a suitable base such as an alkali metal hydroxide, for example lithium or sodium hydroxide. Alternatively an acyl group such as a t-butoxycarbonyl group may be removed, for example, by treatment with a suitable acid as hydrochloric, sulphuric or phosphoric acid or trifluoroacetic acid and an arylmethoxycarbonyl group such as a benzyloxycarbonyl group may be removed, for example, by hydrogenation over a catalyst such as palladium-on-carbon, or by treatment with a Lewis acid for example boron tris(trifluoroacetate). A suitable alternative protecting group for a primary amino group is, for example, a phthaloyl group which may be removed by treatment with an alkylamine, for example dimethylaminopropylamine, or with hydrazine.

A suitable protecting group for a hydroxy group is, for example, an acyl group, for example an alkanoyl group such as acetyl, an aroyl group, for example benzoyl, or an arylmethyl group, for example benzyl. The deprotection conditions for the above protecting groups will necessarily vary with the choice of protecting group. Thus, for example, an acyl group such as an alkanoyl or an aroyl group may be removed, for example, by hydrolysis with a suitable base such as an alkali metal hydroxide, for example lithium or sodium hydroxide. Alternatively an arylmethyl group such as a benzyl group may be removed, for example, by hydrogenation over a catalyst such as palladium-on-carbon.

A suitable protecting group for a carboxy group is, for example, an esterifying group, for example a methyl or an ethyl group which may be removed, for example, by hydrolysis with a base such as sodium hydroxide, or for example a t-butyl group which may be removed, for example, by treatment with an acid, for example an organic acid such as trifluoroacetic acid, or for example a benzyl group which may be removed, for example, by hydrogenation over a catalyst such as palladium-on-carbon.

The protecting groups may be removed at any convenient stage in the synthesis using conventional techniques well known in the chemical art.

As stated hereinbefore the compounds defined in the present invention possess 11βHSD1 inhibitory activity. These properties may be assessed using the following assay.

Assay

HeLa cells (human cervical carcinoma derived cells) were stably transfected with a construct containing four copies of the glucocorticoid response element (GRE) linked to a beta-galactosidase reporter gene (3 kb lac Z gene derived from pSV-B-galactosidase). These cells were then further stably transfected with a construct containing full-length human 11βHSD1 enzyme (in pCMVHyg) to create GRE4-βGal/11βHSD1 cells. The principal of the assay is as follows. Cortisone is freely taken up by the cells and is converted to cortisol by 11βHSD1 oxo-reductase activity and cortisol (but not cortisone) binds to and activates the glucocorticoid receptor. Activated glucocorticoid receptor then binds to the GRE and initiates transcription and translation of β-galactosidase. Enzyme activity can then be assayed with high sensitivity by colourimetric assay. Inhibitors of 11βHSD1 will reduce the conversion of cortisone to cortisol and hence decrease the production of β-galactosidase.

Cells were routinely cultured in DMEM (Invitrogen, Paisley, Renfrewshire, UK) containing 10% foetal calf serum (LabTech), 1% glutamine (Invitrogen), 1% penicillin & streptomycin (Invitrogen), 0.5 mg/ml G418 (Invitrogen) & 0.5 mg/ml hygromycin (Boehringer). Assay media was phenol red free-DMEM containing 1% glutamine, 1% penicillin & streptomycin.

Compounds (1 mM) to be tested were dissolved in dimethyl sulphoxide (DMSO) and serially diluted into assay media containing 10% DMSO. Diluted compounds were then plated into transparent flat-bottomed 384 well plates (Matrix, Hudson N.H., USA).

The assay was carried out in 384 well microtitre plate (Matrix) in a total volume of 50 μl assay media consisting of cortisone (Sigma, Poole, Dorset, UK, 1 μM, HeLa GRE4-βGal/11βHSD1 cells (10,000 cells) plus test compounds (3000 to 0.01 nM). The plates were then incubated in 5% O₂, 95% CO₂ at 37° C. overnight.

The following day plates were assayed by measurement of β-galactosidase production.

A cocktail (25 μl) consisting of 10× Z-buffer (600 mM Na₂HPO₄, 400 mM NaH₂PO₄.2H₂O, 100 mM KCl, 10 MM MgSO₄.7H₂O, 500 mM β-mercaptoethanol, pH 7.0), SDS (0.2%), chlorophenol red-β-D-galactopyranoside (5 mM, Roche Diagnostics) was added per well and plates incubated at 37° C. for 3-4 hours. β-Galactosidase activity was indicated by a yellow to red colour change (absorbance at 570 nm) measured using a Tecan Spectrafluor Ultra.

The calculation of median inhibitory concentration (IC₅₀) values for the inhibitors was performed using Origin 6.0 (Microcal Software, Northampton Mass. USA). Dose response curves for each inhibitor were plotted as OD units at each inhibitor concentration with relation to a maximum signal (cortisone, no compound) and IC₅₀ values calculated. Compounds of the present invention typically show an IC₅₀<10 μM. For example the following results were obtained:

| Example | IC₅₀ |
|---|---|
| 3 | 76.756 nM |
| 79 | 93.53 nM |
| 155 | 153.2 nM |

According to a further aspect of the invention there is provided a pharmaceutical composition which comprises a compound of formula (Ia), (Ib), (Ic), (Id), (Ie), (If), (Ig), (Ih), (Ii), (Ij), (Ik), (Il) or (Im) or a pharmaceutically acceptable salt thereof, or a compound selected from Group A, Group C, Group E or the Examples, or a pharmaceutically acceptable salt thereof, as defined hereinbefore in association with a pharmaceutically-acceptable diluent or carrier.

The composition may be in a form suitable for oral administration, for example as a tablet or capsule, for parenteral injection (including intravenous, subcutaneous, intramuscular, intravascular or infusion) as a sterile solution, suspension or emulsion, for topical administration as an ointment or cream or for rectal administration as a suppository.

In general the above compositions may be prepared in a conventional manner using conventional excipients.

The compound of formula (I), or a pharmaceutically acceptable salt thereof, will normally be administered to a warm-blooded animal at a unit dose within the range 0.1-50 mg/kg that normally provides a therapeutically-effective dose. A unit dose form such as a tablet or capsule will usually contain, for example 1-1000 mg of active ingredient. However the daily dose will necessarily be varied depending upon the host treated, the particular route of administration, and the severity of the illness being treated. Accordingly the optimum dosage may be determined by the practitioner who is treating any particular patient.

We have found that the compounds defined in the present invention, or a pharmaceutically acceptable salt thereof, are effective 11βHSD1 inhibitors, and accordingly have value in the treatment of disease states associated with metabolic syndrome.

It is to be understood that where the term “metabolic syndrome” is used herein, this relates to metabolic syndrome as defined in 1) and/or 2) or any other recognised definition of this syndrome. Synonyms for “metabolic syndrome” used in the art include Reaven's Syndrome, Insulin Resistance Syndrome and Syndrome X. It is to be understood that where the term “metabolic syndrome” is used herein it also refers to Reaven's Syndrome, Insulin Resistance Syndrome and Syndrome X.

According to a further aspect of the present invention there is provided a compound of the formula (Ia), (Ib), (Ic), (Id), (Ie), (If), (Ig), (Ih), (Ii), (Ij), (Ik), (Il) or (Im) or a pharmaceutically acceptable salt thereof, or a compound selected from Group A, Group C, Group E or the Examples, or a pharmaceutically acceptable salt thereof, as defined hereinbefore for use in a method of prophylactic or therapeutic treatment of a warm-blooded animal, such as man.

Thus according to this aspect of the invention there is provided a compound of the formula (Ia), (Ib), (Ic), (Id), (Ie), (If), (Ig), (Ih), (Ii), (Ij), (Ik), (Il) or (Im) or a pharmaceutically acceptable salt thereof, or a compound selected from Group A, Group C, Group E or the Examples, or a pharmaceutically acceptable salt thereof, as defined hereinbefore for use as a medicament.

According to another feature of the invention there is provided the use of a compound of the formula (I), or a pharmaceutically acceptable salt thereof, as defined hereinbefore in the manufacture of a medicament for use in the production of an 11βHSD1 inhibitory effect in a warm-blooded animal, such as man.

According to another feature of the invention there is provided the use of a compound of the formula (Ia), (Ib), (Ic), (Id), (Ie), (If), (Ig), (Ih), (Ii), (Ij), (Ik), (Il) or (Im) or a pharmaceutically acceptable salt thereof, or a compound selected from Group A, Group C, Group E or the Examples, or a pharmaceutically acceptable salt thereof, as defined hereinbefore in the manufacture of a medicament for use in the production of an 11βHSD1 inhibitory effect in a warm-blooded animal, such as man.

According to another feature of the invention there is provided the use of a compound selected from Group B, Group D, Group F or the Reference Examples, or a pharmaceutically acceptable salt thereof, as defined hereinbefore in the manufacture of a medicament for use in the production of an 11βHSD1 inhibitory effect in a warm-blooded animal, such as man.

Where production of or producing an 11βHSD1 inhibitory effect is referred to suitably this refers to the treatment of metabolic syndrome. Alternatively, where production of an 11βHSD1 inhibitory effect is referred to this refers to the treatment of diabetes, obesity, hyperlipidaemia, hyperglycaemia, hyperinsulinemia or hypertension, particularly diabetes and obesity. Alternatively, where production of an 11βHSD1 inhibitory effect is referred to this refers to the treatment of glaucoma, osteoporosis, tuberculosis, dementia, cognitive disorders or depression.

According to a further feature of this aspect of the invention there is provided a method for producing an 11βHSD1 inhibitory effect in a warm-blooded animal, such as man, in need of such treatment which comprises administering to said animal an effective amount of a compound of formula (I), or a pharmaceutically acceptable salt thereof.

According to a further feature of this aspect of the invention there is provided a method for producing an 11βHSD1 inhibitory effect in a warm-blooded animal, such as man, in need of such treatment which comprises administering to said animal an effective amount of a compound of formula (Ia), (Ib), (Ic), (Id), (Ie), (If), (Ig), (Ih), (Ii), (Ij), (Ik), (Il) or (Im) or a pharmaceutically acceptable salt thereof, or a compound selected from Group A, Group C, Group E or the Examples, or a pharmaceutically acceptable salt thereof.

According to a further feature of this aspect of the invention there is provided a method for producing an 11βHSD1 inhibitory effect in a warm-blooded animal, such as man, in need of such treatment which comprises administering to said animal an effective amount of a compound selected from Group B, Group D, Group F or the Reference Examples, or a pharmaceutically acceptable salt thereof.

In addition to their use in therapeutic medicine, the compounds of formula (I), or a pharmaceutically acceptable salt thereof, are also useful as pharmacological tools in the development and standardisation of in vitro and in vivo test systems for the evaluation of the effects of inhibitors of 11βHSD1 in laboratory animals such as cats, dogs, rabbits, monkeys, rats and mice, as part of the search for new therapeutic agents.

The inhibition of 11βHSD1 described herein may be applied as a sole therapy or may involve, in addition to the subject of the present invention, one or more other substances and/or treatments. Such conjoint treatment may be achieved by way of the simultaneous, sequential or separate administration of the individual components of the treatment. Simultaneous treatment may be in a single tablet or in separate tablets. For example agents than might be co-administered with 11βHSD1 inhibitors, particularly those of the present invention, may include the following main categories of treatment:

1) Insulin and insulin analogues;

2) Insulin secretagogues including sulphonylureas (for example glibenclamide, glipizide) and prandial glucose regulators (for example repaglinide, nateglinide);

3) Insulin sensitising agents including PPARγ agonists (for example pioglitazone and rosiglitazone);

4) Agents that suppress hepatic glucose output (for example metformin);

5) Agents designed to reduce the absorption of glucose from the intestine (for example acarbose);

6) Agents designed to treat the complications of prolonged hyperglycaemia; e.g. aldose reductase inhibitors 7) Other anti-diabetic agents including phosotyrosine phosphatase inhibitors, glucose 6-phosphatase inhibitors, glucagon receptor antagonists, glucokinase activators, glycogen phosphorylase inhibitors, fructose 1,6 bisphosphastase inhibitors, glutamine:fructose-6-phosphate amidotransferase inhibitors

8) Anti-obesity agents (for example sibutramine and orlistat);

9) Anti-dyslipidaemia agents such as, HMG-CoA reductase inhibitors (statins, eg pravastatin); PPARα agonists (fibrates, eg gemfibrozil); bile acid sequestrants (cholestyramine); cholesterol absorption inhibitors (plant stanols, synthetic inhibitors); ileal bile acid absorption inhibitors (IBATi), cholesterol ester transfer protein inhibitors and nicotinic acid and analogues (niacin and slow release formulations);

10) Antihypertensive agents such as, β blockers (eg atenolol, inderal); ACE inhibitors (eg lisinopril); calcium antagonists (eg. nifedipine); angiotensin receptor antagonists (eg candesartan), a-antagonists and diuretic agents (eg. furosemide, benzthiazide);

11) Haemostasis modulators such as, antithrombotics, activators of fibrinolysis and antiplatelet agents; thrombin antagonists; factor Xa inhibitors; factor VIIa inhibitors); antiplatelet agents (eg. aspirin, clopidogrel); anticoagulants (heparin and Low molecular weight analogues, hirudin) and warfarin; and

12) Anti-inflammatory agents, such as non-steroidal anti-infammatory drugs (eg. aspirin) and steroidal anti-inflammatory agents (eg. cortisone).

In the above other pharmaceutical composition, process, method, use and medicament manufacture features, the alternative and preferred embodiments of the compounds of the invention described herein also apply.

### EXAMPLES

The invention will now be illustrated in the following non limiting Examples, in which standard techniques known to the skilled chemist and techniques analogous to those described in these Examples may be used where appropriate, and in which, unless otherwise stated:
(i) evaporations were carried out by rotary evaporation in vacuo and work up procedures were carried out after removal of residual solids such as drying agents by filtration;
(ii) all reactions were carried out under an inert atmosphere at ambient temperature, typically in the range 18-25° C., with solvents of HPLC grade under anhydrous conditions, unless otherwise stated;
(iii) column chromatography (by the flash procedure) was performed on Silica gel 40-63 μm (Merck);
(iv) yields are given for illustration only and are not necessarily the maximum attainable;
(v) the structures of the end products of the formula (I) were generally confirmed by nuclear (generally proton) magnetic resonance (NMR) and mass spectral techniques; magnetic resonance chemical shift values were measured in deuterated CDCl₃ (unless otherwise stated) on the delta scale (ppm downfield from tetramethylsilane); proton data is quoted unless otherwise stated; spectra were recorded on a Varian Mercury-300 MHz, Varian Unity plus-400 Mz, Varian Unity plus-600 MHz or on Varian Inova-500 MHz spectrometer unless otherwise stated data was recorded at 400 MHz; and peak multiplicities are shown as follows: s, singlet; d, doublet; dd, double doublet; t, triplet; tt, triple triplet; q, quartet; tq, triple quartet; m, multiplet; br, broad; ABq, AB quartet; ABd, AB doublet, ABdd, AB doublet of doublets; dABq, doublet of AB quartets; LCMS were recorded on a Waters ZMD, LC column xTerra MS C₈(Waters), detection with a HP 1100 MS-detector diode array equipped; mass spectra (MS) (loop) were recorded on VG Platform II (Fisons Instruments) with a HP-1100 MS-detector diode array equipped; unless otherwise stated the mass ion quoted is (MH⁺); unless further details are specified in the text, analytical high performance liquid chromatography (HPLC) was performed on Prep LC 2000 (Waters), Cromasil C₈, 7 μm, (Akzo Nobel); MeCN and de-ionised water 10 mM ammonium acetate as mobile phases, with suitable composition;
(vii) intermediates were not generally fully characterised and purity was assessed by thin layer chromatography (TLC), HPLC, infra-red (IR), MS or NMR analysis;
(viii) where solutions were dried sodium sulphate was the drying agent;
(ix) where an “ISOLUTE” column is referred to, this means a column containing 2 g of silica, the silica being contained in a 6 ml disposable syringe and supported by a porous disc of 54 Å pore size, obtained from International Sorbent Technology under the name “ISOLUTE”; “ISOLUTE” is a registered trade mark;

(x) the following abbreviations may be used hereinbefore or hereinafter:—

| | |
|---|---|
| DCM | dichloromethane; |
| EtOAc | ethyl acetate; |
| DMSO | dimethylsulphoxide; |
| DMF | dimethylformamide; |
| ether | diethyl ether; |
| LDA | lithium diisopropylamine; |
| MeCN | acetonitrile; and |
| THF | tetrahydrofuran. |

### Example 1

### (Thien-3-ylmethyl)-(4-chlorophenyl)-ketone

A solution of 4-chlorophenyl magnesium bromide in ether (6.0 ml of a 1.0 mol solution, 6.0 mmol) was added to a stirred solution of N-methoxy-N-methyl-3-thienylmethanamide (Method 1; 370 mg, 2.0 mmol) in THF (20 ml) at 0° C. The resultant mixture was stirred at ambient temperature overnight, and then quenched with ethanol (50 ml). The resultant mixture was evaporated to dryness and the residue partitioned between water (50 ml) and ether (100 ml). The ether layer was separated, washed with brine, dried and evaporated to dryness. The residue was purified by column chromatography using 5% EtOAc in hexane as eluent to give the title compound, as a solid (170 mg, 0.72 mmol). NMR: 4.3 (s, 2H), 7.0 (d, 1H), 7.1 (d, 1H), 7.3 (dd, 1H), 7.4 (d, 2H), 7.9 (d, 2H).

### Examples 2-3 and Reference Example 1

The procedure described in Example 1 was repeated using the appropriate Grignard reagent and the appropriate Weinreb derivative to obtain the compounds described below.

| Ex | Compound | NMR/m/z |
|---|---|---|
| 2 | (Thien-3-ylmethyl)- | 4.3(s, 2H), 7.0(d, 1H), 7.1(m, |
| | (4-fluorophenyl)-ketone | 3H), 7.3(dd, 1H), 8.0(m, 2H) |
| 3 | (Thien-3-ylmethyl)- | 2.4(s, 3H), 4.3(s, 2H), 7.0(d, |
| | (4-methylphenyl)-ketone | 1H), 7.1(d, 1H), 7.3(m, 3H), |
| | | 7.9(dd, 2H) |
| RE 1¹ | (3-Bromophenyl)- | 305 |
| | (4-methoxybenzyl)-ketone | |

¹This compound was synthesised via p-methoxybenzylchloride Grignard and reacted with a Weinreb amide made from 4-bromobenzoic acid. The Grignard reagent was made with the complex [Mg (anthracene)(THF)₃].

### Reference Example 2

### (4-Methoxybenzyl)-(4-fluorophenyl)-ketone

Diisopropylamine (7.4 ml, 52.8 mmol) was added to anhydrous THF under argon. The solution was stirred and cooled to −35° C. in a dry ice/acetone bath and N-butyl lithium (1.6M, 31.2 ml, 50.4 mmol) was added via a syringe over 2-3 mins, controlling the exotherm to below −20° C. After the addition was complete the reaction was cooled to −70° C. and a solution of 4-methoxyphenylacetic acid (3.89 g, 24 mmol) in THF (48 ml) was transferred to the reaction mixture via a cannula, adding the solution dropwise and keeping the temperature below −55° C. The reaction was stirred for 10 mins at −70° C. and then allowed to warm up to −15° C. A solution of N-methyl-N-methoxy-4-fluorophenylcarbamoyl (4.38 g, 26.4 mmol) in THF (48 ml) was added via a dropping funnel dropwise, while the reaction mixture exothermed to 0° C. After addition was complete, the reaction was allowed to warm up to room temperature and stirred for 1.5 hours. The reaction was added to a stirred solution of concentrated hydrochloric acid (13 ml) in water (250 ml), and was stirred for 10 mins before the addition of ether. The organic layer was separated, washed with water, aqueous sodium bicarbonate and brine and dried (MgSO₄). The solvent removed in vacuo to give a sticky solid. This was triturated with methanol, and the resultant solid was dried under high vacuum to give a white solid (2.2 g). Mp 107-109° C.; NMR(200 MHz) 3.78 (3H, s), 4.17 (2H, s), 6.87 (2H, d), 7.13 (4H, m), 8.03 (2H, dd).

### Reference Example 3

### (4-Bromophenyl)-(4-chlorobenzyl)-ketone

4-Chlorophenylacetic acid (12.78 g) was heated with phosphorus trichloride for 1 hour and then bromobenzene (42.5 ml) was added. The top layer was decanted onto aluminium chloride (11.25 g) in carbon disulfide (50 ml) and the bottom layer was washed with additional bromobenzene (42.5 ml) before this was decanted as well. The reaction mixture was allowed to stir at this temperature for 1.5 hours before heating on a steam bath for 2 hours. The reaction was cooled, poured onto ice/concentrated hydrochloric acid (300 ml) and stirred for 1 hour before extracting with chloroform (3×200 ml). The organic layer was washed with sodium hydroxide (2×200 ml) and water (2×200 ml) and then dried (MgSO₄). The chloroform and any remaining bromobenzene was removed in vacuo. On cooling the resultant black oil crystallised. This solid was recrystallized twice from chloroform/petrol to give 12.4 g, 54%. Mp 128-129° C.; m/z 308 (M⁺).

### Reference Example 3

The procedure described in Reference Example 3 was repeated using the appropriate starting materials to obtain the compound below.

| Ex | Compound | Data |
|---|---|---|
| RE 3 | (4-Bromobenzyl)-(4-chlorophenyl)-ketone | Mp 125-126° C.; 308 |
| | | (M⁺) |

### Example 4

### (4-Chlorophenyl)-(3,4-dichlorophenylmethyl)-ketone

Magnesium turnings (1.95 g) and a crystal of iodine were placed in a reaction vessel and 3,4-dichlorobenzyl chloride (14.63 g) in ether (25 ml) was added slowly ensuring that the temperature of the reaction mixture was kept at the reflux temperature of ether. After the addition was complete the reaction was refluxed for a further 30 mins. 4-Chlorobenzonitrile (8.25 g) in ether (50 ml) was added dropwise while the reaction was at a temperature of ˜30° C. After the addition was complete the reaction was refluxed for 3 hours before being poured onto ice/concentrated sulphuric acid (1 litre) and left to stand overnight. The solid that remained was filtered, washed with water, dissolved in DCM and dried (MgSO₄), filtered and evaporated to dryness. The resulting solid was recrystallized from DCM and petrol twice. (6.05 g, 46%). Mp 99-103° C.; m/z 298 (M⁺).

### Example 5

The procedure described in Example 4 was repeated with the appropriate starting materials to obtain the compound described below.

| Ex | Compound | Mp/Mz |
|---|---|---|
| 5 | (3-Bromophenyl)-(4-chlorophenylmethyl)- | 64-67° C.; 308 (M⁺) |
| | ketone | |

### Example 6

### (4-Bromo-2-hydroxyphenyl)-(4-bromobenzyl)-ketone

(3-Bromophenoxy)-(4-bromobenzyl)-ketone (Method 3; 36.5 g) and aluminium trichloride (26.3 g) were dissolved in nitrobenzene (100 ml) and the mixture was stirred at 100° C. for 2.5 hours. The reaction was left to stand overnight. The reaction mixture was poured onto a mixture of ice/concentrated hydrochloric acid and stirred for 5 mins. The aqueous layer was extracted with EtOAc. The organic layers were combined and the solvent removed in vacuo, and the nitrobenzene was removed by steam distillation. The resultant residue was purified by column chromatography to give the product 31 g. NMR (DMSO-d₆; 400 MHz) 4.30 (s. 2H), 7.05 (dd, 1H), 7.25 (m, 3H), 7.40 (m, 2H), 7.80 (d, 1H), 11.70 (bs, 1H); m/z 370.

### Reference Example 5

### (4-Methoxyphenyl)-(benzyl)-ketone

Sodium hydride (50% dispersion in oil, 960 mg, 20 mmol) was washed with petrol and suspended in anhydrous DMF (10 ml) under nitrogen. N,N-Diethyl-N-(α-cyano-4-methoxybenzyl)amino (Method 4: 3.27 g, 15 mmol) was placed in anhydrous DMF (20 ml) and added to the reaction which was stirred at room temperature for 1 hour. Benzyl chloride (1.89 g, 1.73 ml, 15 mmol) in anhydrous DMF (10 ml) was added dropwise over 1 hour at room temperature (slight exotherm) and the reaction was stirred overnight at room temperature. Methanol (5 ml) was added and the solvent removed in vacuo at 90° C. followed by high vacuum for 2 hours, to give a yellow oil. This was stirred in hydrochloric acid (6M; 40 ml) for 16 hours, and then extracted into chloroform (3×30 ml), washed with water, dried (MgSO₄) and evaporated to dryness to give a yellow oil. This was purified by column chromatography with ether:hexane (2:1). The product was then recrystallized from 40-60° C. petrol to give a colourless solid 410 mg. Mp 68-69° C.; m/z 226.

### Reference Example 6

### (4-Methylthiobenzyl)-(4-fluorophenyl)-ketone

To 10 ml of sieve-dried DCM containing anhydrous zinc iodide (50 mg) was added 4-fluorobenzaldehyde (630 mg, 0.54 ml, 5 mmol). To this stirred mixture, at room temperature and under argon, was added trimethylsilyl cyanide (520 mg, 0.7 ml, 5.25 mmol) and the reaction was stirred overnight (˜20 hours). The solvent was evaporated in vacuo and the residue was treated with anhydrous ether (15 ml) and a little magnesium sulphate. The solution was filtered and the solvent removed in vacuo to give the cyanohydrin as an orange oil (1.14 g). Lithium diisopropylamide was made from n-butyl lithium (2.5M in hexanes, 2.1 ml, 5.25 mmol) and diisopropylamine (500 mg, 0.69 ml, 5 mmol) in THF (5 ml) at −40° C. The reaction was then cooled to −60° C. and the cyanohydrin in THF (5 ml) was added, under argon, at such a rate as to keep the temperature below −55° C. After this addition the reaction was stirred for 30 mins and then 4-methylthiobenzyl chloride (900 mg, 5.25 mmol) was added in THF (2.5 ml). The cooling bath was removed and the reaction stood at room temperature overnight. To the reaction was added saturated ammonium chloride solution (13 ml) and ether (25 ml). The organic phase was separated, washed with saturated ammonium chloride solution, dried (MgSO₄) and the solvent was removed in vacuo to give an orange oil (1.78 g). The oil was taken up in methanol (7 ml) and treated with 2M sulphuric acid (10 ml), the oil precipitated and so acetone (20 ml) was added to give a clear solution. This stood at room temperature overnight. The pH was adjusted to 7.5 with 2M sodium hydroxide solution and the solution was concentrated in vacuo. To the residue was added water and this was extracted with DCM (2×30 ml). The combined extracts were washed with brine and evaporated to give a sticky solid (1.3 g). This was purified by MPLC (1:5 EtOAc:hexane) to give an off-white flaky solid (930 mg). NMR (DMSO-d₆; 400 MHz): 2.40 (s, 3H), 4.30 (s, 2H), 7.20 (s, 4H), 7.35 (tt, 2H), 8.10 (m, 2H); m/z 260.

### Reference Example 7

### (Pyrid-4-ylmethyl)-(pyrid-4-yl)-ketone

A solution of methyl lithium (1.4M) in ether was stirred at room temperature under an argon atmosphere, and 4-picoline (3.72 g, 3.9 ml, 40 mmol) was added dropwise over five mins. When the addition was complete the solution was refluxed gently for 30 mins and then a solution of methyl isonicotinate (2.75 ml, 20 mmol) in ether (5 ml) was added. The resulting sticky suspension was refluxed for 30 mins, cooled and then water (7.5 ml) was added carefully. The reaction mixture was then treated with a cold mixture of 6M hydrochloric acid (10 ml) and water (50 ml). The ether phase was extracted several times with 6M hydrochloric acid, and the combined aqueous phases were treated with 70% sodium hydroxide solution until the solution remained slightly acid. Solid sodium hydrogen carbonate was then added until the mixture was slightly basic (pH 8). The basic mixture was extracted with ether until the extracts no longer gave a brown colour with alcoholic ferric chloride. The combined ether extracts were dried (MgSO₄) and the solvent removed in vacuo to give a yellow solid. This was purified by MPLC (7.5% methanol in DCM) to give a yellow solid (2.0 g). This was recrystallized from toluene/hexane. NMR: 4.35 (s, 2H), 7.20-7.25 (m, 2H), 7.77-7.82 (m, 2H), 8.60-8.65 (m, 2H), 8.85-8.90 (m, 2H); m/z 199.

### Reference Example 8

### (Pyrid-2-ylmethyl)-(phenyl-ketone

A solution of n-butyl lithium (40 ml, 1.6M in hexanes, 66 mmol) in anhydrous ether (50 ml) was stirred in an argon atmosphere. 2-Picoline (6.5 ml, 66 mmol) was added over approximately 10 mins. The resulting red solution was heated gently for 30 mins then cooled to room temperature. To this rapidly stirred solution of 2-picolyllithium was added a solution of benzonitrile (6.8 ml, 66 mmol) in anhydrous ether (10 ml) dropwise, which gave an orange suspension almost immediately. The reaction was stirred at room temperature for 2 hours. The reaction mixture was then treated with water (50 ml) followed by 2M sulphuric acid (50 ml) and the two phase mixture was heated under reflux for 30 mins. After cooling, the reaction mixture was extracted with ether. The aqueous solution was adjusted to pH 7, and then extracted further with ether. The combined ether solutions were washed with water, dried (MgSO₄), and the solvent removed in vacuo to give a deep yellow oil. This was purified by column chromatography (60-80° C. petroleum ether/EtOAc 2:1) and the resulting yellow oil crystallised from warm 60-80° C. petroleum ether to give long yellow needles (410 mg). Mp 57-59° C.; NMR: 3/2 mixture of keto/enol NOTE first two signals are in a ratio for CH₂C(O) and CH═CHOH 4.50 (s), 6.05 (s) 8.60-6.90 (m, 9H), OH offset.

### Reference Example 9

### (Benzyl)-[4-(tetrahydropyran-2-yloxy)phenyl]-ketone

4-Hydroxydeoxybenzoin (20 g) was placed in dihydropyran, with 2 drops of concentrated hydrochloric acid. The mixture was heated for 4 hours at 55° C., and then cooled. The resultant precipitate was taken up in ether:toluene (1:1), heated until the solid went into solution, washed with aqueous sodium hydroxide (2×50 ml), water (2×50 ml), dried, and the solvent removed in vacuo to give a yellow solid. This was taken up in the minimum amount of ethanol with charcoal, and heated. The solution was filtered and as the solution cooled, the product crystallised out. This was separated by filtration and dried in a dessicator. (22.6 g). NMR (DMSO-d_{6;} 400 MHz): 1.50-1.80 (m, 3H), 1.80-1.95 (m, 3H), 3.55-3.60 (m, 1H), 3.75-3.85 (m, 1H), 4.30 (s, 2H), 5.60 (m, 1H), 7.10 (m, 2H), 7.20-7.35 (m, 5H), 8.00 (m, 2H); m/z 297.

### Reference Example 10

### [4-(Benzyl)morpholin-2-ylmethyl]-(phenyl)-ketone

Lithium (2.8 g) was cut into small pieces under an atmosphere of argon, and placed into sodium dried ether (100 ml). A small portion of bromobenzene dissolved in ether was added with vigorous stirring—a reaction commenced within 5 mins and the remaining bromobenzene (21.0 ml in 100 ml of sodium-dried ether) was added dropwise over 30 mins, maintaining a gentle reflux. The reaction was refluxed for a further hour, and then cooled to −20° C. (4-Benzylmorpholin-2-yl)acetonitrile (Journal of Medicinal Chemistry (1990), 33(5), 1406-1321.6 g) was dissolved in sodium dried ether (108 ml) and added dropwise over 15 mins, the temperature not rising above −15° C. The reaction was stirred at this temperature for 15 mins, and then the reaction mixture was poured into 2M hydrochloric acid (800 ml) and ice water (800 ml) with stirring. This was stirred at room temperature for 15 mins, the ether later separated, and the aqueous layer was separated and washed with ether. The aqueous layer was carefully basified with sodium carbonate and extracted with ether. The combined ether extracts were washed with brine, dried (MgSO₄) and the solvent removed in vacuo to give the product as a light brown oil, and as the hydrochloric acid salt. (24.9 g). NMR (DMSO-d₆; 400 MHz) 2.90-3.15 (m, 2H), 3.15-3.50 (m, 2H), 3.80-4.05 (m, 2H), 4.30 (s, 3H), 7.35-7.80 (m, 8H), 7.95 (d, 2H), 11.00 (bs, 1H); m/z 295.

### Example 7

### (Benzimidazol-1-ylmethyl)-(2,4-dichlorophenyl)-ketone

Benzimidazole (5.9 g) was added to a solution of sodium hydride (2.4 g, 50% dispersion in oil) in DMF (55 ml) and stirring was continued until effervescence ceased (25 mins). To this brown solution was added 2,2′,4′-trichloroacetophenone (11.17 g) in DMF (35 ml) over 15 mins and the resulting brown solution was stirred at room temperature for 2 hours. The reaction mixture was poured into water and this was extracted with EtOAc. The extracts were washed with water, dried and the solvent removed in vacuo to give a dark red oil. This was purified by column chromatography (chloroform:MeOH:NH₃ 9:1:0.1) to give an orange oil. This was purified by column chromatography (EtOAc) to give a pale yellow oil (2.5 g). Mp 130-132° C.; NMR (DMSO-d₆; 400 MHz): 5.90 (s, 2H), 7.20-7.30 (m, 2H), 7.55 (m, 1H), 7.65-7.70 (m, 2H), 7.85 (d, 1H), 8.10 (d, 1H), 8.20 (s, 1H); m/z 305.

### Example 8

### [1-Methyl-1-(1,2,4-triazol-1-yl)ethyl]-(4-trifluoromethyl-2-fluorophenyl)-ketone

To (2-fluoro-4-trifluoromethylphenyl)-(2-bromoprop-2-yl)-ketone (Method 5; 9.0 g) was added sodium triazole (2.9 g) in DMF (50 ml). The mixture was heated at 70° C. for 1.5 hours, and the solvent removed. The resulting mixture was purified by MPLC (DCM graduating to 5% methanol in DCM) followed by recrystallization from EtOAc/hexane. NMR (DMSO-d₆; 400 MHz): 1.90 (s, 6H), 7.25 (m, 1H), 7.55 (m, 1H), 7.75 (d, 1H), 8.75 (s, 1H); m/z 302.

### Reference Example 11

### (Benzyl)-(4-methylphenyl)-ketone

A solution of phenylacetyl chloride (77.3 g) in toluene (250 ml) was added dropwise to a suspension of aluminium trichloride (80 g) in toluene (150 ml) over 30 mins with the temperature of the reaction not exceeding 60° C. The reaction was stirred at room temperature for 2.5 hours, then heated at 60° C. for a further 1.5 hours. The reaction mixture was cooled and poured onto ice/hydrochloric acid. The layers were separated and the aqueous layer extracted with toluene. The organic layers were combined, dried, and the solvent removed in vacuo. The solid was recrystallized from 80-100° C. petroleum ether, and dried by heating to give a yield of 77.7 g. Mp 107-111° C.; NMR (DMSO-d₆, 400 MHz): 2.35 (s, 3H), 4.30 (s, 2H), 7.15-7.35 (m, 7H), 7.95 (d, 2H); m/z 211.

### Reference Example 12

### (Imidazol-1-ylmethyl)-(2-chlorothien-5-yl-ketone

To a solution of 2-chloro-5-acetylthiophene (Method 8; 32 g) in chloroform (250 ml) was added bromine (32 g, 10 ml) in chloroform (100 ml) over a period of 1.5 hours. The reaction was catalysed by a few drops of HBr/AcOH and by a UV lamp which also kept the temperature at 40-45° C. during the addition. After the addition was complete, the stirring was continued at room temperature for 2 hours. The reaction mixture was poured onto water and the organic layer was separated and washed with water, dried and the solvent removed in vacuo to give a brown oil which solidified on standing (41.4 g). The solid was dissolved in DMF (100 ml) and added dropwise to a stirred solution of imidazole (68 g) in DMF (200 ml) at 5-10° C., over 30 mins. The resultant brown solution was stirred at room temperature for 18 h. The reaction mixture was poured onto water and extracted with EtOAc. The extracts were washed with water, dried and evaporated to a black gum which crystallised on standing. This solid was re-crystallised from EtOAc to give a tan coloured solid which was filtered, and washed with ether to give 11.5 g. Mp 109-111° C.; NMR (DMSO-d₆): 5.18 (s, 2H), 6.65-7.10 (m, 3H), 7.22-7.48 (m, 2H).

### Example 9

### [2-(4-Chlorophenyl)-1-(pyrid-3-yl)ethyl]-(4-chlorophenyl)-ketone

Sodium hydride (100%) (500 mg) was suspended in anhydrous DMF (30 ml) and the reaction was cooled to 0° C. To the reaction was added (pyrid-3-ylmethyl)-(4-chlorophenyl)-ketone (Reference Example 13) in DMF (20 ml) and the reaction mixture was stirred for 1 hour. 4-Chlorobenzyl chloride (3.2 g) in DMF (10 ml) was added, and the reaction stirred at 0° C. for 3 hours. The reaction was poured onto water, and the resultant crystals were filtered and recrystallized from 60-80° C. petrol with charcoal to give a white solid (2.75 g). Mp 98-99° C.

### Example 10

### [α-(4-Fluorobenzyl)benzyl]-(pyrid-3-yl)-ketone

The procedure described in Example 9 was repeated substituting the 4-chlorobenzyl chloride with 4-fluorobenzyl chloride and using (pyrid-3-yl)-(benzyl)-ketone (Reference Example 14) to give the title compound 7 g. Mp 100-102° C.; m/z 305 (M⁺).

### Reference Example 13

### (Pyrid-3-ylmethyl) -(4-chlorophenyl)-ketone

To diisopropylamine (56 ml) was added n-butyl lithium (2.4M, 166 ml) keeping the temperature of the reaction mixture below 20° C. by cooling in an ice/salt bath. 3-Picoline (37.2 g) was added dropwise, diluted with toluene (30 ml) and the reaction stirred at 0° C. for 30 mins before the addition of 4-chloromethyl benzoate (34 g) in anhydrous toluene (30 ml). The reaction was stirred at 5° C. for 1.5 hours. The reaction mixture was poured onto ice/water, acidified and washed to remove ester, then the acid layer was basified and extracted to give a red oil. This was distilled under vacuum which gave 1 fraction at 154° C., 0.2 mmHg, which resulted in yellow crystals. These were triturated with petrol/ether to give a cream solid (17 g). Mp 61-63° C.

### Reference Example 14

### (Pyrid-3-yl)-(benzyl)-ketone

The title compound was prepared by the procedure of Reference Example 13 using the appropriate starting materials. Mp 132-138° C.

### Example 11

### (4-Chlorophenyl)-{α-hydroxy-α-1-(1,2,4-triazol-1-yl)ethyl]-4-chlorobenzyl}-ketone

A solution of 4-bromo-1-chlorobenzene (17.1 g, 90 mmol) in anhydrous ether (100 ml) was added portionwise to magnesium turnings (2.16 g, 90 mmol) suspended in anhydrous ether (100 ml), with a crystal of iodine. The reaction was refluxed for 4 hours. Anhydrous toluene (150 ml) was added and the temperature increased, with the ether distilling off. When all of the ether had been removed, 1-[1-(4-chlorophenyl)-1-(trimethylsilyloxy)-1-(cyano)prop-2-yl]-1,2,4-triazole (Method 9) in toluene (10 ml) was added and the reaction was refluxed and stirred overnight. After cooling, the reaction mixture was acidified with 3M hydrochloric acid (100 ml) and stirred for 1 hour. The aqueous and organic layers were separated, and the aqueous layers washed with ether. The ether extracts were combined with the organic layer, which was then dried (MgSO₄). After filtration, the solvent was removed in vacuo to give a deep red oil. The was purified by column chromatography (4% methanol in DCM) to give the required product (1.4 g). Mp 181-183° C.; NMR: 1.70 (d, 3H), 5.30 (q, 1H), 5.70 (s, 1H), 7.50 (m, 10H); m/z 376.

### Reference Example 15

### [2,4-Dichloro-α-(1,2,4-triazolylmethyl)benzyl]-(4-chlorophenyl)-ketone

[1-(2,4-Dichlorophenyl)vinyl]-(4-chlorophenyl)-ketone (Method 26; 1.6 g, 5 mmol) was added to ethanol (25 ml) containing triazole (2 g) and triethylamine (20 drops) and the reaction was stirred at room temperature for 2 hours. The reaction mixture was poured onto water and extracted with ether. The extracts were washed with water, and the solvent evaporated in vacuo to give an oil. This was placed in ether/petrol to give a white precipitate which was collected by filtration. This was purified by MPLC to give a free flowing white crystalline solid (1.25 g). Mp 109-111° C.; NMR: 4.35 (q, 1H), 4.95 (q, 1H), 5.65 (q, 1H), 7.20 (m, 5H), 7.75 (m, 2H), 7.85-8.00 (dd, 2H).

### Reference Example 16

### (4-Chlorophenyl)-[α-(1,2,4-triazol-1-ylmethyl)-4-chlorobenzyl]-ketone

Reference Example 15 was repeated with [1-(4-chlorophenyl)vinyl]-(4-chlorophenyl)-ketone (J. Med. Chem. (1972), 15(12), 1243-7) to give the title compound. Mp 126-128° C. 345 (M⁺).

### Reference Example 17

### (2,4-Dichlorobenzyl)-(4-chlorophenyl)-ketone

2,4-Dichlorobenzyl chloride (92.5 g, 0.48 mol) in ether (300 ml) was added over 1 hour to magnesium (13 g) in ether (50 ml) at reflux, and the reaction was allowed to stand at room temperature overnight. 4-Chlorobenzonitrile (0.2 mol) was dissolved in sieve-dried THF and the Grignard reagent (180 ml) was added over 5 min with stirring. The reaction mixture was refluxed for 24 h under argon. The reaction mixture was cooled and poured into 2M hydrochloric acid/ice and extracted with EtOAc. On drying of the solution and evaporation of the solvent gave a yellow solid. This was triturated with 50:50 EtOAc:ether, and the resulting pale yellow solid was filtered (24.7 g). Mp 127-129° C.

### Example 12

### [2-(2-Fluorophenyl)-1-(1,2,4-triazol-1-yl)ethyl]-(thien-2-yl)-ketone

Sodium hydride (610 mg, 26 mmol) was suspended in DMF (10 ml) and (1,2,4-triazol-1-ylmethyl)-(thien-2-yl)-ketone (Journal of Medicinal Chemistry (1987), 30(8), 1497-502; 5 g, 26 mmol) dissolved in DMF (30 ml), was added. The reaction was stirred at room temperature for 3 hours and then cooled in an ice-bath and 2-fluorobenzyl chloride (3.72 g, 26 mmol), in DMF (15 ml) was added dropwise, keeping the temperature between 0 and 5° C. The reaction was left to stir at room temperature overnight, then poured onto water which formed a precipitate. This was filtered and recrystallized from petroleum ether 60-80° C. to give the product (2.95 g). Mp 121-122° C.

### Example 13

### [2-(4-Chlorophenyl)-1-(pyridazin-3yl)ethyl]-(phenyl)-ketone

(Phenyl)-(pyridazin-3-ylmethyl)-ketone (Chemical & Pharmaceutical Bulletin (1978), 26(12), 3633-40.2.5 g, 13 mmol) in DMF (25 ml) was added to a suspension of sodium hydride (610 mg, 50% dispersion in oil, 13 mmol, washed with ether) in DMF (10 ml). After stirring for 2 hours the solution was cooled in an ice/salt bath and 4-chlorobenzyl chloride (2 g, 12.5 mmol) in DMF (15 ml) was added dropwise at 0-5° C. The reaction mixture was warmed to room temperature and stirred for a further 1 hour. The reaction mixture was poured onto water (200 ml) which gave a yellow precipitate, which was filtered, washed with water, dried and recrystallized from EtOAc/60-80° C. petroleum ether to give the product (1.6 g). Mp 140-142° C.; m/z 322 (M⁺).

### Examples 14-16

Following the procedure described in Example 13, the following compounds were made using the appropriate starting materials.

| Ex | Compound | Data |
|---|---|---|
| 14¹ | [2-(2,4-Dichlorophenyl)-1- | NMR(400MHz; DMSO-d₆): 3.35(m, 1H), 3.55(m, |
| | (pyridazin-3-yl)ethyl]- | 1H), 5.50(m, 1H), 7.30(s, 2H), 7.45(m, 2H), |
| | (phenyl)-ketone | 7.50-7.70(m, 3H), 7.95(d, 2H), 9.05(m, 1H); m/z 359 |
| 15² | [2-(4-Chlorophenyl)-1- | Mp 116-118° C. |
| | (pyrazin-2-yl)ethyl]- | |
| | (pyridin-3-yl)-ketone | |
| 16² | [2-(4-Chlorophenyl)-1- | Mp 107-109° C.; m/z 312(M⁺) |
| | (pyrazin-2-yl)ethyl]-(thien- | |
| | 2-yl)-ketone | |

¹Prep of starting material: Chemical & Pharmaceutical Bulletin (1978), 26(12), 3633-40
²The starting materials for Examples 15 and 16 could be prepared according to the procedure described in Chemical & Pharmaceutical Bulletin (1978), 26(12) for (phenyl)-(pyridazin-3-ylmethyl)-ketone.

### Examples 17 and 18

### [2-(4-Chlorophenyl)-1-(pyrazin-2-yl)ethyl]-(pyridin-3-yl)-ketone enantiomer 1 and [2-(4-Chlorophenyl)-1-(pyrazin-2-yl)ethyl]-(pyridin-3-yl)-ketone enantiomer 2

[2-(4-Chlorophenyl)-1-(pyrazin-2-yl)ethyl]-(pyridin-3-yl)-ketone (Example 15) was separated into its 2 enantiomers using the following HPLC conditions.

| Instrument | Perkin Elmer 200 |
|---|---|
| Column | 10 μm Chiralpak AD (4.6 mm × 250 mm) No. |
| | AD00CE-BJ182 |
| Eluent | MeCN/MeOH 95/5 |
| Oven Temperature | Ambient |
| Flow | 1 ml/min |
| Wavelength | 254 nm |
| Sample | 1 mg/ml in EtOH |
| Concentration | |
| Sample Volume | 20 μl |
| Run Time | 30 mins |

### Reference Example 18

### (Phenyl)-(cyclohexylmethyl)-ketone

In a conical flask was placed deoxybenzoin (500 mg, 2.55 mmol), tetrabutylammonium bromide (41 mg, 0.13 mmol), (bromomethyl)cyclohexane (1.35 g, 7.65 mmol), toluene (18 ml) and 45% KOH in water (6 ml). The reaction was sonicated at room temperature for 3 hours and then quenched with saturated ammonium chloride solution (˜5 ml). The volatiles were removed under reduced pressure and the resulting material was partitioned between ether and water. The organic layer was separated and re-extracted with water then washed with brine, dried (MgSO₄), filtered and evaporated to yield an oil which was further purified by prep LCMS to yield the product as a clear oil. NMR: 1.00 (br m, 2H), 1.25 (br m, 3H), 1.70 (br m, 5H), 2.00 (m, 1H), 2.80 (d, 2H), 7.45 (t, 2H), 7.55 (t, 1H), 7.95 (d, 2H); m/z: 202.

### Example 19

### (4-Fluorophenethyl)-(4-trifluoromethylphenyl)-ketone

[2-(4-Fluorophenyl)vinyl]-(4-trifluoromethylphenyl)-ketone (Method 27; 1 g) was hydrogenated over Pd/CaCO₃ in ethanol. The catalyst was filtered off, the solvent removed in vacuo and the residue obtained recrystallized from aqueous ethanol (510 mg). Mp 66-67° C.; m/z 296 (M⁺).

### Examples 20-22 and Reference Example 19

The procedure described in Example 19 was carried out using the appropriate starting materials to obtain the products described below.

| Ex | Compound | Data | SM |
|---|---|---|---|
| 20 | (4-Fluorophenethyl)-(4- | Mp 60° C.; m/z 262(M⁺) | Method |
| | chlorophenyl)-ketone | | 28 |
| 21 | (4-Chlorophenethyl)-(2,4- | Mp 70-71° C.; m/z 280(M⁺) | Method |
| | difluorophenyl)-ketone | | 29 |
| 22 | (4-Fluorophenethyl)-(2,4- | Mp 46° C.; m/z 264(M⁺) | Method |
| | difluorophenyl)-ketone | | 30 |
| RE 19 | (Phenethyl)-(4-methoxyphenyl)- | NMR(400 MHz. DMSO-d₆): | Method |
| | ketone | 2.80(t, 2H), 3.20(t, 2H), 3.85(s, 3H), | 31 |
| | | 6.90(m, 2H), 7.10(m, 1H), 7.20(m, 4H), | |
| | | 7.85(m, 2H); m/z 240(M⁺) | |

### Example 23

### (Phenyl)-[2-(4-methylphenyl)-1-(piperidin-1-yl)ethyl]-ketone

4-Methylbenzylideneacetophenone (11.0 g, 50 mmol) and piperidine (17 ml, 230 mmol) were heated in a sealed tube at 100° C. for 4 hours. The mixture was cooled to room temperature, the solid product filtered and crystallised from hexane to give the title compound as a solid (7.0 g, 23 mmol). Mp. 71-72° C.; m/z 307 (M⁺).

### Example 24

### (α-Methylamino-4-methylbenzyl-(4-methylphenyl)-ketone

To 4,4′-dimethylbenzoin (500 mg, 2.1 mmol) in 40% aq methylamine (1.1 ml) was added methylamine hydrochloride (20 mg). The reaction was warmed to reflux and stirred at this temperature for 2 hours before addition of further 40% aq methylamine (0.5 ml). The reaction was stirred at reflux for a further 3 hours then cooled to room temperature. Saturated sodium hydrogen carbonate (15 ml) was added and the crude mixture was extracted with ether (2×30 ml). The ether layers were combined and washed with brine then dried (MgSO₄), filtered and evaporated under reduced pressure to yield an oil. This crude product was dissolved in ether and then acidified with hydrochloric acid in ether (˜0.2M), the resulting precipitate was filtered off and recrystallized from EtOH to give the product as a white solid (154 mg, 25%). NMR (DMSO-d₆): 2.25 (s, 3H), 2.35 (s, 3H), 2.45 (s, 3H), 6.35 (s, 1H), 7.25 (d, 2H), 7.30 (d, 2H), 7.45 (d, 2H), 7.90 (d, 2H), 9.90 (br s, 2H); m/z 254.

### Examples 25-28

The procedure described in Example 24 was repeated using the appropriate starting materials to obtain the compounds described below.

| Ex | Compound | M/z | NMR(DMSO-d₆) |
|---|---|---|---|
| 25 | (α-Methylamino-4- | 294 | 2.45(s, 3H), 6.50(s, 1H), 7.50(d, 2H), |
| | chlorobenzyl)-(4-chlorophenyl)- | | 7.60(d, 4H), 8.05(d, 2H), 9.80(br s, 1H), |
| | ketone | | 10.2(br s, 1H) |
| 26 | (α-Ethylamino-4-chlorobenzyl)- | 308 | 1.30(t, 3H), 2.80(br s, 1H), 2.95(br s, 1H), |
| | (4-chlorophenyl)-ketone | | 6.50(br s, 1H), 7.55(d, 2H), 7.60(m, 4H), |
| | | | 8.10(d, 2H), 9.65(br s, 1H), 9.90(br s, 1H) |
| 27 | (α-Isopropylamino-4- | 322 | 1.30(m, 6H), 3.05(br s, 1H), 6.40(br s, |
| | chlorobenzyl)-(4-chlorophenyl)- | | 1H), 7.50(d, 2H), 7.60(d, 2H), 7.70(d, |
| | ketone | | 2H), 8.15(d, 2H), 9.50(br s, 1H) |
| 28 | (α-Ethylamino-4-methylbenzyl)- | 268 | 1.25(t, 3H), 2.25(s, 3H), 2.30(s, 3H), |
| | (4-methylphenyl)-ketone | | 2.70(br s, 1H), 2.90(br s, 1H), 6.35(s, 1H), |
| | | | 7.20(d, 2H), 7.30(d, 2H), 7.45(d, 2H), 7.95(d, |
| | | | 2H), 9.50(br s, 1H), 9.95(br s, 1H) |

### Example 29

### (1,3-Benzodioxol-5-yl)-[1-(1,3-benzodioxol-5-yl)-1-(ethylamino)methyl]-ketone

A suspension of piperoin (250 mg, 0.83 mmol) and ethylamine hydrochloride (40 mg, 0.5 mmol) in 70% aq ethylamine (4 ml) was heated in a microwave at 125° C. for 10 minutes. Volatiles were removed under reduced pressure and the resulting crude oil was purified by column chromatography (DCM to 5% MeOH/DCM). This material was dissolved in ether and treated with hydrochloric acid in ether. The resulting solid was filtered off and recrystallized from ethanol to yield a solid (50 mg, 20%). NMR (DMSO-d₆): 1.25 (t, 3H), 2.85 (m, 2H), 6.00 (d, 2H), 6.10 (s, 2H), 6.15 (s, 1H), 6.90 (d, 1H), 7.00 (d, 1H), 7.10 (m, 2H), 7.50 (s, 1H), 7.70 (d, 1H); m/z: 328.

### Example 30

### (Thien-2-yl)-[4-(4-chlorobenzoyl)piperidin-1-ylmethyl]-ketone

To a stirred suspension of (4-chlorophenyl)(4-piperidyl)methanone hydrochloride (100 mg, 0.41 mmol) in DCM (5 ml) was added triethylamine (104 mg, 1.03 mmol) and 2-bromo-1-(2-thienyl)-1-ethanone (76 mg, 0.37 mmol). The reaction was stirred at room temperature for 1 hour. The crude reaction mixture was transferred to a separating funnel and washed with 2M hydrochloric acid. The organic layer was separated and washed with water then evaporated to yield an impure solid. This material was partitioned between DCM and saturated sodium hydrogencarbonate solution. The organic layer was separated and washed with brine then dried (MgSO₄), filtered and evaporated to give a solid. This solid was dissolved in ether and treated with hydrochloric acid in ether. The resulting solid was filtered off to yield the product as a solid (24 mg, 17%). NMR (DMSO-d₆): 2.00 (m, 4H), 3.20 (m, 2H), 3.50 (m, 1H), 3.60 (m, 2H), 5.00 (s, 2H), 7.35 (s, 1H), 7.60 (d, 2H), 8.05 (d, 2H), 8.10 (s, 1H), 8.20 (d, 1H), 10.20 (br s, 1H); m/z: 348.

### Example 31

### (α-Methyl-α-hydroxy-4-fluorobenzyl)-(4-fluorophenyl)-ketone

A solution of methyl magnesium chloride in THF (0.67 ml of a 3.0 mol solution, 2.0 mmol) was added to a stirred solution of 4,4′-difluorobenzil (492 mg, 2.0 mmol) in ether (20 ml) during 30 mins at ambient temperature. The resultant mixture was stirred at ambient temperature for 30 mins and then quenched with a saturated aqueous solution of ammonium chloride (2.0 ml) and water (3.0 ml). The ether layer was separated, washed with brine, dried and evaporated to dryness. The residue was purified by column chromatography using 20% EtOAc in hexane as eluent to give the title compound as a solid (300 mg, 1.15 mmol). NMR: 1.9 (s, 3H), 4.5 (s, 1H), 7.0 (m, 4H), 7.4 (dd, 2H), 7.75 (dd, 2H).

### Examples 32-34 and Reference Example 20

The procedure described in Example 31 was repeated using the appropriate Grignard reagent to replace the methyl magnesium chloride and the appropriate benzil to replace the 4,4′-difluorobenzil to obtain the compounds described below.

| Ex | Compound | NMR |
|---|---|---|
| 32 | (α-Benzyl-α-hydroxy-4- | 3.3(d, 1H), 3.7(d, 1H), 3.7(s, 1H), 6.95(m, |
| | fluorobenzyl)-(4-fluorophenyl)- | 4H), 7.05(m, 2H), 7.2(m, 3H), |
| | ketone | 7.45(dd, 2H), 7.8(dd, 2H) |
| 33 | (α-Ethyl-α-hydroxy-4- | 0.9(t, 3H), 2.4(q, 2H), 4.5(s, 1H), |
| | fluorobenzyl)-(4-fluorophenyl)- | 7.0(m, 4H), 7.4(dd, 2H), 7.7(dd, 2H) |
| | ketone | |
| RE 20 | (α-Methyl-α-hydroxy-4- | 1.9(s, 3H), 4.4(s, 1H), 7.3(m, 6H), |
| | chlorobenzyl)-(4-chlorophenyl)- | 7.6(m, 2H) |
| | ketone | |
| 34 | (α-Methyl-α-hydroxy-2- | 2.0(s, 3H), 4.75(s, 1H), 7.0(m, 4H), |
| | thienylmethyl)-(2-thienyl)-ketone | 7.15(d, 1H), 7.3(d, 1H), 7.6(m, 2H) |

### Example 35

### (α-Ethoxy-4-fluorobenzyl)-(4-fluorophenol)-ketone

A solution of ethyl magnesium bromide in THF (6.0 ml of a 1.0 mol solution, 6.0 mmol) was added to a stirred solution of 4,4′-difluorobenzil (492 mg, 2.0 mmol) in ether (20 ml) during 30 minutes at ambient temperature. The resultant mixture was stirred at ambient temperature for 30 minutes and then quenched with a saturated aqueous solution of ammonium chloride (6.0 ml) and water (6.0 ml). The ether layer was separated, washed with brine, dried and evaporated to dryness. The residue was purified by column chromatography using 10% EtOAc in hexane as eluent to give the title compound as a solid (58 mg, 0.21 mmol). NMR: 1.2 (t, 3H), 3.6 (q, 4H), 5.4 (s, 1H), 7.0 (m, 4H), 7.4 (dd, 2H), 8.0 (dd, 2H).

### Example 36

### (α-Isopropoxy-4-fluorobenzyl)-(4-fluorophenyl)-ketone

A solution of isopropyl magnesium chloride in THF (3.0 ml of a 2.0 mol solution, 6.0 mmol) was added to a stirred solution of 4,4′-difluorobenzil (492 mg, 2.0 mmol) in ether (50 ml) during 30 minutes at ambient temperature. The resultant mixture was stirred at ambient temperature for 30 minutes and then quenched with a saturated aqueous solution of ammonium chloride (6.0 ml) and water (6.0 ml). The ether layer was separated, washed with brine, dried and evaporated to dryness. The residue was purified by column chromatography using 20% EtOAc in hexane as eluent to give the title compound as a solid (130 mg, 0.45 mmol). NMR: 0.75 (d, 3H), 0.95 (d, 3H), 2.2 (m, 1H), 5.2 (s, 1H), 7.0 (m, 4H), 7.2 (dd, 2H), 7.4 (dd, 2H).

### Example 37

### (α-Methoxy-4-fluorobenzyl)-(4-fluorophenyl)-ketone

Sodium tert-butoxide (125 mg, 1.3 mmol) was added to a stirred solution of 4-fluoro-1-bromobenzene (176 mg, 1.0 mmol), 1-(4-fluorophenyl)-2-methoxyethanone (Method 25; 185 mg), 2-dicyclohexylphosphino-2′-(N,N-dimethylamino)-biphenyl (8 mg, 0.02 mmol) and palladium acetate (2.2 mg, 0.01 mmol) in dry toluene (1 ml) under argon. The resultant mixture was heated at 80° C. for 16 hours, cooled to room temperature and partitioned between water (10.0 ml) and ether (25 ml). The aqueous layer was extracted with ether (2×10 ml), the combined ether extracts washed with brine, dried and evaporated to dryness. The residue was purified by column chromatography using 10% EtOAc in hexane as eluent to give the title compound as a solid (178 mg, 0.68 mmol). NMR: 3.4 (s, 3H), 5.4 (s, 1H), 7.0 (m, 4H), 7.4 (dd, 2H) and 8.0 (dd, 2H).

### Examples 38-41

The procedure described in Example 37 was repeated using the appropriate bromobenzene to replace the 4-fluoro-1-bromobenzene to obtain the compounds described below.

| Ex | Compound | NMR |
|---|---|---|
| 38 | (α-Methoxy-4-methylbenzyl)-(4- | 2.3(s, 3H), 3.4(s, 3H), 5.4(s, 1H), 7.0(dd, |
| | fluorophenyl)-ketone | 2H), 7.1(d, 2H), 7.3(d, 2H), 8.0(dd, |
| | | 2H) |
| 39 | (α-Methoxy-4-methoxybenzyl)-(4- | 3.4(s, 3H), 3.8(s, 3H), 5.4(s, 1H), 6.8(d, |
| | fluorophenyl)-ketone | 2H), 7.0(dd, 2H), 7.3(d, 2H), 8.0(dd, |
| | | 2H) |
| 40 | (α-Methoxy-4[N,N- | 2.7(s, 6H), 3.5(s, 3H), 5.4(s, 1H), 7.1(dd, |
| | dimethylsulphamoyl)benzyl]-(4- | 2H), 7.6(d, 2H), 7.8(d, 2H), 8.0(dd, |
| | fluorophenyl)-ketone | 2H) |
| 41 | [α-Methoxy-4-(methoxymethyl)benzyl]- | 3.2(s, 2H), 3.4(s, 3H), 3.45(s, 3H), 5.4(s, |
| | (4-fluorophenyl)-ketone | 1H), 7.0(m, 4H), 7.4(dd, 2H), 8.0(dd, |
| | | 2H) |

### Reference Example 21

### (4-Methyl-α-hydroxbenzyl)-(4-chlorophenyl)-ketone

A solution of sodium methoxide in methanol (10.0 ml of a 0.5 mol solution, 5.0 mmol) was added to a stirred solution of 2-bromo-1-(4-chlorophenyl)-2-(4-methylphenyl)-ethan-1-one (323 mg, 1.0 mmol) in methanol (10 ml) during 30 minutes at ambient temperature. The resultant mixture was stirred at ambient temperature for 3 hours and then quenched with 1M hydrochloric acid (5.0 ml). The methanol was evaporated and the aqueous residue treated with ether (20 ml). The ether layer was separated, washed with brine, dried and evaporated to dryness. The residue was purified by column chromatography using 10% EtOAc in hexane as eluent to give the title compound as a solid (215 mg, 0.78 mmol). NMR: 2.3 (s, 3H), 4.4 (d, 1H), 5.8 (d, 1H), 7.1-7.2 (m, 4H), 7.4 (dd, 2H) and 7.8 (dd, 2H).

### Example 42

### (4-Fluorophenyl)-[α-(5-chloropyrimidin-2-yloxy)-4-fluorobenzyl]-ketone

To a stirred solution of 5-chloro-2-hydroxypyrimidine (130 mg, 1.0 mmol), 4,4′-difluorobenzoin (372 mg, 1.5 mmol) and triphenylphosphine (524 mg, 2 mmol) in dry THF (10 ml) was added a solution of di-isopropylazodicarboxylate (445 mg, 2.2 mmol) at 0° C. under argon. The resultant mixture was stirred at ambient temperature for 16 hours, partitioned between water (25 ml) and ether (25 ml). The aqueous layer was extracted with ether (25 ml), and the combined ether extracts were washed with brine, dried and evaporated to dryness. The residue was purified by column chromatography using 50% EtOAc in hexane as eluent to give the title compound as a solid (74 mg, 0.21 mmol). NMR: 7.1 (m, 4H), 7.3 (m, 2H), 7.4 (d, 1H), 7.5 (s, 1H), 8.0 (m, 2H) and 8.5 (d, 1H); m/z 359 (M+H)⁻.

### Example 43

### (α-Hydroxy-4-methoxybenzyl)-(naphth-2-yl)-ketone

2-Naphthaldehyde (3.75 g, 24 mmol) was dissolved in DCM (50 ml) and zinc diiodide (250 mg) was added. This was stirred at room temperature under argon and trimethylsilyl cyanide (6.65 ml, 25 mmol) was added via syringe. The reaction mixture was stirred overnight. The solvent was removed in vacuo to leave an orange oil. LDA was prepared by adding diisopropylamine (3.35 ml, 24 mol) in THF (25 ml) and cooling to −60° C., before adding n-butyl lithium (1.54 ml) under argon. This was stirred for 15 mins before adding the orange oil—the cyanohydrin—in THF (20 ml) and stirring at −60° C. for 30 mins. Para-anisaldehyde (2.92 ml, 24 mmol) in THF (15 ml) was added and the reaction was allowed to stir and warm up to room temperature overnight. Saturated aqueous ammonium chloride (65 ml) was added to the reaction mixture followed by ether (100 ml). The organic phase was separated, washed with saturated ammonium chloride, dried (MgSO₄) and the solvent removed in vacuo to give an orange oil. This was taken up in methanol (30 ml) and 1M sulphuric acid (10 ml) was added. The reaction mixture was left to stand overnight. The pH was adjusted to pH 7-8, and the mixture was concentrated and extracted with DCM. The organic layers were combined, washed, dried and evaporated to give an orange oil, which was purified by column chromatography (EtOAc:hexane, 10:1) to give a pale yellow solid (56 mg, 0.8%). Mp 121-128° C.; NMR (200 MHz, DMSO-d₆): 3.65 (s, 3H), 5.8-5.9 (bs, 1H), 6.20 (s, 1H), 6.80 and 7.35 (AB q, 4H), 7.60-7.90 (m, 4H).

### Reference Example 22

### (α-Hydroxy-4-methoxybenzyl)-(4-methoxyphenyl)-ketone

Anisaldehyde (20 g) was dissolved in methanol (25 ml) and water (16 ml). Potassium cyanide (4 g) was added and the mixture was refluxed for 2 hours. Further potassium cyanide (4 g) was added and the reaction refluxed for a further 2 hours. On allowing to stand, an oil separated. The solvent was removed in vacuo and the residue was taken up in water, and extracted with ether. The extracts were combined, washed with water, dried (MgSO₄) and the solvent removed in vacuo to give an oil. This was extracted with hot petroleum ether 60-80° C. to remove the anisaldehyde, and the residue triturated with ethanol to give a solid (1.6 g). This was recrystallized from aqueous ethanol to give the product (820 mg). Mp 110-112° C.; m/z 272 (M⁺).

### Examples 44-45 and Reference Example 23

Following the procedure of Reference Example 22 using the appropriate starting materials the following compounds were prepared.

| Ex | Compound | Data |
|---|---|---|
| RE | (α-Hydroxybenzyl)-(4- | Mp 105.5-106° C.(lit. 106° C.); m/z 242 |
| 23 | methoxyphenyl)-ketone | (M⁺) |
| 44 | (1-Napth-2-yl-1-hydroxymethyl)- | Mp 128-132° C.; NMR(200 MHz, DMSO- |
| | [4-(N,N-dimethylamino)phenyl]- | d₆): 2.95(s, 6H), 5.85(bs, 1H), 6.10(s, 1H), |
| | ketone | 6.60(AB q, 2H), 7.50(m, 3H), 7.90(m, 6H) |
| 45¹ | (α-Hydroxy-3,4-dichlorobenzyl)- | Mp 100-102° C.; m/z 332(M⁺) |
| | (3,4-dichlorophenyl)-ketone | |

¹This compound was prepared with sodium cyanide, not potassium cyanide.

### Example 46

### [α-Hydroxy-α-(N,N-diisopropylaminomethyl)benzyl]-(phenyl)-ketone

Diisopropylamine (11.6 g, 115 mmol) was added to a solution of 2-hydroxy-1,2-diphenyl-ethanone (21 g, 100 mmol) and 40% aqueous formaldehyde (10 ml, 140 mmol) in ethanol (40 ml) and the mixture was heated under reflux for 2 hours. The mixture was cooled to room temperature and partitioned between water (200 ml) and ether (600 ml). The ether layer was washed with water (2×200 ml) and extracted with 1M hydrochloric acid (3×150 ml). The combined acidic extracts were basified with concentrated aqueous sodium hydroxide solution and extracted with ether (3×150 ml). The combined ether extracts were dried, treated with hydrogen chloride in ethanol until acidic and evaporated to dryness. The residue was crystallised from ethanol to give the title compound as a solid (3.9 g, 10.8 mmol). M/z 325 (M⁺).

### Example 47

### (2-Thien-2-ylethyl)-(4-chlorophenyl)-ketone

A solution of 4-chlorophenyl magnesium bromide in ether (6.0 ml of a 1.0 mol solution, 6.0 mmol) was added to a stirred solution of N-methoxy-N-methyl-2-thienylethanamide (Method 2; 398 mg, 2.0 mmol) in THF (20 ml) at 0° C. The resultant mixture was stirred at ambient temperature overnight and then quenched with ethanol (50 ml). The resultant mixture was evaporated to dryness and the residue partitioned between water (50 ml) and ether (100 ml). The ether layer was separated, washed with brine, dried and evaporated to dryness. The residue was purified by column chromatography using 5% EtOAc in hexane as eluent to give the title compound as a solid (250 mg, 1.0 mmol). NMR: 3.3 (m, 4H), 6.8 (dd, 1H), 6.9 (dd, 1H), 7.1 (dd, 1H), 7.4 (d, 2H), 7.9 (d, 2H).

### Reference Examples 24-26 and Examples 48-50

The procedure described in Example 47 was repeated using the appropriate N-methoxy-N-methyl amide to replace the N-methoxy-N-methyl-2-thienylethanamide and the appropriate Grignard or lithium reagent to replace the 4-chlorophenyl magnesium bromide to obtain the compounds described below.

| Ex | Compound | NMR |
|---|---|---|
| RE | (4-Fluorophenethyl)-(4- | 3.0(t, 2H), 3.2(t, 2H), 6.9(dd, 2H), 7.1(dd, |
| 24 | fluorophenyl)-ketone | 2H), 7.2(dd, 2H), 8.0(dd, 2H) |
| RE | (4-Chlorophenethyl)-(4- | 3.0(t, 2H), 3.2(t, 2H), 7.0(dd, 2H), 7.1(dd, |
| 25 | fluorophenyl)-ketone | 2H), 7.2(dd, 2H), 8.0(dd, 2H) |
| RE | (2-Thien-2-ylethyl)-(4- | 3.3(m, 4H), 6.8(d, 1H), 6.9(dd, 1H), 7.1(m, |
| 26 | fluorophenyl)-ketone | 3H), 8.0(dd, 2H) |
| 48 | (2-Thien-2-ylethyl)-(4- | 2.4(s, 3H), 3.3(m, 4H), 6.8(d, 1H), 6.9(dd, |
| | methylphenyl)-ketone | 1H), 7.1(dd, 1H), 7.2(dd, 2H), 7.8(dd, 2H) |
| 49 | (4-Chlorophenethyl)-(thiazol-2-yl)- | 3.0(t, 2H), 3.5(t, 2H), 7.2(m, 4H), 7.6(d, 1H), |
| | ketone | 8.0(d, 1H) |
| 50 | (2-Thien-2-ylethyl)-(thiazol-2-yl)- | 3.3(t, 2H), 3.6(t, 2H), 6.9(dd, 2H), 7.1(d, |
| | ketone | 1H), 7.6(d, 1H), 8.0(d, 1H) |

### Reference Examples 27-28 and Examples 51-65

The following compounds were made by the procedure of J.Med.Chem.; EN; 30; 12; 1987; 2232-2239.

| Ex | Compound | M/z | NMR |
|---|---|---|---|
| RE | (Morpholinosulphonylmethyl)- | 286 | 3.3(dd, 4H), 3.7(dd, 4H), 4.5(s, 2H), |
| 27 | (4-fluorophenyl)-ketone | | 7.2(m, 2H), 8.0(m, 2H) |
| 51 | (Piperidin-1-ylsulphonyl | 284 | 1.6(m, 6H), 3.3(m, 4H), 4.5(s, 2H), 7.2(m, |
| | methyl)-(4-fluorophenyl)- | | 2H), 8.0(m, 2H) |
| | ketone | | |
| 52 | [4-(4-Fluorophenyl)piperidin-1- | 378 | |
| | ylsulphonylmethyl]-(4- | | |
| | fluorophenyl)-ketone | | |
| RE | [N-Methylanilinolsulphonyl | 288 | 3.35(s, 3H), 4.60(s, 2H), 7.35(m, 1H), |
| 28 | methyl]-(phenyl)-ketone | (M − H)⁻ | 7.40(m, 2H), 7.50(m, 4H), 7.60(m, |
| | | | 1H), 8.00(d, 2H) |
| 53 | (Morpholinosulphonylmethyl)- | 284 | 2.70(m, 4H), 3.65(m, 2H), 4.60(s, 2H), |
| | (phenyl)-ketone | (M − H)⁻ | 7.55(t, 2H), 7.70(t, 1H), 8.05(d, 2H) |
| 54 | (Morpholinosulphonylmethyl)- | 327 | 3.20(m, 4H), 3.60(m, 4H), 5.15(d, 2H), |
| | (benzothiazol-2-yl)-ketone | 325 | 7.60-7.75(m, 2H), 8.25-8.35(m, 2H). |
| | | (M − H)⁻ | |
| 55 | (Morpholinosulphonylmethyl)- | 326 | 3.25(m, 4H), 3.65(m, 4H), 5.00(d, 2H), |
| | (benzothien-2-yl)-ketone | | 7.45-7.60(m, 2H), 8.00-8.10(m, 2H), |
| | | | 8.60(s, 1H). |
| 56 | (Morpholinosulphonylmethyl)- | 348 | 3.20(m, 4H), 3.60(m, 4H), 5.00(s, 2H), |
| | (4-bromophenyl)-ketone | (M − H)⁻, | 7.80(d, 2H), 8.0(d, 2H) |
| | | 350 | |
| 57 | (Morpholinosulphonylmethyl)- | 328 | 1.30(d, 6H), 3.20(m, 4H), 3.60(m, 4H), |
| | (3-isopropoxyphenyl)-ketone | | 4.60-4.80(hept, 1H), 4.95(s, 2H), 7.20(dd, |
| | | | 1H), 7.40-7.55(m, 2H), 7.60(d, |
| | | | 1H). |
| 58 | (Morpholinosulphonylmethyl)- | 277 | 3.20(m, 4H), 3.60(m, 4H), 5.00(s, 2H), |
| | (thiazol-2-yl)-ketone | 275 | 8.20(d, 1H), 8.35(d, 1H) |
| | | (M − H)⁻ | |
| 59 | (Morpholinosulphonylmethyl)- | 328 | 1.30(d, 6H), 3.20(m, 4H), 3.60(m, 4H), |
| | (4-isopropoxyphenyl)-ketone | | 4.75(hept, 1H), 4.85(s, 2H), 7.0-7.05(d, |
| | | | 2H), 7.95-8.0(d, 2H) |
| 60 | (Benzothiazol-2-yl)-(pyrrolidin- | 311 | 1.77-1.92(4H, m), 3.22-3.41(4H, m), |
| | 1-ylsulphonylmethyl)-ketone | | 5.11(2H, s), 7.63-7.74(2H, m), 8.24-8.33(2H, m) |
| 61 | (Benzothien-2-yl)-(pyrrolidin-1- | 310 | 1.79-1.94(4H, m), 3.19-3.39(4H, m), |
| | ylsulphonylmethyl)-ketone | | 4.98(2H, s), 7.43-7.62(2H, m), 8.02-8.12(2H, |
| | | | d), 8.58(1H, s) |
| 62 | (Thiazol-2-yl)-(pyrrolidin-1- | 260 | 1.76-1.91(4H, m), 3.21-3.36(4H, m), |
| | ylsulphonylmethyl)-ketone | | 4.99(2H, s), 8.19-8.24(1H, d), 8.29-8.35(1H, d) |
| 63 | (4-Bromophenyl)-(pyrrolidin-1- | | 1.77-1.96(4H, m), 3.20-3.40(4H, m), |
| | ylsulphonylmethyl)-ketone | | 4.93(2H, s), 7.73-7.84(d, 2H), 7.93-8.04(2H, d) |
| 64 | (3-Isopropoxyphenyl)- | 312, | 1.23-1.37(6H, d), 1.76-1.91(4H, m), |
| | (pyrrolidin-1- | 310 | 3.22-3.38(4H, m), 4.63-3.78(1H, m), |
| | ylsulphonylmethyl)-ketone | (M − H)⁻ | 4.91(2H, s), 7.18-7.26(1H, m), 7.39-7.48(1H, |
| | | | app t), 7.51(1H, bs), 7.55-7.63(1H, m) |
| 65 | (4-Isopropoxyphenyl)- | 312 | 1.21-1.31(6H, d), 1.76-1.90(4H, m), |
| | (pyrrolidin-1- | | 3.17-3.37(4H, m), 4.71-4.82(1H, m), |
| | ylsulphonylmethyl)-ketone | | 4.82(2H, s), 6.97-7.06(2H, d), 7.93-8.03(2H, d) |

### Example 66

### [1-(Morpholinosulphonyl)-2-phenylethyl]-(4-fluorophenyl)-ketone

To a stirred solution of (Morpholinosulphonylmethyl)-(4-fluorophenyl)-ketone (Reference Example 27; 287mg, 1.0 mmol) in acetone (5 ml) was added potassium carbonate (200 mg, 1.5 mmol) followed by benzyl bromide (130 μl, 1.1 mmol). The resulting suspension was stirred at room temperature for overnight; tlc analysis showed only partial reaction. DMF (0.5 ml) and potassium iodide (trace) were added and the suspension stirred overnight at 30-35° C. The solvent was partially removed in vacuo and the reaction mixture then quenched with EtOAc (˜20 ml). The resulting suspension was washed with water (two portions), brine, then dried (MgSO₄), filtered and evaporated to yield the product as a colourless solid (318 mg). This was crystallised from ethanol (˜5 ml) to give the title product as colourless needles (131 mg). NMR: 3.2-3.3 (m, 4H), 3.3-3.5 (m, 2H), 3.5-3.6 (m, 4H), 6.0 (dd, 1H), 7.1-7.4 (m, 7H), 8.0-8.1 (m, 2H); m/z (LC-MS): 441 (M+MeCN+Na).

### Examples 67-70

The procedure described in Example 66 was repeated using the appropriate reagents in place of benzyl bromide to give the following Examples:

| Ex | Compound | M/z | NMR |
|---|---|---|---|
| 67 | [1-(Morpholinosulphonyl)- | 394 | 3.2-3.3(m, 4H), 3.3-3.5(m, 2H), 3.5-3.6(m, |
| | 2-(4-fluorophenyl)ethyl]-(4- | (M − H)⁻ | 4H), 5.95(dd, 1H), 7.0(m, 2H), 7.3-7.4(m, |
| | fluorophenyl)-ketone | | 4H), 8.0-8.1(m, 2H) |
| 68¹ | [1-(Morpholinosulphonyl)- | 455 | 2.1-2.3(m, 1H), 2.4-2.6(m, 2H), 2.6-2.8(m, |
| | 3-phenylpropyl]-(4- | (M + MeCN + Na) | 1H), 3.2(m, 4H), 3.5(m, 4H), 5.6(dd, |
| | fluorophenyl)-ketone | | 1H), 7.1-7.3(m, 5H), 7.4(t, 2H), 8.1-8.2(m, 2H) |
| 69² | [1-(Morpholinosulphonyl) | 302 | 1.48(d, 3H), 3.2-3.3(m, 4H), 3.5-3.6(m, |
| | ethyl]-(4-fluorophenyl)- | | 4H), 7.4(ap t, 2H), 8.15(dd, 2H) |
| | ketone | | |
| 70³ | [1-(Morpholinosulphonyl)- | 315 | 1.62(s, 6H), 3.3(t, 4H), 3.6(t, 4H), 7.30(t, |
| | 1-methylethyl]-(4- | (M⁺) | 2H), 7.92(dd, 2H) |
| | fluorophenyl)-ketone | | |

¹After initial reaction and work-up as described above, the crude reaction mixture was chromatographed eluting with hexane:EtOAc 85:15 rising to 80:20 to give the title compound as a colourless solid which was recrystallised from ethanol.
²After initial reaction and work up as described above, the crude reaction mixture was chromatographed eluting with hexane:EtOAc 85:15 rising to 80:20 to give the title compound as a colourless solid.
³The reaction was carried out as described above, however, the quantity of potassium carbonate was increased to 4.6 eq (4.6 mmol) and acetone was replaced with DMF to achieve dimethylation, using iodomethane. The crude reaction mixture was chromatographed eluting with hexane:EtOAc 85:15 rising to 80:20 to give the title compound as a colourless solid.

### Example 71

### (4-Fluorophenyl)-[N-(cyclohexyl)-N-(isopropyl)sulphamoylmethyl]-ketone

To a stirred solution of N-(isopropyl)-N-(mesyl)cyclohexylamino (Method 12; 225 mg, 1.03 mmol) in anhydrous THF (5 ml) at ˜−20° C. was added a 1M solution of lithium bis(trimethylsilyl)amide (2.06 ml, 2.06 mmol). The reaction was stirred at ˜−20° C. for 30 mins before the addition of a solution of methyl-4-fluoro benzoate (206 mg, 1.33 mmol) in anhydrous THF (2 ml). The reaction was allowed to warm to room temperature and then stirred at this temperature for 1 hour. The reaction was quenched with saturated ammonium chloride (˜5ml) and the organic layer was separated. The aqueous layer was reextracted with EtOAc. The combined organic layers were washed with brine then dried (MgSO₄), filtered and evaporated to yield an oil. This oil was purified by column chromatography (DCM to 5% MeOH/DCM) to yield the product as on oil which crystallised on standing (153 mg, 44%). NMR: 1.00 (m, 1H), 1.20 (m, 3H), 1.25 (d, 6H), 1.55 (m, 1H), 1.65 (m, 2H), 1.75 (m, 3H), 3.15 (m, 1H), 3.65 (m, 1H), 4.45 (s, 2H), 7.10 (t, 2H), 8.05 (m, 2H); m/z: 340 (M−H)⁻.

### Examples 72-76 and Reference Example 29

The procedure described in Example 71 was repeated using the appropriate starting materials to obtain the compounds described below.

| Ex | Compound | NMR | M/z | SM |
|---|---|---|---|---|
| 72 | (4-Fluorophenyl)-[N-(4- | 3.30(s, 3H), 4.50(s, 2H), | 340 | Method 16 |
| | chlorophenyl)-N- | 7.15(t, 2H), 7.35(d, 2H), | (M − H)⁻ | |
| | (methyl)sulphamoylmethyl]- | 7.50(d, 2H), 8.05(m, 2H) | | |
| | ketone | | | |
| 73 | (4-Fluorophenyl)-[N-(pyrid-2- | 3.45(s, 3H), 5.05(s, 2H), | 307 | Method 13 |
| | yl)-N- | 7.15(t, 3H), 7.25(d, 1H), | (M − H)⁻ | |
| | (methyl)sulphamoylmethyl]- | 7.75(t, 1H), 8.05(m, 2H), | | |
| | ketone | 8.40(m, 1H) | | |
| 74 | (4-Fluorophenyl)-[N-(4- | 1.05(t, 3H), 3.65(q, 2H), | 350 | Method 18 |
| | methoxyphenyl)-N- | 3.85(s, 3H), 4.55(s, 2H), | (M − H)⁻ | |
| | (ethyl)sulphamoylmethyl]- | 6.95(d, 2H), 7.20(t, 2H), | | |
| | ketone | 7.45(d, 2H), 8.10(m, 2H) | | |
| 75¹ | (4-Fluorophenyl)-[N-(4- | 4.50(s, 2H), 7.00(br s, | 326 | Method 19 |
| | chlorophenyl)sulphamoylmethyl]- | 1H), 7.20(t, 2H), 7.30(m, | (M − H)⁻ | |
| | ketone | 4H), 7.95(m 2H) | | |
| 76 | (4-Fluorophenyl)-[N-(4- | 3.30(s, 3H), 3.80(s, 3H), | 336 | Method 17 |
| | methoxyphenyl)-N- | 4.55(s, 2H), 6.90(d, 2H), | (M − H)⁻ | |
| | (methyl)sulphamoylmethyl]- | 7.15(t, 2H), 7.45(d, 2H), | | |
| | ketone | 8.05(m, 2H) | | |
| RE | (4-Fluorophenyl)-(4- | 2.30(s, 3H), 2.45(m, 4H), | 299 | Method 14 |
| 29 | methylpiperazin-1- | 3.35(m, 4H), 4.50(s, 2H), | (M − H)⁻ | |
| | ylsulphamoylmethyl]-ketone | 7.20(t, 2H), 8.10(m, 2H) | | |

¹In this example 3 equivalents of lithium bis(trimethylsilyl)amide were used and the final product was crystallised from ether

### Example 77

### (4-Fluorophenyl)-[4-(2-hydroxyethyl)piperidin-1-ylsulphonylmethyl]-ketone

2-Piperidin-4-yl-ethanol (1.5 mmol) was stirred with polymer-supported diisopropylethylamine (804 mg, 3 mmol) in 4 ml dry THF under an inert atmosphere. Methane sulphonyl chloride (93 μl, 1.2 mmol) was added and the reaction mixture was stirred for 16 hours. The reaction mixture was filtered and washed with THF, and the resulting filtrate was shaken with polymer-supported isocyanate (500 mg, 0.5 mmol). The resin was filtered and washed with THF. 1M solution of lithium bis(trimethylsilyl)amide (3.6 ml, 3.6 mmol) was added to the stirred filtrate at room temperature under an inert atmosphere. After 1.5 hours, methyl-4-fluoro benzoate (185 mg, 1.2 mmol) was added as a solution in 1 ml THF and the resulting mixture was stirred at room temperature for 2 hours. Saturated ammonium chloride solution (5 ml) was added to the reaction followed by DCM (5 ml). The organic phase was separated and solvent removed before being purified by preparative LCMS. Yielded the product as a gum (29 mg). LCMS; 330, 328 (M−H)⁻.

### Examples 78-82

The procedure described in Example 77 was repeated using the appropriate starting materials to obtain the compounds described below.

| Ex | Compound | M/z |
|---|---|---|
| 78 | (4-Fluorophenyl)-(4-hydroxy-4-phenylpiperidin- | 376 |
| | 1-ylsulphonylmethyl)-ketone | (M − H)⁻ |
| 79 | (4-Fluorophenyl)-(pyrrolidin-1-ylsulphonylmethyl)- | 272 |
| | ketone | |
| 80 | (4-Fluorophenyl)-(4-hydroxypiperidin-1- | 300 |
| | ylsulphonylmethyl)-ketone | (M − H)⁻ |
| 81 | (4-Fluorophenyl)-[4-(2-methoxyethyl)piperazin-1- | 344 |
| | ylsulphonylmethyl]-ketone | |
| 82 | (4-Fluorophenyl)-(1,3-dihydroisoindol-2- | 318 |
| | ylsulphonylmethyl)-ketone | (M − H)⁻ |

### Example 83

### (4-Fluorophenyl)-4-(isopropyl)piperazin-1-ylsulphonylmethyl-ketone

1-(Isopropyl)-4-(mesyl)piperazine (Method 15; 1.8 mmol) was stirred in 2 ml THF under an inert atmosphere at room temperature. 1M solution of lithium bis(trimethylsilyl)amide (4.4 ml, 4.4 mmol) was added to the mixture and the reaction was stirred for 3 hours. Methyl-4-fluoro benzoate (185 mg, 1.2 mmol) was added (as a solution in 2 ml THF) and the resulting mixture was stirred at room temperature for 16 hours. Saturated ammonium chloride solution (6 ml) was added to the reaction followed by DCM (6 ml). The organic phase was separated and solvent removed in vacuo. The residue was purified by chromatography (eluent: EtOAc) to give the title compound as a solid (217 mg). LCMS; 329, 327 (M−H)⁻.

### Examples 84-87

The procedure described in Example 83 was repeated using the appropriate starting materials to obtain the compounds described below.

| Ex | Compound | M/z | NMR |
|---|---|---|---|
| 84 | (Benzothien-2-yl)-[4- | 367, 365 | 1.05(d, 6H), 2.58(m, 4H), 2.75(m, 1H), |
| | (isopropyl)piperazin-1- | (M − H)⁻ | 3.40(m, 4H), 4.56(s, 2H), 7.48(m, 2H), |
| | ylsulphonylmethyl]-ketone | | 7.87(d, 1H), 7.95(d, 1H), 8.17(s, 1H) |
| 85 | (Thiazol-2-yl)-[4- | 318, 316 | 1.05(d, 6H), 2.58(m, 4H), 2.75(m, 1H), |
| | (isopropyl)piperazin-1- | (M − H)⁻ | 3.38(m, 4H), 4.84(s, 2H), 7.79(d, 1H), |
| | ylsulphonymethyl]-ketone | | 8.09(d, 1H) |
| 86 | (4,5-Dichlorothiazol-2-yl)-[4- | 386 | 1.03(d, 6H), 2.58(m, 4H), 2.75(m, 1H), |
| | (isopropyl)piperazin-1- | | 3.38(m, 4H), 4.70(s, 2H) |
| | ylsulphonylmethyl]-ketone | | |
| 87 | (5-Chlorothien-2-yl)-[4- | | 1.05(d, 6H), 2.60(m, 4H), 2.75(m, 1H), |
| | (isopropyl)piperazin-1- | | 3.37(m, 4H), 4.39(s, 2H), 7.02(d, 1H), |
| | ylsulphonylmethyl]-ketone | | 7.68(d, 1H) |

### Example 88

### (4-Bromophenylsulphonylmethyl)-(4-cyanophenyl)-ketone

To a stirred solution of methyl 4-cyanobenzoate (150 mg, 0.93 mmol) and 4-bromophenyl methyl sulphone (200 mg, 0.84 mmol) in 1,2-dimethoxyethane (10 ml) was added sodium hydride (40%) (120 mg, 3 mmol). The reaction was warmed to 85° C. and stirred at this temperature for 6 hours. The reaction was allowed to cool to room temperature and then quenched with water (˜50 ml). The solution was transferred to a separating funnel and washed with ether, the layers were separated and the organic layer was extracted with 1M sodium hydroxide solution. The aqueous layers were combined and acidified to ˜pH3 with concentrated hydrochloric acid. The resulting suspension was extracted with DCM (2×50 ml), the organic layers were combined and washed with brine then dried (MgSO₄), filtered and evaporated to yield an oil. The oil was purified by column chromatography (10 g silica, DCM) to yield a clear oil which crystallised on standing. NMR: 4.65 (s, 2H1), 7.65 (m, 4H), 7.75 (d, 2H), 8.00 (d, 2H); m/z 363 (M−H⁻.

### Examples 89-99 and Reference Example 30

The procedure described in Example 88 was repeated using the appropriate starting materials.

| EX | Compound | M/z | NMR |
|---|---|---|---|
| 89 | (4-Bromophenylsulphonylmethyl)-(4- | 406 | 4.75(s, 2H), 7.70(m, 4H), 7.80(d, |
| | trifluoromethylphenyl)-ketone | (M − H)⁻ | 2H), 8.10(d, 2H) |
| 90 | (4-Fluorophenylsulphonylmethyl)-(4- | 345 | 4.70(s, 2H), 7.20(m, 2H), 7.70(d, |
| | trifluoromethylphenyl)-ketone | (M − H)⁻ | 2H), 7.85(m, 2H), 8.00(d, 2H) |
| 91 | (Thien-2-ylsulphonylmethyl)-(thien-2- | 271 | 4.70(s, 2H), 7.15(m, 2H), 7.75(br |
| | yl)-ketone | (M − H)⁻ | m, 4H) |
| 92 | (Thien-2-ylsulphonylmethyl)-(4- | 290 | 4.85(s, 2H), 7.15(m, 1H), 7.65(m, |
| | cyanophenyl)-ketone | (M − H)⁻ | 1H), 7.80(m, 3H), 8.10(d, 2H) |
| 93 | (Thien-2-ylsulphonylmethyl)-(4- | 333 | 4.80(s, 2H), 7.10(m, 1H), 7.60(d, |
| | trifluoromethylphenyl)-ketone | (M − H)⁻ | 1H), 7.70(m, 3H), 8.00(d, 2H) |
| 94 | (4-Bromophenylsulphonylmethyl)- | 344 | 4.60(s, 2H), 7.20(m 1H), 7.75(br |
| | (thien-2-yl)-ketone | (M − H)⁻ | m, 6H) |
| 95 | (4-Methylphenylsulphonylmethyl)-(4- | 298 | 4.75(s, 2H), 7.35(d, 2H), 7.75(m, |
| | cyanophenyl)-ketone | (M − H)⁻ | 4H), 8.05(d, 2H) |
| 96 | (4-Fluorophenylsulphonylmethyl)-(4- | 295 | 4.65(s, 2H), 7.20(m, 4H), 7.90(m, |
| | fluorophenyl)-ketone | (M − H)⁻ | 2H), 8.00(m, 2H) |
| 97 | (Thien-2-ylsulphonylmethyl)-(4- | 283 | 4.80(s, 2H), 7.20(m, 3H), 7.70(m, |
| | fluorophenyl)-ketone | (M − H)⁻ | 2H), 8.00(m, 2H) |
| 98 | (Thien-2-ylsulphonylmethyl)-(fur-2- | 255 | 4.70(s, 2H), 6.60(m, 1H), 7.15(m, |
| | yl)-ketone | (M − H)⁻ | 1H), 7.35(m, 1H), 7.60(s, 1H), |
| | | | 7.70(d, 1H), 7.75(d, 1H) |
| RE | (4-Methylphenylsulphonylmethyl)- | 263 | 2.45(s, 3H), 4.55(s, 2H), 6.60(m, |
| 30 | (fur-2-yl)-ketone | (M − H)⁻ | 1H), 7.35(m, 3H), 7.60(s, 1H), |
| | | | 7.80(d, 2H) |
| 99¹ | (4-Methoxyphenylsulphonylmethyl)- | 279 | 3.90(s, 3H), 4.55(s, 2H), 6.60(m, |
| | (fur-2-yl)-ketone | (M − H)⁻ | 1H), 7.00(d, 2H), 7.30(m, 1H), |
| | | | 7.60(s, 1H), 7.80(d, 2H) |

¹In this example the sulphone used was 4-fluorophenyl methyl sulphone, the fluorine is displaced by methoxide during the reaction.

### Example 100

### (Phenylsulphonylmethyl)-(pyrid-2-yl)-ketone

A solution of methylphenyl sulphone (3 g, 19.2 mmol) in THF was added dropwise to a solution of lithium diisopropyl amine (2.7 ml diisopropylamine and 12 ml of 1.6M n-butyl lithlium) in THF under argon at −78° C. The resultant pink-orange solution was stirred at −78° C. for 15 mins. A solution of pyridine 2-methylcarboxylate (1.32 g, 9.6 mmol) in THF was added. The reaction mixture was stirred at −78° C. for 2 hours, then allowed to warm to room temperature and was stirred overnight. The reaction was quenched with water, filtered and evaporated to dryness to give an oil. This oil was taken up in EtOAc and purified with flash chromatography (2:1 EtOAc:petrol) to give the product which was recrystallized from EtOAc/petrol to give the solid (840 mg). Mp 101-103° C.; m/z 261 (M⁺).

### Reference Example 31

### (4-Bromophenyl)-(4-methylphenylsulphonylmethyl)-ketone

4-Sulphotoluene sodium salt (21.4 g) and 2,4′-dibromoacetophenone (27.8 g) were mixed in ethanol (100 ml) and refluxed overnight. The reaction was cooled after which the product crystallised. The crystals were filtered and dried. The filtrate was evaporated to give another crop of crystals. The first crop were triturated with water (200 ml) and filtered and combined with the second crop. The combined mass was taken up in ethanol (200 ml) and refluxed until all material dissolved. The resulting crystals were separated by filtration and dried. NMR (400 MHz, DMSO-d₆): 2.30 (s, 3H), 5.15 (s, 2H), 7.30 (d, 2H), 7.60 (m, 4H), 7.75 (d, 2H); m/z 354.

### Examples 101-104 and Reference Examples 32-34

The following compounds were made by the procedure of Syn.Lett.; EN; 10; 2000; 1500-1502 (except 1.2 eq of NaI was added to the reaction mixture) using the appropriate starting materials.

| Ex | Compound | M/z | NMR(DMSO-d₆) |
|---|---|---|---|
| 101 | [α-(2-Methylthiazol-4- | 308 | 2.50(s, 3H), 3.05(dd, 1H), 3.50(dd, 1H), |
| | ylmethyl)benzyl]-(phenyl)- | | 5.35(t, 1H), 6.95(s, 1H), 7.15(m, 1H), |
| | ketone | | 7.25(m, 2H), 7.35(m, 2H), 7.45(t, 2H), |
| | | | 7.55(t, 1H), 8.00(d, 2H) |
| 102 | [α-(2-Chlorothiazol-5- | 328 | 3.25(dd, 1H), 3.55(dd, 1H), 5.15(t, 1H), |
| | ylmethyl)benzyl]-(phenyl)- | | 7.15(m, 1H), 7.30(m, 5H), 7.45(t, 2H), |
| | ketone | | 7.55(t, 1H), 8.00(d, 2H) |
| RE | [α-(Cyanomethyl)benzyl]- | 493 | 2.90(m, 1H), 3.10(m, 1H), 4.85(t, 1H), |
| 32 | (phenyl)-ketone | [2M + Na] | 7.30(br m, 7H), 7.50(t, 1H), 7.90(d, 2H) |
| RE | [α-(Benzyl)benzyl]- | 287 | 3.00(m, 1H), 3.45(m, 1H), 5.20(t, 1H), |
| 33 | (phenyl)-ketone | | 7.10(m, 3H), 7.15(m, 3H), 7.30(br m, |
| | | | 4H), 7.40(t, 2H), 7.50(t, 1H), 8.00(d, 2H) |
| RE | [α-(Propyl)benzyl]- | 239 | 0.90(t, 3H), 1.25(m, 2H), 1.80(m, 1H), |
| 34 | (phenyl)-ketone | | 2.15(m, 1H), 4.55(t, 1H), 7.20(m, 1H), |
| | | | 7.30(m, 4H), 7.40(t, 2H), 7.45(m, 1H), |
| | | | 7.95(d, 2H) |
| 103¹ | (5-Methylfur-2-yl)-[2-(4- | 327 | |
| | chlorophenyl)-1-(pyrazin-2- | | |
| | yl)ethyl]-ketone | | |
| 104 | (4-Fluorophenyl)-[2-(2- | 352 | 3.25(dd, 1H), 3.60(dd, 1H), 4.85(t, 1H), |
| | chlorothiazol5-yl)-1-(thien- | | 6.95(d, 1H), 7.15(t, 3H), 7.20(s, 1H), 7.30(m, |
| | 3-yl)ethyl]-ketone | | 1H), 7.95(m, 2H) |

¹This example required purification by prep LCMS after column chromatography.

### Reference Example 35

### (N-Methyl-4-methylanilinosulphonylmethyl)-(4-chlorophenyl)-ketone

To a stirred solution of N-methyl-4-methylanilinosulphonylmethyl (EP 495594; 199 mg, 1.0 mmol) in dry THF (1 ml) at −78° C. was added a solution of 1.6M n-butyl lithium in hexane (1.25 ml, 2.0 mmol). The reaction was stirred at room temperature for 1 hour then cooled to −78° C. and treated with a solution of methyl-4-chlorobenzoate (170 mg, 1.0 mmol) in dry THF (1 ml). The mixture was stirred at −78° C. for 2 hours then stirred at room temperature for 1 hour. The reaction was quenched with saturated ammonium chloride solution (5 ml) and water (5 ml) and extracted with ether (2×20 ml). The combined organic extracts were washed with brine, dried (MgSO₄), filtered and evaporated to dryness. The residue was purified by column chromatography using 10% EtOAc in hexane as eluent to give a solid which was crystallised from ether/hexane to give the title compound (280 mg, 0.83 mmol). NMR: 2.4 (s, 3H), 3.3 (s, 3H), 4.5 (s, 2H), 7.2 (m, 2H), 7.4 (m, 4H), 8.00 (d, 2H).

### Examples 105-107

The procedure described in Reference Example 35 was repeated using the appropriate sulphonamide and ester to yield the desired product.

| Ex | Compound | NMR/m/z |
|---|---|---|
| 105¹ | (N-Methyl-4- | NMR: 3.3(s, 3H), |
| | methoxyanilinosulphonylmethyl)-(4- | 3.8(s, 3H), 4.5(s, |
| | chlorophenyl)-ketone | 2H), 6.9(m, 2H), |
| | | 7.4(m, 4H), 8.00(d, |
| | | 2H) |
| 106^{2,3} | (4-Bromo-2- | m/z: 582 |
| | methoxycarbonylanilinosulphonylmethyl)- | |
| | (4-bromo-2-mesylaminophenyl)-ketone | |
| 107⁴ | (4-fluorophenyl)-(N-isopropyl-4- | NMR: 1.1(d, 6H), |
| | chloroanilinosulphonylmethyl)-ketone | 4.3(m, 1H), 4.6(s, |
| | | 2H), 7.2(m, 2H), |
| | | 7.4(m, 4H), 8.00(m, |
| | | 2H); m/z: 368 |

¹N-methyl-4-methoxyanilinosulphonylmethyl: Advanced Synthesis and Catalysis 2001, 343(1), 71-74; synthesized by reaction of methanesulphonyl chloride and 4-methoxy-N-methylaniline in pyridine
²The base used was LDA.
³Condensation reaction with 4-bromo-2-methoxycarbonylanilinosulphonylmethyl.
⁴Starting material described in Method 20.

### Example 108

### [α-(4-Methoxycarbonylbenzyl)benzyl]-(phenyl)-ketone

To deoxybenzoin (50 mg, 0.25 mmol) in THF (2 ml) at 0° C. under a nitrogen atmosphere was added dropwise a 1M solution of lithium bis(trimethylsilyl)amide in THF (0.28 ml, 0.28 mmol). The reaction was stirred at 0° C. for 3 hours 30 mins before being added dropwise to a solution of methyl 4-(bromomethyl)benzoate (229 mg, 0.28 mmol) in TBF (2 ml) at 0° C. under a nitrogen atmosphere. The reaction was stirred in the melting ice bath for 16 hours. Water (5 ml) was added slowly to the reaction, which was then extracted with DCM (3×15 ml). The combined organic layers were concentrated in vacuo. The crude product was chromatographed on Kieselgel 60, eluting with 15% EtOAc in iso-hexane, to give the product as a white solid (57 mg, 66%). NMR (300 MHz, DMSO-d₆) 3.05 (1H, dd), 3.45 (1H, dd), 3.80(3H, s), 5.25 (1H, t), 7.35 (10H, m), 7.75 (2H, d), 7.95 (2H, d); m/z 345.

### Reference Examples 36-37 and Examples 109-120

The procedure described in Example 108 was repeated using the appropriate starting materials.

| Ex | Compound | M/z | NMR(300MHz, DMSO-d₆) |
|---|---|---|---|
| RE | (α-Methylbenzyl)-(4- | 245 | 1.40(3H, d), 4.90(1H, q), 7.30(5H, m), 7.50(2H, |
| 36^{A} | chlorophenyl)-ketone | | d), 7.95(2H, d) |
| 109 | [α-(Benzyl)benzyl]-(5- | 371 | 3.00(1H, dd), 3.40(1H, dd), 5.05(1H, t), 7.25(11H, |
| | bromothien-2-yl)-ketone | | m), 7.95(1H, d) |
| RE | [α-(Benzyl)benzyl]-(4- | 321 | 3.00(1H, dd), 3.45(1H, dd), 5.20(1H, t), 7.20(10H, |
| 37 | chlorophenyl)-ketone | | m), 7.50(2H, d), 8.00(2H, d) |
| 110 | (1-Phenyl-3- | 316 | 2.30(2H, m), 2.45(3H, m), 2.65(1H, m), 2.85(2H, |
| | morpholinoprop-2-yl)- | | m), 3.40(4H, m), 3.95(1H, m), 7.15(6H, |
| | (thien-2-yl)-ketone | | m), 7.90(2H, m) |
| 111 | [α-(Benzyl)benzyl]-(thien- | 293 | 3.00(1H, dd), 3.40(1H, dd), 5.05(1H, t), 7.15(7H, |
| | 2-yl)-ketone | | m), 7.25(2H, t), 7.40(2H, d), 7.90(1H, |
| | | | d), 8.05(1H, d) |
| 112 | [α-(Pyrid-3- | 288 | 3.05(1H, dd), 3.40(1H, dd), 5.25(1H, t), 7.35(10H, |
| | ylmethyl)benzyl]-(phenyl)- | | m), 8.00(2H, d), 8.35(2H, m) |
| | ketone | | |
| 113 | [α-(Pyrid-2-ylmethyl) | 288 | 3.15(1H, dd), 3.65(1H, dd), 5.50(1H, dd), |
| | benzyl]-(phenyl)-ketone | | 7.35(11H, m), 8.00(2H, d), 8.35(1H, d) |
| 114 | [α-(3-Methoxycarbonyl | 313 | 3.05(1H, dd), 3.45(1H, dd), 3.80(3H, s), 5.25(1H, |
| | benzyl)benzyl]-(phenyl)- | [M−OMe]⁺ | t), 7.35(10H, m), 7.70(1H, d), 7.80(1H, |
| | ketone | | s), 7.95(2H, d) |
| 115 | [α-(Pyrid-4-ylmethyl) | 288 | 3.05(1H, dd), 3.40(1H, dd), 5.30(1H, t), 7.35(10H, |
| | benzyl]-(phenyl)-ketone | | m), 8.00(2H, d), 8.35(2H, d) |
| 116 | [α-(2-Ethoxycarbonyl | 345 | 1.25(3H, t), 3.05(1H, dd), 3.45(1H, dd), 4.25(2H, |
| | benzyl)benzyl]-(phenyl)- | | q), 5.25(1H, t), 7.35(10H, m), 7.75(2H, |
| | ketone | | d), 7.95(2H, d) |
| 117 | [α-(2-Nitrobenzyl)benzyl]- | 332 | 3.25(1H, dd), 3.65(1H, dd), 5.20(1H, t), 7.35(11H, |
| | (phenyl)-ketone | | m), 7.85(1H, d), 7.95(2H, d) |
| 118 | [α-(3-Nitrobenzyl)benzyl]- | 332 | 3.15(1H, dd), 3.35(1H, dd), 5.30(1H, t), 7.40(10H, |
| | (phenyl)-ketone | | m), 8.00(4H, m) |
| 119 | [α-(3-Nitro-6- | 362 | 3.10(1H, dd), 3.40(1H, dd), 3.85(3H, s), 5.15(1H, |
| | methoxybenzyl)benzyl]- | | t), 7.15(6H, m), 7.45(2H, m), 7.95(5H, |
| | (phenyl)-ketone | | m) |
| 120 | [α-(5-Nitrofur-2- | | 3.20(1H, dd), 3.35(1H, dd), 5.35(1H, t), 6.50(1H, |
| | ylmethyl)benzyl]-(phenyl)- | | d), 7.35(9H, m), 8.00(2H, d) |
| | ketone | | |

^{A}Methyl iodide was the alkylating reagent.

### Example 121

### (4-Cyanophenoxymethyl)-(4-chlorophenyl)-ketone

2-Bromo-4′-chloroacetophenone (500 mg, 2.15 mmol), 4-cyanophenol (256.4 mg, 2.15 mmol) and potassium carbonate (297.4 mg, 2.15 mmol) were placed in acetone and the reaction mixture was stirred and heated at reflux overnight. On cooling, the solvent was evaporated in vacuo and the residue was partitioned between EtOAc and water. The organic layer was separated, dried (MgSO₄) and the organics removed in vacuo to give a brown solid. This was triturated with a 1:1 mixture of EtOAc and hexane to give a white solid, which was collection by filtration, 327.7 mg, 56%. NMR (300 MHz): 5.25 (s, 2H), 6.95 (d, 2H), 7.45 (d, 2H), 7.55 (d, 2H), 7.90 (d, 2H); m/z 270 for (M−H)⁻.

### Examples 122-155 and Reference Examples 38-42

The procedure described in Example 121 was repeated using the appropriate starting materials.

| Ex | Compound | M/z | NMR |
|---|---|---|---|
| 122 | (4-Ethoxyphenoxymethyl)-(4- | 291 | 1.30(t, 3H), 3.90(q, 2H), 5.05(s, |
| | chlorophenyl)-ketone | | 2H), 6.75(m, 4H), 7.40(d, 2H), |
| | | | 7.85(d, 2H) |
| 123 | (4-Phenylphenoxymethyl)-(4- | 323 | 5.05(s, 2H), 6.90(d, 2H), 7.25(t, |
| | chlorophenyl)-ketone | | 1H), 7.35(t, 2H), 7.45(m, 6H), |
| | | | 7.90(d, 2H) |
| 124 | (4-Mesylphenoxymethyl)-(4- | 323 | 2.95(s, 3H), 5.25(s, 2H), 6.95(d, |
| | chlorophenyl)-ketone | (M−H)⁻ | 2H), 7.45(d, 2H), 7.80(d, 2H), |
| | | | 7.85(d, 2H) |
| 125 | (4-Fluoro-3-chlorophenoxymethyl)- | 297 | 5.20(s, 2H), 6.80(m, 1H), 6.95-7.05(m, |
| | (4-chlorophenyl)-ketone | (M−H)⁻ | 2H), 7.50(d, 2H), 7.90(d, |
| | | | 2H) |
| 126 | (4-Fluoro-2-chlorophenoxymethyl)- | 297 | 5.20(s, 2H), 6.80(m, 2H), 7.10(m, |
| | (4-chlorophenyl)-ketone | (M−H)⁻ | 1H), 7.45(dd, 2H), 7.95(dd, 2H) |
| 127 | (4-Cyanomethylphenoxymethyl)-(4- | 286 | 3.70(s, 2H), 5.20(s, 2H), 6.90(d, |
| | chlorophenyl)-ketone | | 2H), 7.20(d, 2H), 7.50(d, 2H), |
| | | | 7.90(d, 2H) |
| 128 | [4-(2-Thiazolin-2- | 332 | 3.40(t, 2H), 4.40(t, 2H), 5.25(s, |
| | yl)phenoxymethyl]-(4- | | 2H), 6.90(d, 2H), 7.50(d, 2H), 7.75(d, |
| | chlorophenyl)-ketone | | 2H), 7.95(d, 2H) |
| 129 | (4-Cyanophenoxymethyl)-(2,4- | 305 | 5.50(s, 2H), 7.15(dt, 2H), 7.65(dd, |
| | dichlorophenyl)-ketone | | 1H), 7.75-7.85(m, 3H), 7.95(d, |
| | | | 1H) |
| 130 | (2-Methylpyrid-5-yloxymethyl)- | 228 | 2.38(s, 3H), 5.62(s, 2H), 7.14(d, |
| | (phenyl)-ketone | | 1H), 7.30(m, 3H), 7.65(d, 1H), |
| | | | 7.68(d, 1H), 8.0(d, 1H), 8.19(d, |
| | | | 1H) |
| 131 | (2-Carbamoylphenoxymethyl)-(4- | 334 | 7.20(td, 1H), 7.40(d, 1H), 7.60(td, |
| | bromophenyl)-ketone | | 1H), 7.80(bs, 1H), 7.95(dt, 2H), |
| | | | 8.05-8.15(m, 3H), 8.45(bs, 1H) |
| 132¹ | (4-fluorophenoxymethyl)-(4- | 264 | 5.40(s, 2H), 6.80-6.90(m, 2H), |
| | chlorophenyl)-ketone | | 6.95-7.05(m, 2H), 7.55(m, 2H), |
| | | | 7.90(m, 2H) |
| RE | (Naphth-2-yloxymethyl)-(phenyl)- | 261 | 5.65(s, 2H), 7.25(dd, 1H), 7.30-7.35(m, |
| 38 | ketone | | 2H), 7.45(td, 1H), 7.60(t, |
| | | | 2H), 7.65-7.75(m, 2H), 7.85(m, |
| | | | 2H), 8.05(m, 2H) |
| RE | (4-t-Butylphenoxymethyl)-(4- | 303 | 1.25(m, 9H), 1.15(s, 2H), 6.80(m, |
| 39 | chlorophenyl)-ketone | | 2H), 7.30(m, 2H), 7.45(m, 2H), |
| | | | 7.95(M, 2H) |
| RE | (4-Phenylphenoxymethyl)-(phenyl)- | 289 | 5.25(s, 2H), 7.00(m, 2H), 7.20-7.25(m, |
| 40 | ketone | | 1H), 7.40(t, 2H), 7.50-7.60(m, |
| | | | 6H), 7.60(m, 1H), 8.00(d, |
| | | | 2H) |
| RE | (Phenoxymethyl)-(4-phenylphenyl)- | 289 | 5.25(s, 2H), 6.95(m, 3H), 7.30(M, |
| 41 | ketone | | 2H), 7.40-7.50(m, 3H), 7.60(m, |
| | | | 2H), 7.70(m, 2H), 8.10(d, 2H) |
| RE | (4-Chloro-2- | 308 | 5.40(s, 2H), 5.90(bs, 1H), 6.90(d, |
| 42 | carbamoylphenoxymethyl)-(4- | | 1H), 7.20(m, 2H), 7.40(m, 1H), |
| | fluorophenyl)-ketone | | 8.00(m, 2H), 8.30(m, 1H), |
| | | | 8.70(bs, 1H) |
| 133 | [2-(N-Phenylcarbamoyl) | 350 | 5.50(s, 2H), 7.05-7.25(m, 5H), |
| | phenoxymethyl]-(4-fluorophenyl)- | | 7.40(m, 2H), 7.50(m, 1H), |
| | ketone | | 8.00-8.10(m, 4H), 8.40(d, 1H), |
| | | | 10.65(bs, 1H) |
| 134 | [2-(N-Isopropylcarbamoyl) | 316 | 1.35(d, 6H), 4.40(quin, 1H), 5.40(s, |
| | phenoxymethyl]-(4-fluorophenyl)- | | 2H), 6.95(d, 1H), 7.10-7.30(m, |
| | ketone | | 3H), 7.40(m, 1H), 8.00-8.01(m, |
| | | | 2H), 8.30(d, 1H), 8.70(bs, 1H) |
| 135 | [2-(N-Isobutylcarbamoyl) | 330 | 1.00(d, 6H), 2.00(quin, 1H), 3.40(t, |
| | phenoxymethyl]-(4-fluorophenyl)- | | 2H), 5.40(s, 2H), 6.95(d, 1H), |
| | ketone | | 7.10-7.30(m, 3H), 7.45(m, 1H), |
| | | | 8.00(m, 2H), 8.30(dd, 1H), |
| | | | 8.80(bs, 1H) |
| 136 | (2,4-Dichloro-6- | 344 | 5.40(s, 2H), 5.80(bs, 1H), 7.20(m, |
| | carbamoylphenoxymethyl)-(4- | | 2H), 7.60(m, 1H), 7.95(m, 2H), |
| | fluorophenyl)-ketone | | 8.10(m, 1H), 8.40(bs, 1H) |
| 137 | [2-(N,N-Dimethylcarbamoyl) | 302 | 2.90(s, 3H), 3.10(s, 3H), 5.30(s, |
| | phenoxymethyl]-(4-fluorophenyl)- | | 2H), 6.80(d, 1H), 7.05(t, 1H), 7.20(m, |
| | ketone | | 2H), 7.30(d, 2H), 8.05(m, 2H) |
| 138 | (2-Acetylamino-4- | 322 | 2.25(s, 3H), 5.35(s, 2H), 6.90(s, |
| | chlorophenoxymethyl)-(4- | | 1H), 7.00(dd, 1H), 7.20(m, 2H), |
| | fluorophenyl)-ketone | | 7.95(m, 2H), 8.30(d, 1H), 8.65(bs, |
| | | | 1H) |
| 139 | (2-Carbamoylphenoxymethyl)-(4- | 290 | 5.40(s, 2H), 5.90(bs, 1H), 6.95(d, |
| | chlorophenyl)-ketone | | 1H), 7.15(t, 1H), 7.45-7.60(m, |
| | | | 3H), 7.90(m, 2H), 8.30(dd, 1H), |
| | | | 8.70(bs, 1H) |
| 140 | (2,4-Dichloro-5- | 356 | 2.20(s, 2H), 5.35(s, 2H), 7.10-7.20(m, |
| | acetylaminophenoxymethyl)-(4- | | 2H), 7.40(s, 1H), 7.55(bs, |
| | fluorophenyl)-ketone | | 1H), 8.10(m, 2H), 8.20(bs, 1H) |
| 141 | (3-Acetylaminophenoxymethyl)-(4- | 302 | 2.10(s, 3H), 5.20(s, 2H), 6.70(d, |
| | chlorophenyl)-ketone | | 1h), 6.95(d, 1H), 7.20-7.25 M, |
| | | | 2H), 7.35(bs, 1H), 7.45(d, 2H), |
| | | | 7.95(d, 2H) |
| 142 | (3-Carbamoylphenoxymethyl)-(4- | 289 | 2.60(s, 3H), 5.30(s, 2H), 7.10(m, |
| | chlorophenyl)-ketone | | 1H), 7.30-7.50(m, 5H), 7.95(d, |
| | | | 2H) |
| 143 | (3-Acetylaminophenoxymethyl)-(4- | 286 | 2.10(s, 3H), 5.20(s, 2H), 6.70(m, |
| | fluorophenyl)-ketone | | 1H), 6.95(d, 1H), 7.10-7.20(m, |
| | | | 3H), 7.30(m, 2H), 8.05(m, 2H) |
| 144 | (3-Carbamoylphenoxymethyl)-(4- | 272 | 5.30(s, 2H), 7.10-7.20(m, 3H), |
| | fluorophenyl)-ketone | | 7.40(m, 2H), 7.45(s, 1H), 8.05(m, |
| | | | 2H) |
| 145 | (3-Acetylphenoxymethyl)-(4- | | 2.60(s, 3H), 5.30(s, 2H), 7.10-7.25(m, |
| | fluorophenyl)-ketone | | 3H), 7.40(t, 1H), 7.50(m, |
| | | | 1H), 7.60(m, 1h), 8.05(m, 2H) |
| 146 | (3-Morpholinophenoxymethyl)-(4- | 316 | 3.15(t, 4H), 3.85(t, 4H), 5.20(s, |
| | fluorophenyl)-ketone | | 2H), 6.40(m, 1H), 6.55(m, 2H), |
| | | | 7.10(m, 3H), 8.05(m, 2H) |
| 147 | (2-Morpholinophenoxymethyl)-(4- | 314 | 3.10(t, 4H), 3.80(t, 4H), 5.30(s, |
| | fluorophenyl)-ketone | | 2H), 6.85(m, 1H), 6.95(m, 3H), |
| | | | 7.20(m, 2H), 8.05(m, 2H) |
| 148 | (4-Acetylaminophenoxymethyl)-(4- | 288 | 2.15(s, 3H), 5.20(s, 2H), 6.85(d, |
| | fluorophenyl)-ketone | | 2H), 7.00-7.20(m, 2H), 7.40(d, |
| | | | 2H), 8.05(m, 2H) |
| 149² | (4-Chlorophenoxymethyl)-(3,5- | 282(M⁺) | |
| | difluorophenyl)-ketone | | |
| 150³ | (2-Morpholinomethyl-3,5- | 339(M⁺) | |
| | dimethylphenoxymethyl)-(phenyl)- | | |
| | ketone | | |
| 151⁴ | (2,4-Dibromophenoxymethyl)- | 368(M⁺) | |
| | (phenyl)-ketone | | |
| 152⁵ | (2,4-Difluorophenoxymethyl)-(4- | 282 | |
| | chlorophenyl)-ketone | | |
| 153⁶ | (2,4,6-Triiodophenoxymethyl)- | 590(M⁺) | |
| | (phenyl)-ketone | | |
| 154⁴ | (2-Methoxy-4-propyl-5- | 362(M⁺) | |
| | bromophenoxymethyl)-(phenyl)- | | |
| | ketone | | |
| 155 | (4-Chlorophenyl)-(4- | 304 | 2.15(s, 3H), 5.19(s, 2H), 6.90(d, |
| | acetylaminophenoxymethyl)-ketone | (M+) | 2H), 7.05(s, 1H), 7.40(d, 2H), 7.25(d, |
| | | | 2H), 7.90(d, 2H) |

¹MeCN instead of acetone was used as the solvent.
²MeCN at room temperature rather than acetone at reflux was used as the solvent.
³Using NaH in DMF as base.
⁴Using KOH in ethanol.
⁵DMF at room temperature as the solvent
⁶KOH in n-butanol.

### Reference Example 43

### (4-Nitrophenoxymethyl)-(4-chlorophenyl)-ketone

To a solution of 4-nitrophenol (42 mg, 0.3 mmol) and 4-chlorophenacyl bromide (84 mg, 0.36 mmol) in DCM was added MP-CO₃ resin solid resins (257 mg, 0.9 mmol). The reaction was shaken overnight. Scavenging reagents were added (131 mg PS-thiophenol again a resin, 34 mg MP-CO₃) and the reaction was again shaken overnight. The reaction was filtered and the solvent was removed under reduced pressure. The resulting oil was purified by column chromatography (eluting with 10% EtOAc/isohexane to 50% EtOAc/isohexane) to yield an oil (35 mg, 36%). NMR: 7.00 (d, 2H), 7.50 (d, 2H), 7.95 (d, 2H), 8.20 (d, 2H); m/z: 290 (M−H)⁻.

### Examples 156-186 and Reference Examples 44-50

The procedure described in Reference Example 43 was repeated using the appropriate starting materials.

| Ex | Compound | M/z |
|---|---|---|
| 156 | (4-Methoxyphenoxymethyl)-(4-chlorophenyl)-ketone | 275(M−H)⁻ |
| RE 44 | (2-Cyanophenoxymethyl)-(4-chlorophenyl)-ketone | 270(M−H)⁻ |
| RE 45 | (4-Chlorophenoxymethyl)-(4-chlorophenyl)-ketone | 280(M−H)⁻ |
| 157 | (4-Chlorophenoxymethyl)-(2-methoxyphenyl)-ketone | 275(M−H)⁻ |
| 158 | (4-Chlorophenoxymethyl)-(3-methoxyphenyl)-ketone | 275(M−H)⁻ |
| RE 46 | (4-Chlorophenoxymethyl)-(4-methoxyphenyl)-ketone | 275(M−H)⁻ |
| RE 47 | (4-Chlorophenoxymethyl)-(4-methylphenyl)-ketone | 259(M−H)⁻ |
| RE 48 | (4-Chlorophenoxymethyl)-(4-fluorophenyl)-ketone | 263(M−H)⁻ |
| 159 | (4-Chlorophenoxymethyl)-(4-pentylphenyl)-ketone | 317 |
| 160 | (2-Fluorophenoxymethyl)-(4-chlorophenyl)-ketone | 263(M−H)⁻ |
| RE 49 | (2-Chlorophenoxymethyl)-(4-chlorophenyl)-ketone | 280(M−H)⁻ |
| 161 | (2-Phenylphenoxymethyl)-(4-chlorophenyl)-ketone | 321(M−H)⁻ |
| 162 | (2-Methylphenoxymethyl)-(4-chlorophenyl)-ketone | 259(M−H)⁻ |
| 163 | (2-Propylphenoxymethyl)-(4-chlorophenyl)-ketone | 287(M−H)⁻ |
| 164 | (3-Cyanophenoxymethyl)-(4-chlorophenyl)-ketone | 270(M−H)⁻ |
| 165 | (3-Methoxyphenoxymethyl)-(4-chlorophenyl)-ketone | 275(M−H)⁻ |
| 166 | (3-Acetylphenoxymethyl)-(4-chlorophenyl)-ketone | 287(M−H)⁻ |
| 167 | (3-t-Butylphenoxymethyl)-(4-chlorophenyl)-ketone | 301(M−H)⁻ |
| 168 | (4-Acetylphenoxymethyl)-(4-chlorophenyl)-ketone | 287(M−H)⁻ |
| 169 | (4-Cyclopentylphenoxymethyl)-(4-chlorophenyl)-ketone | 313(M−H)⁻ |
| 170 | (3-Morpholinophenoxymethyl)-(4-chlorophenyl)-ketone | 332(M−H)⁻ |
| 171 | (4-Butylphenoxymethyl)-(4-chlorophenyl)-ketone | 303 |
| 172 | (4-Morpholinophenoxymethyl)-(4-chlorophenyl)-ketone | 332 |
| 173 | [4-(2-methoxyethoxy)phenoxymethyl]-(4-chlorophenyl)-ketone | 321 |
| 174 | (4-Fluorophenoxymethyl)-(4-fluorophenyl)-ketone | 249 |
| 175 | (2-Isopropylphenoxymethyl)-(4-fluorophenyl)-ketone | 273 |
| 176 | (3-Chlorophenoxymethyl)-(4-fluorophenyl)-ketone | 263(M−H)⁻ |
| 177 | [3-(N,N-Diethylamino)phenoxymethyl]-(4-fluorophenyl)-ketone | 302 |
| 178 | (3-Methoxyphenoxymethyl)-(4-fluorophenyl)-ketone | 261 |
| 179 | (4-Fluorophenoxymethyl)-(4-fluorophenyl)-ketone | 247(M−H)⁻ |
| RE 50 | (4-Methoxyphenoxymethyl)-(4-fluorophenyl)-ketone | 259(M−H)⁻ |
| 180 | (4-Butylphenoxymethyl)-(4-fluorophenyl)-ketone | 257(M−H)⁻ |
| 181 | (3-Isopropylphenoxymethyl)-(4-fluorophenyl)-ketone | 273 |
| 182 | (4-Morpholinophenoxymethyl)-(4-fluorophenyl)-ketone | 316 |

### Example 183

### (2-Hydroxymethylpyrid-5-yloxymethyl)-(phenyl)-ketone

(N-Oxy-2-methylpyrid-5-yloxymethyl)-(phenyl)-ketone (Method 21; 4.72 g, 19.4 mmol) was dissolved in DMF (15 ml) then chilled in an ice bath before the addition of trifluoroacetic anhydride (15 ml). The solution was stirred at room temperature overnight. DCM (100 ml) was added and reaction was carefully quenched with 2M sodium carbonate. The biphasic mixture was stirred at room temperature, and the resultant deep red solution was partitioned, the organic layer collected, dried (MgSO₄), and the solvent was removed in vacuo to give an oil. This was purified by column chromatography (EtOAc 100% to 2% methanol in EtOAc) to give the product (2.64 g). NMR (DMSO-d_{6;} 400 MHz): 4.50 d, 2H), 5.25 (t, 1H), 5.80 (s, 2H), 7.35-7.45 (m, 2H), 7.55 (m, 2H), 7.80 (t, 1H), 8.05 (dd, 2H), 8.25 (d, 1H); m/z 224.

### Reference Example 51

### (Phenoxymethyl)-(4-methylphenyl)-ketone

Sodium hydride (oil free, 1.85 g, 77 mmol) was added portionwise to phenol (6.6 g, 70 mmol) in DMF (100 ml) at 0° C. When hydrogen evolution ceased, -bromo-4-methylacetophenone (14.9 g, 70 mmol) was added portionwise over 15 mins. The resulting dark solution was left to stand at 20° C. for 4 hours. Water (500 ml) was added and the mixture was extracted with EtOAc (2×200 ml). The extracts were washed with water (200 ml) and brine (200 ml) and dried (MgSO₄). The solvent was removed in vacuo to give an oil (16 g). This was purified by column chromatography (5% EtOAc in hexane), and the resultant product recrystallized from hexane (5.44 g, 34%). NMR: 2.40 (s, 3H), 5.2 (s, 2H), 6.9-7.0 (m, 2H), 7.2-7.35 (m, 5H), 7.9 (d, 2H); m/z 227.

### Reference Example 52

### (1-Methylbenzimidazol-2-ylthiomethyl)-(4-bromophenyl)-ketone

(Benzimidazol-2-ylthiomethyl)-(4-bromophenyl)-ketone (Reference Example 53; 7 g) was treated with sodium hydroxide (0.9 g) in water (10 ml). The reaction was stirred and methyl iodide was added (5 ml). An insoluble solid formed which went into a sticky lump, and this was stirred overnight, during which it became a brownish colour. The solid was filtered under gravity, then stirred with DCM, then filtered under gravity again. The filtrate was discarded. The remaining solid was heated with ethanol (50 ml), and filtered under gravity. The filtrate crystallised on standing overnight and the crystals were collected and dried in a warm cupboard. NMR (DMSO-d₆): 3.75 (s, 3H), 5.08 (s, 2H), 7.1-7.6 (m, 6H), 7.8-8.2 (q, 2H).

### Reference Example 53

### (Benzimidazol-2-ylthiomethyl)-(4-bromophenyl)-ketone

2-Mercaptobenzimidazole (3 g) was suspended in acetone (100 ml). 4-Bromophenacyl bromide was added and the whole amount set solid. A further 100 ml of acetone was added and the reaction mixture swirled. The reaction mixture was filtered, to give a solid. Mp 241-245° C.

### Example 184

### (1-Methylimidazol-2-ylthiomethyl)-(4-chlorophenyl)-ketone

4-Chloro-α-bromophenyl acetophenone (2.338 g) and 1-methyl-2-mercaptoimidazole (1.14 g) were dissolved in ethanol (50 ml) and the reaction was stirred for 30 hours. The reaction was cooled in ice and quenched with 10% sodium acetate solution (80 ml). The solid was filtered off to give 2 g product. This was recrystallized to give 140 mg. Mp 68-70° C.; NMR 3.6 (s, 3H), 4.50 (s, 2H), 6.9-8.0 (m, 7H).

### Example 185

### (Pyrimidin-2-ylthiomethyl)-(4-bromophenyl)-ketone

The procedure used above for Example 184 was repeated using 4-bromo-α-bromophenyl acetophenone and 2-mercatopyrimidine to give the title compound. NMR (400 MHz, DMSO-d₆): 5.00 (s, 2H), 7.40 (m, 1H), 8.00 (m, 2H), 8.20 (m, 2 H), 8.80 (d, 2H); m/z 309.

### Example 186

### {α-[N-(Ethyl)-6-(bromo)napth-2-ylsulphonylamino]benzyl}-(phenyl)-ketone

To [α-(ethylamino)benzyl]-(phenyl)-ketone (Organic Reactivity (Tartu) (1984), 21(4), 418-27; 0.5 mmol) was added DCM (3 ml), followed by triethylamine (2 mmol) in DCM (1 ml). The mixture was vortexed for 10 seconds. 6-Bromonapth-2-ylsulphonylchloride (0.5 mmol) in DCM (1 ml) was then added. The reaction mixture was stirred at room temperature for 5 hours. The reaction mixture was washed with 2M hydrochloric acid (2×2 ml) then water (2 ml). The organic layer was separated in a Savant centrifugal evaporator to give the product (112.6 mg). M/z 507 (M−H)⁻.

### Reference Example 54

### (N-Methyl-4-chloroanilinomethyl)-(4-chlorophenyl)-ketone

2-Bromo-4′-chloroacetophenone (233 mg, 1.0 mmol) was added to a solution of 4-chloro-N-methylaniline (295 mg, 2.1 mmol) in ethanol (8 ml) and the mixture was stirred at room temperature overnight. The solid was filtered, washed with cold ethanol and dried to give the title compound as a solid (160 mg, 0.55 mmol). NMR: 3.1 (s, 3H), 4.7 (s, 2H), 6.6 (d, 2H), 7.1 (d, 2H), 7.4 (d, 2H), 7.9 (d, 2H); m/z 294.

### Examples 187-190

The procedure described in Reference Example 54 was repeated using the appropriate starting materials to obtain the compounds described below.

| Ex | Compound | M/z | NMR |
|---|---|---|---|
| 187 | (N-Methyl-4- | 274 | |
| | methylanilinomethyl)-(4- | | |
| | chlorophenyl)-ketone | | |
| 188 | (N-Methoxy-4- | 290 | 3.0(s, 3H), 3.7(s, 3H), |
| | methylanilinomethyl)-(4- | | 4.6(s, 2H), 6.7(d, 2H), |
| | chlorophenyl)-ketone | | 6.8(d, 2H), 7.4(d, 2H), |
| | | | 7.9(d, 2H) |
| 189 | (N-Isopropylyanilinomethyl)- | 288 | 1.2(d, 6H), 4.2(m, 1H), |
| | (4-chlorophenyl)-ketone | | 4.6(s, 2H), 6.6(d, 2H), |
| | | | 6.7(t, 1H), 7.1(m, 2H), |
| | | | 7.4(d, 2H), 8.0(d, 2H) |
| 190¹ | (N-Methylanilinomethyl)-(4- | 352 | |
| | iodophenyl)-ketone | | |

¹Solvent used was dioxane not ethanol.

### Reference Example 55

### {4-[1-(Pyrid-4-yl)piperazin-4-yl]phenethyl}-(4-methylphenyl)-ketone

To a solution of 4-(4-pyridyl-1-piperazinyl)-benzaldehyde (WO 9728128; 1.0 g, 3.75 mmol) and 4-methylacetophenone (503 mg, 3.75 mmol) in ethanol (25 ml) was added concentrated aqueous sodium hydroxide solution (2 drops) and the mixture was stirred at room temperature overnight. The precipitated solid was filtered, washed with a small amount of cold ethanol, dried, taken up in ethanol (100 ml) and hydrogenated over 10% Pd on charcoal. The catalyst was removed by filtration and the ethanol evaporated to leave a residue which was crystallised from EtOAc/hexane to give the title compound as a solid (320 mg, 2.6 mmol). Mp 114-115° C.; C₂₅H₂₇N₃O requires C; 77.9%; H;7.1%; N;10.9%; found C; 77.6%; H;7.1%; N;10.5%; m/z 386.

### Example 191

### (N-Mesyl-4-cyanoanilinomethyl)-(4-chlorophenyl)-ketone

Sodium hydride (80 mg of a 60% dispersion; 2.0 mmol) was suspended in DMF (2 ml) and treated with 4-methanesulphonamino-1-benzonitrile (400 mg, 2.04 mmol) at 0° C. under argon. The mixture was stirred for 20 minutes then treated with a solution of 2-bromo-4′-chloroacetophenone (400 mg, 1.72 mmol) in DMF (2 ml). The mixture was stirred at room temperature for 2 hours and then poured onto water (60 ml). The aqueous layer was extracted with EtOAc (3×30 ml), and the combined organic extracts washed with brine, dried and evaporated. The residue was purified by column chromatography using 30% EtOAc in hexane as eluent to give the title compound as a solid 500 mg. NMR (300 MHz): 3.2 (s, 3H), 5.2 (s, 2H), 7.5 (d, 2H), 7.55 (d, 2H), 7.6 (d, 2H), 7.90 (d, 2H); m/z 347 (M−H)⁻.

### Example 192

### (Benzyl)-[4-(morpholinosulphonyl)phenyl]-ketone

To 4-(morpholinosulphonyl)benzoylchloride (Method 22; 869 mg-3 mmol) in dimethoxyethane (10 ml) was added bis(triphenylphosphine)-palladium chloride (211 mg; 0.3 mmol) and activated zinc (392 mg; 6 mmol). The mixture was degassed under argon and with stirring a solution of benzyl bromide (513 mg; 3 mmol) was added over 45 mins. After overnight at 20° C. the mixture was diluted with EtOAc, washed with aqueous hydrochloric acid (2M, 25 ml) and brine and dried (MgSO₄). After concentration the residue was purified by chromatography with EtOAc-DCM as eluent to give a solid (524 mg-51%). NMR: 3.00 (t, 4H), 3.75 (t, 4H), 4.3 (s, 2H), 7.3 (m, 5H), 7.8 (d, 2H), 8.15 (d, 2H).

### Example 193

### [2-(Methoxymethylthio)benzoylaminomethyl]-(4-bromophenyl)-ketone

2-(Methoxymethylthio)benzoicacid (Method 24; 376 mg, 2 mmol), dimethylaminopyridine (610 mg, 5 mmol), 1(3-dimethylaminopropyl)-3-ethylcarbodiimide hydrochloride (400 mg, 2.1 mmol), 4-bromophenacylamine and hydrochloric acid (525 mg, 2.1 mmol) were dissolved in DMF (4 ml). The reaction mixture was stirred overnight, evaporated and EtOAc was added (100 ml). The EtOAc was washed with citric acid (3×50 ml), saturated sodium hydrogencarbonate solution (50 ml) and brine (50 ml) and dried (MgSO₄). The organic layer was evaporated to yield a yellow semi solid (670 mg). This was purified by column chromatography (hexane: EtOAc 1:1) to give the title compound (219 mg, 28%). M/z 396.

### Example 194

### [N-(Thien-2-ylcarbonyl)anilinomethyl]-(4-fluorophenyl)-ketone

To a solution of (anilinomethyl)-(4-fluorophenyl)-ketone (Reference Example 56; 230 mg) in DCM (10 ml) was added diisopropyl ethylamine (342 μl) followed by 2-thiophene carbonyl chloride (106 μl). The reaction was stirred at room temperature for 2 hours, then washed with hydrochloric acid (1M), saturated bicarbonate, water and brine. The organic layers were dried and evaporated. The solid was triturated with ether to give the title compound as a pale yellow solid (250 mgs, 73%). M/z 340.

### Reference Example 56

### (Anilinomethyl)-(4-fluorophenyl)-ketone

To a suspension of aniline (910 μl) and sodium bicarbonate (840 mg) stirred at room temperature in ethanol (50 ml) was added 4-fluorophenacyl bromide (2.17 g). The reaction was stirred for 1 hour and the yellow suspension evaporated. The resultant slurry was taken up in water and extracted with EtOAc (2×50 ml). The organics were dried, filtered and evaporated to give a yellow solid. This was triturated with isohexane to give a cream solid (1.45 g, 63%).

### Reference Example 57

### (1,3-Diphenylprop-2-yl)-(phenyl)-ketone

To a stirred suspension of finely ground KOH in acetophenone was added Aliquat 336. The reaction was stirred for 5 mins at room temperature and then benzylbromide was added. The reaction was left to stir for 48 hours and then extracted with DCM. The DCM was washed with water and brine then dried (MgSO₄), filtered and evaporated to yield a yellow oil. This material was first purified by column chromatography (50 g silica, DCM) then prep HPLC to yield the product as an oil (107 mg, 4%). NMR (DMSO-d₆): 2.75 (m, 2H), 3.00 (m, 2H), 4.25 (m, 1H), 7.15 (m, 10H), 7.35 (t, 2H), 7.50 (m, 1H), 7.80 (d, 2H).

### Reference Example 58

### (N-Methyl-4-methylphenolsulphonylaminomethyl)-(4-chlorophenyl)-ketone

To a stirred solution of sodium hydride (60% suspension in mineral oil, 80 mg, 2 mmol) in anhydrous DMF (2 ml) at 0° C. under argon was added N-methyl-p-toluenesulphonamide (378 mg, 2 mmol). The reaction was stirred at 0° C. for 20 mins and then a solution of 4-chlorophenacyl bromide (400 mg, 1.7 mmol) in DMF (2 ml) was added. The reaction was allowed to warm to room temperature and left to stir for 2 hours. The reaction was quenched with cold water (60 ml) and extracted with EtOAc (2×40 ml), the combined organic extracts were washed with brine, dried (MgSO₄), filtered and evaporated to give an oil. This oil was purified by column chromatography (eluting with 20% EtOAc/isohexane) to yield the product as a solid (98 mg, 17%). NMR(DMSO-d₆): 2.40 (s, 3H), 2.70 (s, 2H), 4.70 (s, 2H), 7.40 (d, 2H), 7.60 (d, 2H), 7.70 (d, 2H), 8.00 (d, 21); m/z: 338.

### Example 195

### (N-Ethyl-4-methylphenylsulphonylaminomethyl)-(4-fluorophenyl)-ketone

Using the procedure of Reference Example 58 using the appropriate starting materials the title compound was synthesised. NMR(DMSO-d₆): 1.00 (t, 3H), 2.40 (s, 3H), 3.20 (q, 2H), 4.80 (s, 2H), 7.40 (m, 4H), 7.75 (d, 2H), 8.10 (m, 2H); m/z: 336.

### Reference Example 59

### (Phenyl)-[2-phenyl-2-(1,2,4-triazol-1-yl)ethyl]-ketone

Trans-chalcone (600 mg, 2.88 mmol) was mixed with 1,2,4-triazole (200 mg, 2.88 mmol) and the reaction was heated to 120° C. After 6 hours the reaction was cooled to room temperature. The product was isolated by chromatography on silica gel eluting with 10-50% EtOAc in iso-hexane as a clear oil, 497 mg, 62%. NMR (DMSO-d₆): 3.90 (1H, dd), 4.35 (1 H, dd), 6.40 (1H, dd), 7.25-8.05 (11H, m), 8.75 (1H, s); m/z 278.

### Example 196

### (4-Chlorophenyl)-[3-(2-trifluoromethylbenzoylamino)propyl)]-ketone

Aqueous 2M hydrochloric acid (3 ml, 6 mmol) was added to a solution of N-{3-[2-(4-chlorophenyl)-1,3-dioxolan-2-yl]propyl}-2-(trifluoromethyl)benzamide (Method 32; 500 mg, 1.2 mmol) in tetrahydrofuran (10 ml). After stirring for 3 days at ambient temperature the solvent was removed under reduced pressure and the resultant material triturated with a minimal amount of methanol to give the title compound (203 mgs, 0.55 mmol). NMR: 2.00-2.16 (2H, m), 3.04-3.17 (2H, t), 3.47-3.63 (2H, m), 6.04 (1H, bs), 7.39-7.64 (5H, m), (7.65-7.75 (1H, d), 7.85-7.95 (2H, m); m/z 370 and 368 (M−H)⁻.

### Examples 197-198

The following compounds were prepared by the procedure of Example 196 using the appropriate reagents.

| Ex | Compound | NMR | M/z | SM |
|---|---|---|---|---|
| 197 | (4-Chlorophenyl)-[3-(2,6- | 2.01-2.16(2H, m), 3.06-3.17(2H, t), 3.53-3.64(2H, | 338 | Meth |
| | difluorobenzoylamino) | m), 6.17(1H, bs), 6.87-7.00(2H, | | 34 |
| | propyl)]-ketone | m), 7.28-7.49(3H, m), 7.87-7.94(2H, d) | | |
| 198 | (4-Chlorophenyl)-[3-(4- | 1.84-1.99(2H, m), 2.96-3.12(4H, m), 3.84(3H, | 368 | Meth |
| | methoxyphenylsulphonyl | s), 4.60-4.71(1H, t), 6.89-6.98(2H, m), | | 33 |
| | amino)propyl)]-ketone | 7.38-7.46(2H, m), 7.73-7.80(2H, m), | | |
| | | 7.80-7.9(2H, m) | | |

Preparation of Starting Materials

The starting materials for the above Examples and Reference Examples are either commercially available or are readily prepared by standard methods from known materials. For example the following reactions are illustrations but not limitations of the preparation of some of the starting materials used in the above reactions.

Method 1

### N-Methoxy-N-methyl-3-thienylmethanamide

Pyridine (5.0 ml) was added to a solution of N,O-dimethylhydroxylamine hydrochloride (3.00 g, 30.88 mmol) and 2-(3-thienyl)-acetyl chloride (3.55 g, 25.0 mmol) in DCM (100 ml) at 0° C. The resultant mixture was stirred at ambient temperature for 1 hour, washed with water (50 ml), dried and evaporated to dryness. The residue was purified by column chromatography using 30% EtOAc in hexane as eluent to give the title compound as a liquid (3.2 g, 17.3 mmol). NMR 3.2 (s, 3H), 3.6 (s, 3H), 3.8 (s, 2H), 7.0 (d, 1H), 7.1 (s, 1H), 7.2 (d, 1H).

Method 2

### N-Methoxy-N-methyl-2-thienylethanamide

Pyridine (0.5 ml) was added to a solution of N,O-dimethylhydroxylamine hydrochloride (300 mg, 3.08 mmol) and 3-(2-thienyl)-propionyl chloride (435 mg, 2.5 mmol) in DCM (10 ml) at 0° C. The resultant mixture was stirred at ambient temperature for 1 hour, washed with water (5 ml), dried and evaporated to dryness. The residue was purified by column chromatography using 30% EtOAc in hexane as eluent to give the title compound as a liquid (390 mg, 1.96 mmol); NMR: 2.8 (t, 2H), 3.2 (t, 2H), 3.2 (s, 3H), 3.6 (s, 3H), 6.8 (dd, 1H), 6.9 (dd, 1H), 7.1 (dd, 1H).

Method 3

### (3-Bromophenoxy)-(4-bromobenzyl)-ketone

3-Bromophenol (15 g) and pyridine (12 ml) were dissolved in DCM (75 ml) and (4-bromophenyl)-acetyl chloride, dissolved in DCM (100 ml), was added dropwise. The mixture was stirred overnight, then the reaction mixture was washed with water, 2M hydrochloric acid, 2M sodium hydroxide solution and brine. The reaction mixture was dried and the solvent removed in vacuo. The residue was purified by column chromatography (100% toluene) to give the required product (42 g).

Method 4

### N,N-Diethyl-N-(α-cyano-4-methoxybenzyl)amino

Para-anisaldehyde (2.76 g, 20 mmol) was dissolved in methanol and added over 1 hour to a solution of diethylamine hydrochloride (2.74 g, 25 mmol) and sodium cyanide (1.23 g, 25 mmol) in water (5 ml). The solution was stirred at 30° C. for 4 hours and then quenched with water (100 ml) and extracted with ether. The extracts were washed with water, saturated sodium metabisulfite solution, water and brine. The solvent was removed in vacuo to give the product as a yellow oil (3.6 g, 81%).

Method 5

### (2-Fluoro-4-trifluoromethylphenyl)-(2-bromoprop-2-yl)-ketone

(2-Fluoro-4-trifluoromethylphenyl)-(prop-2-yl)-ketone (Method 6) was placed in 48% hydrobromic acid (10 ml), and bromine (1.8 ml) was added dropwise over 6 hours. The reaction mixture was washed with water to remove excess bromine, and then passed through phase separation paper. The reaction mixture was then evaporated to dryness to give a crude yield of 10 g. This product was used without further purification.

Method 6

### (2-Fluoro-4-trifluoromethylphenyl)-(prop-2-yl)-ketone

1-(1-Hydroxy-2-methylpropyl)-2-fluoro-4-trifluoromethyl (Method 7; 10 g, 42.7 mmol) was dissolved in DCM (50 ml). Pyridinium chlorochromate (13.8 g, 64.1 mmol) was added and the reaction was stirred at room temperature overnight. The reaction mixture was filtered through diatomaceous earth, triturating the remaining tar with DCM (3×30 ml) and then evaporating to dryness. The resulting oil was dissolved in ether, filtering through diatomaceous earth, and evaporated to dryness to give a crude yield of 8 g. The product was used without further purification.

Method 7

### 1-(1-Hydroxy-2-methylpropyl)-2-fluoro-4-trifluoromethyl

To n-butyl lithium, (28.5 ml) in anhydrous ether (50 ml) was added 4-bromo-3-fluorobenzotrifluoride (10 g) in ether (50 ml), dropwise at −70° C. and under argon. The reaction was stirred for 15 mins, and a solution of butyraldehyde (2.95 g) in ether (20 ml) was added, and the reaction was stirred for a further 30 mins. Acetic acid (10 ml) in ether (20 ml) was added while allowing the reaction mixture to warm to room temperature. Water (20 ml) was added and the solution partitioned. The aqueous layer was washed with ether, the organic layers combined, dried and the solvent removed in vacuo to give a crude yield of 10 g. The product was used without further purification.

Method 8

### 2-Chloro-5-acetylthiophene

To a suspension of anhydrous aluminium trichloride (26.58 g) in carbon tetrachloride (100 ml) was added with vigorous stirring acetyl chloride (15.7 g) over 20 mins with cooling in an ice bath. The resulting mixture was then treated with 2-chlorothiophene (23.7 g), in carbon tetrachloride (25 ml), with cooling in an ice bath over a 35 min period. The dark red solution was stirred a further 1 hour at 0° C. and then poured onto ice/water/hydrochloric acid. The organic layer was separated and washed with water, dried and evaporated to give a mauve oil, which gradually solidified to a low melting solid (32 g). This was used without further purification.

Method 9

### 1-[1-(4-Chlorophenyl)-1-(trimethylsilyloxy)-1-(cyano)prop-2-yl]-1,2,4-triazole

(4-Chlorophenyl)-[1-(1,2,4-triazol-1-yl)ethyl]-ketone (Method 10; 2.36 g, 10 mmol) was dissolved in toluene (15 ml), under nitrogen. To this was added a catalytic amount of zinc iodide (200 mg) and trimethylsilyl cyanide (1.2 g, 1.6 ml, 12 mmol) and the reaction mixture stirred at room temperature for 2 hours. The mixture was heated 85° C. and then left stirring at this temperature overnight. After cooling, ether was added to the reaction, and then the reaction mixture was washed with brine. The organic layer was separated, dried (MgSO₄) and the solvent removed in vacuo to gave an orange oil. The was purified by column chromatography to give the required product. M/z 334 (M⁺).

Method 10

### (4-Chlorophenyl)-[1-(1,2,4-triazol-1-yl)ethyl]-ketone

Sodium hydride (62.5 g, 50% dispersion, 2 mol) was suspended in petroleum ether and washed. It was then freed from solvent using alternating vacuum and argon until dry and powder-like. Anhydrous DMF (200 ml) was added under argon. A solution of triazole (90 g, 2 mol) in DMF (200 ml) was added keeping the temperature between 20 and 30° C. by cooling with an ice-bath. This was stirred for about 1.5 h until there was no further effervescence. A solution of (4-chlorophenyl)-(1-bromoethyl)-ketone (Method 11) in DMF (250 ml) was added dropwise with stirring and cooling keeping the temperature between 20 and 30° C. The mixture was then stirred at room temperature overnight. The DMF was removed in vacuo and the resultant residue was portioned between ether and water. The ether extracts were washed with water, dried and the ether removed in vacuo to give a brown gum. A portion was triturated with petroleum ether to give a colourless solution which slowly deposited long needles. The rest of the material was purified by column chromatography (EtOAc) to give the product.

Method 11

### (4-Chlorophenyl)-(1-bromoethyl)-ketone

4-Chloropropiophenone (122.5 g) was dissolved in chloroform (500 ml). Bromine (2 ml) was added and the solution irradiated with a photoflood lamp until reaction began. This was cooled to about 10° C. in an ice bath and the remainder of the bromine (35.5 ml) was added dropwise, reacting almost immediately. After the addition, the ice bath was removed and the reaction was left to stand overnight. The reaction mixture was washed with water (2×200 ml), saturated sodium hydrogen carbonate and water. The organic layer was then dried (MgSO₄), and the solvent removed in vacuo to give the crude product. This was recrystallized from cyclohexane to give the product as white crystals.

Method 12

### N-(Isopropyl)-N-(mesyl)cyclohexylamino

To a stirred solution of N-isopropylcyclohexylamine (2 g, 0.014 mol) and triethylamine (1.49 g, 0.015 mol) in DCM (80 ml) at 0° C. was added mesylchloride (1.62 g, 0.014 mol). The reaction was stirred at 0° C. for 10 minutes then allowed to warm to room temperature and left to stir for a further 30 minutes. The reaction mixture was transferred to a separating funnel and diluted to ˜150 ml with DCM. The solution was then washed with hydrochloric acid (2M; 50 ml), water (50 ml) and brine (30 ml), dried (MgSO₄), filtered and evaporated to yield the product as a clear oil (2.26 g, 74%). NMR: 1.05 (br m, 1H), 1.30 (br d, 8H), 1.60 (br s, 2H), 1.80 (br s, 6H), 2.85 (s, 3H), 3.30 (br m, 1H), 3.80 (m, 1H); m/z: 219.

Method 13

### N-(Methyl)-N-(mesyl)pyrid-2-ylamino

To a stirred solution of 2-(methylamino)pyridine (2 g, 0.018 mol) and triethylamine (1.82 g, 0.018 mol) in anhydrous DCM (80 ml) at 0° C. was slowly added mesyl chloride (2.06 g, 0.018 mol). The reaction was stirred at 0° C. for 10 mins then allowed to warm to room temperature and stirred for a further 30 mins. The solvent was removed under reduced pressure and resulting solid was partitioned between ether and water. The organic layer was separated and reextracted with ether. The combined organic layers were washed with brine then dried (MgSO₄), filtered and evaporated to yield an oil (2.2 g, 67%). NMR: 3.00 (s, 3H), 3.40 (s, 3H), 7.15 (br m, 1H), 7.45 (br m, 1H), 7.75 (br m, 1H), 8.45 (br s, 1H).

Method 14

### 1-(Methyl)-4-(mesyl)piperazine

To a stirred solution of 1-methylpiperazine (1 g, 10 mmol) and triethylamine (1.11 g, 11 mmol) in anhydrous DCM (70 ml) at 0° C. was added mesyl chloride (1.5 g, 10 mmol). The reaction was stirred at 0° C. for ten minutes then allowed to warm to room temperature and stirred for a further 30 minutes. The volatiles were removed under reduced pressure and the resulting material was partitioned between DCM and 2M NaOH. The organic layer was separated and washed with brine, dried (MgSO₄), filtered and evaporated to yield an oil (962 mg, 53%). NMR: 2.35 (s, 3H), 2.50 (t, 4H), 2.75 (s, 311), 3.30 (m, 4H).

Method 15

### 1-(Isopropyl)-4-(mesyl)piperazine

Method 14 was applied, using 1-isopropylpiperazine instead of 1-methylpiperazine. NMR (DMSO-d⁶): 0.97 (d, 6H), 2.70 (m, 1H), 2.84 (s, 3H), 3.08 (m, 4H), 3.32 (m, 4H).

Method 16

### N-(Methyl)-N-(mesyl)-4-chloroaniline

To a stirred solution of 4-chloro-N-methylaniline (705 mg, 5 mmol) in anhydrous pyridine (4 ml) at 0° C. was added dropwise methanesulphonyl chloride (0.41 ml, 5.25 mmol). The reaction was allowed to warm to room temperature and was stirred for 1 hour. The reaction mixture was partitioned between ether (25 ml) and 2M hydrochloric acid (30 ml). The aqueous layer was extracted with ether (2×25 ml). The combined organic extracts were washed with 1M hydrochloric acid, saturated sodium bicarbonate and brine, dried (MgSO₄), filtered and evaporated. The resulting material was crystallised from EtOAc/iso hexane to yield a white solid (900 mg, 83%). NMR: 2.85 (s, 3H), 3.30 (s, 3H), 7.35 (m, 4H).

Methods 17-20

The procedure described in Method 16 was repeated using the appropriate anilines to replace the 4-chloro-N-methylaniline to obtain the compounds described below.

| Ex | Compound | NMR |
|---|---|---|
| 17 | N-(Methyl)-N-(mesyl)-4-methoxyaniline | 2.80(s, 3H), 3.30(s, 3H), |
| | | 3.80(s, 3H), 6.90(d, 2H), |
| | | 7.30(d, 2H) |
| 18 | N-(Ethyl)-N-(mesyl)-4-methoxyaniline | 1.10(t, 3H), 2.85(s, 3H), |
| | | 2.65(q, 2H), 3.80(s, 3H), |
| | | 6.90(d, 2H), 7.25(d, 2H) |
| 19 | N-(mesyl)-4-chloroaniline | 3.00(s, 3H), 6.70(br s, |
| | | 1H), 7.20(d, 2H), 7.35(d, |
| | | 2H) |
| 20 | N-(isoPropyl)-N-(mesyl)-4-chloroaniline | 1.2(d, 6H), 2.95(s, 3H), |
| | | 4.5(m, 1H), 7.2(d, 2H), |
| | | 7.4(d, 2H) |

Method 21

### (N-Oxy-2-methylpyrid-5-yloxymethyl)-(phenyl)-ketone

(2-Methylpyrid-5-yloxymethyl)-(phenyl)-ketone (Example 130; 5.7 g, 25.1 mmol) was stirred in DCM (50 ml) then chilled in an ice bath before adding meta-chloroperoxybenzoic acid (9.5 g, 27.6 mmol @ 50%) and the reaction was stirred at room temperature overnight. A thick precipitate formed, this was filtered off and washed with ether to give the title compound. The filtrate was evaporated to give a solid which was washed with ether to give a second crop of the product (5.22 g).

Method 22

### 4-(Morpholinosulphonyl)benzoylchloride

4-(Morpholinosulphonyl)benzoic acid (Method 23; 3.6 g; 0.0133 mol) was heated at reflux for 10 hours in thionyl chloride (50 ml) containing 1 drop of DMF. After concentration the title compound was obtained as a solid ( 3.54 g-92%) which was used without purification.

Method 23

### 4-(Morpholinosulphonyl)benzoic acid

To a suspension of 4-carboxyphenylsulphonylchloride (2.2 g; 0.01 mol) in DCM (25 ml) was slowly added morpholine (4.35 ml; 0.05 mol) at gentle reflux. After 2 hours at room temperature the DCM was evaporated off. The residue was acidified with excess of aqueous hydrochloric acid. The solid was filtered, washed with water and dried under vacuum over P₂O₅. A solid was obtained (2.2 g-81%). NMR (DMSO-d₆) 2.9 (t, 4H), 3.6 (t, 4H), 7.8 (d, 2H), 8.2 (d, 2H).

Method 24

### 2-(Methoxymethylthio)benzoicacid

To a stirred solution of powdered potassium hydroxide (1.68 g, 30 mmol), triethylbenzylammonium bromide (0.23 g, 10 mol %) in dry methanol (40 ml) and bromochloromethane (40 ml) was added a solution of thiosaliclic acid (1.54 g, 10 mmol) in dry methanol (40 ml). The solution was stirred overnight at room temperature under an inert atmosphere. Hydrochloric acid (1M; 75 ml) was added and the organics were extracted with DCM: ether (1:1). The organics were pooled and washed with brine (50 ml) dried (MgSO₄) and evaporated to yield a pale yellow solid (1.85 g, 93%). M/z 197.

Method 25

### 1-(4-Fluorophenyl)-2-methoxyethanone

To a 1.0 M solution of 4-fluorophenylmagnesium bromide in THF (24.0 ml, 24.0 mmol) at 0° C. was added a solution of methoxyacetonitrile (1.42 g, 20.0 mmol) in ether (15 ml). The resultant mixture was stirred at ambient temperature for 2 hours and then quenched with 1M aqueous hydrochloric acid (40 ml). This mixture was stirred at ambient temperature for 2 hours and the aqueous layer was extracted with ether (2×40 ml). The combined extracts were washed with saturated aqueous sodium hydrogen carbonate solution, brine, dried and evaporated. The residue was purified by column chromatography using 10% EtOAc in hexane as eluent to give the title compound as a solid (2.0 g, 11.9 mmol). NMR 3.5 (s, 3H), 4.7 (s, 2H), 7.1 (m, 2H), 8.0 (m, 2H).

Method 26

### [1-(2,4-Dichlorophenyl)vinyl]-(4-chlorophenyl)-ketone

(2,4-Dichlorobenzyl)-(4-chlorophenyl)-ketone (Reference Example 17; 15 g, 50 mmol) was stirred in tetramethyldiaminomethane (25 ml) and cooled in an ice-bath. Acetic anhydride (25 ml) was added dropwise, keeping the temperature below 40° C. When the addition was complete, the ice bath was removed and the reaction stirred at room temperature for 1 hour. The reaction mixture was poured slowly onto crushed ice in water (500 ml) with stirring. The product precipitated and this was collected by filtration and dried in a dessicator. Mp 90-93° C.; NMR: 6.00 (d, 2H), 7.35 (m, 3H), 7.75 (q, 4H).

Method 27

### [2-(4-Fluorophenyl)vinyl]-(4-trifluoromethylphenyl)-ketone

Sodium hydroxide (1 g, 25 mmol) was dissolved in a mixture of water (80 ml) and ethanol (20 ml). 4-Trifluoromethylacetophenone (3.6 g, 20 mmol) was added followed by the rapid addition of 4-fluorobenzaldehyde (2.14 ml, 2.48 g, 20 mmol) during which the temperature of the reaction was maintained at about room temperature with a water bath. The reaction was stirred at room temperature for 2 hours during which time an oily solid precipitated. The reaction vessel was stored in the fridge overnight. The product was filtered off, washed with water until the pH was neutral and then recrystallized from ethanol with a few drops of water to give a yellowish solid (3.25 g, 55%). Mp 89-92° C.

Methods 28-31

The procedure described in Method 27 was carried out using the appropriate starting materials to obtain the products described below.

| Meth | Compound | Data |
|---|---|---|
| 28 | [2-(4-Fluorophenyl)vinyl]-(4-chlorophenyl)-ketone | Mp 135° C. |
| 29 | [2-(4-Chlorophenyl)vinyl]-(2,4-difluorophenyl)- | Mp 102° C. |
| | ketone | |
| 30 | [2-(4-Fluorophenyl)vinyl]-(2,4-difluorophenyl)- | Mp |
| | ketone | 75-76° C. |
| 31¹ | [2-(4-Methoxyphenyl)vinyl]-(phenyl)-ketone | |

¹This compound was prepared using lithium hydroxide as the base.

Method 32

### N-{3-[2-(4-chlorophenyl)-1,3-dioxolan-2-yl]propyl}-2-(trifluoromethyl)benzamide

Sodium metal (1.25 g, 54 mmol), was slowly dissolved in methanol (37.5 ml) and the resultant solution cooled in an ice bath and a solution of hydroxylamine hydrochloride (314 mgs, 4.5 mmol) in methanol (8.5 ml) added drop wise. After stirring the mixture for approx 10 mins 2-{3-[2-(4-chlorophenyl)-1,3-dioxolan-2-yl]propyl}-1H-isoindole-1,3(2H)-dione (Method 35; 525 mgs 1.4 mmol) was added. After 6 hours the reaction mixture was reduced in volume, quenched with water and extracted into diethyl ether and dried (MgSO₄). To this solution was added 2-(trifluoromethyl)benzoyl chloride (324 mgs, 1.55 mmol). After stirring overnight at ambient temperature the solvent was removed under reduced pressure and the resultant material subjected to column chromatography (silica, eluent 4% MeOH in CH₂Cl₂) to give the desired product (522 mgs, 1.3 mmol). NMR: 1.59-1.75 (2H, m), 1.90-2.04 (2H, m), 3.40-3.51 (2H, m), 3.68-3.81 (2H, m), (3.91-4.06 (2H, m), 5.88(1H, bs), 7.22-7.41 (4H, m), 7.46-7.63 (3H, m), 7.65-7.71 (1H, d); m/z 414.

Methods 33 and 34

The following compounds were prepared by the procedure of Method 32.

| Meth | Compound | NMR | M/z |
|---|---|---|---|
| 33 | N-{3-[2-(4-chlorophenyl)-1,3- | 1.47-1.62(2H, m), 1.78-1.90(2H, m), | 434 |
| | dioxolan-2-yl]propyl}-4- | 2.89-2.99(2H, m), 3.67-3.76(2H, m), | (M+Na)⁺ |
| | methoxybenzenesulfonamide | 3.87(3H, s), 3.93-4.02(2H, m), | |
| | | 4.53-4.65(1H, t), 6.91-7.00(2H, d), | |
| | | 7.23-7.35(4H, m), 7.73-7.80(2H, d) | |
| 34¹ | N-{3-[2-(4-chlorophenyl)-1,3- | (CDCl₃+CD₃CO₂D) | 382 |
| | dioxolan-2-yl]propyl}-2,6- | 1.62-1.79(2H, m), 1.90-2.06(2H, m), | |
| | difluorobenzamide | 3.41-3.56(2H, m), 3.68-3.83(2H, m), | |
| | | 3.93-4.10(2H, m), 6.87-7.01(2H, m), | |
| | | 7.19-7.44(5H, m) | |

¹The reaction mixture was partitioned between water and diethyl ether, the organic phase was separated, dried (MgSO₄) and the solvent removed under reduced pressure. The resultant material was subjected to column chromatography eluting with MeOH in DCM. The resultant material was triturated with hexane.

Method 35

### 2-{3-[2-(4-chlorophenyl)-1,3-dioxolan-2-yl]propyl}-1H-isoindole-1,3(2H)-dione

A mixture of 2-(4-chlorophenyl)-2-(3-chloropropyl)-1,3-dioxolane (Method 36; 10 g, 38.3 mmol), potassium phthalamide (7.1 g, 38.3 mmol) and potassium iodide (0.95 g, 5.7 mmol) was stirred at approximately 100 ° C. in dry dimethylformamide (160 ml) for 8 hours. A further quantity of potassium phthalamide (3.5 g, 18.9 mmol) was added and the mixture stirred for a further 3 hours at 100° C. After cooling to ambient temperature, the mixture was poured into water (900 ml) and the resultant precipitate filtered off. This material was washed with water, triturated with hexane and recrystallized from ethanol to give the desired product (8.56 g, 23 mmol). NMR: 1.68-1.82 (2H, m), 1.87-1.99(2H, m), 3.63-3.76 (4H, m), 3.93-4.06 (2H, m), 7.22-7.31 (2H, d), 7.31-7.40 (2H, d), 7.65-7.74 (2H, m), 7.72-7.86 (2H, m); m/z 372.

Method 36

### 2-(4-chlorophenyl)-2-(3-chloropropyl)-1,3-dioxolane

A stirred mixture of 4,4′-dichlorobutyrophenone (10 g, 46.1 mmol), ethylene glycol (13.7 ml, 16 g, 258 mmol) and 4-toluenesulfonic acid (3.17 g, 18.4 mmol) was refluxed in toluene (200 ml) with azeotropic removal of water (Dean & Stark). When no further water was distilled off the reaction mixture was cooled, washed with saturated aqueous sodium bicarbonate, dried (MgSO₄) and the solvent removed under reduced pressure to give the desired product (11.88 g, 45.5 mmol) (used without any further purification). NMR: 1.76-1.91 (2H, m), 1.94-2.07 (2H, m), 3.47-3.58 (2H, t), 3.70-3.82 (2H, m), 3.98-4.06 (2H, m), 7.26-7.34 (2H, d), 7.34-7.41 (2H, d).

1. A method for inhibiting 11βHSD1, comprising administering a compound of formula (I): 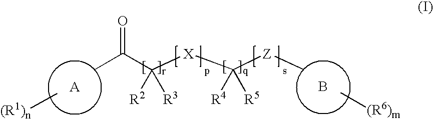
wherein:
Ring A is selected from amyl or heteroaryl;
R¹ is selected from halo, nitro, cyano, hydroxy, amino, carboxy, carbamoyl, mercapto, sulphamoyl, C₁₋₆alkyl, C₂₋₆alkenyl, C₂₋₆alkynyl, C₁₋₆alkoxy, C₁₋₆alkanoyl, C₁₋₆alkanoyloxy, N—(C₁₋₆alkyl)amino, N,N—(C₁₋₆alkyl)₂amino, C₁₋₆alkanoylamino, N—(C₁₋₆alkyl)carbamoyl, N,N—(C₁₋₆alkyl)₂carbamoyl, C₁₋₆alkylS(O)ₐ wherein a is 0 to 2, C₁₋₆alkoxycarbonyl, N—(C₁₋₆alkyl)sulphamoyl, N,N—(C₁₋₆alkyl)₂sulphamoyl, C₁₋₆alkylsulphonylamino, carbocyclyl, heterocyclyl, carbocyclylC₀₋₆alkylene-Y—, and heterocyclylC₀₋₆alkylene-Y—; or two R¹ groups on adjacent carbons may form an oxyC₁₋₄alkoxy group or a C₃₋₅alkylene group; wherein R¹ may be optionally substituted on carbon with one or more R⁷ groups; and wherein if said heterocyclyl contains an —NH-moiety, that nitrogen may be optionally substituted with an R⁸ group;
n is 0-3; wherein the values of R¹ may be the same or different;
R², R³, R⁴, and R⁵ are independently selected from hydrogen, hydroxy, amino, cyano, C₁₋₄alkyl, C₁₋₄alkoxy, N—(C₁₋₄alkyl)amino, N,N—(C₁₋₄alkyl)₂amino, C₁₋₄alkylS(O)ₐ wherein a is 0 to 2, C₁₋₄alkoxycarbonyl, C₁₋₄alkoxycarbonylamino, C₁₋₄alkanoyloxy, carbocyclyl, heterocyclyl, carbocyclylC₁₋₄alkyl, and heterocyclylC₁₋₄alkyl; or
R² and R³ together form oxo or a spiro attached heterocyclyl; wherein R², R³, R⁴, and R⁵ may be independently optionally substituted on carbon with one or more R⁹ groups; and wherein if said heterocyclyl contains an —NH— moiety, that nitrogen may be optionally substituted with an R¹⁰ group;
X and Z are independently selected from —CR¹¹R¹³—, —S(O)ₐ—, —O—, —NR¹³—, —C(O)—, —C(O)NR¹⁴, —NR¹⁵C(O)—, —OC(O)—, —C(O)O—, —SO₂NR¹⁶—, and —NR¹⁶SO₂—; wherein a is 0 to 2;
r is 1 or 2;
q is 0 or 1;
p is 0 or 1;
s is 0 or 1;
Ring B is carbocyclyl or heterocyclyl; wherein if said heterocyclyl contains an —NH— moiety, that nitrogen may be optionally substituted by an R¹⁷ group;
R⁶ is a substituent on carbon and is selected from halo, nitro, cyano, hydroxy, amino, carboxy, carbamoyl, mercapto, sulphamoyl, trifluoromethyl, trifluoromethoxy, C₁₋₄alkyl, C₂₋₄alkenyl, C₂₋₄alkynyl, C₁₋₄alkoxy, C₁₋₄alkanoyl, C₁₋₄alkanoyloxy, N—(C₁₋₄alkyl)amino, N,N—(C₁₋₄alkyl)₂amino, C₁₋₄alkanoylamino, N—(C₁₋₄alkyl)carbamoyl, N,N—(C₁₋₄alkyl)₂carbamoyl, C₁₋₄alkylS(O)ₐ wherein a is 0 to 2, C₁₋₄alkoxycarbonyl, N—(C₁₋₄alkyl)sulphamoyl, N,N—(C₁₋₄alkyl)₂sulphamoyl, C₁₋₄alkylsulphonylamino, carbocyclyl, heterocyclyl, carbocyclylC₀₋₄alkylene-Y—, and heterocyclylC₀₋₄alkylene-Y—; wherein R⁶ may be optionally substitute on carbon with one or more R¹⁸ groups; and wherein if said heterocyclyl contains an —NH— moiety, that nitrogen may be optionally substituted with an R¹⁹ group;
m is 0- 3; wherein the values of R⁶ maybe the same or different;
Y is —S(O)ₐ—, —O—, —NR²⁰—, —C(O)—, —C(O)NR²¹—, —NR²²C(O)—, or —SO₂NR²³—; wherein a is 0 to2;
R⁷, R⁹, and R¹⁸ are independently selected from halo, nitro, cyano, hydroxy, amino, carboxy, carbamoyl, mercapto, sulphamoyl, trifluoroethyl, trifluoromethoxy, C₁₋₄alkyl, C₂₋₄alkenyl, C₂₋₄alkynyl, C₁₋₄alkoxy, C₁₋₄alkanoyl, C₁₋₄alkanoyloxy, N—(C₁₋₄alkyl)amino, N,N—(C₁₋₄alkyl)₂amino, C₁₋₄alkanoylamino, N—(C₁₋₄alkyl)carbamoyl, N,N—(C₁₋₄alkyl)₂carbamoyl, C₁₋₄alkylS(O)ₐ wherein a is 0 to 2, C₁₋₄alkoxycarbonyl N—(C₁₋₄alkyl)sulphamoyl, N,N—(C₁₋₄alkyl)₂sulphamoyl, C₁₋₄alkylsulphonylamino, carbocyclyl, and heterocyclyl; wherein R⁷, R⁹, and R¹⁸ may be independently optionally substituted on carbon with one or more R²⁶ groups;
R¹¹ and R¹² are independently selected from hydrogen, hydroxy, amino, cyano, C₁₋₄alkyl, C₁₋₄alkoxy, N—(C₁₋₄alkyl)amino, N,N—(C₁₋₄alkyl)₂amino, carbocyclyl, heterocyclyl, carbocyclylC₁₋₄alkyl, and heterocyclylC₁₋₄alkyl; wherein R¹¹ and R¹² may be independently optionally substituted on carbon with one or more R²⁴ groups; and wherein if said heterocyclyl contains an —NH— moiety, that nitrogen may be optionally substituted with an R²⁵ group;
R²⁴ is selected from halo, nitro, cyano, hydroxy, amino, carboxy, carbamoyl, mercapto, sulphamoyl, trifluoromethyl, trifluoromethoxy, C₁₋₄alkyl, C₂₋₄alkynyl, C₂₋₄alkynyl, C₁₋₄alkoxy, C₁₋₄alkanoyl, C₁₋₄alkanoyloxy, N—(C₁₋₄alkyl)amino, N,N—(C₁₋₄alkyl)₂amino, C₁₋₄alkanoylamino, N—-(C₁₋₄alkyl)carbamoyl, N,N—(C₁₋₄alkyl)₂carbamoyl, C₁₋₄alkylS(O)ₐ wherein a is 0 to 2, C₁₋₄alkoxycarbonyl, N—(C₁₋₄alkyl)sulphamoyl, N,N—(C₁₋₄alkyl)₂sulphamoyl, and C₁₋₄alkylsulphonylamino;
R⁸, R¹⁰, R¹⁷, R¹⁹, and R²⁵ are independently selected from C₁₋₄alkyl, C₁₋₄alkanoyl, C₁₋₄alkylsulphonyl, C₁₋₄alkoxycarbonyl, carbamoyl, N—(C₁₋₄alkyl)carbamoyl, N,N—(C₁₋₄alkyl)carbamoyl, benzyl, benzyloxycarbonyl, benzoyl carbocyclyl, heterocyclyl, and phenylsulphonyl; wherein R⁸, R¹⁰, R¹⁷, R¹⁹, and R²⁵ may be independently optionally substituted on carbon with one or more R²⁷ groups;
R¹³, R¹⁴, R¹⁵, R¹⁶, R²⁰, R²¹, R²², and R²³ are independently selected from hydrogen, phenyl, C₁₋₄alkylsulphonyl, and C₁₋₄alkyl;
R²⁶ and R²⁷ are independently selected from selected from halo, nitro, cyano, hydroxy, trifluoromethoxy, trifluoromethyl, amino, carboxy, carbamoyl, mercapto, sulphamoyl methyl, ethyl, methoxy, ethoxy, acetyl, acetoxy, methylamino, ethylamino, dimethylamino, diethylamino, N-methyl-N-ethylamino, acetylamino, N-methylcarbamoyl, N-ethylcarbamoyl, N,N-dimethylcarbamoyl, N,N-diethylcarbamoyl, N-methyl-N-ethylcarbamoyl, methylthio, ethylthio, methylsulphinyl, ethylsulphinyl, mesyl, ethylsulphonyl, methoxycarbonyl, ethoxycarbonyl, N-methylsulphamoyl, N-ethylsulphamoyl, N,N-dimethylsulphamoyl, N,N-diethylsulphamoyl, and N-methyl-N-ethylsulphamoyl;
or a pharmaceutically acceptable salt thereof, with the proviso that said compound is not (1-methyl-1-pyrid-3-ylethyl)-(pyrid-3-yl)-ketone.

2. The methods of claim 1, wherein Ring A is selected from phenyl, naphthyl, thienyl, furyl, thiazolyl, pyridyl, imidazolyl, benzothiazolyl, and benzothienyl.

3. The method of claim 1, wherein R¹ is selected from halo, cyano, hydroxy, C₁₋₆alkyl, C₁₋₆alkoxy, N,N—(C₁₋₆alkyl)₂amino, C₁₋₆alkylsulphonylamino, carbocyclyl, and heterocyclylC₀₋₆alkylene-Y—; or two R¹ groups on adjacent carbons may form an oxyC₁₋₄alkoxy group; wherein R¹ may be optionally substituted on carbon with one or more R⁷ groups;
Y is —S(O)ₐ—, or —O—; wherein a is 0 to 2; and
R⁷ is halo.

4. The method of claim 1, wherein R², R³, R⁴, and R⁵ are independently selected from hydrogen, hydroxy, C₁₋₄alkyl, C₁₋₄alkoxy, N—(C₁₋₄alkyl)amino, carbocyclyl, carbocyclylC₁₋₄alkyl, and heterocyclylC₁₋₄alkyl; wherein R², R³, R⁴, and R⁵ may be independently optionally substituted on carbon with one or more R⁹ groups;
R⁹ is selected from halo, cyano, C₁₋₄alkyl, and N,N—(C₁₋₄alkyl)₂amino.

5. The method of claim 1, wherein X is —S(O)ₐ—, —O—, —NR¹³—, —NR¹⁵C(O)—, —SO₂NR¹⁶—, or —NR¹⁶SO₂—; wherein a is 0 or 2; and
R¹³, R¹⁵, and R¹⁶ are independently selected from hydrogen, phenyl, C₁₋₄alkylsulphonyl, and C₁₋₄alkyl.

6. The method claim 1, wherein Ring B is phenyl, thienyl, furyl, thiazolyl, piperidinyl, piperazinyl, pyrrolidinyl, 1,3-dihydroisoindolyl morpholinyl, naphthyl, cyclohexyl, pyridyl imidazolyl, 1,2,4-thiazolyl, 1,3-benzodioxolyl, thiomorpholinyl, pyrimidinyl, pyrazinyl, pyridazinyl, benzimidazolyl, or pyrimidinyl; wherein if Ring B contains an —NH— moiety, that nitrogen may be optionally substituted with an R¹⁷ group;
R¹⁷ is C₁₋₄alkyl or benzyl; wherein R¹⁷ may be optionally substituted on carbon with one or more R²⁷ groups; and
R²⁷ is methoxy.

7. The method of claim 1, wherein R⁶ is a substituent on carbon and is selected from halo, hydroxy, nitro, cyano, carbamoyl, C₁₋₄alkyl, C₁₋₄alkoxy, C₁₋₄alkanoyl, N,N—(C₁₋₄alkyl)₂amino, C₁₋₄alkanoylamino, N—(C₁₋₄alkyl)carbamoyl, N,N—(C₁₋₄alkyl)₂carbamoyl, C₁₋₄alkylS(O)ₐ wherein a is 0 or 2, C₁₋₄alkoxycarbonyl, N,N—(C₁₋₄alkyl)₂sulphamoyl, carbocyclyl, heterocyclyl, and carbocyclylC₀₋₄alkylene-Y—; wherein R⁶ may be optionally substituted on carbon with one or more R¹⁸ groups; and wherein if said heterocyclyl contains an —NH— moiety, that nitrogen may be optionally substituted with an R¹⁹ group;
Y is —C(O) or —C(O)NR²¹;
R¹⁸ is selected from halo, cyano, hydroxy, C₁₋₄alkoxy, and heterocyclyl;
R¹⁹ is heterocyclyl; and
R²¹ is hydrogen

8. The method claim 1, wherein:
Ring A is selected from phenyl, naphthyl, thienyl, furyl, thiazolyl, pyridyl, imidazolyl, benzothiazolyl, and benzothienyl;
R¹ is selected from halo, cyano, hydroxy, C₁₋₆alkyl, C₁₋₆alkoxy, N,N—(C₁₋₆alkyl)₂amino, C₁₋₆alkylsulphonylamino, carbocyclyl, and heterocyclyl]C₀₋₆alkylene-Y—; or two R¹ groups on adjacent carbons may form an oxyC₁₋₄alkoxy group; wherein R¹ may be optionally substituted on carbon with one or more R⁷ groups;
Y is —S(O)ₐ—, or —O—; wherein a is 0 to 2; and
R⁷ is halo;
n is 0-3; wherein the values of R¹ may be the same or different;
r is 1 or 2;
s is 0;
R², R³, R⁴, and R⁵ are independently selected from hydrogen, hydroxy, C₁₋₄alkyl, C₁₋₄alkoxy, N—(C₁₋₄alkyl)amino, carbocyclyl, carbocyclylC₁₋₄alkyl, and heterocyclylC₁₋₄alkyl;
wherein R², R³, R⁴, and R⁵ may be independently optionally substituted on carbon with one or more R⁹ groups;
R⁹ is selected from halo, cyano, C₁₋₄alkyl, and N,N—(C₁₋₄alkyl)₂amino;
X is —S(O)ₐ—, —O—, —NR¹³—, —NR¹⁵C(O)—, —SO₂NR¹⁶—, or —NR¹⁶SO₂—; wherein a is 0 or 2;
R¹³, R¹⁵, and R¹⁶ are independently selected from hydrogen, phenyl, C₁₋₄alkylsulphonyl, and C₁₋₄alkyl;
q is 0 or 1;
p is 0 or 1;
Ring B is phenyl thienyl, furyl, thiazolyl, piperidinyl, piperazinyl, pyrrolidinyl, 1,3-dihydroisoindolyl, morpholinyl, naphthyl, cyclohexyl, pyridyl, imidazolyl, 1,2,4-triazolyl, 1,3-benzodioxolyl, thiomorpholinyl, pyrimidinyl, pyrazinyl, pyridazinyl, benzimidazolyl, or pyrimidinyl; wherein if Ring B contains an —NH— moiety, that nitrogen may be optionally substituted by a group selected from R¹⁷;
R¹⁷ is C₁₋₄alkyl or benzyl; wherein R¹⁷ may be optionally substituted on carbon with one or more R²⁷ groups;
R²⁷ is methoxy,
R⁶ is a substituent on carbon and is selected from halo, hydroxy, nitro, cyano, carbamoyl, C₁₋₄alkyl, C₁₋₄alkoxy, C₁₋₄alkanoyl, N,N—(C₁₋₄alkyl)₂amino, C₁₋₄alkanoylamino, N—(C₁₋₄alkyl)carbamoyl, N,N—(C₁₋₄alkyl)₂carbamoyl, C₁₋₄alkylS(O)ₐ wherein a is 0 or 2, C₁₋₄alkoxycarbonyl, N,N—(C₁₋₄alkyl)₂sulphamoyl, carbocyclyl, heterocyclyl, and carbocyclylC₀₋₄alkylene-Y—; wherein R⁶ may be optionally substituted on carbon with one or more R¹⁸ groups; and wherein if said heterocyclyl contains an —NH— moiety, that nitrogen may be optionally substituted with an R¹⁹ group;
Y is —C(O) or —C(O)NR²¹—;
R¹⁸ is selected from halo, cyano, hydroxy, C₁₋₄alkoxy, and heterocyclyl;
R¹⁹ is heterocyclyl,
R²¹ is hydrogen; and
m is 0-3; wherein the values of R⁶ may be the same or different;

9. A compound selected from:
[2-(4-chlorophenyl)-1-(pyrid-3-yl)ethyl]-(4-chlorophenyl)-ketone;
[2-(4-chlorophenyl)-1-(pyrazin-2-yl)ethyl]-(pyridin-3-yl)-ketone;
(αt-methylamino-4-chlorobenzyl)-(4-chlorophenyl)-ketone;
(benzothiazol-2-yl)-(pyrrolidin-1-ylsulphonylmethyl)-ketone;
(thiazol-2-yl)-(pyrrolidin-1-ylsulphonylmethyl)-ketone;
[1-(morpholinosulphonyl)-1-methylethyl]-(4-fluorophenyl)-ketone;
(4-fluorophenyl)-[N-(cyclohexyl)-N-(isopropyl)sulphamoylmethyl]-ketone;
(4-fluorophenyl)-[N-(pyrid-2-yl)-N-(methyl)sulphamoylmethyl]-ketone;
(4-methylphenylsulphonylmethyl)-(4-cyanophenyl)-ketone;
(4-ethoxyphenoxymethyl)-(4-chlorophenyl)-ketone;
(4-chlorophenyl)-[3-(2,6-difluorobenzoylamino) propyl)]-ketone; and
(4-chlorophenyl)-[3-(4-methoxyphenylsulphonylamino)propyl)]-ketone; or a pharmaceutically acceptable salt thereof

10. The method of claim 1, wherein tie compound of formula (I) is selected from:
(α-methyl-α-hydroxy-4-chlorobenzyl)-(4-chlorophenyl)-ketone;
(morpholinosulphonyhmethyl)-(4-fluorophenyl)-ketone;
(N-methyl-4-methylanilinosulphonylmethyl)-(4-chlorophenyl)-ketone; and
(N-methyl-4-chloroanilinomethyl)-(4chlorophenyl)-ketone; or a pharmaceutically acceptable salt thereof.

11. A compound of formula (Ij): 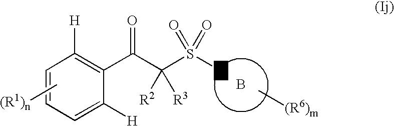
wherein:
R¹ is selected from halo, nitro, cyano, hydroxy, amino, carboxy, carbamoyl, mercapto, sulphamoyl, C₁₋₆alkyl, C₂₋₆alkenyl, C₂₋₆alkynyl, C₁₋₆alkoxy, C₁₋₆alkanoyl, C₁₋₆alkanoyloxy, N—(C₁₋₆alkyl)amino, N,N—(C₁₋₆alkyl)₂amino, C₁₋₆alkanoylamino, N—(C₁₋₆alkyl)carbamoyl, N,N—(C₁₋₆alkyl)₂carbamoyl, C₁₋₆alkylS(O)ₐ wherein a is 0 to 2, C₁₋₆alkoxycarbonyl, N—(C₁₋₆alkyl)sulphamoyl, N,N—(C₁₋₆alkyl)₂sulphamoyl, C₁₋₆alkylsulphonylamino, carbocyclyl, heterocyclyl, carbocyclylC₀₋₆alkylene-Y—, and heterocyclylC₀₋₆alkylene-Y—; or two R¹ groups on adjacent carbons may form an oxyC₁₋₄alkoxy group or a C₃₋₅alkylene group; wherein R¹ may be optionally substituted on carbon with one or more R⁷ groups; and wherein if said heterocyclyl contains an —NH— moiety, that nitrogen may be optionally substituted by an R⁸ group;
n is 0-3; wherein the values of R¹ may be the same or different;
R² and R³ are independently selected from hydrogen, hydroxy, amino, cyano, C₁₋₄alkyl, C₁₋₄alkoxy, N—(C₁₋₄alkyl)amino, N,N—(C₁₋₄alkyl)₂amino, C₁₋₄alkylS(O)ₐ wherein a is 0 to 2, C₁₋₄alkoxycarbonyl, C₁₋₄alkoxycarbonylamino, C₁₋₄alkanoyloxy, carbocyclyl, heterocyclyl, carbocyclylC₁₋₄alkyl, and heterocyclylC₁₋₄alkyl; or
R² and R³ together form oxo or a spiro attached heterocyclyl; wherein R² and R³ may be independently optionally substituted on carbon with one or more R⁹ groups; and wherein if said heterocyclyl contains an —NH— moiety, that nitrogen may be optionally substituted with an R^{10 l group;}
Ring B is a heterocyclyl linked to the sulphonyl of the compound of formula (Ij) via a nitrogen atom; wherein if said heterocyclyl contains an —NH— moiety, that nitrogen may be optionally substituted with an R¹⁷ group;
R⁶ is a substituent on carbon and is selected from halo, nitro, cyano, hydroxy, amino, carboxy, carbamoyl, mercapto, sulphamoyl, trifluoromethyl, trifluoromethoxy, C₁₋₄alkyl, C₂₋₄alkenyl, C₂₋₄alkynyl, C₁₋₄alkoxy, C₁₋₄alkanoyl, C₁₋₄alkanoyloxy, N—(C₁₋₄alkyl)amino, N,N—(C₁₋₄alkyl)₂amino, C₁₋₄alkanoylamino, N—(C₁₋₄alkyl)carbamoyl, N,N—(C₁₋₄alkyl)₂carbamoyl, C₁₋₄alkylS(O)ₐ wherein a is 0 to 2, C₁₋₄alkoxycarbonyl, N—(C₁₋₄alkyl)sulphamoyl, N,N—(C₁₋₄alkyl)₂sulphamoyl, C₁₋₄alkylsulphonylamino, carbocyclyl, heterocyclyl, carbocyclylC₀₋₄alkylene-Y—, and heteroclylC₀₋₄alkylene-Y—; wherein R⁶ maybe optionally substituted on carbon with one or more R¹⁸ groups; and wherein if said heterocyclyl contains an —NH— moiety, that nitrogen may be optionally substituted with an R¹⁹ group;
m is 0-3; wherein the values of R⁶ may be the same or different;
Y is —S(O)ₐ—, —O—, —NR²⁰—, —C(O)—, —C(O)NR²¹—, —NR²²C(O)—, or —SO₂—; wherein a is 0 to 2;
R⁷, R⁹, and R¹⁸ are independently selected from halo, nitro, cyano, hydroxy, amino, carboxy, carbamoyl, mercapto, sulphamoyl, trifluoromethyl, tribromomethoxy, C₁₋₄alkyl, C₂₋₄alkenyl, C₂₋₄alkynyl, C₁₋₄alkoxy, C₁₋₄alkanoyl, C₁₋₄alkanoyloxy, N—(C₁₋₄alkyl)amino, N,N—(C₁₋₄alkyl)₂amino, C₁₋₄alkanoylamino, N—(C₁₋₄alkyl)carbamoyl, N,N—(C₁₋₄alkyl)carbanamoyl, C₁₋₄alkylS(O)ₐ wherein a is 0 to 2, C₁₋₄alkoxycarbonyl, N—(C₁₋₄alkyl)sulphamoyl, N,N—(C₁₋₄alkyl)₂sulphamoyl, C₁₋₄alkylsulphonylamino, carbocyclyl, and heterocyclyl; wherein R⁷, R⁹, and R¹⁸ may be independently optionally substituted on carbon with one or more R²⁶ groups;
R⁸, R¹⁰, R¹⁷, and R¹⁹ are independently selected from C₁₋₄alkyl, C₁₋₄alkanoyl, C₁₋₄alkylsulphonyl, C₁₋₄alkoxycarbonyl, carbamoyl, N—(C₁₋₄alkyl)carbamoyl, N,N—(C₁₋₄alkyl)carbamoyl, benzyl, benzyloxycarbonyl, benzoyl, carbocyclyl, heterocyclyl, and phenylsulphonyl; wherein R⁸, R¹⁰, R¹⁷, and R¹⁹ may be independently optionally substituted on carbon with one or more R²⁷ groups;
R²⁰, R²¹, R²², and R²³ are independently selected from hydrogen, phenyl, C₁₋₄alkylsulphonyl, and C₁₋₄alkyl;
R²⁶ and R²⁷ are independently selected from selected from halo, nitro, cyano, hydroxy, trifluoromethoxy, trifluoromethyl, amino, carboxy, carbamoyl, mercapto, sulphamoyl, methyl, ethyl, methoxy, ethoxy, acetyl, acetoxy, methylthio, ethylamino, dimethylamino, diethylamino, N-methyl-N-ethylamino, acetylamino, N-methylcarbamoyl, N-ethylcarbamoyl, N,N-dimethylcarbamoyl, N,N-diethylcarbamoyl, N-methyl-N-ethylcarbamoyl, methylthio, ethylthio, methylsulphinyl, ethylsulphinyl, mesyl, ethylsulphonyl, methoxycarbonyl, ethoxycarbonyl, N-methylsulphamoyl, N-ethylsulphamoyl, N,N-dimethylsulphamoyl, N,N-diethylsulphamoyl, and N-methyl-N-ethylsulphamoyl;
or a pharmaceutically acceptable salt thereof,
with the proviso that said compound is not
(phenyl)-[α-(pyrrolidin-1-ylsulphonyl)benzyl]-ketone;
(phenyl)-[α-(morpholinosulphonyl)benzyl]-ketone;
(4-carbamoylphenyl)-[4-(5-chloropyridin-2-yloxy)piperidin-1-ylsulphonylmethyl]-ketone;
(4-carbamoylphenyl)-[4-(4-fluorophenyl)piperidin-1-ylsulphonylmethyl]-ketone;
(4-fluorophenyl)-[4-(5-chloropyridin-2-yloxy)piperidin-1-ylsulphonylmethyl]-ketone;
(phenyl)-[4-(5-chloropyridin-2-yloxy)piperidin-1-ylsulphonylmethyl]-ketone;
(4-chlorophenyl)-(piperazin-1-ylsulphonylmethyl)-ketone;
(4-chlorophenyl)-[4-(t-butoxycoonyl)piperazin-1-ylsulphonylmethyl]-ketone;
(4-hydroxyphenyl)-(morpholinosulphonylmethyl)-ketone; or
(phenyl)-1,2,3,4-tetrahydroisoquinoline-2-ylsulphonylmethyl)-ketone;
when R² and R³ are hydrogen, m is 0, and Ring B is 4-methylpiperazin-1-yl, then (R¹)ₙ is not hydrogen, 4-fluoro, 4-nitro, 3,4-dimethoxy, 4-methoxy, 4-t-butyl, 4-trifluoromethyl, or 4-chloro; and
when R² and R³ were hydrogen, m is 0, and Ring B is morpholino, then (R¹)ₙ is not hydrogen, 4-dimethylamino, 4-nitro, 4-methoxy, 4-t-butyl, 4-trifluoromethyl, or 4-fluoro or 4-chloro.

12. A compound of formula (Ik): 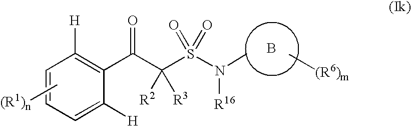
wherein:
R¹ is selected from halo, nitro, cyano, hydroxy, ammo, carboxy, carbamoyl, mercapto, sulphamoyl, C₁₋₆alkyl, C₂₋₆alkenyl, C₂₋₆alkynyl, C₁₋₆alkoxy, C₁₋₆alkanoyl, C₁₋₆alkanoyloxy, N—(C₁₋₆alkyl)amino, N,N—(C₁₋₆alkyl)₂amino, C₁₋₆alkanoylamino, N—(C₁₋₆alkyl)carbamoyl, N,N—(C₁₋₆alkyl)₂carbamoyl, C₁₋₆alkylS(O)ₐ wherein a is 0 to 2, C₁₋₆alkoxycarbonyl, N—(C₁₋₆alkyl)sulphamoyl, N,N—(C₁₋₆alkyl)₂sulphamoyl, C₁₋₆alkylsulphonylamino, carbocyclyl, heterocyclyl, carbocyclylC₀₋₆alkylene-Y—ₐ and heterocyclyC₀₋₆alkylene-Y—; or
two R¹ groups on adjacent carbons may form an oxyC₁₋₄alkoxy group or a C₃₋₅alkylene group;
wherein R¹ may be optionally substituted on carbon with one or more R⁷ groups; and wherein if said heterocyclyl contains an —NH— moiety, that nitrogen may be optionally substituted with an R⁸ group;
n is 0-3; wherein the values of R¹ may be the same or different;
R² and R³ are independently selected from hydrogen, hydroxy, amino, cyano, C₁₋₄alkyl, C₁₋₄alkoxy, N—(C₁₋₄alkyl)amino, N,N—(C₁₋₄alkyl)₂amino, C₁₋₄alkylS(O)ₐ wherein a is 0 to 2, C₁₋₄alkoxycarbonyl, C₁₋₄alkoxycarbonylamino, C₁₋₄alkanoyloxy, carbocyclyl, heterocyclyl, carbocyclylC₁₋₄alkyl, and heterocyclylC₁₋₄alkyl; or
R² and R³ together form oxo or a spiro attached heterocyclyl; wherein R² and R³ may be independently optionally substituted on carbon with one or more R⁹ groups; and wherein if said heterocyclyl contains an —NH— moiety, that nitrogen may be optionally substituted with an R¹⁰ group;
Ring B is carbocyclyl or heterocyclyl; wherein if said heterocyclyl contains an —NH— moiety, that nitrogen maybe optionally substituted with an R¹⁷ group;
R⁶ is a substituent on carbon and is selected from halo, nitro, cyano, hydroxy, amino, carboxy, carbamoyl, mercapto, sulphamoyl, trifluoromethyl, trifluoromethoxy, C₁₋₄alkyl, C₂₋₄alkenyl, C₂₋₄alkynyl, C₁₋₄alkoxy, C₁₋₄alkanoyl, C₁₋₄alkanoyloxy, N—(C₁₋₄alkyl)amino, N,N—(C₁₋₄alkyl)₂amino, C₁₋₄alkanoylamino, N—(C₁₋₄alkyl)carbamoyl, N,N—(C₁₋₄alkyl)₂carbamoyl, C₁₋₄alkylS(O)ₐ wherein a is 0 to 2, C₁₋₄alkoxycarbonyl, N—(C₁₋₄alkyl)sulphamoyl, N,N—(C₁₋₄alkyl)₂sulphamoyl, C₁₋₄alkylsulphonylamino, carbocyclyl, heterocyclyl, carbocyclylC₀₋₄alkylene-Y—, and heterocyclylC₀₋₄alkylene-Y—; wherein R⁶ may be optionally substituted on carbon with one or more R¹⁸ groups; and wherein if said heterocyclyl contains an —NH— moiety that nitrogen may be optionally substituted with an R¹⁹ group;
m is 0-3; wherein the values of R⁶ may be the same or different;
Y is —S(O)ₐ—, —O—, —NR²⁰—, —C(O)—, —C(O)NR²¹—, —NR²²C(O)—, or —SO₂NR²³—; wherein a is 0 to 2;
R⁷, R⁹, and R¹⁸ are independently selected from halo, nitro, cyano, hydroxy, amino, carboxy, carbamoyl mercapto, sulphamoyl, trifluoromethyl trifluoromethoxy, C₁₋₄alkyl, C₂₋₄alkenyl, C₂₋₄alkynyl, C₁₋₄alkoxy, C₁₋₄alkanoyl, C₁₋₄alkanoyloxy, N—(C₁₋₄alkyl)amino, N,N—(C₁₋₄alkyl)₂amino, C₁₋₄alkanoylamino, N—(C₁₋₄alkyl)carbamoyl, N,N—(C₁₋₄alkyl)₂carbamoyl, C₁₋₄alkylS(O)ₐ wherein a is 0 to 2, C₁₋₄alkoxycarbonyl, N—(C₁₋₄alkyl)sulphamoyl, N,N—(C₁₋₄alkyl)₂ sulphamoyl, C₁₋₄alkylsulphonylamino, carbocyclyl, and heterocyclyl; wherein R⁷, R⁹, and R¹⁸ may be independently optionally substituted on carbon with one or more R²⁶ groups;
R⁸, R¹⁰, R¹⁷, and R¹⁹ are independently selected from C₁₋₄alkyl, C₁₋₄alkanoyl, C₁₋₄alkylsulphonyl, C₁₋₄alkoxycarbonyl, carbamoyl, N—(C₁₋₄alkyl)carbamoyl, N,N—(C₁₋₄alkyl)carbamoyl, benzyl, benzyloxycarbonyl, benzoyl, carbocyclyl, heterocyclyl, and phenylsulphonyl; wherein R⁸, R¹⁰, R¹⁷, and R¹⁹ may be independently optionally substituted on carbon with one or more R²⁷ groups;
R¹⁶, R²⁰, R²¹, R²², and R²³ are independently selected from hydrogen, phenyl, C₁₋₄alkylsulphonyl, and C₁₋₄alkyl;
R²⁶ and R²⁷ are independently selected from selected from halo, nitro, cyano, hydroxy, trifluoromethoxy, trifluoromethyl, amino, carboxy, carbamoyl, mercapto, sulphamoyl, methyl, ethyl, methoxy, ethoxy, acetyl, acetoxy, methylamino, ethylamino, dimethylamino, diethylamino, N-methyl-N-ethylamino, acetylamino, N-methylcarbamoyl, N-ethylcarbamoyl, N,N-dimethylcarbamoyl, N,N-diethylcarbamoyl, N-methyl-N-ethylcarbamoyl, methylthio, ethylthio, methylsulphinyl, ethylsulphinyl, mesyl, ethylsulphonyl, methoxycarbonyl, ethoxycarbonyl, N-methylsulphamoyl N-ethylsulphamoyl, N,N-dimethylsulphamoyl, N,N-diethylsulphamoyl, N-methyl-N-ethylsulphamoyl;
or a pharmaceutically acceptable salt thereof;
with the proviso that said compound is not
(phenyl)-(5-methylpyrazol-3-ylaminosulphonylmethyl)-ketone;
(phenyl)-[(2-methyl-6-methoxy-2,3-dihydrobenzofuran-4-yl)aminosulphonylmethyl]-ketone;
(phenyl)-(1-phenyl-3-methylpyrazol-5-ylaminosulphonylmethyl)-ketone;
(phenyl)-[1-(cyclohexyl-N-methylaminosulphonyl)ethyl]-ketone;
(phenyl)-[1-(phenyl-N-methylaminosulphonyl)ethyl]-ketone;
(phenyl)-(cyclohexylaminosulphonylmethyl)-ketone;
(phenyl)-[(2-phenyl-4-acetyl-5-methylimidazol-3-yl]-N-methylaminosulphonyl methyl]-ketone;
(phenyl)-[(2-phenyl-4-acetyl-5-methylimidazol-3-yl]aminosulphonylmethyl]-ketone;
(phenyl)-(2,4,5,6,7,8-hexahydrocycloheptapyrazol-3-ylaminosulphonylmethyl]-ketone;
(phenyl)-(4,5,6,7-tetrahydro-2H-indazol-3-ylaminosulphonylmethyl]-ketone;
(phenyl)-[(4-phenyl-5-methylpyrazol-3-yl)aminosulphonylmethyl]-ketone;
(phenyl)-[3-(1-carboxymethyl-3-methyl-4oxo-1,2,3,4-tetrahydrophthalic-2-yl)anilinosulphonylmethyl]-ketone;
(phenyl)-{3-[1-(methoxycarbonylmethyl)-3-methyl-4-oxo-1,2,3,4-tetrahydrophthalic-2-yl]anil inosulphoyhmethyl}-ketone; (phenyl)-(4-methylanilinosulphonylmethyl)-ketone;
(phenyl)-(2-benzoyl-4-chloroanilinosulphonylmethyl)-ketone;
(phenyl)-(2,3-dimethylanilinosulphonylmethyl)-ketone;
(phenyl)-(3,4-dimethylanilinosulphonylmethyl)-ketone;
(phenyl)-(3-methylanilinosulphonylmethyl)-ketone;
(phenyl)-(3-methylanilinosulphonylmethyl)-ketone;
(phenyl)-(anilinosulphonylmethyl)-ketone; (phenyl)-2-acetylanilinosulphonylmethyl)-ketone; or
(phenyl)-[α-(N-ethylanlinosuphonyl)benzyl]-ketone.

13. A pharmaceutical composition which comprises a compound of any one of claims 9, 11 or 12, or a pharmaceutically acceptable salt thereof, in association with a pharmaceutically acceptable diluent or carrier.

14. A method for inhibiting 11βHSD1, comprising administering to a warm-blooded animal, a therapeutically effective amount of a compound of any one of claims 9, 11 or 12.

15-16. (canceled)

17. A method for the treatment of a metabolic syndrome, comprising inhibiting 11βHSD1as claimed in claim 1, or 10.

18. A method for the treatment of a disease selected from diabetes, obesity, hyperlipidaemia, hyperglycaemia, hyperinsulidemia, and hypertension, comprising inhibiting 11βHSD1 as claimed in claim 1 or 10.

19. A method for the treatment of a disease selected from glaucoma, osteoporosis, tuberculosis, dementia, cognitive disorders or depression, comprising inhibiting 11βHSD1 as claimed in claim 1 or 10.

20. (canceled)
